(19) United States
(12) Patent Application Publication
(10) Pub. No.: US 2023/0173091 A1
(43) Pub. Date: Jun. 8, 2023

(54) TARGETED CATALYTIC COMPLEMENT-ACTIVATING MOLECULES AND METHODS OF USE THEREOF

(71) Applicant: Omeros Corporation, Seattle, WA (US)

(72) Inventors: Mohammed Youssif Ibrahim Ali, Cambridge (GB); Gregory A. Demopulos, Mercer Island, WA (US); Christiana Doulami, Seattle, WA (US); Hans-Wilhelm Schwaeble, Cambridge (GB); Munehisa Yabuki, Seattle, WA (US)

(21) Appl. No.: 17/938,421

(22) Filed: Oct. 6, 2022

Related U.S. Application Data
(60) Provisional application No. 63/253,211, filed on Oct. 7, 2021.

(51) Int. Cl. A61K 47/68; G01N 33/569; G01N 33/543; A61P 31/04; A61P 31/10

(52) U.S. Cl. CPC A61K 47/6849; G01N 33/56983; G01N 33/56911; G01N 33/54386; C12Y 304/21; A61K 47/6815; A61P 31/04; A61K 47/6841; A61P 31/10; G01N 2333/95

(57) ABSTRACT

In one aspect, the present disclosure provides targeted complement-activating molecules comprising a target-binding domain and a complement-activating serine protease effector domain. In some embodiments, the target-binding domain is derived from an antibody or an antigen-binding fragment thereof. Also provided are compositions and methods for treating cancer, autoimmune disease, or microbial infection, including bacterial, viral, fungal, or parasitic infection, using targeted complement-activating molecules.

### FIELD OF THE INVENTION

The present invention relates to targeted complement-activating molecules comprising a targeting domain and a serine protease domain for use in targeting complement activation, and related compositions and methods.

### STATEMENT REGARDING SEQUENCE LISTING

The sequence listing associated with this application is provided in .xml format in lieu of a paper copy and is hereby incorporated by reference into the specification. The name of the .xml file containing the sequence listing is MP_1_0303_US2_Sequence_Listing_20221003_ST26.xml, the file is 216 KB, was created on Oct. 3, 2022; and is being submitted via the Patent Center with the filing of the specification.

### BACKGROUND

The complement system supports innate host defense against pathogens and other acute insults (M. K. Liszewski and J. P. Atkinson, 1993, in Fundamental Immunology, Third Edition, edited by W. E. Paul, Raven Press, Ltd., New York), and also has a role in immune surveillance against cancer (P. Macor, et al., Front. Immunol., 9:2203, 2018). More than 30 fluid-phase and membrane-bound glycoproteins, cofactors, receptors, and regulatory proteins are involved in the complement system (S. Meyer, et al., mAbs, 6:1133, 2014). Many of them are serine proteases, which form a highly regulated cascade of activation events. The complement system responds rapidly to molecular stress signals through a cascade of sequential proteolytic reactions initiated by the binding of pattern recognition receptors (PRRs) to distinct structures on damaged cells, biomaterial surfaces, or microbial intruders (Reis et al., Nat. Rev. Immunol., 18:5, 2018). Activation of the complement cascade induces diverse immune effector functions, such as cell lysis, phagocytosis, chemotaxis, and immune activation (S. Meyer, et al., 2014). Furthermore, the complement system also acts as a bridge between the innate immune response and the subsequent activation of adaptive immunity. In addition to its anti-infectious properties, the complement system is also involved in the clearance of immune complexes and apoptotic cells, tissue regeneration, mobilization of hematopoietic progenitor cells, and angiogenesis (T. M. Pierpont et al., Front. Oncol., 8:163, 2018).

The complement system can be activated through three distinct pathways: the classical pathway, the alternative pathway, and the lectin pathway. See FIG. 1. Activation of the classical pathway is triggered by a conformational change of the classical pathway initiation complex C1, composed of C1q, a hexamer of trimeric chains, and a heterotetramer of the C1q-associated serine proteases C1r and C1s, as detailed below. The binding of C1q to complexes composed of host antibodies bound to a foreign particle (i.e., an antigen) initiates the activation of C1 complex. Since activation of the classical pathway largely depends on a prior adaptive immune response by the host, the classical pathway is an effector mechanism of the acquired immune system. In contrast, both the lectin and alternative pathways are independent of adaptive immunity and are part of the innate immune system.

The classical pathway (CP) is primarily initiated by antibody-antigen complexes. Antibodies of subclasses IgM and IgG bind to an antigen on the surface of a pathogen or a target cell and recruit the C1 complex, which is composed of the multimolecular recognition subcomponent C1q (composed of six heterotrimers of the C1q A-chain, B-chain, and C-chain) and the C1q-associated serine proteases C1r and C1s. Upon binding of C1q to the Fc-region of either an IgM bound to an antigen or to at least two IgG antibodies bound to their antigens, the serine protease C1r is converted from its zymogen form into its enzymatically active form and subsequently cleaves and activates its substrate C1s. Once activated, C1s cleaves C4 into its fragments C4a and C4b. C4b binds to complement component C2 and this complex, C4bC2, is cleaved by C1s in a second cleavage step to release C2b, forming the complement C3 converting enzyme complex C4bC2a, a so-called C3 convertase, which cleaves the abundant plasma complement component C3 into C3a and C3b.

The lectin pathway is triggered by the binding of pattern recognition molecules, such as mannose-binding lectin (MBL), ficolins or collectin-11 and collectin-10, to pathogen-associated molecular patterns (PAMPs) or apoptotic or distressed host cells. The recognition molecules form a complex with the MBL-associated serine proteases, MASP-1 and MASP-2, and activate them upon binding, which results in the cleavage of C2 and C4 and the formation of the C3 convertase (C4bC2a).

The alternative pathway (AP) is initiated by spontaneous hydrolysis of C3 (“tickover”) to C3(H₂O), which binds to factor B (fB). The conversion of the resulting C3(H₂O)fB complexes requires the enzymatic activity of another highly specific serine protease called factor D. The availability of enzymatically active factor D is thought to be a limiting factor for the alternative pathway amplification loop and the availability of factor D requires the action of another enzyme, MASP-3, which is required for conversion of pro-Factor D (proCFD) into its active form, mature factor D (matCFD) (Dobó et al., 2016). MatCFD, another serine protease, cleaves the C3(H₂O)-bound fB into Ba and Bb. Bb is also a serine protease and participates in the formation of alternative C3 convertase C3(H₂O)Bb, which cleaves C3 into C3a and C3b. By this mechanism, the alternative pathway is constitutively active at low levels. The AP amplification loop is formed when freshly generated C3b, formed either by C3(H₂O)Bb or by the classical and lectin pathway C3 convertase C4bC2a, binds to the target surfaces and sequesters fB to form C3bfB complexes that, upon cleavage by matCFD, create another C3 convertase complex C3bBb. This convertase can be further stabilized by properdin, which prevents decay of the complex and conversion of C3b by factor H and factor I. C3bBb is the functional convertase of the alternative pathway.

The three pathways converge after formation of the C3 convertases C4bC2a and C3bBb. The C3 cleavage fragment C3a is an anaphylatoxin which promotes inflammation. C3b functions as an opsonin by binding covalently through a thioester bond on the surface of target cells, marking them for circulating complement receptor (CR)-displaying effector cells, such as NK cells and macrophages, which contribute to complement-dependent cellular cytotoxicity (CDCC) and complement-dependent cellular phagocytosis (CDCP), respectively. C3b also binds to the C3 convertase (either C4bC2a or C3bBb) to form a C5 convertase (C4bC2a(C3b)n or C3bBb(C3b)n, respectively), which leads to MAC formation and subsequent CDC. Additionally, C3b's cell-bound degradation fragments, iC3b and C3dg, can promote complement-receptor-mediated cytotoxicity (CDCC and CDCP) as well as adaptive immune response through B cell activation (M. C. Carroll, Nat. Immunol ., 5:981, 2004).

Formation of C5 convertase leads to the cleavage of C5 into C5a and C5b. C5a is another anaphylatoxin. C5b recruits C6-9 to form the membrane attack complex (MAC, or C5b-9 complex). The MAC causes pore formation resulting in membrane destruction of the target cell and cell lysis (so called complement-dependent cytotoxicity, CDC). Direct cell lysis through the MAC formation has been traditionally recognized as a terminal effector mechanism of the complement system, however, C3b mediated opsonization and pro-inflammatory signaling as well as the anaphylatoxin function of C3a are thought to play a significant role in the mediation of complement dependent inflammatory pathology.

Complement regulatory proteins (CRPs) prevent unwanted complement activation and consumption of complement components. These proteins are present in most cells and via tight control they play an important role in protecting the host cells from complement-mediated damage. CRPs can be soluble proteins (sCRPs) or membrane-bound complement regulatory proteins (mCRPs) (P. F. Zipfel and C. Skerka, Nat. Rev. Immunol. 9:729, 2009). One of the most abundant protease inhibitors in circulating blood is the C1 inhibitor (C1inh), with an average plasma concentration of 0.25 g/L (H. Gregorek, Comp. and Inflamm. 8:310, 1991). C1inh binds to and inactivates C1r, C1s, and two of the MBL-associated serine proteases, MASP-1 and MASP-2; hence it is the primary inhibitor for the classical and lectin pathway. Other sCRPs include C4 binding protein (C4BP), and factors H and I (P. F. Zipfel and C. Skerka, 2009).

In contrast to sCRPs, the mCRPs regulate the complement pathways by targeting both C3 and C4 (P. F. Zipfel and C. Skerka, 2009). For example, CD46 (membrane cofactor protein; MCP) is a co-factor for factor I, which mediates cleavage of C3b and C4b into their inactive degradation products, iC3b and iC4b, respectively, and thereby leads to inhibition of all three complement pathways. CD55 (decay acceleration factor; DAF) accelerates the decay of C3 and C5 convertases, which inhibits all three complement pathways. CD59 (protectin) prevents assembly of the MAC by inhibiting the polymerization of C9 and its subsequent binding to C5b-8, thus inhibiting all three pathways.

For most microbial organisms, the first line of defense provided by complement is sufficient to prevent infection and preserve the integrity of the host organism. Pathogens are micro-organisms that have acquired ways to undermine the host's immune system, break through the barriers that protect the host against microbial invasion, and establish an infection. Pathogens have developed various ways to undermine the host's immune defense. For example, the bacterium Neisseria meningitidis has a surface protein called Factor H-binding protein that sequesters and binds the host's negative complement regulatory component factor H (fH) to the bacterial surface. This, in turn, protects the bacteria from complement activation since surface bound factor H decays and inactivates complement C3 and C5 convertases that have formed on the pathogen surface and thereby prevents the host complement system from neutralizing, killing, or opsonizing the pathogen. Other strategies that pathogens have developed to evade complement attack include the sequestration of host C4-binding protein by bacterial surface proteins like PspA and PspC of Streptococcus pneumoniae to prevent the formation of classical and lectin pathway C3 and C5 convertases C4bC2a and C4bC2a(C3b)n respectively on the bacterial surface (Haleem K S, et al. Infect Immun. 2018 Dec. 19; 87(1):e00742-18.), and the release of complement-activating bacterial toxins that consume complement away from the vulnerable pathogen surface.

The complement system is also involved in suppression of cancer. Neoplastic transformation leads to several genetic and epigenetic alterations which change the morphology and composition of the cell membrane. During this process of transformation, normal cells express tumor-specific markers and produce pro-inflammatory signals that are recognized by the cancer immunosurveillance network. Complement is considered a part of the cancer immune surveillance network (Pio et al., 2014). It has been demonstrated that all three complement pathways are activated in malignant tumors (Macor, Capolla and Tedesco, 2018). Complement proteins, C3 degradation products, and complement activation products (i.e., C5a, C3a, and C5b-9) have been detected in several types of cancer (Afshar-Kharghan, 2017). Besides complement components, CRPs have also been found in cancer. In fact, mCRPs and sCRPs are overexpressed on cancer cells among different cancer types (Meyer, Leusen and Boross, 2014). Thus, the complement system is a host mechanism against cancer and cancer cells may resist complement attack by overexpressing CRPs.

Complement's central role in multiple physiological processes requires that complement activation be tightly regulated. Pathogens (P. F. Zipfel and C. Skerka, 2009) and cancer cells (A. Geller and J. Yan, Front. Immunol. 10:1, 2019), however, have been shown to use evasion strategies to block complement activity, including expression of negative complement regulatory proteins. Thus, there is a need for therapies that enhance complement activity against pathogens or dysfunctional self cells (e.g., cancer cells or autoimmune cells), such as by targeting complement activation to the pathogens or dysfunctional cells, or to tissues where the pathogens or dysfunctional cells are present.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides targeted complement-activating molecules comprising a targeting or target-binding domain and a complement-activating serine protease effector domain. In some embodiments, the target-binding domain is derived from an antibody. In some embodiments, the target-binding domain comprises an antigen-binding fragment of an antibody. In some embodiments, the complement-activating serine protease effector domain is catalytically active, while in other embodiments the complement-activating serine protease effector domain is in a zymogen form. In some embodiments, the target-binding domain binds to an antigen present on a cell, such as CD20, CD38, or CD52. In other embodiments, the target-binding domain binds to an antigen present on a microbial pathogen, such as a bacterial pathogen, a viral pathogen, a fungal pathogen, or a parasitic pathogen.

In some embodiments, the targeted complement-activating molecules comprise a fusion protein comprising the N-terminus of the serine protease effector domain fused to the C-terminus of the antibody heavy chain or fragment thereof or to the C-terminus of the antibody light chain or fragment thereof. In other embodiments, the fusion protein comprises the C-terminus of the serine protease effector domain fused to the N-terminus of the antibody heavy chain or fragment thereof or to the N-terminus of the antibody light chain or fragment thereof. In some embodiments, the targeted complement-activating molecules comprise such a fusion protein and a second antibody chain, which is a light chain or fragment thereof if the fusion protein comprises a heavy chain or fragment thereof and which is a heavy chain or fragment thereof if the fusion protein comprises a light chain or fragment thereof. In some embodiments, the targeted complement-activating molecules comprise a fusion protein comprising the N-terminus of the serine protease effector domain fused to the C-terminus of a single-chain antibody or fragment thereof, or a fusion protein comprising the C-terminus of the serine protease effector domain fused to the N-terminus of a single-chain antibody or fragment thereof.

In some embodiments, the serine protease effector domain comprises complement factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

Also provided herein are polynucleotides encoding targeted complement-activating molecules or portions thereof, and cloning vectors or expression cassettes comprising such polynucleotides.

Further provided herein are host cells expressing targeted complement-activating molecules and methods of producing targeted complement-activating molecules comprising culturing the host cells under conditions allowing for expression of the molecules and isolating the molecules.

Also provided herein are methods of activating at least one complement pathway in a mammalian subject using the targeted complement-activating molecules. In some embodiments, the targeted complement-activating molecules may be used to induce complement dependent cytotoxicity (CDC), complement-dependent cellular cytotoxicity (CDCC), or complement-dependent cellular phagocytosis (CDCP) in a target cell. In some embodiments, the targeted complement-activating molecules may be used to treat cancer, autoimmune disease, or a microbial infection, such as a bacterial infection, a viral infection, a fungal infection, or a parasitic infection.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating the classical, lectin, and alternative complement pathways.

FIG. 2 graphically illustrates the surface levels of CD20, CD55, and CD59 on CD20+ cancer cell lines Ramos, Kasumi-2, and SU-DHL-8. Cells were stained with primary antibody targeted against CD20 (RTX), CD55 (CBL511, Millipore) and CD59 (MAB1759, Millipore) and the secondary antibody, an anti-human IgG Fc Ab conjugated with fluorophore (Biolegend). Controls are shown as light grey lines. Expression levels are shown as dark grey lines. Fluorescence was measured by FACS.

FIG. 3 is a diagram illustrating certain formats for the targeted complement-activating molecules described herein. Such molecules may comprise a targeting domain derived from an antibody and a serine protease effector domain fused to either the heavy chain or light chain of the antibody. Shown are an unmodified antibody (far left) and targeted complement-activating molecules comprising a serine protease effector domain fused to: the C-terminus of the heavy chain (second from left), the N-terminus of the heavy chain (center), the C-terminus of the light chain (second from right), or the N-terminus of the light chain (far right).

FIG. 4 shows the results of SDS-PAGE analysis of certain targeted complement-activating molecules comprising a serine protease effector domain derived from MASP-1, MASP-2, or MASP-3, as described herein. Polyacrylamide gel with a gradient concentration of 4-12% (NuPAGE Bis-Tris gel, Invitrogen) was used to separate subunits of each fusion protein, and polypeptide sizes were estimated using molecular weight marker (SeeBlue Plus 2, Invitrogen). The abbreviations for the various molecules analyzed are provided in Table 1. rituximab (RTX) was used as a control.

FIG. 5 shows the results of SDS-PAGE analysis of certain targeted complement-activating molecules comprising a serine protease effector domain derived from C1r, C1s, C2a, Bb, or complement factor D (CFD), as described herein. Polyacrylamide gel with a gradient concentration of 4-12% (NuPAGE Bis-Tris gel, Invitrogen) was used to separate subunits of each fusion protein, and polypeptide sizes were estimated using molecular weight marker (SeeBlue Plus 2, Invitrogen). The abbreviations for the various molecules analyzed are provided in Table 1. Rituximab (RTX) was used as a control.

FIG. 6 shows SDS-PAGE analysis of the activation of certain targeted complement-activating molecules comprising a serine protease effector domain derived from MASP-3, as described herein. Zymogen RTX(H)^{ΔK}-M3 (2 μM) was diluted in 10 mM HEPES, pH 7.4, 140 mM NaCl, 0.1 mM EDTA buffer and was incubated at 37° C. alone (negative control) or with the addition of MASP-2 (CCP1/2SP) (91 nM) in various timepoints (0, 10, 20, 40, 60, 90, 120, 150 and 190 minutes). The samples were removed in each timepoint and placed at −20° C. to stop the reaction. SDS-PAGE analysis with reducing conditions was performed to verify the cleavage of the MASP-3 fusion protein. The bands corresponding to the zymogen and the active form of RTX-MASP-3 are shown.

FIG. 7 shows SDS-PAGE analysis of potential degradation-resistant variants of certain targeted complement-activating molecules, as described herein. Polyacrylamide gel with a gradient concentration of 4-12% (NuPAGE Bis-Tris gel, Invitrogen) was used to separate subunits of each fusion protein, and polypeptide sizes were estimated using molecular weight marker (SeeBlue Plus 2, Invitrogen). The abbreviations for the various molecules analyzed are provided in Table 1. Rituximab (RTX) was used as a control.

FIG. 8 shows SDS-PAGE analysis of additional potential degradation-resistant variants of certain targeted complement-activating molecules comprising a serine protease effector domain derived from C1r or C1s, as described herein. Polyacrylamide gel with a gradient concentration of 4-12% (NuPAGE Bis-Tris gel, Invitrogen) was used to separate subunits of each fusion protein, and polypeptide sizes were estimated using molecular weight marker (SeeBlue Plus 2, Invitrogen). The abbreviations for the various molecules analyzed are provided in Table 1. The targeted complement-activating molecules shown in lanes 6 and 8 (RTX(H)^{N297G,ΔK}-C1s^{D456W} and RTX(H)^{N297G,ΔK}-C1s^{P458W}, respectively) were incubated with C1r for one or three hours to convert the serine protease effector domain to the active form, followed by SDS-PAGE analysis to check for degradation (right panel).

FIG. 9 shows binding of certain targeted complement-activating molecules described herein to target cells. B cell line Ramos cells (ATCC) (0.5×10⁶ cells) were stained with rituximab or with one of twelve targeted compliment-activating molecules comprising a targeting domain derived from rituximab as the primary antibody. Anti-human IgG Fc Ab conjugated with fluorophore (BioLegend) was used as the secondary antibody. Unstained cells and cells stained only with secondary Ab were included as controls. Fluorescence was measured by FACS. Controls are shown as light grey lines. Rituximab binding is shown as dark grey lines. Binding of the targeted complement-activating molecules is shown as a solid dark grey area.

FIG. 10 shows the kinetics of binding to CD20 for certain targeted complement-activating molecules described herein, as measured by biolayer interferometry (BLI). The binding assay was performed with AHC biosensors. 69 nM of targeted complement-activating molecule diluted in Kinetic Buffer (PBS, 0.02% Tween 20, 1% BSA, 0.05% DDM, 0.01% CHS) was loaded (loading phase) and antigen CD20 diluted in Kinetic Buffer was added in two-fold series starting at 0, 6.25, 12.5, 25, 50, 100, and 200 nM (association phase). The assay was performed with Octet RED96 system (ForteBio Inc.) and analyzed by Octet CFR Software (ForteBio Inc.). Noisy and smooth lines distinguish measured data from the global fit. Data for anti-CD20 antibodies rituximab (RTX) and obinutuzumab (OBZ) is shown for comparison.

FIGS. 11A and 11B show the results of serine protease activity assays for certain targeted complement-activating molecules described herein. Substrate C4 (FIG. 11A) or C3 (FIG. 11B) was diluted in PBS (1×), pH 7.4, and incubated at 37° C. alone (“none”) or with the addition of the indicated targeted complement-activating molecules at an enzyme/substrate ratio of 1:20. Samples were removed after 3 hours to stop the reaction. SDS-PAGE analysis under reducing conditions was performed to verify the cleavage of C4 or C3. Cleavage products C4b and C3b are indicated by an arrow.

FIGS. 12A and 12B show the results of C4 deposition assays for certain targeted complement-activating molecules described herein. ELISA plates were coated with 100 μl of mannan (50 μg/mL) and either 215 nM (FIG. 12A) or 69 nM (FIG. 12B) of rituximab or the indicated targeted complement-activating molecule suspended in coating buffer. Plates were incubated at 4° C. overnight. Remaining protein binding sites were then blocked by the addition of 250 μl of 1% BSA in PBS buffer to each well and two hours incubation at room temperature. The plates were washed three times with PBS containing 0.05% Tween 20. Hirudin plasma from MASP-2 knockout (KO) mice (left panel of FIG. 12A) or wild-type (WT) mice (right panel of FIG. 12A) was diluted with PBS (no calcium, no magnesium) to obtain a final concentration of 10%. Normal human serum (NETS) (FIG. 12B) was diluted with PBS (no calcium, no magnesium) to a final concentration of 1%. The plates were incubated with the plasma for 15 minutes at 4° C. A C4 (0.2 μg/mL) antibody diluted in wash buffer was added to the plates and incubated 30 minutes at 37° C. and 200 rpm. A secondary antibody (0.043 μg/mL) diluted in wash buffer was added to the plates and incubated 30 minutes at room temperature. Absorbance at 450 nm was measured following addition of colorimetric substrate TMB.

FIG. 13 shows the results of C3 deposition assays for certain targeted complement-activating molecules described herein. ELISA plates were coated with 215 nM rituximab or the indicated targeted complement-activating molecule suspended in coating buffer. Plates were incubated at 4° C. overnight. Remaining protein binding sites were then blocked by the addition of 250 μl of 1% BSA in PBS buffer to each well and two hours incubation at room temperature. The plates were washed three times with PBS containing 0.05% Tween 20. Hirudin plasma from MASP-1/3 knockout (KO) mice (left panel) or wild-type (WT) mice (second panel from the left) was diluted with MgEGTA buffer (10 mM EGTA, 5 mM MgCl₂, 5 mM Barbital, 145 mM NaCl [pH 7.4]) to obtain a final concentration of 10%. Normal human serum (NHS) was diluted with MgEGTA to a final concentration of 3% (third panel from the left) or 10% (right panel). The plates were incubated with the plasma for 20 minutes (mouse plasma and 3% NHS) or 25 minutes (10% NHS) at 37° C. A C3 antibody (2.4 μg/mL) diluted in wash buffer was added to the plates and incubated 30 minutes at 37° C. and 200 rpm. A secondary antibody (0.043 μg/mL) diluted in wash buffer was added to the plates and incubated 30 minutes at room temperature. Absorbance at 450 nm was measured following addition of colorimetric substrate TMB.

FIGS. 14A and 14B show detection of complement factors C3b (FIG. 14A) or MAC (FIG. 14B) deposited on Kasumi-2 target cells after treatment with certain targeted complement-activating molecules described herein. Rituximab or the indicated targeted complement-activating molecule was diluted in Assay Buffer to a concentration of 12.5 nM. Normal human serum (NHS) was diluted into Assay Buffer to obtain a final concentration of 15%. Kasumi-2 cells were resuspended into Assay Buffer to a final concentration of 300,000 cells/ml and were transferred to a 6-well assay plate. The diluted proteins and NHS were added to the wells. Plates were incubated at 37° C. in a humidified incubator for two hours. The cells were then resuspended into FACS buffer, blocked to prevent non-specific binding, and stained with primary antibodies (rabbit anti-human C3c or monoclonal mouse anti-human C5b-9). After 20 minutes incubation in ice, the cells were washed twice and resuspended in FACS buffer containing secondary antibody (APC anti-rabbit IgG or PE anti-mouse IgG). The cells were incubated a further 20 minutes on ice, then washed three times and resuspended in FACS buffer. The stained cell samples were analyzed by FACS (FACSCalibur).

FIG. 15 shows detection of CD52 or CD38 using anti-CD52 or anti-CD38 antibodies or certain targeted complement-activating molecules described herein (columns labeled “CD52” and “CD38”), and detection of complement factor C3b deposited on HT target cells after treatment with certain targeted complement-activating molecules described herein (columns labeled “C3b”). For detection of CD52 or CD38, about 500,000 cells of human B cell lymphoma line HT (ATCC) were harvested and resuspended in FACS buffer. To prevent non-specific binding, 5 μl of blocking solution was added to 100 μl of cell suspension, which was then incubated 15 minutes at room temperature. Antibodies alemtuzumab (targeting CD52) and daratumumab (targeting CD38) or the indicated targeted complement-activating molecules were added to the cell suspension and incubated 20 minutes on ice. The cells were then washed twice and resuspended in FACS buffer containing secondary antibody (mouse anti-human IgG1 conjugated with Alexa Fluor 647). The cells were incubated on ice for 20 minutes, then washed three times and resuspended in FACS buffer. The stained cell samples were analyzed by FACS (FACSCalibur). For detection of C3b, alemtuzumab, daratumumab, or the indicated targeted complement-activating molecule was diluted in Assay Buffer to a concentration of 12.5 nM. Normal human serum (NETS) was diluted into Assay Buffer to obtain a final concentration of 15%. HT cells were resuspended into Assay Buffer to a final concentration of 300,000 cells/ml and were transferred to a 6-well assay plate. The diluted proteins and NHS were added to the wells. Plates were incubated at 37° C. in a humidified incubator for two hours. The cells were then resuspended into FACS buffer, blocked to prevent non-specific binding, and stained with primary antibody (rabbit anti-human C3c). After 20 minutes incubation in ice, the cells were washed twice and resuspended in FACS buffer containing secondary antibody (APC anti-rabbit IgG). The cells were incubated a further 20 minutes on ice, then washed three times and resuspended in FACS buffer. The stained cell samples were analyzed by FACS (FACSCalibur).

FIG. 16 shows the results of complement dependent cytotoxicity (CDC) assays using rituximab or certain targeted complement-activating molecules as described herein. Ramos cells (ATCC) were resuspended in Assay Buffer to a final concentration of 10,000 cells per well and were transferred to a 96-well plate. To each well were added 15% NHS and 12.5 nM rituximab or the indicated targeted complement-activating molecule. Controls with no antibody or targeted complement-activating molecule and with no cells were included. The plates were incubated two hours at 37° C., following which CytoTox-Glo (Promega) was added. After a further 15 minutes incubation at room temperature, luminescence was measured using a Luminoskan plate reader.

FIG. 17 shows the results of complement dependent cytotoxicity (CDC) assays using rituximab or certain targeted complement-activating molecules as described herein. Ramos cells (ATCC) were resuspended in Assay Buffer to a final concentration of 10,000 cells per well and were transferred to a 96-well plate. To each well were added 15% NHS and 12.5 nM rituximab or the indicated targeted complement-activating molecule (left panel) or the concentration of rituximab or targeted complement-activating molecule shown on the x-axis (right panel). Controls with no antibody or targeted complement-activating molecule and with no cells were included. The plates were incubated two hours at 37° C., following which CytoTox-Glo (Promega) was added. After a further 15 minutes incubation at room temperature, luminescence was measured using a Luminoskan plate reader.

FIG. 18 shows the results of complement dependent cytotoxicity (CDC) assays using rituximab or certain targeted complement-activating molecules as described herein. Ramos cells (ATCC) were resuspended in Assay Buffer to a final concentration of 10,000 cells per well and were transferred to a 96-well plate. To each well were added 15% NHS and 37.5 nM rituximab or the indicated targeted complement-activating molecule. Controls with no antibody or targeted complement-activating molecule and with no cells were included. The plates were incubated two hours (left panel) or three hours (right panel) at 37° C., following which CytoTox-Glo (Promega) was added. After a further 15 minutes incubation at room temperature, luminescence was measured using a Luminoskan plate reader.

FIGS. 19A and 19B show the results of complement dependent cytotoxicity (CDC) assays using rituximab or certain targeted complement-activating molecules as described herein. Three different concentrations of rituximab or the indicated targeted complement-activating molecule were prepared in Assay Buffer (Opti-MEM cell culture medium): 112.5 nM, 37.5 nM, and 12.5 nM. Normal human serum (NHS) was diluted into Assay Buffer to obtain a final concentration of 10%. Ramos cells were washed with PBS, resuspended with Assay Buffer to a final concentration of 150,000 cells per well, and transferred to a 96-well assay plate. The diluted proteins and human serum were added to the wells. The plates were incubated at 37° C. in a humidified incubator for two hours. Propidium iodide (5 μl) was added and the stained cells were immediately analyzed by flow cytometry (FACSCalibur). Cells treated only with NHS and unstained cells were included as controls. Dotted lines show NETS-only controls, solid lines show 12.5 nM concentrations, light grey areas show 37.5 nM concentrations, and dark grey areas show 112.5 nM concentrations. FIG. 19A shows the results of assays using rituximab (left panel) or MatCFD-RTX (right panel) at several different concentrations. FIG. 19B shows a comparison of the results of assays using rituximab or MATCFD-RTX at 112.5 nM.

FIG. 20 shows the results of complement dependent cytotoxicity (CDC) assays using rituximab or certain targeted complement-activating molecules in the presence of antibodies against one or both of the complement regulatory proteins (CRPs) CD55 (clone BRIC 216, Sigma-Aldrich) and CD59 (clone BRIC 229, IBGRL). Monoclonal antibodies rituximab (RTX) and a modified version of rituximab (RTX^{N297G}) were tested, as were targeted complement activating molecules comprising mature Factor D (MatCFD) and either RTX or RTX^{N297G}. RTX antibodies, RTX^{N297G} antibodies, or the targeted complement activating molecules were prepared in Assay Buffer (RPMI 1640 medium [-] L-glutamine, 5% FBS (heat-inactivated), 100× GlutaMax and 25 mM HEPES) to a final concentration of 337.5 nM. Anti-CD55 antibody was prepared with Assay Buffer to a final concentration of 10 μg/mL. Anti-CD59 antibody was prepared with Assay Buffer to a final concentration of 2 μg/mL. Normal human serum (NHS) was diluted into Assay Buffer to obtain a final concentration of 15%. Ramos cells were resuspended with Assay Buffer to a final concentration of 300,000 cells per well and transferred to a 96-well assay plate. The diluted proteins and NHS were added to the wells. The plates were incubated at 37° C. in a humidified incubator for two hours. Propidium iodide (5 μL, Invitrogen) was added and the stained cells were immediately analyzed by flow cytometry (FACSCalibur). Cells treated with NHS and RTX antibodies, RTX^{N297G} antibodies, or the targeted complement activating molecules but without the addition of anti-CD55 or anti-CD59 (no inh) were included as controls.

FIG. 21 shows the binding of three different mouse monoclonal antibodies to factor H binding protein (fHbP) of Neisseria meningitidis (N. meningitidis). The left panel shows binding of each of the three antibodies to recombinant fHbP on the surface of an ELISA plate. The right panel shows binding of each of the three antibodies to N. meningitidis on the surface of an ELISA plate.

FIG. 22 shows the binding of mouse-human chimera versions of the three mouse monoclonal antibodies to N. meningitidis on the surface of an ELISA plate.

FIG. 23 shows the binding of certain targeted complement-activating molecules described herein to N. meningitidis on the surface of an ELISA plate. The targeted complement activating molecules tested are Clone 19-C1r, which comprises a binding domain derived from a chimeric mouse monoclonal antibody to N. meningitidis fHbP and a serine protease effector domain derived from C1r, and Clone 19-C1s, which comprises a binding domain derived from a chimeric mouse monoclonal antibody to N. meningitidis fHbP and a serine protease effector domain derived from C1s. Binding of chimeric antibody Clone 19 is shown for comparison.

FIG. 24 shows the assessment of antibody titer against N. meningitidis serotype B (MC58) in a variety of human sera. ELISA plates were coated with N. meningitidis and incubated with different serum dilutions from twelve different human sera. Antibodies against N. meningitidis were detected using horseradish peroxidase (HRP)-conjugated anti-human IgG antibody.

FIG. 25 shows the detection of complement factor C5b-9 (also referred to as MAC) deposited on N. meningitidis cells after treatment with certain complement-activating molecules described herein. A mixture containing 10 μg/mL of targeted complement-activating molecule in 5% normal human serum (NHS) previously identified as having low titer of anti-Neisseria antibodies was incubated for the times shown on the x-axis. Controls used NHS alone or with Clone 19 anti-fHbP antibody. Detection was carried out using monoclonal antibody against MAC.

FIGS. 26A and 26B show the results of an N. meningitidis serum bactericidal assay using serum samples from four different individuals. Bacteria were incubated with buffer alone (BBS) or with 2.5% normal human serum (NHS) alone or in the presence of 10 μg/mL of anti-fHbp antibody Clone 19 or targeted complement-activating molecules Clone 19-C1r or Clone 19-C1s. Samples were taken at predetermined time points and plated on blood agar plates overnight at 37° C. and 5% CO₂. Serum bactericidal activity was calculated by measuring the decrease in the viable bacterial count recovered compared to the original bacterial count at zero time point and heat inactivated serum. Results are shown as colony-forming units (cfu)/ml. Each row shows results for a different serum sample. For each serum sample, the left chart shows the results after 30 minutes and the right chart shows the results after 60 minutes.

FIGS. 27A-27C show the results of complement component deposition assays using serum from four different individuals. Complement component deposition was assayed in a similar manner as described for FIGS. 26A and 26B, but with varied serum concentrations. FIG. 27A shows C3b deposition, FIG. 27B shows C4b deposition, and FIG. 27C shows C5b deposition.

FIG. 28 shows the results of a complement C3b deposition assay using anti-fHbp antibody Clone 19 and targeted complement-activating molecules comprising chimeric antibody Clone 19 and one of C1r, C1s, MASP-2, MASP-3, and Factor D. A Maxisorp polystyrene microtiter ELISA plate was coated with N. meningitidis antigen MC58 overnight at 4° C. The next day, the residual binding sites were blocked using 5% skimmed milk. Wild-type mouse serum (5%) with 150 nM of Clone 19 antibody, targeted complement-activating molecule, or isotype control antibody was added to the plate at different time points at room temperature. After incubation, the ELISA plate was washed and complement C3b deposition was detected using rabbit anti-C3b antibodies followed by goat anti-rabbit HRP conjugated antibodies.

FIG. 29 shows the results of assays of binding of monoclonal antibodies against Streptococcus pneumoniae antigen PspA to S. pneumoniae. Anti-PspA antibodies 5C6.1 and RX1MI005 were tested. S. pneumoniae strain D39 was incubated with either 5C6.1 or RX1MI005 at a concentration of 10 μg/mL for 30 minutes at room temperature, then washed and incubated with Alexa Fluor goat anti-human IgG for 30 minutes. Binding was measured by FACS analysis.

FIG. 30 shows the results of assays of binding of chimeric anti-PspA antibody RX1MI005 and targeted complement-activating molecules comprising RX1MI005 and either C1r or C1s to S. pneumoniae. An ELISA plate was coated with S. pneumoniae strain D39 in coating buffer and blocked with 5% skimmed milk. Serial dilutions of antibody or targeted complement-activating molecules were added to the plate and incubated for 30 minutes at room temperature then washed. Bound antibodies and targeted complement-activating molecules were detected using HRP conjugated anti-human IgG. An unrelated isotype antibody was included as a control.

FIG. 31 shows the results of assays of complement C3b deposition using anti-PspA antibody RX1MI005, targeted complement-activating molecules comprising RX1MI005 and either C1r or C1s, and an isotype control antibody. S. pneumoniae bacteria were washed twice with TBS buffer and resuspended in BBS⁺⁺ buffer (4 mM barbital, 145 mM NaCl, 2 mM CaCl₂, 1 mM MgCl₂, pH 7.4) buffer to a final concentration of 10⁶ cfu in 100 mL. The bacterial suspension (100 μL) was opsonized with 1% (vol/vol) NHS for 15 minutes at room temperature with antibody or targeted complement-activating molecules. Nonopsonized bacteria served as a negative control. After opsonization, the bacterial samples were washed twice with TBS buffer, and bound C3b was detected using FITC-conjugated rabbit anti-human C3c (Dako). Fluorescence intensity was measured with a FACSCalibur cell analyzer (BD Biosciences).

FIG. 32 shows the results of assays of binding of antibody and a targeted complement-activating molecule to Candida albicans. Antibody 1A2, which binds a fungal mannan epitope present on C. albicans, was used along with a targeted complement-activating molecule comprising antibody 1A2 and C1r. An unrelated isotype antibody was used as a control. An ELISA plate was coated with C. albicans in coating buffer and blocked with 5% skimmed milk. Serial dilutions of antibody 1A2 and the targeted complement-activating molecule were added to the plate and incubated for 30 minutes at room temperature, then washed. Bound antibodies were detected using HRP conjugated anti-human IgG.

FIG. 33 shows the results of assays of binding of antibody and a targeted complement-activating molecule to Candida albicans. Fungal cells were incubated with antibody 1A2 or a targeted complement-activating molecule comprising antibody 1A2 and C1r for 30 minutes at room temperature, then washed and incubated with Alexa Fluor goat anti-human IgG for 30 minutes. Binding was measured by FACS analysis. An unrelated isotype antibody was used as a control.

FIG. 34 shows the results of assays measuring C3b deposition triggered by certain antibodies and targeted complement-activating molecules on the surface of C. albicans. The left panel shows the assessment of antibody titer against C. albicans in a variety of human sera. ELISA plates were coated with C. albicans and incubated with sera from five different individuals. Antibodies against C. albicans were detected using horseradish peroxidase (HRP)-conjugated anti-human IgG antibody. The serum having the lowest measured titer of C. albicans antibodies, indicated as “GC”, was used in a C3b deposition assay, which results are shown in the right panel. For the C3b deposition assay, Maxisorp polystyrene microtiter ELISA plates were coated with formalin-fixed C. albicans in carbonate buffer (15 mM Na₂CO₃, 35 mM NaHCO₃, pH 9.6). The next day, wells were blocked with 5% skimmed milk in TBS buffer (10 mM Tris-HCl, 140 mM NaCl, pH7.4) for 2 hours then washed with TBS buffer containing 0.05% (v/v) Tween 20 and 5 mM CaCl₂. 1% NHS serum “GC” containing 150 nM of antibodies or targeted complement-activating molecules diluted in BBS⁺⁺ buffer (4 mM barbital, 145 mM NaCl, 2 mM CaCl₂, 1 mM MgCl₂, pH 7.4) and added to the plate and incubated for 5, 10, 15, 20 and 25 minutes at room temperature then washed. Deposition of C3b, detected using rabbit anti-C3c (Dako) followed by peroxidase-conjugated goat anti-rabbit IgG. After 1 hour, wells were washed and 100 μL of 1-Step Ultra TMB Solution (Thermo Fisher Scientific) was then added to each well and incubated for 5 minutes at room temperature. The reaction was stopped by the addition of 2 M H₂SO₄ and the optical density at 450 nm was immediately measured.

FIG. 35 shows the results of assays measuring binding of 11 different anti-Fnbp antibodies to Staphylococcus aureus. Antibodies were raised by injecting mice with S. aureus antigen fibronectin binding protein (Fnbp). Hybridomas were formed and supernatant samples were taken for screening using ELISA, which resulted in identification of 11 candidate antibodies. An ELISA plate was coated with S. aureus and residual binding sites were blocked using 5% skimmed milk. Fc receptors of S. aureus were blocked with Fc blocking agent. Serial concentrations of purified monoclonal antibodies were incubated with the ELISA plates for one hour at room temperature. Binding of antibodies and was detected using rabbit anti-mouse HRP conjugated antibodies. Clone G was identified as showing the best binding to S. aureus.

FIG. 36 shows the results of assays measuring binding of anti-FnbpB antibody Clone G to S. aureus strain MSSA. S. aureus MSSA bacteria were washed twice with TBS buffer and resuspended in BBS++ buffer (4 mM barbital, 145 mM NaCl, 2 mM CaCl2, 1 mM MgCl2, pH 7.4) buffer to a final concentration of 10⁷ cfu/mL. Fc receptors of S. aureus were blocked with Fc blocking agent. Bacterial suspension (100 μL) was incubated with 150 nM of mouse monospecific antibody 30 minutes at room temperature. Bacteria opsonized with an isotype control antibody were used as a negative control. After incubation, the bacterial samples were washed twice with TBS buffer, and bound antibodies were detected using FITC-conjugated rabbit anti-mouse IgG. Fluorescence intensity was measured with a FACSCalibur cell analyzer (BD Biosciences).

FIG. 37 shows the results of assays measuring binding of anti-Fnbp antibody Clone G to three different S. aureus MRSA isolates. S. aureus MRSA bacteria of each of three different isolates were washed twice with TBS buffer and resuspended in BBS⁺⁺ buffer (4 mM barbital, 145 mM NaCl, 2 mM CaCl₂, 1 mM MgCl₂, pH 7.4) buffer to a final concentration of 10⁷ cfu/mL. Fc receptors of S. aureus were blocked with Fc blocking agent. Bacterial suspension (100 μL) was incubated with 150 nM of antibodies for 30 minutes at room temperature. Bacteria opsonized with an isotype control antibody were used as a negative control. After incubation, the bacterial samples were washed twice with TBS buffer, and bound antibodies were detected using FITC-conjugated rabbit anti-mouse IgG. Fluorescence intensity was measured with a FACSCalibur cell analyzer (BD Biosciences).

FIGS. 38A and 38B show the results of assays measuring binding of antibody and targeted complement-activating molecules to S. aureus. Chimeric monoclonal anti-FnbpB antibody Clone G was tested, along with targeted complement-activating molecules comprising Clone G and C1r or C1s. ELISA plates were coated with either recombinant FnbpB (FIG. 38A) or with S. aureus (MRSA strain) (FIG. 38B), then residual binding sites were blocked using 5% skimmed milk. Fc receptors of S. aureus were blocked with Fc blocking agent. Serial concentrations of purified monoclonal antibody Clone G and targeted complement-activating molecules comprising Clone G and C1r or C1s were incubated with the ELISA plates for one hour at room temperature. Binding of antibodies and targeted complement-activating molecules was detected using HRP-conjugated rabbit anti-human IgG antibodies. The targeted complement-activating molecules showed good binding with both recombinant FnbpB (FIG. 38A) and S. aureus (MRSA strain) (FIG. 38B).

FIG. 39 shows the results of an assay measuring binding of certain antibodies and targeted complement-activating molecules to an antigen from Plasmodium falciparum. The antigen used is P. falciparum reticulocyte binding protein homologue 5 (PfRH5). Anti-PfRH5 antibodies R5.004 and R5.016 were tested, as were targeted complement-activating molecules comprising R5.004 and C1r, R5.004 and C1s, R5.016 and C1r, or R5.016 and C1s. Monoclonal antibody rituximab was used as a negative control. Maxisorp polystyrene microtiter ELISA plates were coated with 50 μL per well of cell supernatant from cells transfected with PfRH5. The next day, the wells were blocked with 1% BSA in PBS (1×) for two hours, then washed with PBS buffer containing 0.05% (v/v) Tween 20. Two-fold serial dilutions of antibodies and targeted complement-activating molecules were prepared in buffer containing 0.1% BSA in PBS (1×) with the highest concentration being 13.9 nM. Samples of 100 μL each were transferred to the ELISA plate and incubated at room temperature. After 1 hour, the plate was washed and 100 μL of goat HRP-conjugated anti-human IgG detection antibody was added to the plate followed by 30 minutes incubation at room temperature. The plate was washed and 100 μL of 1-step Ultra TMB Solution (Thermo Fisher Scientific) was added to each well and incubated for two minutes at room temperature. The reaction was stopped by the addition of 2M H₂SO₄ and the optical density at 450 nm was immediately measured.

FIG. 40 shows the results of an assay measuring complement C3b deposition triggered by certain antibodies and targeted complement-activating molecules on the surface of PfRH5 coated wells. Anti-PfRH5 antibodies R5.004 and R5.016 were tested, as were targeted complement-activating molecules comprising R5.004 and C1r, R5.004 and C1s, R5.016 and C1r, or R5.016 and C1s. Monoclonal antibody rituximab was used as a negative control. Maxisorp polystyrene microtiter ELISA plates were coated with 50 μL per well of cell supernatant from cells transfected with PfRH5. The next day, the wells were blocked with 1% BSA in PBS (1×) for two hours, then washed with PBS buffer containing 0.05% (v/v) Tween 20. Normal human serum (NETS) containing 13.9 nM of antibody or targeted complement-activating molecules was diluted in BBS⁺⁺ buffer (4 mM barbital, 145 mM NaCl, 2 mM CaCl₂, 1 mM MgCl₂, pH 7.4) to a concentration of 3%, and added to the wells. The plate was incubated for either 5, 10, 15, 20, or 25 minutes at room temperature, then washed three times. C3b deposition was detected using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well and incubated for two minutes at room temperature. The reaction was stopped by the addition of 2 M H₂SO₄ and the optical density at 450 nm was immediately measured.

FIG. 41 shows bacterial load in blood samples from mice infected with Neisseria meningitidis. 12-week-old female C57BL/6 wild-type mice (Charles River Laboratory) were used in this study. Mice were injected intraperitoneally (i.p.) with iron dextran (400 mg/kg; Sigma-Aldrich) 12 hours before infection. The next day, mice were injected i.p with 100 μL of passaged N. meningitidis B-MC58 suspension containing 5×10⁶ cfu in PBS and with iron dextran (400 mg/kg). Monoclonal antibody Clone 19 or a targeted complement-activating molecule comprising Clone 19 and C1r or C1s were injected i.p. at 18 hours before infection. Mice treated with an isotype control antibody served as a control. The inoculum dose was confirmed by viable count after plating on blood agar with 5% (vol/vol). Blood samples were obtained at pre-determined time points, and viable counts were calculated after serial dilution in PBS and plating out on blood agar plates. Mice treated with targeted complement-activating molecules comprising Clone 19 and C1r showed a significantly lower bacterial load in blood compared to mice that received Clone 19 antibody. Results are means±SEM. *P<0.05 and **P<0.01 by the Student t test.

FIG. 42 shows survival time of mice treated with antibody Clone 19, targeted complement-activating molecules comprising Clone 19 and C1r or C1s, and an isotype control antibody prior to infection with N. meningitidis. Mice were treated and infected as described in FIG. 43. Mice were monitored for progression of clinical signs and euthanized when they became lethargic. A significantly longer survival time was observed in mice treated with targeted complement-activating molecules comprising Clone 19 and C1r as compared to mice treated with Clone 19 antibody. Mantel-Cox log-rank test; n=12 mice/group; *P<0.05.

FIG. 43 shows the results of assays measuring binding of antibodies and certain targeted complement-activating molecules to HIV-1 envelope glycoprotein GP120. Antibody PGT121 was used, along with targeted complement-activating molecules comprising PGT121 and C1r or C1s. An unrelated isotype antibody was used as a control. Maxisorp polystyrene microtiter ELISA plates were coated with 2 μg/mL of recombinant GP120 in coating buffer. The next day, wells were blocked with 5% skimmed milk in PBS for two hours, then washed with TBS buffer containing 0.05% (v/v) Tween 20. Two-fold serial dilutions of antibodies and targeted complement-activating molecules were prepared in TB S buffer starting from 15 μg/mL. Samples of 100 μL were transferred to the ELISA plate and were incubated at room temperature. After one hour, the plate was washed and 100 μL of HRP-conjugated goat anti-human IgG detection antibody was added to the plate and incubated 30 minutes at room temperature. The plate was washed and 100 μL of 1-Step Ultra TMB Solution (Thermo Fisher Scientific) was added to each well and incubated for two minutes at room temperature. The reaction was stopped by the addition of 2 M H₂SO₄ and the optical density at 450 nm was immediately measured.

FIG. 44 shows the results of assays measuring C3b deposition triggered by antibody PGT121 and targeted complement-activating molecules comprising PGT121 and C1r or C1s on the surface of GP120-coated ELISA wells. Maxisorp polystyrene microtiter ELISA plates were coated with 2 μg/mL of recombinant GP120 in coating buffer. The next day, the wells were blocked with 5% skimmed milk in PBS for two hours, then washed with TBS buffer containing 0.05% (v/v) Tween 20. NHS containing 7.5 μg of antibodies or targeted complement-activating molecules was diluted in BBS++ buffer (4 mM barbital, 145 mM NaCl, 2 mM CaCl₂, 1 mM MgCl₂, pH 7.4) to a concentration of 2.5%, added to the plate, and incubated for 5, 10, 15, 20 and 25, and 25 minutes at room temperature then washed three times. C3b deposition was detected by using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured.

FIG. 45 shows the results of assays measuring binding of anti-FnbpB antibody Clone G and targeted complement-activating molecules comprising Clone G and C1r or C1s to Fnbp-coated ELISA plates. Maxisorp polystyrene microtiter ELISA plates were coated with 2 μg/mL of recombinant S. aureus FnbpB in coating buffer. The next day, wells were blocked with 5% skimmed milk in PBS for two hours, then washed with TBS buffer containing 0.05% (v/v) Tween 20. Two-fold serial dilutions of antibodies and targeted complement-activating molecules were prepared in TBS buffer starting from 15 μg/mL. Samples of 100 μL were transferred to the ELISA plate and were incubated at room temperature. After one hour, the plate was washed and 100 μL of HRP-conjugated goat anti-human IgG detection antibody was added to the plate, followed by 30 minutes incubation at room temperature. The plate was washed and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured.

FIG. 46 shows the results of assays measuring C3b deposition triggered by anti-FnbpB antibody Clone G and targeted complement-activating molecules comprising Clone G and C1r or C1s on the surface of FnbpB-coated ELISA wells. Maxisorp polystyrene microtiter ELISA plates were coated with 2 μg/mL of recombinant FnbpB in coating buffer. The next day, wells were blocked with 5% skimmed milk in PBS for two hours, then washed with TBS buffer containing 0.05% (v/v) Tween 20. NHS containing 7.5 μg of antibody or targeted complement-activating molecules was diluted in BBS⁺⁺ buffer (4 mM barbital, 145 mM NaCl, 2 mM CaCl₂, 1 mM MgCl₂, pH 7.4) to a concentration of 2.5%, added to the plate, and incubated for 5, 10, 15, 20 and 25, and 25 minutes at room temperature then washed three times. C3b deposition was detected by using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured.

FIG. 47 shows the results of assays measuring binding of anti-S protein antibody bebtelovimab and targeted complement-activating molecules comprising bebtelovimab and C1r or C1s to SARS-CoV-2 S protein. Maxisorp polystyrene microtiter ELISA plates were coated with 2 μg/mL of recombinant SARS-CoV-2 S protein in coating buffer. The next day, wells were blocked with 5% skimmed milk in PBS for 2 hours then washed with TBS buffer containing 0.05% (v/v) Tween 20. Two-fold serial dilutions of antibodies or targeted complement-activating molecules were prepared in TB S buffer starting from 15 μg/mL. 100 μL of samples were transferred to the ELISA plate and were incubated at room temperature. After 1 hour, the plate was washed and 100 μL of goat anti-human HRP detection antibody was added to the plate followed by 30 minutes incubation at room temperature. The plate was washed and 100 μL of 1-Step Ultra TMB Solution (Thermo Fisher Scientific) was added to each well and incubated for 2 minutes at room temperature. The reaction was stopped by the addition of 2M H₂SO₄ and the optical density at 450 nm was immediately measured.

FIG. 48 shows the results of assays measuring C3b deposition triggered by bebtelovimab or targeted complement-activating molecules comprising bebtelovimab and C1r or C1s on the surface of S protein-coated ELISA plates. Maxisorp polystyrene microtiter ELISA plates were coated with 2 μg/mL of recombinant S protein in coating buffer. The next day, wells were blocked with 5% skimmed milk in PBS for 2 hours then washed with TBS buffer containing 0.05% (v/v) Tween 20. 2.5% NHS containing 7.5 μg of antibodies or targeted complement-activating molecules were diluted in BBS⁺⁺ buffer (4 mM barbital, 145 mM NaCl, 2 mM CaCl₂, 1 mM MgCl₂, pH 7.4), were added to the plate and incubated for 5, 10, 15, 20 and 25, and 25 minutes at room temperature then washed 3 times. C3b deposition was detected by using rabbit anti-human C3c (Dako) followed by peroxidase-conjugated goat anti-rabbit IgG (Southern Biotech). After 1 hour, the plate was washed 3 times and 100 μL of 1-Step Ultra TMB Solution (Thermo Fisher Scientific) was added to each well at room temperature. The reaction was stopped by the addition of 2M H₂SO₄ and the optical density at 450 nm was immediately measured.

FIG. 49 shows the results of assays measuring binding of anti-M protein antibodies RB572 and RB574, along with targeted complement-activating molecules comprising either BR572 or RB574 and one of C1r and C1s. Maxisorp polystyrene microtiter ELISA plates were coated with 2 μg/mL SARS-CoV-2 M protein in PBS (1×). The next day, wells were blocked with 1% BSA in PBS (1×) for 2 hours then washed with PBS buffer containing 0.05% (v/v) Tween 20. Two-fold serial dilutions of antibodies and targeted complement-activating molecules were prepared in buffer containing 0.1% BSA in PBS (1×) with highest concentration 400 nM. 100 μL of samples were transferred to the ELISA plate and were incubated at room temperature. After 1 hour, the plate was washed and 100 μL of HRP-conjugated goat anti-human IgG detection antibody (American Qualex Antibodies, A130PD) was added to the plate followed by 30 minutes incubation at room temperature. The plate was washed and 100 μL of 1-Step Ultra TMB Solution (Thermo Fisher Scientific) was added to each well and incubated for 2 minutes at room temperature. The reaction was stopped by the addition of 2 M H₂SO₄ and the optical density at 450 nm was immediately measured. Unrelated antibody RTX was used as a control.

FIG. 50 shows the results of assays measuring C3b deposition triggered by anti-M protein antibody RB574 or targeted complement-activating molecules comprising RB574 and C1r. Maxisorp polystyrene microtiter ELISA plates were coated with 2 ug/mL M protein of SARS-CoV-2. The next day, wells were blocked with 1% BSA in PBS (1×) for 2 hours then washed with PBS buffer containing 0.05% (v/v) Tween 20. 3% NHS containing 200 nM of antibodies or targeted complement-activating molecules were diluted in BBS⁺⁺ buffer (4 mM barbital, 145 mM NaCl, 2 mM CaCl₂, 1 mM MgCl₂, pH 7.4), added to the plate and incubated for 5, 10, 15, 20, 25 and 30 minutes at room temperature then washed 3 times. C3b deposition was detected by using rabbit anti-human C3c (Dako) followed by HRP-conjugated goat anti-rabbit IgG (Southern Biotech). After 1 hour, the plate was washed 3 times and 100 μL of 1-Step Ultra TMB Solution (Thermo Fisher Scientific) was added to each well and incubated for 2 minutes at room temperature. The reaction was stopped by the addition of 2M H₂SO₄ and the optical density at 450 nm was immediately measured. Unrelated antibody RTX was used as a control.

FIG. 51 shows the results of assays measuring binding of anti-Aspergillus antibody hJF5 or targeted complement-activating molecules comprising hJF5 and C1r or C1s to Aspergillus fumigatus. Maxisorp polystyrene microtiter ELISA plates were coated with Aspergillus fumigatus in coating buffer. The next day, wells were blocked with 5% skimmed milk in PBS for 2 hours then washed with TBS buffer containing 0.05% (v/v) Tween 20. Two-fold serial dilutions of antibodies and targeted complement-activating molecules were prepared in TB S buffer starting from 15 μg/mL. 100 μL of samples were transferred to the ELISA plate and were incubated at room temperature. After 1 hour, the plate was washed and 100 μL of goat anti-human HRP detection antibody was added to the plate followed by 30 minutes incubation at room temperature. The plate was washed and 100 μL of 1-Step Ultra TMB Solution (Thermo Fisher Scientific) was added to each well and incubated for 2 minutes at room temperature. The reaction was stopped by the addition of 2M H₂SO₄ and the optical density at 450 nm was immediately measured.

FIG. 52 shows the results of assays measuring C3b deposition triggered by anti-Aspergillus antibody hJF5 or targeted complement-activating molecules comprising hJF5 and C1r or C1s on the surface of Aspergillus fumigatus. Maxisorp polystyrene microtiter ELISA plates were coated with Aspergillus fumigatus in coating buffer. The next day, wells were blocked with 5% skimmed milk in PBS for 2 hours then washed with TBS buffer containing 0.05% (v/v) Tween 20. 2.5% NHS containing 7.5 μg of antibodies or targeted complement-activating molecules were diluted in BBS⁺⁺ buffer (4 mM barbital, 145 mM NaCl, 2 mM CaCl₂, 1 mM MgCl₂, pH 7.4), were added to the plate and incubated for 5, 10, 15, 20 and 25, and 25 minutes at room temperature then washed 3 times. C3b deposition was detected by using rabbit anti-human C3c (Dako) followed by peroxidase-conjugated goat anti-rabbit IgG (Southern Biotech). After 1 hour, the plate was washed 3 times and 100 μL of 1-Step Ultra TMB Solution (Thermo Fisher Scientific) was added to each well at room temperature. The reaction was stopped by the addition of 2 M H₂SO₄ and the optical density at 450 nm was immediately measured.

### DETAILED DESCRIPTION

### I. DEFINITIONS

Unless specifically defined herein, all terms used herein have the same meaning as would be understood by those of ordinary skill in the art of the present invention. The following definitions are provided in order to provide clarity with respect to the terms as they are used in the specification and claims to describe the present invention. Additional definitions are set forth throughout this disclosure.

In the present descriptions, any concentration range, percentage range, ratio range, or integer range is to be understood to include the value of any integer within the recited range and, when appropriate, fractions thereof (such as one tenth and one hundredth of an integer), unless otherwise indicated or evident from the context. Any number range recited herein relating to any physical feature, such as polymer subunits, size, or thickness, is to be understood to include any integer within the recited range and, when appropriate, fractions thereof, unless otherwise indicated or evident from the context. As used herein, the term “about” is meant to specify that the range or value provided may vary by ±10% of the indicated range or value, unless otherwise indicated.

It should be understood that the terms “a”, “an”, and “the” as used herein refer to one or more of the referenced components. The use of the alternative (e.g., “or”) should be understood to mean either one, both, or any combination of the alternatives. As used herein, the terms “include”, “have”, and “comprise” are used synonymously, which terms and variants thereof are intended to be construed as non-limiting.

“Optional” or “optionally” means that the subsequently described element, component, event, or circumstance may or may not occur, and that the description includes instances in which the element component, event, or circumstance occurs and instances in which it does not.

It should be understood that the individual constructs or groups of constructs derived from the various combinations of the structures and subunits described herein are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target-binding affinity of a binding protein).

As used herein, the terms “treat”, “treatment”, or “ameliorate” refer to medical management of a disease, disorder, or condition of a subject. In general, an appropriate dose or treatment regimen comprising a targeted complement-activating molecule or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof.

A “therapeutically effective amount” or “effective amount” of a targeted complement-activating molecule, polynucleotide, vector, host cell, or composition of this disclosure refers to an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient, administered alone, a therapeutically effective amount refers to the effects of that ingredient or a cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially, sequentially, or simultaneously.

As used herein, “a subject” includes all mammals, including without limitation humans, non-human primates, dogs, cats, horses, sheep, goats, cows, rabbits, pigs, and rodents. A subject may be male or female, and can be any suitable age, including infant, juvenile, adolescent, adult, and geriatric subjects.

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

In the broadest sense, the naturally occurring amino acids can be divided into groups based on the chemical characteristic of the side chain of the respective amino acids. By “hydrophobic” amino acid is meant either Ile, Leu, Met, Phe, Trp, Tyr, Val, Ala, Cys or Pro. By “hydrophilic” amino acid is meant either Gly, Asn, Gln, Ser, Thr, Asp, Glu, Lys, Arg or His.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “peptide” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

“Nucleic acid molecule” or “oligonucleotide” or “polynucleotide” or “polynucleic acid” refers to an oligomeric or polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes, for example, mRNA, microRNA, siRNA, viral genomic RNA, and synthetic RNA, and polydeoxyribonucleic acid (DNA), which includes, for example, cDNA, genomic DNA, and synthetic DNA. Both RNA and DNA may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

“Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX), or Megalign (DNASTAR) software. The mathematical algorithm used in the BLAST programs can be found in Altschul et al., Nucleic Acids Res. 25:3389-3402, 1997. Appropriate parameters for measuring alignment, including any algorithms needed to achieve maximal alignment over the full-length of the sequences being compared, can be determined by known methods.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such a nucleic acid could be part of a vector and/or such a nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, antigen-binding fragment, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one or more base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, 100% level of activity of the parent polypeptide, or a level of activity greater than that of the parent polypeptide. In other words, a functional variant of a polypeptide or encoded polypeptide of this disclosure has “similar binding,” “similar affinity” or “similar activity” when the functional variant displays an improvement in performance, or no more than a 50% reduction in performance, in a selected assay as compared to the parent or reference polypeptide, such as an assay for measuring enzymatic activity or binding affinity.

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, 100% level of activity of the parent polypeptide, or a level of activity greater than that of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays an improvement in performance, or no more than a 50% reduction in performance, in a selected assay as compared to the parent or reference polypeptide (preferably no more than 20% or 10% reduction, or no more than a log difference as compared to the parent or reference with regard to affinity).

As used herein, the term “engineered,” “recombinant,” or “non-natural” refers to an organism, microorganism, cell, protein, polypeptide, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding an antibody or antigen-binding fragment (or other polypeptide), or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of different encoding nucleic acid molecules or the number of different protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mates et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” or “vector” refers to a DNA construct containing a nucleic acid molecule that is operably linked to a suitable control sequence capable of effecting the expression of the nucleic acid molecule in a suitable host. Such control sequences typically include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection”, “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to affect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest may also be considered operatively linked.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and a-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as Sleeping Beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Plasmid vectors, including DNA-based plasmid vectors for expression of one or more proteins in vitro or for direct administration to a subject, are also known in the art. Such vectors may comprise a bacterial origin of replication, a viral origin of replication, genes encoding components required for plasmid replication, and/or one or more selection markers, and may also contain additional sequences allowing for bicistronic or multicistronic expression.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

As used herein, the term “complement-activating” refers to a molecule that is capable of participating in one or more complement pathways in such a manner as to lead to deposition of complement components on a target cell surface and, optionally, to target cell death. The precise sequence of events that results from complement activation depends on the complement pathway activated (i.e., classical, lectin, or alternative) and the role of the specific complement-activating molecule within that pathway. As described above, each of the complement pathways entails the sequential activation of a series of serine proteases. Thus, the serine proteases of the complement pathway, such as mannan-binding lectin-associated serine proteases (MASP) MASP-1, MASP-2, and MASP-3, C1r, C1s, C2a, complement factor D (CFD), and complement factor Bb, are examples of complement-activating molecules.

“Antigen”, as used herein, refers to an immunogenic molecule that provokes an immune response. This immune response may involve antibody production, activation of specific immunologically competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen. Antigens can also be present in or on an infectious agent, such as present in a virion, or expressed or presented on the surface of a cell infected by infectious agent.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

The term “antibody” refers to an immunoglobulin molecule consisting of one or more polypeptides that specifically binds an antigen through at least one epitope recognition site. For example, the term “antibody” encompasses an intact antibody comprising at least two heavy chains and two light chains connected by disulfide bonds, as well as any antigen-binding portion or fragment of an intact antibody that has or retains the ability to bind to the antigen target molecule recognized by the intact antibody, such as an scFv, Fab, or Fab′2 fragment. The term also encompasses full-length or fragments of antibodies of any class or sub-class, including IgG and sub-classes thereof (such as IgG1, IgG2, IgG3, and IgG4), IgM, IgE, IgA, and IgD.

The term “antibody” is used herein in the broadest sense, encompassing antibodies and antibody fragments thereof, derived from any antibody-producing mammal (e.g., mouse, rat, rabbit, and primate including human), or from a hybridoma, phage selection, recombinant expression, or transgenic animals (or other methods of producing antibodies or antibody fragments). It is not intended that the term “antibody” be limited as regards to the source of the antibody or manner in which it is made (e.g., by hybridoma, phage selection, recombinant expression, transgenic animal, peptide synthesis, etc.). Exemplary antibodies include polyclonal, monoclonal and recombinant antibodies; multispecific antibodies (e.g., bispecific antibodies); humanized antibodies; fully human antibodies, murine antibodies; chimeric, mouse-human, mouse-primate, primate-human monoclonal antibodies; and anti-idiotype antibodies, and may be any intact molecule or fragment thereof. As used herein, the term “antibody” encompasses not only intact polyclonal or monoclonal antibodies, but also fragments thereof (such as dAb, Fab, Fab′, F(ab′)₂, Fv), single-chain (such as ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. The term encompasses genetically engineered and-or otherwise modified forms of immunoglobulins such as intrabodies, peptibodies, diabodies, triabodies, tetrabodies, tandem di-scFv, tandem tri-scFv, and the like, including antigen-binding fragments thereof.

The terms “VH” and “VL” refer to the variable binding regions from an antibody heavy chain and an antibody light chain, respectively. A VL may be a kappa class chain or a lambda class chain. The variable binding regions comprise discrete, well-defined sub-regions known as complementarity determining regions (CDRs) and framework regions (FRs). The CDRs are located within a hypervariable region (HVR) of the antibody and refer to sequences of amino acids within antibody variable regions which, in general, together confer the antigen specificity and/or binding affinity of the antibody. Consecutive CDRs (i.e., CDR1 and CDR2, and CDR2 and CDR3) are separated from one another in primary structure by a framework region.

As used herein, a “chimeric antibody” is a recombinant protein that contains the variable domains and complementarity determining regions derived from a non-human species (e.g., rodent) antibody, while the remainder of the antibody molecule is derived from a human antibody. In some embodiments, a chimeric antibody is comprised of an antigen-binding domain of one antibody operably linked or otherwise fused to heterologous constant regions of a different antibody. For example, a mouse-human chimeric antibody may comprise an antigen-binding domain of a mouse antibody fused to a constant region derived from a human antibody. In some embodiments, the heterologous constant region may be from a different Ig class from the parent antibody, including IgA (including subclasses IgA1 and IgA2), IgD, IgE, IgG (including subclasses IgG1, IgG2, IgG3 and IgG4) and IgM.

As used herein, a “humanized antibody” is a molecule, generally prepared using recombinant techniques, having an antigen-binding site derived from an immunoglobulin from a non-human species and the remaining immunoglobulin structure of the molecule based upon the structure and/or sequence of a human immunoglobulin. A humanized antibody differs from a chimeric antibody in that typically only the CDRs from the non-human species are used, grafted onto appropriate framework regions in a human variable domain. Antigen binding sites may be wild-type or may be modified by one or more amino acid substitutions. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

As used herein, the term “antibody fragment” refers to a portion derived from or related to a full-length antibody, generally including the antigen-binding or variable region thereof. Illustrative examples of antibody fragments include Fab, Fab′, F(ab)2, F(ab′)2 and Fv fragments, scFv fragments, diabodies, linear antibodies, single-chain antibody molecules and multispecific antibodies formed from antibody fragments.

As used herein, the term “antigen-binding fragment” refers to a polypeptide fragment that contains at least one CDR of an immunoglobulin heavy and/or light chains that specifically binds to the antigen to which the antibody was raised. An antigen-binding fragment may comprise 1, 2, 3, 4, 5, or all 6 CDRs of a VH and VL sequence from an antibody.

A “Fab” (fragment antigen binding) is the part of an antibody that binds to antigens and includes the variable region and CH1 of the heavy chain linked to the light chain via an inter-chain disulfide bond. Each Fab fragment is monovalent with respect to antigen binding, i.e., it has a single antigen-binding site. Pepsin treatment of an antibody yields a single large F(ab′)2 fragment that roughly corresponds to two disulfide-linked Fab fragments having divalent antigen-binding activity and is still capable of cross-linking antigen. Both the Fab and F(ab′)2 are examples of “antigen-binding fragments.” Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments are often produced as pairs of Fab′ fragments that have hinge cysteines between them. Other chemical couplings of antibody fragments are also known.

Fab fragments may be joined, e.g., by a peptide linker, to form a single-chain Fab, also referred to herein as “scFab.” In these embodiments, an inter-chain disulfide bond that is present in a native Fab may not be present, and the linker serves in full or in part to link or connect the Fab fragments in a single polypeptide chain. A heavy-chain derived Fab fragment (e.g., comprising, consisting of, or consisting essentially of VH+CH1, or “Fd”) and a light chain-derived Fab fragment (e.g., comprising, consisting of, or consisting essentially of VL+CL) may be linked in any arrangement to form a scFab. For example, a scFab may be arranged, in N-terminal to C-terminal direction, according to (heavy chain Fab fragment-linker-light chain Fab fragment) or (light chain Fab fragment-linker-heavy chain Fab fragment).

“Fv” is a small antibody fragment that contains a complete antigen-recognition and antigen-binding site. This fragment generally consists of a dimer of one heavy- and one light-chain variable region domain in tight, non-covalent association. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. The scFv polypeptide may comprise a polypeptide linker disposed between and linking the VH and VL domains that enables the scFv to retain or form the desired structure for antigen binding, although a linker is not always required. Such a peptide linker can be incorporated into a fusion polypeptide using standard techniques well known in the art. Additionally, or alternatively, Fv can have a disulfide bond formed between and stabilizing the VH and the VL. For a review of scFv, see Pluckthun in The Pharmacology of Monoclonal Antibodies, vol. 113, Rosenburg and Moore eds., Springer-Verlag, New York, pp. 269-315 (1994). In certain embodiments, the antibody or antigen-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C)). Alternatively, in some embodiments, a linker may be linked to an N-terminal portion or end of the VH domain, the VL domain, or both.

Peptide linker sequences for use in scFv or in other fusion proteins, such as the targeted complement-activating molecules described herein, may be chosen, for example, based on: (1) their ability to adopt a flexible extended conformation; (2) their inability or lack of ability to adopt a secondary structure that could interact with functional epitopes on the first and second polypeptides and/or on a target molecule; and/or (3) the lack or relative lack of hydrophobic or charged residues that might react with the polypeptides and/or target molecule. Other considerations regarding linker design (e.g., length) can include the conformation or range of conformations in which the VH and VL can form a functional antigen-binding site. In certain embodiments, peptide linker sequences contain, for example, Gly, Asn and Ser residues. Other near neutral amino acids, such as Thr and Ala, may also be included in a linker sequence. Other amino acid sequences which may be usefully employed as linker include those disclosed in Maratea et al., Gene 40:39 46(1985); Murphy et al., Proc. Natl. Acad. Sci. USA 83:8258 8262 (1986); U.S. Pat. Nos. 4,935,233, and 4,751,180. Other illustrative and non-limiting examples of linkers may include, for example, the pentamer Gly-Gly-Gly-Gly-Ser (SEQ ID NO:99) when present in a single iteration or repeated one to five times or more, and may begin or end in a partial iteration; see, e.g., SEQ ID NO:100. Any suitable linker may be used, and in general can be about 3, 4, 5, 6, 7, 8, 9, 10, 11, 12, 13, 14, 15, 16, 17, 18, 19, 20, 21, 22, 15 23, 24, 25, 26, 27, 28, 29, 30, 40, 50, 60, 70, 80, 90, 100 amino acids in length, or less than about 200 amino acids in length, and will preferably comprise a flexible structure (can provide flexibility and room for conformational movement between two regions, domains, motifs, fragments, or modules connected by the linker), and will preferably be biologically inert and/or have a low risk of immunogenicity in a human.

Antibodies may be monospecific (e.g., binding to a single epitope) or multispecific (e.g., binding to multiple epitopes and/or target molecules). A bispecific or multispecific antibody or antigen-binding fragment may, in some embodiments, comprise one, two, or more antigen-binding domains (e.g., a VH and a VL). Two or more binding domains may be present that bind to the same or different epitopes, and a bispecific or multispecific antibody or antigen-binding fragment as provided herein can, in some embodiments, two or more binding domains, that bind to different antigens or pathogens altogether.

Antibodies and antigen-binding fragments may be constructed in various formats. Exemplary antibody formats disclosed in Spiess et al., Mol. Immunol. 67(2):95 (2015), and in Brinkmann and Kontermann, mAbs 9(2):182-212 (2017), which formats and methods of making the same are incorporated herein by reference and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-Igs (e.g., U.S. Pat. No. 8,258,268, which formats are incorporated herein by reference in their entirety), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one), as well as so-called FIT-Ig (e.g., PCT 5 Publication No. WO 2015/103072, which formats are incorporated herein by reference in their entirety), so-called WuxiBody formats (e.g., PCT Publication No. WO 2019/057122, which formats are incorporated herein by reference in their entirety), and so-called In-Elbow-Insert Ig formats (IEI-Ig; e.g., PCT Publication Nos. WO 2019/024979 and WO 2019/025391, which formats are incorporated herein by reference in their entirety).

An antibody or antigen-binding fragment may comprise two or more VH domains, two or more VL domains, or both (i.e., two or more VH domains and two or more VL domains). In particular embodiments, an antigen-binding fragment comprises the format (N-terminal to C-terminal direction) VH-linker-VL-linker-VH-linker-VL, wherein the two VH sequences can be the same or different and the two VL sequences can be the same or different. Such linked scFvs can include any combination of VH and VL domains arranged to bind to a given target, and in formats comprising two or more VH and/or two or more VL, one, two, or more different epitopes or antigens may be bound. It will be appreciated that formats incorporating multiple antigen-binding domains may include VH and/or VL sequences in any combination or orientation. For example, the antigen-binding fragment can comprise the format VL-linker-VH-linker-VL-linker-VH, VH-linker-VL-linker-VL-linker-VH, or VL-linker-VH-linker-VH-linker-VL.

As used herein, the modifier “monoclonal” indicates the character of the antibody as being obtained from a substantially homogenous population of antibodies and is not intended to be limited as regards the source of the antibody or the manner in which it is made (e.g., by hybridoma, phage selection, recombinant expression, transgenic animals, etc.). The term “monoclonal antibody” encompasses not only intact monoclonal antibodies and full-length monoclonal antibodies, but also fragments thereof (such as Fab, Fab′, F(ab′)2, Fv), single-chain variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. Monoclonal antibodies can be obtained using any technique that provides for the production of antibody molecules by continuous cell lines in culture, such as the hybridoma method described by Kohler, G., et al., Nature 256:495, 1975, or they may be made by recombinant DNA methods (see, e.g., U.S. Pat. No. 4,816,567 to Cabilly). Monoclonal antibodies may also be isolated from phage antibody libraries using the techniques described in Clackson, T., et al., Nature 352:624-628, 1991, and Marks, J. D., et al., J. Mol. Biol. 222:581-597, 1991. Such antibodies can be of any immunoglobulin class including IgG, IgM, IgE, IgA, IgD and any subclass thereof.

The recognized immunoglobulin polypeptides include the kappa and lambda light chains and the alpha, gamma (IgG1, IgG2, IgG3, IgG4), delta, epsilon and mu heavy chains, or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids) similarly comprise a variable region (of about 116 amino acids) and one of the aforementioned heavy chain constant regions, e.g., gamma (of about 330 amino acids).

The basic four-chain antibody unit is a heterotetrameric glycoprotein composed of two identical light (L) chains and two identical heavy (H) chains. An IgM antibody differs from this plan in that it consists of five of the basic heterotetramer units along with an additional polypeptide called the J chain, and therefore contains 10 antigen-binding sites. Secreted IgA antibodies also differ from the basic structure in that they can polymerize to form polyvalent assemblages comprising two to five of the basic four-chain units along with a J chain. Each L chain is linked to an H chain by one covalent disulfide bond, while the two H chains are linked to each other by one or more by one or more disulfide bonds, depending on the H chain isotype. Each H and L chain also has regularly spaced intrachain disulfide bridges. The pairing of a VH and VL together forms a single antigen-binding site.

Each H chain has, at the N-terminus, a variable domain (VH) followed by three constant domains (CH1, CH2, CH3), in the case of alpha, gamma, and delta chains, or four CH domains (CH1, CH2, CH3, CH4), in the case of mu and epsilon chains.

Each L chain has, at the N-terminus, a variable domain (VL) followed by a constant domain (CL) at its other end. When an L chain and an H chain are paired, the VL is aligned with the VH, and the CL is aligned with the first constant domain of the heavy chain (CH1). The L chain from any vertebrate species can be assigned to one of two types, called kappa (κ) and lambda (λ), based on the amino acid sequences of their constant domains (CL).

Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated alpha (α), delta (δ), epsilon (ε), gamma (γ) and mu (μ), respectively. The y and a classes are further divided into subclasses on the basis of minor differences in CH sequence and function, for example, humans express the following subclasses: IgG1, IgG2, IgG3, IgG4, IgA1 and IgA2.

For the structure and properties of the different classes of antibodies, see, e.g., Basic and Clinical Immunology, 8th Edition, Daniel P. Stites, Abba I. Terr and Tristram G. Parslow (eds); Appleton and Lange, Norwalk, Conn., 1994, page 71 and Chapter 6.

The term “variable” refers to that fact that certain segments of the V domains differ extensively in sequence among antibodies. The V domain mediates antigen binding and defines specificity of a particular antibody for its particular antigen. However, the variability is not evenly distributed across the 110 amino acid span of the variable domains. Rather, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a beta-sheet configuration, connected by three hypervariable regions, which form loops connecting, and in some cases forming part of, the n-sheet structure. The hypervariable regions in each chain are held together in close proximity by the FRs and, with the hypervariable regions from the other chain, contribute to the formation of the antigen-binding site of antibodies (see Kabat, et al., Sequences of Proteins of Immunological Interest, 5th Ed. Public Health Service, National Institutes of Health, Bethesda, Md (1991)). The constant domains are not involved directly in binding an antibody to an antigen, but exhibit various effector functions.

As used herein, “effector functions” refer to those biological activities attributable to the Fc region of an antibody. Examples of antibody effector functions include participation in antibody-dependent cellular cytotoxicity (ADCC), C1q binding and complement-dependent cytotoxicity, Fc receptor binding, phagocytosis, down-regulation of cell surface receptors, and B cell activation. Modifications such as amino acid substitutions may be made to an Fc domain in order to modify (e.g., enhance or reduce) one or more functions of an Fc-containing polypeptide. Such functions include, for example, Fc receptor binding, antibody half-life modulation, ADCC function, protein A binding, protein G binding, and complement binding. Amino acid modifications that modify Fc functions include, for example, T250Q/M428L, M252Y/S254T/T256E, H433K/N434F, M428L/N434S, E233P/L234V/L235A/G236Δ/A327G/A330S/P331S, E333A, S239D/A330L/I332E, P257I/Q311, K326W/E333S, S239D/I332E/G236A, N297Q, K322A, S228P, L235E/E318A/K320A/K322A, L234A/L235A, and L234A/L235A/P329G mutations. Other Fc modifications and their effect on Fc function are known in the art.

As used herein, the term “hypervariable region” refers to the amino acid residues of an antibody that are responsible for antigen binding. The hypervariable region contains several “complementarity determining regions” (CDRs). The heavy chain comprises three CDR sequences (CDRH1, CDRH2, and CDRH3) and the light chain comprises three CDR sequences (CDRL1, CDRL2, and CDRL3). A variety of systems exist for identifying and numbering the amino acids that make up the CDRs. For example, the hypervariable region generally comprises CDRs at around about residues 24-34 (L1), 50-56 (L2) and 89-97 (L3) in the light chain variable domain, and at around about 31-35 (H1), 50-65 (H2) and 95-102 (H3) in the heavy chain variable domain when numbering in accordance with the Kabat numbering system as described in Kabat, et al., Sequences of Proteins of Immunological Interest, 5th Ed. Public Health Service, National Institutes of Health, Bethesda, Md. (1991); and/or at about residues 24-34 (L1), 50-56 (L2) and 89-97 (L3) in the light chain variable domain, and 26-32 (H1), 52-56 (H2) and 95-102 (H3) in the heavy chain variable domain when numbered in accordance with the Chothia numbering system, as described in Chothia and Lesk, J. Mol. Biol. 196:901-917 (1987); and/or at about residues 27-38 (L1), 56-65 (L2) and 105-117 (L3) in the VL, and 27-38 (H1), 56-65 (H2), and 105-117 (H3) in the VH when numbered in accordance with the IMGT numbering system as described in Lefranc, J. P., et al., Nucleic Acids Res 2 7: 209-212; Ruiz, M., et al., Nucleic Acids Res 28:219-221 (2000). Equivalent residue positions can be annotated and compared for different molecules using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

As used herein, “specifically binds” refers to an antibody or antigen-binding fragment that binds to an antigen with a particular affinity, while not significantly associating or uniting with any other molecules or components in a sample. Affinity may be defined as an equilibrium association constant (Kₐ), calculated as the ratio of kₒₙ/k_{off}, with units of 1/M or as an equilibrium dissociation constant K_{d}), calculated as the ratio of k_{off}/kₒₙ with units of M.

In some contexts, antibody and antigen-binding fragments may be described with reference to affinity and/or to avidity for antigen. Unless otherwise indicated, avidity refers to the total binding strength of an antibody or antigen-binding fragment thereof to antigen, and reflects binding affinity, valency of the antibody or antigen-binding fragment (e.g., whether the antibody or antigen-binding fragment comprises one, two, three, four, five, six, seven, eight, nine, ten, or more binding sites), and, for example, whether another agent is present that can affect the binding (e.g., a non-competitive inhibitor of the antibody or antigen-binding fragment).

Each embodiment in this specification is to be applied mutatis mutandis to every other embodiment unless expressly stated otherwise. It is contemplated that any embodiment discussed in this specification can be implemented with respect to any method, kit, reagent, or composition of the invention, and vice versa. Furthermore, compositions of the invention can be used to achieve methods of the invention.

### II. OVERVIEW

The present disclosure provides compositions and methods for the targeted activation of the complement pathway. Previously, therapeutic antibodies have been explored as a treatment for cancer, as it is well known that cell-mediated immunity, through the action of natural killer cells and cytotoxic T lymphocytes, plays a critical role in tumor suppression. However, recent studies have shown that the complement system has also an important role in immune surveillance against cancer. Deposits of activated complement components have been reported in several human tumors, along with overexpression of complement-regulatory proteins (CRPs) (Macor et al, Front. Immunol. 9:2203 (2018)). The effector mechanisms that lead to cytotoxic activity on cancer cells are primarily Fc-mediated; these include antibody-dependent cytotoxicity (ADCC), antibody-dependent phagocytosis (ADCP), complement-dependent cytotoxicity (CDC), complement-dependent cell-mediated cytotoxicity (CDCC), and complement-dependent cellular phagocytosis (CDCP).

Several strategies have been developed to direct activation of complement on tumor cell surfaces using monoclonal antibodies (mAbs); many of these mAbs have shown only suboptimal activity to drive complement activation via the classical pathway. Factors that influence cytotoxicity include the required hexamerization of the Fc region of the antibody for more efficient Cl binding, the epitope density of the target cell, and the expression of complement regulatory proteins (CRPs).

For antibody/antigen complexes (i.e., immune complexes) to initiate activation of the classical pathway in response to a pathogen infection or the presence of any non-self antigen, an antibody of the IgM subclass must bind to the antigen or at least two IgG subclass antibodies must bind to antigens in a manner that allows at least two of the six C-terminal immunoglobulin Fc region-binding “globular head” domains of C1q to bind the IgG immune complexes. This requires a stoichiometrically suitable distribution of antigen-binding immune complexes on the surface of target cells. Immune complexes that bind too distantly from each other fail to activate the classical pathway since they fail to form a pattern that allows the C1q recognition component to bind to at least two IgG immune complexes in close proximity to each other. The distribution of immune complexes on the activating surfaces is dependent on the distribution of antibody ligands, which therefore determines whether or not IgG complexes can trigger classical pathway activation. This is the reason why many monoclonal antibodies of the IgG immunoglobulin class fail to activate complement.

The present disclosure provides a novel, broadly applicable mAb platform called ‘targeted complement activation therapy’ (T-CAT) that exploits the full potential of complement to maximize the activity of therapeutic mAbs. This platform is appropriate not just for targeting cancer cells, but may also be used to target complement activity to any cell expressing an antigen to which antibody can be generated. Thus, the T-CAT platform may be used for a wide variety of applications, including treatment of cancers, autoimmune disorders, and pathogenic infections, including bacterial, viral, fungal, and parasitic infections. Targeted complement-activating molecules, which comprise fusion proteins having both a targeting domain derived from an antibody and a serine protease effector domain capable of activating one or more complement pathways, deliver targeted complement activation activity to the location of the antigen targeted by the antibody. The cells or tissues targeted are determined by the antigen-binding domain selected for use in the fusion protein.

The T-CAT platform supports the host's natural immune defense by combining the specificity of host immunoglobulins against microbial surface components with the ability to initial complement activation directly on microbial target surface without relying on the tightly controlled and complex pattern recognition-dependent activation pathways that can be undermined by pathogens' escape mechanisms. Similarly, the T-CAT platform supports the immune system's attack on malignant cells by activation of complement on the surface of the malignant cells despite their overexpression of negative regulatory complement components.

The T-CAT technology overcomes the steric requirements of antibodies to drive complement activation, since none of the pattern recognition molecules of the classical and lectin pathways are required to initiate the activation of complement. Rather, single targeted complement-activating molecules can activate complement to target the activator surface that expresses single ligands/antigens targeted by the antigen-binding site present in the targeted complement-activating molecule.

Another advantage of the T-CAT platform is that the targeted complement-activating molecules do not require a plasma recognition complex such as the C1 complex. Both the classical and the lectin pathways ordinarily require the formation of immune complexes bound to activating surfaces within a defined distance of each other in order to trigger the conformational changes that initiate conversion of serine proteases to their enzymatically active form and drive the cascade of cleavage events that result in complement activation. Such activation elicits the innate immune defense that targets pathogens or foreign cells, including single-cell or multi-cellular parasites and host cells that have become transformed, malignant, oxygen-deprived, hypothermic, virally infected, MHC mismatched, or otherwise injured. As a single targeted complement-activating molecule can initiate complement activation on the target surface, the ability of injured, mutant or virally infected host cells, parasitic foreign cells, or pathogenic bacteria to interfere with the highly regulated activation of the host's complement system may be overcome. Examples of such strategies are molecular mimicry (a cell or pathogen coating itself with negative complement regulatory proteins) or having evolved pathogenicity factors that facilitate infectivity, such as glycoprotein C of the Herpes viruses or calreticulin on the surface of Trypanosoma species cells.

### III. TARGETED COMPLEMENT-ACTIVATING MOLECULES

Provided herein are targeted complement-activating molecules comprising a) a target-binding domain and b) a complement-activating serine protease effector domain. Such molecules have the ability to deliver targeted complement activation activity to a cell surface, thereby leading to complement-mediated lysis of the targeted cell. The complement activation activity may be delivered to individual cells expressing the target antigen, or to tissues within which the target antigen is expressed.

A. Complement-Activating Serine Protease Effector Domains

In certain embodiments, the complement-activating serine protease effector domain of the targeted complement-activating molecules are derived from components of the complement system. In some embodiments, the complement-activating serine protease effector domain comprises MASP-1, MASP-2, MASP-3, C1r, C1s, complement factor D (CFD), C2a, or factor Bb. In some embodiments, the complement-activating serine protease effector domain comprises a fragment of any of the aforementioned proteases having serine protease activity. For example, the serine protease domain may comprise the CCP1-CCP2-SP domains of MASP-1, MASP-2, MASP-3, C1r, or C1s. Any serine protease that activates any of the classical, lectin, or alternative complement pathways may be used, as may any fragment of such a serine protease that retains such activity. In some embodiments, the complement-activating serine protease effector domain comprises a serine protease effector domain of MASP-1 (SEQ ID NO:67), MASP-2 (SEQ ID NO:57), MASP-3 (SEQ ID NO:66), C1r (SEQ ID NO:69), C1s (SEQ ID NO:76), C2a (SEQ ID NO:88), Bb (SEQ ID NO:89), mature CFD (SEQ ID NO:90), or pro-CFD (SEQ ID NO:92).

In some embodiments, the complement-activating serine protease effector domain is in an inactive, zymogen form that requires activation in order to form an active serine protease. Such activation may be provided by other molecules comprising the same serine protease effector domain, by molecules comprising a different serine protease effector domain, or by any other chemical or enzymatic means. In some embodiments, the complement-activating serine protease effector domain is in a catalytically active form. One example of a complement-activating serine protease effector domain in zymogen form is pro-CFD, which is converted to the active form, mature CFD, by removal of a 6 amino acid activation peptide. Many other complement-activating serine proteases, including MASP-1, MASP-2, MASP-3, C1r, and C1s, also have both active and zymogen forms.

In some embodiments, the complement-activating serine protease effector domain comprises one or more mutations relative to a wild-type serine protease. Any number of mutations may be present in the complement-activating serine protease effector domain, provided that it retains some level of serine protease activity. Accordingly, in some embodiments, the complement-activating serine protease effector domain comprises a sequence having at least 80%, at least 85%, at least 90%, at least 91%, at least 92%, at least 93%, at least 94%, at least 95%, at least 96%, at least 97%, at least 98% or at least 99% identity with the wild-type sequence of the corresponding serine protease effector domain. Such mutations may confer a beneficial effect on the targeted complement-activating molecule, such as increased resistance to protein degradation or increased resistance to inhibition by endogenous serpins, such as C1 inhibitor, or other inhibitors of serine protease activity. In some embodiments, the complement-activating serine protease effector domain comprises one or more mutations relative to a wild-type serine protease, such as MASP-2^{R444K} (SEQ ID NO:58), MASP-2^{K317Q, R444K} (SEQ ID NO:61), MASP-2^{K321Q, R444K} (SEQ ID NO:62), MASP-2^{K342Q, R444K} (SEQ ID NO:63), MASP-2^{K350Q, R444K} (SEQ ID NO:64), MASP-2^{K356Q, R444K} (SEQ ID NO:65), MASP-1^{R504Q} (SEQ ID NO:68), C1r^{K374Q} (SEQ ID NO:70), C1r^{R380Q} (SEQ ID NO:71), C1r^{H484W} (SEQ ID NO:72), C1r^{G485W} (SEQ ID NO:73), C1r^{R486W} (SEQ ID NO:74), C1s^{K308Q} (SEQ ID NO:78), C1s^{K310Q} (SEQ ID NO:79), C1s^{R314Q} (SEQ ID NO:80), C1s^{R331Q} (SEQ ID NO:81), C1s^{K346Q} (SEQ ID NO:82), C1s^{K351Q} (SEQ ID NO:83), C1s^{K353Q} (SEQ ID NO:84), C1s^{D456W} (SEQ ID NO:85), C1s^{N457W} (SEQ ID NO:86), and C1s^{P458W} (SEQ ID NO:87).

B. Target-Binding Domains

In certain embodiments, the target-binding domain of the targeted complement-activating molecule is derived from an antibody. The antibody may be a naturally occurring antibody of any class or sub-class, or any type of engineered antibody. For example, the target-binding domain may be derived from an antibody Fab fragment, F(ab′)2 fragment, Fab′ fragment, Fv fragment, a single-chain antibody fragment, a single-chain variable fragment (scFv), a single-domain antibody (e.g., sdAb, sdFv, or nanobody) or a fragment thereof, or an intrabody, peptibody, chimeric antibody, humanized antibody, multispecific antibody, or a fragment thereof. In some embodiments, the target-binding domain of the targeted complement-activating molecule comprises an antibody or an antigen-binding fragment thereof. In some embodiments, the target-binding domain comprises an antibody VH and/or VL. In some embodiments, the target-binding domain comprises from one to six CDRs of an antibody.

In some embodiments, the target-binding domain comprises an Fc region, or fragment thereof. In some embodiments, the Fc region comprises one or more mutations that modify (e.g., enhance or reduce) one or more functions of an Fc-containing polypeptide. Such functions include, for example, Fc receptor binding, antibody half-life modulation, ADCC function, protein A binding, protein G binding, and complement binding.

In certain embodiments, the target-binding domain binds to an antigen present on a cell. In some embodiments, the antigen is present on a cancer cell. In some embodiments, the cancer is a solid tumor cancer or a hematological cancer. For example, the cancer may be brain cancer, bladder cancer, breast cancer, cervical cancer, colorectal cancer, esophageal cancer, gastrointestinal cancer, liver cancer, kidney cancer, lymphoma, leukemia, lung cancer, melanoma, metastatic melanoma, mesothelioma, myeloma, neuroblastoma, ovarian cancer, prostate cancer, pancreatic cancer, renal cancer, skin cancer, or uterine cancer. In some embodiments, the cancer is acoustic neuroma, anal cancer (including carcinoma in situ), squamous cell carcinoma, adrenal tumor (including adenoma, hyperaldosteronism, adrenalcortical cancer), Cushing's syndrome, benign paraganglioma, appendix cancer (including pseudomyxoma peritonei, carcinoid tumors, non-carcinoid appendix tumors), bile duct cancer (including intrahepatic bile duct cancer, extrahepatic bile duct cancer, perihilar bile duct cancer, distal bile duct cancer), gallbladder cancer, bone cancer (including chondrosarcoma, osteosarcoma, malignant fibrous histiocytoma, fibrosarcoma, chordoma), brain tumor (including craniopharyngioma, dermoid cysts, epidermoid tumors, glioma, astrocytoma, low-grade astrocytoma, anaplastic astrocytoma, ependymoma, glioblastoma, oligodendrogliomas, hemangioblastoma, pineal gland tumors, pituitary tumors, sarcoma, chordoma), breast cancer (including lobular carcinoma, triple negative breast cancer, recurrent breast cancer, brain metastases), bladder cancer (including transitional cell bladder cancer, squamous cell carcinoma, adenocarcinoma), cancer of unknown primary (CUP), (including adenocarcinoma, poorly differentiated carcinoma, squamous cell carcinoma, poorly differentiated malignant neoplasm, neuroendocrine carcinoma), cervical cancer (including squamous cell carcinoma, adenocarcinoma, mixed carcinoma), a carcinoid tumor, a childhood germ cell tumor (including yolk sac tumors, teratoma, embryonal carcinoma, polyembryoma, germinoma), a childhood brain tumor (including ependymoma, craniopharyngioma, chordoma, pleomorphic xanthoastrocytoma, meningioma, primitive neuroectodermal tumors, ganglioglioma, pineoblastoma, germ cell tumors, mixed glial and neuronal tumors, astrocytoma, choroid plexus tumors), childhood leukemia (including lymphoblastic leukemia, myeloid leukemia), a childhood hematology disorder (including Fanconi anemia, Diamond-Blackfan anemia, aplastic anemia, Shwachman-Diamond syndrome, Kostmann's syndrome, neutropenia, thrombocytopenia, hemoglobinopathies, erythrocytosis, histiocytic disorders, iron overload, clotting and bleeding disorders), childhood liver cancer (including hepatoblastoma, hepatocellular carcinoma), childhood lymphoma (including Hodgkin's lymphoma, Non-Hodgkin's lymphoma, Burkitt's lymphoma, lymphoblastic lymphoma, large cell lymphoma), childhood osteosarcomas; childhood melanomas; childhood soft tissue sarcomas, colon cancer (including adenocarcinoma, hereditary nonpolyposis colorectal cancer syndrome, familial adenomatous polyposis), desmoplastic small round cell tumors (DSRCT); esophageal cancers (including adenocarcinoma, squamous cell carcinoma), Ewing's sarcoma (including Ewing's Sarcoma of the bone, extraosseous Ewing tumor, peripheral primitive neuroectodermal tumors), eye cancer (including uveal melanoma, basal cell carcinoma, squamous cell carcinoma, melanoma of the eyelid, melanoma of the conjunctiva, sebaceous carcinoma, Merkle cell carcinoma, mucosa-associated lymphoid tissue lymphoma, orbital lymphoma, orbital sarcoma, orbital and optic nerve meningioma, metastic orbital tumors, lacrimal gland lymphoma, adenoid cystic carcinoma, pleomorphic adenoma, transitional cell carcinoma, lacrimal sac lymphoma); fallopian tube cancer (including endometrioid adenocarcinoma, serous adenocarcinoma, leiomyosarcoma, transitional cell fallopian tube cancer); Hodgkin's lymphoma (including classical Hodgkin's lymphoma, nodular sclerosing Hodgkin's lymphoma, lymphocyte-rich classical Hodgkin's lymphoma, mixed cellularity Hodgkin's lymphoma, lymphocyte depletion Hodgkin's lymphoma, lymphocyte-predominant Hodgkin's lymphoma), implant-associated anaplastic large cell lymphoma (ALCL); inflammatory breast cancer (IBC); kidney cancer (including renal cell carcinoma, urothelial cancer of the kidney, pelvis and ureter); leukemia, (including acute lymphocyte leukemia, acute myeloid leukemia, chronic lymphoblastic leukemia, chronic myeloid leukemia), liver cancer (including hepatocellular carcinoma, fibrolamellar hepatocellular carcinoma, angiosarcoma, hepatoblastoma, hemangiosarcoma), lung cancer (including non-small cell lung cancer, adenocarcinoma, squamous cell carcinoma, large cell carcinoma, small cell lung cancer, carcinoid tumor, salivary gland carcinoma, lung metastases, sarcoma); medulloblastoma; melanoma (including cutaneous melanoma, superficial spreading melanoma, nodular melanoma, lentigo maligna melanoma, acral lentiginous melanoma, ocular melanoma, mucosal melanoma); mesothelioma (including sarcomatoid mesothelioma, biphasic mesothelioma), multiple endocrine neoplasias (MEN), (including multiple endocrine neoplasia type 1, multiple endocrine neoplasia type 2); multiple myeloma; myelodysplastic syndrome (MDS) (including refractory anemia, refractory cytopenia with multilineage dysplasia, refractory anemia with ringed sideroblasts, refractory anemia with excess blasts, refractory cytopenia with multilineage dysplasia and ringed sideroblasts); a myeloproliferative disorders (MPD) (including polycythemia vera, primary myelofibrosis, essential thrombocythemia, systemic mastocytosis, hypereosinophilic syndrome); neuroblastoma; neurofibromatosis (including neurofibromatosis type 1, neurofibromatosis type 2, schwannomatosis); non-Hodgkin's lymphoma (including b-cell lymphoma, t-cell lymphoma, NK-cell lymphoma, mucosa-associated lymphoid tissue lymphoma, follicular lymphoma, mantle cell lymphoma, diffuse large cell lymphoma, primary mediastinal large cell lymphoma, anaplastic large cell lymphoma, Burkitt's lymphoma, lymphoblastic lymphoma, marginal zone lymphoma); oral cancer (including squamous cell carcinoma); ovarian cancer (including epithelial ovarian cancer, germ cell ovarian cancer, stromal ovarian cancer, primary peritoneal ovarian cancer); pancreatic cancer (including islet cell carcinoma, sarcoma, lymphoma, pseudopapillary neoplasms, ampullary cancer, pancreatoblastoma, adenocarcinoma); parathyroid disease (including hyperparathyroidism, hypoparathyroidism, parathyroid cancer), penile cancer (including squamous cell carcinoma, Kaposi sarcoma, adenocarcinoma, melanoma, basal cell carcinoma); pituitary tumor (including non-functioning tumors, functioning tumors, pituitary cancer), prostate cancer (including adenocarcinoma, prostatic intraepithelial neoplasia), rectal cancer (including adenocarcinoma), retinoblastoma (including unilateral retinoblastoma, bilateral retinoblastoma, PNET retinoblastoma), skin cancer (including basal cell carcinoma, squamous cell carcinoma, actinic (solar) keratosis); skull base tumor (including meningioma, pituitary adenoma, acoustic neuroma, glomus tumors, squamous cell carcinoma, basal cell carcinoma, adenoid cystic carcinoma, adenocarcinoma, chondrosarcoma, rhabdomyosarcoma, osteosarcoma, esthesioblastoma, neuroendocrine carcinoma, mucosal melanoma), Soft tissue sarcomas; spinal tumor (including intramedullary spinal tumors, intradural extramedullary spinal tumors, extradural spinal tumors, osteoblastoma, enchondroma, aneurysmal bone cysts, giant cell tumors, hangioma, eosinophilic granuloma, osteosarcoma, chordoma, chondrosarcoma, plasmacytoma); stomach cancer (including lymphoma, gastrointestinal stromal tumors, carcinoid tumors); testicular cancer (including germ cell tumors, nonseminoma, seminoma, embryonal carcinoma, yolk sac tumors, teratoma, sertoli cell tumors, choriocarcinoma, stromal tumors, leydig cell tumors); throat cancer (including squamous cell carcinoma); thyroid cancer (including papillary thyroid cancer, follicular thyroid cancer, hurthle cell carcinoma, medullary thyroid cancer, anaplastic thyroid cancer); uterine cancer (including endometrioid adenocarcinoma, uterine carcinosarcoma, uterine sarcoma); vaginal cancer (including squamous cell carcinoma, adenocarcinoma, melanoma, sarcoma); vulvar cancer (including squamous cell carcinoma, adenocarcinoma, melanoma, sarcoma); von Hippel Lindau disease; Waldenstrom's macroglobulinemia; and Wilms' tumor. In some embodiments, the antigen is a cancer-associated antigen. For example, the antigen may be CD20, CD38, or CD52. Other cancer-associated antigens are known in the art and may also be targeted by the target-binding domain.

In some embodiments, the target-binding domain binds to a cell surface antigen on an immune cell that causes an autoimmune disease. In some embodiments, the immune cell is a B or T cell. Some cancer-associated antigens are also target antigens for autoimmune diseases, for example, CD20, CD38, and CD52. Examples of autoimmune diseases include rheumatoid arthritis, systemic lupus erythematosus, multiple sclerosis, autoimmune diabetes, autoimmune encephalitis, pemphigus vulgaris, vasculitis, Sjögren syndrome, and myasthenia gravis. Additional autoimmune diseases are known in the art.

In some embodiments, the target-binding domain binds to an antigen present on a microbial pathogen. The pathogen may be a bacterial pathogen, a viral pathogen, a fungal pathogen, or a parasitic pathogen. Examples of bacterial pathogens include Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, Salmonella species, Helicobacter species, Shigella species, Campylobacter species, and Listeria species. Examples of viral pathogens include Epstein-Barr virus, Human Immunodeficiency Virus 1 (HIV-1), Herpesviruses, Influenza viruses, West Nile virus, Cytomegaloviruses, and Coronaviruses, including SARS-CoV-2. Examples of fungal pathogens include Candida albicans and Aspergillus species. Examples of parasitic pathogens include Schistosoma mansoni, Plasmodium falciparum, and Trypanosoma cruzei. In some embodiments, the antigen is expressed on the surface of a microbial pathogen or on the surface of a cell infected by a microbial pathogen. For example, the antigen may be N. meningitidis factor H binding protein (fHbP), S. pneumoniae pneumococcal surface protein A (PspA), S. aureus protein A, S. aureus fibronectin-binding protein, HIV-1 surface glycoprotein 120, SARS-CoV-2 S or M protein, P. falciparum reticulocyte binding protein homologue 5, or a mannan epitope on the surface of a fungal organism such as C. albicans.

In some embodiments, the target-binding domain comprises an anti-CD20 antibody or antigen-binding fragment thereof, or an anti-CD38 antibody or antigen-binding fragment thereof, or an anti-CD52 antibody or antigen-binding fragment thereof, or an anti-fHbP antibody or antigen-binding fragment thereof, or an anti-PspA antibody or antigen-binding fragment thereof, or an anti-Fnbp antibody or antigen-binding fragment thereof, or an anti-PfRH5 antibody or antigen-binding fragment thereof, or anti-HIV-1 GP120 or an antigen-binding fragment thereof, or anti-SARS-CoV-2 S protein or antigen-binding fragment thereof, or anti-SARS-CoV-2 M protein or antigen-binding fragment thereof, or an anti-C. albicans fungal mannan epitope antibody or antigen-binding fragment thereof. In some embodiments, the target-binding domain comprises rituximab or an antigen-binding fragment thereof, alemtuzumab or an antigen-binding fragment thereof, daratumumab or an antigen-binding fragment thereof, or anti-fHbP antibody clone 19 or antigen-binding fragment thereof, or anti-PspA antibody RX1MI005 or an antigen-binding fragment thereof, or anti-Fnbp antibody Clone G or antigen-binding fragment thereof, or anti-PfRH5 antibody R5.004 or antigen-binding fragment thereof, or anti-PfRH5 antibody R5.016 or antigen-binding fragment thereof, or anti-GP120 antibody PGT121 or antigen-binding fragment thereof, or bebtelovimab or antigen-binding fragment thereof, or anti-fungal mannan antibody 1A2 or antigen-binding fragment thereof. In some embodiments, the target-binding domain comprises a rituximab heavy chain (SEQ ID NO:1) and/or a rituximab light chain (SEQ ID NO:2); an alemtuzumab heavy chain (SEQ ID NO:93) and/or an alemtuzumab light chain (SEQ ID NO:94); a daratumumab heavy chain (SEQ ID NO:95) and/or a daratumumab light chain (SEQ ID NO:96); an anti-fHbP clone 19 heavy chain (SEQ ID NO:103) and/or an anti-fHbP clone 19 light chain (SEQ ID NO:104); an RX1MI005 heavy chain (SEQ ID NO:120) and/or an RX1MI005 light chain (SEQ ID NO:121); a Clone G heavy chain (SEQ ID NO:124) and/or a Clone G light chain (SEQ ID NO:125); an R5.004 heavy chain (SEQ ID NO:136) and/or an R5.004 light chain (SEQ ID NO:137); an R5.016 heavy chain (SEQ ID NO:140) and/or an R5.016 light chain (SEQ ID NO:141); a PGT121 heavy chain (SEQ ID NO:144) and/or a PGT light chain (SEQ ID NO:145); a bebtelovimab heavy chain (SEQ ID NO:148) and/or a bebtelovimab light chain (SEQ ID NO:149), a 1A2 heavy chain (SEQ ID NO:128) and/or a 1A2 light chain (SEQ ID NO:129); or a hJF5 heavy chain (SEQ ID NO:132) and/or a hJF5 light chain (SEQ ID NO:133). In some embodiments, the target-binding domain comprises one or more mutations relative to the wild-type sequence of the corresponding antibody domain. For example, the target-binding domain may comprise mutations that inhibit protein degradation, inhibit glycosylation, enhance or reduce binding affinity or avidity, or increase in vivo half-life of the targeted complement-activating molecule. Accordingly, in some embodiments, the target-binding domain comprises a sequence having at least 80%, at least 85%, at least 90%, at least 91%, at least 92%, at least 93%, at least 94%, at least 95%, at least 96%, at least 97%, at least 98% or at least 99% identity with the wild-type sequence of the corresponding antibody domain.

C. Fusion Proteins and Multi-Chain Molecules

In certain embodiments, the targeted complement-activating molecule comprises a fusion protein. The fusion protein comprises a complement-activating serine protease effector domain fused to a target-binding domain. The fusion protein may have any of several configurations: a) the N-terminus of the complement-activating serine protease effector domain fused to the C-terminus of an antibody heavy chain or fragment thereof, b) the C-terminus of the complement-activating serine protease effector domain fused to the N-terminus of an antibody heavy chain or fragment thereof, c) the N-terminus of the complement-activating serine protease effector domain fused to the C-terminus of an antibody light chain or fragment thereof, d) the C-terminus of the complement-activating serine protease effector domain fused to the N-terminus of an antibody light chain or fragment thereof, e) the N-terminus of the complement-activating serine protease effector domain fused to the C-terminus of a single-chain or single-domain antibody or fragment thereof, or f) the C-terminus of the complement-activating serine protease effector domain fused to the N-terminus of a single-chain or single-domain antibody or fragment thereof.

In some embodiments the target-binding domain and the serine protease effector domain within the fusion protein are connected by a linker. Any suitable linker may be used. An example of one such linker is the pentamer Gly-Gly-Gly-Gly-Ser (SEQ ID NO:99), which may be present in a single iteration or repeated one to five times or more, and may begin or end in a partial iteration; see, e.g., SEQ ID NO:100.

In some embodiments, the fusion protein comprises a target-binding domain derived from rituximab and a serine protease effector domain derived from MASP-2. In some embodiments, the fusion protein comprises a target-binding domain comprising any one of SEQ ID NOs:1, 2, 3, 20, and 54-56 and a serine protease effector domain comprising any one of SEQ ID NOs:57, 58, and 61-65. In some embodiments, the fusion protein comprises the sequence set forth as SEQ ID NO:4, 5, 6, 7, 8, 9, 10, 33, 34, 35, 36, 37, or 38. In some embodiments, the fusion protein comprises a target-binding domain derived from rituximab and a serine protease effector domain derived from MASP-3. In some embodiments, the fusion protein comprises a target-binding domain comprising any one of SEQ ID NOs:1, 2, 3, 20, and 54-56 and a serine protease effector domain comprising SEQ ID NO:66. In some embodiments, the fusion protein comprises the sequence set forth as SEQ ID NO:12, 13, 14, or 15. In some embodiments, the fusion protein comprises a target-binding domain derived from rituximab and a serine protease effector domain derived from MASP-1. In some embodiments, the fusion protein comprises a target-binding domain comprising any one of SEQ ID NOs:1, 2, 3, 20, and 54-56 and a serine protease effector domain comprising any one of SEQ ID NOs:67 and 68. In some embodiments, the fusion protein comprises the sequence set forth as SEQ ID NO:16 or 17. In some embodiments, the fusion protein comprises a target-binding domain derived from rituximab and a serine protease effector domain derived from C1r. In some embodiments, the fusion protein comprises a target-binding domain comprising any one of SEQ ID NOs:1, 2, 3, 20, and 54-56 and a serine protease effector domain comprising any one of SEQ ID NOs:69-74. In some embodiments, the fusion protein comprises the sequence set forth as SEQ ID NO:18, 21, 39, 40, 48, 49, or 50. In some embodiments, the fusion protein comprises a target-binding domain derived from rituximab and a serine protease effector domain derived from C1s. In some embodiments, the fusion protein comprises a target-binding domain comprising any one of SEQ ID NOs:1, 2, 3, 20, and 54-56 and a serine protease effector domain comprising any one of SEQ ID NOs:76 and 78-87. In some embodiments, the fusion protein comprises the sequence set forth as SEQ ID NO:19, 23, 41, 42, 43, 44, 45, 46, 47, 51, 52, or 53. In some embodiments, the fusion protein comprises a target-binding domain derived from rituximab and a serine protease effector domain derived from CFD. In some embodiments, the fusion protein comprises a target-binding domain comprising any one of SEQ ID NOs:1, 2, 3, 20, and 54-56 and a serine protease effector domain comprising SEQ ID NO:90 or 92. In some embodiments, the fusion protein comprises the sequence set forth as SEQ ID NO:27, 28, 29, 30, or 32. In some embodiments, the fusion protein comprises a target-binding domain derived from rituximab and a serine protease effector domain derived from C2a. In some embodiments, the fusion protein comprises a target-binding domain comprising any one of SEQ ID NOs:1, 2, 3, 20, and 54-56 and a serine protease effector domain comprising SEQ ID NO:88. In some embodiments, the fusion protein comprises the sequence set forth as SEQ ID NO:25. In some embodiments, the fusion protein comprises a target-binding domain derived from rituximab and a serine protease effector domain derived from Bb. In some embodiments, the fusion protein comprises a target-binding domain comprising any one of SEQ ID NOs:1, 2, 3, 20, and 54-56 and a serine protease effector domain comprising SEQ ID NO:89. In some embodiments, the fusion protein comprises the sequence set forth as SEQ ID NO:26.

In some embodiments, the fusion protein comprises a target-binding domain derived from alemtuzumab and a serine protease effector domain derived from MASP-2. In some embodiments, the fusion protein comprises a target-binding domain comprising SEQ ID NO:93 or 94 and a serine protease effector domain comprising any one of SEQ ID NOs: 57, 58, and 61-65. In some embodiments, the fusion protein comprises a target-binding domain derived from alemtuzumab and a serine protease effector domain derived from MASP-3. In some embodiments, the fusion protein comprises a target-binding domain comprising SEQ ID NO:93 or 94 and a serine protease effector domain comprising SEQ ID NO:66. In some embodiments, the fusion protein comprises a target-binding domain derived from alemtuzumab and a serine protease effector domain derived from MASP-1. In some embodiments, the fusion protein comprises a target-binding domain comprising SEQ ID NO:93 or 94 and a serine protease effector domain comprising SEQ ID NO:67 or 68. In some embodiments, the fusion protein comprises a target-binding domain derived from alemtuzumab and a serine protease effector domain derived from C1r. In some embodiments, the fusion protein comprises a target-binding domain comprising SEQ ID NO:93 or 94 and a serine protease effector domain comprising any one of SEQ ID NOs:69-74. In some embodiments, the fusion protein comprises a target-binding domain derived from alemtuzumab and a serine protease effector domain derived from C1s. In some embodiments, the fusion protein comprises a target-binding domain comprising SEQ ID NO:93 or 94 and a serine protease effector domain comprising any one of SEQ ID NOs:77-87. In some embodiments, the fusion protein comprises a target-binding domain derived from alemtuzumab and a serine protease effector domain derived from CFD. In some embodiments, the fusion protein comprises a target-binding domain comprising any one of SEQ ID NOs:93 and 94 and a serine protease effector domain comprising SEQ ID NO:90or 92. In some embodiments, the fusion protein comprises the sequence set forth as SEQ ID NO:97. In some embodiments, the fusion protein comprises a target-binding domain derived from alemtuzumab and a serine protease effector domain derived from C2a. In some embodiments, the fusion protein comprises a target-binding domain comprising SEQ ID NO:93 or 94 and a serine protease effector domain comprising SEQ ID NO:88. In some embodiments, the fusion protein comprises a target-binding domain derived from alemtuzumab and a serine protease effector domain derived from Bb. In some embodiments, the fusion protein comprises a target-binding domain comprising SEQ ID NO:93 or 94 and a serine protease effector domain comprising SEQ ID NO:89.

In some embodiments, the fusion protein comprises a target-binding domain derived from daratumumab and a serine protease effector domain derived from MASP-2. In some embodiments, the fusion protein comprises a target-binding domain comprising SEQ ID NO:95 or 96 and a serine protease effector domain comprising any one of SEQ ID NOs: 57, 58, and 61-65. In some embodiments, the fusion protein comprises a target-binding domain derived from daratumumab and a serine protease effector domain derived from MASP-3. In some embodiments, the fusion protein comprises a target-binding domain comprising SEQ ID NO:95 or 96 and a serine protease effector domain comprising SEQ ID NO:66. In some embodiments, the fusion protein comprises a target-binding domain derived from daratumumab and a serine protease effector domain derived from MASP-1. In some embodiments, the fusion protein comprises a target-binding domain comprising SEQ ID NO:95 or 96 and a serine protease effector domain comprising SEQ ID NO:67 or 68. In some embodiments, the fusion protein comprises a target-binding domain derived from daratumumab and a serine protease effector domain derived from C1r. In some embodiments, the fusion protein comprises a target-binding domain comprising SEQ ID NO:95 or 96 and a serine protease effector domain comprising any one of SEQ ID NOs:69-74. In some embodiments, the fusion protein comprises a target-binding domain derived from daratumumab and a serine protease effector domain derived from C1s. In some embodiments, the fusion protein comprises a target-binding domain comprising SEQ ID NO:95 or 96 and a serine protease effector domain comprising any one of SEQ ID NOs:77-87. In some embodiments, the fusion protein comprises a target-binding domain derived from daratumumab and a serine protease effector domain derived from CFD. In some embodiments, the fusion protein comprises a target-binding domain comprising any one of SEQ ID NOs:95 and 96 and a serine protease effector domain comprising SEQ ID NO:90 or 92. In some embodiments, the fusion protein comprises the sequence set forth as SEQ ID NO:98. In some embodiments, the fusion protein comprises a target-binding domain derived from daratumumab and a serine protease effector domain derived from C2a. In some embodiments, the fusion protein comprises a target-binding domain comprising SEQ ID NO:95 or 96 and a serine protease effector domain comprising SEQ ID NO:88. In some embodiments, the fusion protein comprises a target-binding domain derived from daratumumab and a serine protease effector domain derived from Bb. In some embodiments, the fusion protein comprises a target-binding domain comprising SEQ ID NO:95 or 96 and a serine protease effector domain comprising SEQ ID NO:89.

In some embodiments, the fusion protein comprises a target-binding domain derived from anti-fHbP clone 19 and a serine protease effector domain derived from MASP-3. In some embodiments, the fusion protein comprises a target-binding domain comprising SEQ ID NO:103, 104, or 114 and a serine protease effector domain comprising SEQ ID NO: 66. In some embodiments, the fusion protein comprises the sequence sets forth as SEQ ID NO:117. In some embodiments, the fusion protein comprises a target-binding domain derived from anti-fHbP clone 19 and a serine protease effector domain derived from MASP-2. In some embodiments, the fusion protein comprises a target-binding domain comprising SEQ ID NO: 103, 104, or 114 and a serine protease effector domain comprising any one of SEQ ID NOs:57-65. In some embodiments, the fusion protein comprises the sequence set forth as SEQ ID NO:116. In some embodiments, the fusion protein comprises a target-binding domain derived from anti-fHbP clone 19 and a serine protease effector domain derived from MASP-1. In some embodiments, the fusion protein comprises a target-binding domain comprising SEQ ID NO:103, 104, or 114 and a serine protease effector domain comprising SEQ ID NO:67 or 68. In some embodiments, the fusion protein comprises a target-binding domain derived from anti-fHbP clone 19 and a serine protease effector domain derived from C1r. In some embodiments, the fusion protein comprises a target-binding domain comprising SEQ ID NO:103, 104, or 114 and a serine protease effector domain comprising any one of SEQ ID NOs:69-74. In some embodiments, the fusion protein comprises the sequence set forth as SEQ ID NO:108. In some embodiments, the fusion protein comprises a target-binding domain derived from anti-fHbP clone 19 and a serine protease effector domain derived from C1s. In some embodiments, the fusion protein comprises a target-binding domain comprising SEQ ID NO:103, 104, or 114 and a serine protease effector domain comprising any one of SEQ ID NOs:76-87. In some embodiments, the fusion protein comprises the sequence set forth as SEQ ID NO:111. In some embodiments, the fusion protein comprises a target-binding domain derived from anti-fHbP clone 19 and a serine protease effector domain derived from CFD. In some embodiments, the fusion protein comprises a target-binding domain comprising SEQ ID NO:103, 104, or 114 and a serine protease effector domain comprising SEQ ID NO:90 or 92. In some embodiments, the fusion protein comprises the sequence set forth as SEQ ID NO:118 or 119. In some embodiments, the fusion protein comprises a target-binding domain derived from anti-fHbP clone 19 and a serine protease effector domain derived from C2a. In some embodiments, the fusion protein comprises a target-binding domain comprising SEQ ID NO:103, 104, or 114 and a serine protease effector domain comprising SEQ ID NO:88. In some embodiments, the fusion protein comprises a target-binding domain derived from anti-fHbP clone 19 and a serine protease effector domain derived from Bb. In some embodiments, the fusion protein comprises a target-binding domain comprising SEQ ID NO:103, 104, or 114 and a serine protease effector domain comprising SEQ ID NO:89.

In some embodiments, the fusion protein comprises a target-binding domain derived from anti-PspA RX1MI005 and a serine protease effector domain derived from MASP-3. In some embodiments, the fusion protein comprises a target-binding domain comprising SEQ ID NO:120 or 121 and a serine protease effector domain comprising SEQ ID NO: 66. In some embodiments, the fusion protein comprises a target-binding domain derived from anti-PspA RX1MI005 and a serine protease effector domain derived from MASP-2. In some embodiments, the fusion protein comprises a target-binding domain comprising SEQ ID NO: 120 or 121 and a serine protease effector domain comprising any one of SEQ ID NOs:57-65. In some embodiments, the fusion protein comprises a target-binding domain derived from anti-PspA RX1MI005 and a serine protease effector domain derived from MASP-1. In some embodiments, the fusion protein comprises a target-binding domain comprising SEQ ID NO:120 or 121 and a serine protease effector domain comprising SEQ ID NO:67 or 68. In some embodiments, the fusion protein comprises a target-binding domain derived from anti-PspA RX1MI005 and a serine protease effector domain derived from C1r. In some embodiments, the fusion protein comprises a target-binding domain comprising SEQ ID NO:120 or 121 and a serine protease effector domain comprising any one of SEQ ID NOs:69-74. In some embodiments, the fusion protein comprises the sequence set forth as SEQ ID NO:122. In some embodiments, the fusion protein comprises a target-binding domain derived from anti-PspA RX1MI005 and a serine protease effector domain derived from C1s. In some embodiments, the fusion protein comprises a target-binding domain comprising SEQ ID NO:120 or 121 and a serine protease effector domain comprising any one of SEQ ID NOs:76-87. In some embodiments, the fusion protein comprises the sequence set forth as SEQ ID NO:123. In some embodiments, the fusion protein comprises a target-binding domain derived from anti-PspA and a serine protease effector domain derived from CFD. In some embodiments, the fusion protein comprises a target-binding domain comprising SEQ ID NO:120 or 121 and a serine protease effector domain comprising SEQ ID NO:90 or 92. In some embodiments, the fusion protein comprises a target-binding domain derived from anti-PspA and a serine protease effector domain derived from C2a. In some embodiments, the fusion protein comprises a target-binding domain comprising SEQ ID NO:120 or 121 and a serine protease effector domain comprising SEQ ID NO:88. In some embodiments, the fusion protein comprises a target-binding domain derived from anti-PspA and a serine protease effector domain derived from Bb. In some embodiments, the fusion protein comprises a target-binding domain comprising SEQ ID NO:120 or 121 and a serine protease effector domain comprising SEQ ID NO:89.

In some embodiments, the fusion protein comprises a target-binding domain derived from anti-Fnbp clone G and a serine protease effector domain derived from MASP-3. In some embodiments, the fusion protein comprises a target-binding domain comprising SEQ ID NO:124 or 125 and a serine protease effector domain comprising SEQ ID NO: 66. In some embodiments, the fusion protein comprises a target-binding domain derived from anti-Fnbp clone G and a serine protease effector domain derived from MASP-2. In some embodiments, the fusion protein comprises a target-binding domain comprising SEQ ID NO: 124 or 125 and a serine protease effector domain comprising any one of SEQ ID NOs:57-65. In some embodiments, the fusion protein comprises a target-binding domain derived from anti-Fnbp clone G and a serine protease effector domain derived from MASP-1. In some embodiments, the fusion protein comprises a target-binding domain comprising SEQ ID NO:124 or 125 and a serine protease effector domain comprising SEQ ID NO:67 or 68. In some embodiments, the fusion protein comprises a target-binding domain derived from anti-Fnbp clone G and a serine protease effector domain derived from C1r. In some embodiments, the fusion protein comprises a target-binding domain comprising SEQ ID NO:124 or 125 and a serine protease effector domain comprising any one of SEQ ID NOs:69-74. In some embodiments, the fusion protein comprises the sequence set forth as SEQ ID NO:126. In some embodiments, the fusion protein comprises a target-binding domain derived from anti-Fnbp clone G and a serine protease effector domain derived from C1s. In some embodiments, the fusion protein comprises a target-binding domain comprising SEQ ID NO:124 or 125 and a serine protease effector domain comprising any one of SEQ ID NOs:76-87. In some embodiments, the fusion protein comprises the sequence set forth as SEQ ID NO:127. In some embodiments, the fusion protein comprises a target-binding domain derived from anti-Fnbp clone G and a serine protease effector domain derived from CFD. In some embodiments, the fusion protein comprises a target-binding domain comprising SEQ ID NO:124 or 125 and a serine protease effector domain comprising SEQ ID NO:90 or 92. In some embodiments, the fusion protein comprises a target-binding domain derived from anti-Fnbp clone G and a serine protease effector domain derived from C2a. In some embodiments, the fusion protein comprises a target-binding domain comprising SEQ ID NO:124 or 125 and a serine protease effector domain comprising SEQ ID NO:88. In some embodiments, the fusion protein comprises a target-binding domain derived from anti-Fnbp clone G and a serine protease effector domain derived from Bb. In some embodiments, the fusion protein comprises a target-binding domain comprising SEQ ID NO:124 or 125 and a serine protease effector domain comprising SEQ ID NO:89.

In some embodiments, the fusion protein comprises a target-binding domain derived from anti-C. albicans antibody 1A2 and a serine protease effector domain derived from MASP-3. In some embodiments, the fusion protein comprises a target-binding domain comprising SEQ ID NO:128 or 129 and a serine protease effector domain comprising SEQ ID NO: 66. In some embodiments, the fusion protein comprises a target-binding domain derived from anti-C. albicans antibody 1A2 and a serine protease effector domain derived from MASP-2. In some embodiments, the fusion protein comprises a target-binding domain comprising SEQ ID NO: 128 or 129 and a serine protease effector domain comprising any one of SEQ ID NOs:57-65. In some embodiments, the fusion protein comprises a target-binding domain derived from anti-C. albicans antibody 1A2 and a serine protease effector domain derived from MASP-1. In some embodiments, the fusion protein comprises a target-binding domain comprising SEQ ID NO:128 or 129 and a serine protease effector domain comprising SEQ ID NO:67 or 68. In some embodiments, the fusion protein comprises a target-binding domain derived from anti-C. albicans antibody 1A2 and a serine protease effector domain derived from C1r. In some embodiments, the fusion protein comprises a target-binding domain comprising SEQ ID NO:128 or 129 and a serine protease effector domain comprising any one of SEQ ID NOs:69-74. In some embodiments, the fusion protein comprises the sequence set forth as SEQ ID NO:130. In some embodiments, the fusion protein comprises a target-binding domain derived from anti-C. albicans antibody 1A2 and a serine protease effector domain derived from C1s. In some embodiments, the fusion protein comprises a target-binding domain comprising SEQ ID NO:128 or 129 and a serine protease effector domain comprising any one of SEQ ID NOs:76-87. In some embodiments, the fusion protein comprises the sequence set forth as SEQ ID NO:131. In some embodiments, the fusion protein comprises a target-binding domain derived from anti-C. albicans antibody 1A2 and a serine protease effector domain derived from CFD. In some embodiments, the fusion protein comprises a target-binding domain comprising SEQ ID NO:128 or 129 and a serine protease effector domain comprising SEQ ID NO:90 92. In some embodiments, the fusion protein comprises a target-binding domain derived from anti-C. albicans antibody 1A2 and a serine protease effector domain derived from C2a. In some embodiments, the fusion protein comprises a target-binding domain comprising SEQ ID NO:128 or 129 and a serine protease effector domain comprising SEQ ID NO:88. In some embodiments, the fusion protein comprises a target-binding domain derived from anti-C. albicans antibody and a serine protease effector domain derived from Bb. In some embodiments, the fusion protein comprises a target-binding domain comprising SEQ ID NO:128 and 129 and a serine protease effector domain comprising SEQ ID NO:89.

In some embodiments, the fusion protein comprises a target-binding domain derived from anti-PfRH5 antibody R5.004 and a serine protease effector domain derived from MASP-3. In some embodiments, the fusion protein comprises a target-binding domain comprising SEQ ID NO:136 or 137 and a serine protease effector domain comprising SEQ ID NO: 66. In some embodiments, the fusion protein comprises a target-binding domain derived from anti-PfRH5 antibody R5.004 and a serine protease effector domain derived from MASP-2. In some embodiments, the fusion protein comprises a target-binding domain comprising SEQ ID NO: 136 or 137 and a serine protease effector domain comprising any one of SEQ ID NOs:57-65. In some embodiments, the fusion protein comprises a target-binding domain derived from anti-PfRH5 antibody R5.004 and a serine protease effector domain derived from MASP-1. In some embodiments, the fusion protein comprises a target-binding domain comprising SEQ ID NO:136 or 137 and a serine protease effector domain comprising SEQ ID NO:67 or 68. In some embodiments, the fusion protein comprises a target-binding domain derived from anti-PfRH5 antibody R5.004 and a serine protease effector domain derived from C1r. In some embodiments, the fusion protein comprises a target-binding domain comprising SEQ ID NO:136 or 137 and a serine protease effector domain comprising any one of SEQ ID NOs:69-74. In some embodiments, the fusion protein comprises the sequence set forth as SEQ ID NO:138. In some embodiments, the fusion protein comprises a target-binding domain derived from anti-PfRH5 antibody R5.004 and a serine protease effector domain derived from C1s. In some embodiments, the fusion protein comprises a target-binding domain comprising SEQ ID NO:136 or 137 and a serine protease effector domain comprising any one of SEQ ID NOs:76-87. In some embodiments, the fusion protein comprises the sequence set forth as SEQ ID NO:139. In some embodiments, the fusion protein comprises a target-binding domain derived from anti-PfRH5 antibody R5.004 and a serine protease effector domain derived from CFD. In some embodiments, the fusion protein comprises a target-binding domain comprising SEQ ID NO:136 or 137 and a serine protease effector domain comprising SEQ ID NO:90 or 92. In some embodiments, the fusion protein comprises a target-binding domain derived from anti-PfHR5 antibody R5.004 and a serine protease effector domain derived from C2a. In some embodiments, the fusion protein comprises a target-binding domain comprising SEQ ID NO:136 or 137 and a serine protease effector domain comprising SEQ ID NO:88. In some embodiments, the fusion protein comprises a target-binding domain derived from anti-PfHR5 antibody R5.004 and a serine protease effector domain derived from Bb. In some embodiments, the fusion protein comprises a target-binding domain comprising SEQ ID NO:136 or 137 and a serine protease effector domain comprising SEQ ID NO:89.

In some embodiments, the fusion protein comprises a target-binding domain derived from anti-PfHR5 antibody R5.016 and a serine protease effector domain derived from MASP-3. In some embodiments, the fusion protein comprises a target-binding domain comprising SEQ ID NO:140 or 141 and a serine protease effector domain comprising SEQ ID NO: 66. In some embodiments, the fusion protein comprises a target-binding domain derived from anti-PfHR5 antibody R5.016 and a serine protease effector domain derived from MASP-2. In some embodiments, the fusion protein comprises a target-binding domain comprising SEQ ID NO: 140 or 141 and a serine protease effector domain comprising any one of SEQ ID NOs:57-65. In some embodiments, the fusion protein comprises a target-binding domain derived from anti-PfHR5 antibody R5.016 and a serine protease effector domain derived from MASP-1. In some embodiments, the fusion protein comprises a target-binding domain comprising SEQ ID NO:140 or 141 and a serine protease effector domain comprising SEQ ID NO:67 or 68. In some embodiments, the fusion protein comprises a target-binding domain derived from anti-PfRH5 antibody R5.016 and a serine protease effector domain derived from C1r. In some embodiments, the fusion protein comprises a target-binding domain comprising SEQ ID NO:140 or 141 and a serine protease effector domain comprising any one of SEQ ID NOs:69-74. In some embodiments, the fusion protein comprises the sequence set forth as SEQ ID NO:142. In some embodiments, the fusion protein comprises a target-binding domain derived from anti-PfRH5 antibody R5.016 and a serine protease effector domain derived from C1s. In some embodiments, the fusion protein comprises a target-binding domain comprising SEQ ID NO:140 or 141 and a serine protease effector domain comprising any one of SEQ ID NOs:76-87. In some embodiments, the fusion protein comprises the sequence set forth as SEQ ID NO:143. In some embodiments, the fusion protein comprises a target-binding domain derived from anti-PfRH5 antibody R5.016 and a serine protease effector domain derived from CFD. In some embodiments, the fusion protein comprises a target-binding domain comprising SEQ ID NO:140 or 141 and a serine protease effector domain comprising SEQ ID NO:90 or 92. In some embodiments, the fusion protein comprises a target-binding domain derived from anti-PfRH5 antibody R5.016 and a serine protease effector domain derived from C2a. In some embodiments, the fusion protein comprises a target-binding domain comprising SEQ ID NO:140 or 141 and a serine protease effector domain comprising SEQ ID NO:88. In some embodiments, the fusion protein comprises a target-binding domain derived from anti-PfRH5 antibody R5.016 and a serine protease effector domain derived from Bb. In some embodiments, the fusion protein comprises a target-binding domain comprising SEQ ID NO:140 or 141 and a serine protease effector domain comprising SEQ ID NO:89.

In some embodiments, the fusion protein comprises a target-binding domain derived from anti-GP120 antibody PGT121 and a serine protease effector domain derived from MASP-3. In some embodiments, the fusion protein comprises a target-binding domain comprising SEQ ID NO:144 or 145 and a serine protease effector domain comprising SEQ ID NO: 66. In some embodiments, the fusion protein comprises a target-binding domain derived from anti-GP120 antibody PGT121 and a serine protease effector domain derived from MASP-2. In some embodiments, the fusion protein comprises a target-binding domain comprising SEQ ID NO: 144 or 145 and a serine protease effector domain comprising any one of SEQ ID NOs:57-65. In some embodiments, the fusion protein comprises a target-binding domain derived from anti-GP120 antibody PGT121 and a serine protease effector domain derived from MASP-1. In some embodiments, the fusion protein comprises a target-binding domain comprising SEQ ID NO:144 or 145 and a serine protease effector domain comprising SEQ ID NO:67 or 68. In some embodiments, the fusion protein comprises a target-binding domain derived from anti-GP120 antibody PGT121 and a serine protease effector domain derived from C1r. In some embodiments, the fusion protein comprises a target-binding domain comprising SEQ ID NO:144 or 145 and a serine protease effector domain comprising any one of SEQ ID NOs:69-74. In some embodiments, the fusion protein comprises the sequence set forth as SEQ ID NO:147. In some embodiments, the fusion protein comprises a target-binding domain derived from anti-GP120 antibody PGT121 and a serine protease effector domain derived from C1s. In some embodiments, the fusion protein comprises a target-binding domain comprising SEQ ID NO:144 or 145 and a serine protease effector domain comprising any one of SEQ ID NOs:76-87. In some embodiments, the fusion protein comprises the sequence set forth as SEQ ID NO:146. In some embodiments, the fusion protein comprises a target-binding domain derived from anti-GP120 antibody PGT121 and a serine protease effector domain derived from CFD. In some embodiments, the fusion protein comprises a target-binding domain comprising SEQ ID NO:144 or 145 and a serine protease effector domain comprising SEQ ID NO:90 or 92. In some embodiments, the fusion protein comprises a target-binding domain derived from anti-GP120 antibody PGT121 and a serine protease effector domain derived from C2a. In some embodiments, the fusion protein comprises a target-binding domain comprising SEQ ID NO:144 or 145 and a serine protease effector domain comprising SEQ ID NO:88. In some embodiments, the fusion protein comprises a target-binding domain derived from anti-GP120 antibody PGT121 and a serine protease effector domain derived from Bb. In some embodiments, the fusion protein comprises a target-binding domain comprising SEQ ID NO:144 or 145 and a serine protease effector domain comprising SEQ ID NO:89.

In some embodiments, the fusion protein comprises a target-binding domain derived from bebtelovimab and a serine protease effector domain derived from MASP-3. In some embodiments, the fusion protein comprises a target-binding domain comprising SEQ ID NO:148 or 149 and a serine protease effector domain comprising SEQ ID NO: 66. In some embodiments, the fusion protein comprises a target-binding domain derived from bebtelovimab and a serine protease effector domain derived from MASP-2. In some embodiments, the fusion protein comprises a target-binding domain comprising SEQ ID NO: 148 or 149 and a serine protease effector domain comprising any one of SEQ ID NOs:57-65. In some embodiments, the fusion protein comprises a target-binding domain derived from bebtelovimab and a serine protease effector domain derived from MASP-1. In some embodiments, the fusion protein comprises a target-binding domain comprising SEQ ID NO:148 or 149 and a serine protease effector domain comprising SEQ ID NO:67 or 68. In some embodiments, the fusion protein comprises a target-binding domain derived from bebtelovimab and a serine protease effector domain derived from C1r. In some embodiments, the fusion protein comprises a target-binding domain comprising SEQ ID NO:148 or 149 and a serine protease effector domain comprising any one of SEQ ID NOs:69-74. In some embodiments, the fusion protein comprises the sequence set forth as SEQ ID NO:150. In some embodiments, the fusion protein comprises a target-binding domain derived from bebtelovimab and a serine protease effector domain derived from C1s. In some embodiments, the fusion protein comprises a target-binding domain comprising SEQ ID NO:148 or 149 and a serine protease effector domain comprising any one of SEQ ID NOs:76-87. In some embodiments, the fusion protein comprises the sequence set forth as SEQ ID NO:151. In some embodiments, the fusion protein comprises a target-binding domain derived from bebtelovimab and a serine protease effector domain derived from CFD. In some embodiments, the fusion protein comprises a target-binding domain comprising SEQ ID NO:148 or 149 and a serine protease effector domain comprising SEQ ID NO:90 or 92. In some embodiments, the fusion protein comprises a target-binding domain derived from bebtelovimab and a serine protease effector domain derived from C2a. In some embodiments, the fusion protein comprises a target-binding domain comprising SEQ ID NO:148 or 149 and a serine protease effector domain comprising SEQ ID NO:88. In some embodiments, the fusion protein comprises a target-binding domain derived from bebtelovimab and a serine protease effector domain derived from Bb. In some embodiments, the fusion protein comprises a target-binding domain comprising SEQ ID NO:148 or 149 and a serine protease effector domain comprising SEQ ID NO:89.

In some embodiments, the fusion protein comprises a target-binding domain derived from anti-Aspergillus antibody hJF5 and a serine protease effector domain derived from MASP-3. In some embodiments, the fusion protein comprises a target-binding domain comprising SEQ ID NO:132 or 133 and a serine protease effector domain comprising SEQ ID NO: 66. In some embodiments, the fusion protein comprises a target-binding domain derived from anti-Aspergillus antibody hJF5 and a serine protease effector domain derived from MASP-2. In some embodiments, the fusion protein comprises a target-binding domain comprising SEQ ID NO: 1132 or 133 and a serine protease effector domain comprising any one of SEQ ID NOs:57-65. In some embodiments, the fusion protein comprises a target-binding domain derived from anti-Aspergillus antibody hJF5 and a serine protease effector domain derived from MASP-1. In some embodiments, the fusion protein comprises a target-binding domain comprising SEQ ID NO:132 or 133 and a serine protease effector domain comprising SEQ ID NO:67 or 68. In some embodiments, the fusion protein comprises a target-binding domain derived from anti-Aspergillus antibody hJF5 and a serine protease effector domain derived from C1r. In some embodiments, the fusion protein comprises a target-binding domain comprising SEQ ID NO:132 or 133 and a serine protease effector domain comprising any one of SEQ ID NOs:69-74. In some embodiments, the fusion protein comprises the sequence set forth as SEQ ID NO:134. In some embodiments, the fusion protein comprises a target-binding domain derived from anti-Aspergillus antibody hJF5 and a serine protease effector domain derived from C1s. In some embodiments, the fusion protein comprises a target-binding domain comprising SEQ ID NO:132 or 133 and a serine protease effector domain comprising any one of SEQ ID NOs:76-87. In some embodiments, the fusion protein comprises the sequence set forth as SEQ ID NO:135. In some embodiments, the fusion protein comprises a target-binding domain derived from anti-Aspergillus antibody hJF5 and a serine protease effector domain derived from CFD. In some embodiments, the fusion protein comprises a target-binding domain comprising SEQ ID NO:132 or 133 and a serine protease effector domain comprising SEQ ID NO:90 or 92. In some embodiments, the fusion protein comprises a target-binding domain derived from anti-Aspergillus antibody hJF5 and a serine protease effector domain derived from C2a. In some embodiments, the fusion protein comprises a target-binding domain comprising SEQ ID NO:132 or 133 and a serine protease effector domain comprising SEQ ID NO:88. In some embodiments, the fusion protein comprises a target-binding domain derived from anti-Aspergillus antibody hJF5 and a serine protease effector domain derived from Bb. In some embodiments, the fusion protein comprises a target-binding domain comprising SEQ ID NO:132 or 133 and a serine protease effector domain comprising SEQ ID NO:89.

Although the fusion proteins listed above are provided as examples of antibody binding domain and serine protease effector domain fusion proteins, it is contemplated that other antibody binding domains could be used in place of those listed. Alternative antibody binding domains include those derived from other antibodies to the listed antigens, those derived from antibodies that bind other antigens present on the targets listed above (e.g., cancer cells, immune cells, bacteria, fungi, viruses, and parasites), and those derived from antibodies that bind antigens present on any other appropriate targets, including other types of cancer cells, immune cells, bacteria, fungi, viruses, and parasites. The present disclosure contemplates targeted complement-activating molecules comprising target-binding domains derived from such antibodies and a serine protease effector domain as described herein.

In certain embodiments, the fusion protein comprises a target-binding domain derived from an antibody heavy chain or an antibody light chain. In such cases, the targeted complement-activating molecule may comprise an additional polypeptide that enhances antigen binding, effector function, stability, etc. of the molecule. In some embodiments, the targeted complement-activating molecule comprises: a) a fusion protein comprising a target-binding domain derived from an antibody heavy chain and b) an antibody light chain or fragment thereof. In some embodiments, the targeted complement-activating molecule comprises: a) a fusion protein comprising a target-binding domain derived from an antibody light chain and b) and antibody heavy chain or fragment thereof. In some embodiments, the antibody heavy chain and the antibody light chain are derived from the same antibody. In some embodiments, the targeted complement-activating molecule may comprise a) a fusion protein comprising a target-binding domain derived from an antibody heavy chain and b) a fusion protein comprising a target-binding domain derived from an antibody light chain.

In some embodiments, the targeted complement-activating molecule comprises a) a fusion protein comprising a target-binding domain derived from a rituximab heavy chain and b) a rituximab light chain or fragment thereof. In some embodiments, the targeted complement-activating molecule comprises a fusion protein comprising the sequence set forth as any one of SEQ ID NOs:4-6, 9, 12, 13, 16, 18, 19, 21, 23, 25-28, 32-47, and 48-53 and an antibody light chain comprising the sequence set forth as SEQ ID NO:2. In some embodiments, the targeted complement-activating molecule comprises a) a fusion protein comprising a target-binding domain derived from a rituximab light chain and b) a rituximab heavy chain or fragment thereof. In some embodiments, the targeted complement-activating molecule comprises a fusion protein comprising the sequence set forth as any one of SEQ ID NOs:7, 8, 14, 15, 17, 29, and 30 and an antibody heavy chain comprising the sequence set forth as any one of SEQ ID NOs:1, 3, 20, and 54-56.

In some embodiments, the targeted complement-activating molecule comprises a) a fusion protein comprising a target-binding domain derived from an alemtuzumab heavy chain and b) an alemtuzumab light chain or fragment thereof. In some embodiments, the targeted complement-activating molecule comprises a fusion protein comprising the sequence set forth as SEQ ID NO:97 and an antibody light chain comprising the sequence set forth as SEQ ID NO: 94. In some embodiments, the targeted complement-activating molecule comprises a) a fusion protein comprising a target-binding domain derived from an alemtuzumab light chain and b) an alemtuzumab heavy chain or fragment thereof.

In some embodiments, the targeted complement-activating molecule comprises a) a fusion protein comprising a target-binding domain derived from a daratumumab heavy chain and b) a daratumumab light chain or fragment thereof. In some embodiments, the targeted complement-activating molecule comprises a fusion protein comprising the sequence set forth as SEQ ID NO:98 and an antibody light chain comprising the sequence set forth as SEQ ID NO: 96. In some embodiments, the targeted complement-activating molecule comprises a) a fusion protein comprising a target-binding domain derived from a daratumumab light chain and b) a daratumumab heavy chain or fragment thereof.

In some embodiments, the targeted complement-activating molecule comprises a) a fusion protein comprising a target-binding domain derived from an anti-fHbP clone 19 heavy chain and b) an anti-fHbP clone light chain or fragment thereof. In some embodiments, the targeted complement-activating molecule comprises a fusion protein comprising the sequence set forth as SEQ ID NO:108, 111, 116, 117, 118, or 119 and an antibody light chain comprising the sequence set forth as SEQ ID NO: 104. In some embodiments, the targeted complement-activating molecule comprises a) a fusion protein comprising a target-binding domain derived from an anti-fHbP clone light chain and b) an anti-fHbP clone heavy chain or fragment thereof.

In some embodiments, the targeted complement-activating molecule comprises a) a fusion protein comprising a target-binding domain derived from a RX1MI005 heavy chain and b) a RX1MI005 light chain or fragment thereof. In some embodiments, the targeted complement-activating molecule comprises a fusion protein comprising the sequence set forth as SEQ ID NO:122 or 123 and an antibody light chain comprising the sequence set forth as SEQ ID NO: 121. In some embodiments, the targeted complement-activating molecule comprises a) a fusion protein comprising a target-binding domain derived from a RX1MI005 light chain and b) a RX1MI005 heavy chain or fragment thereof.

In some embodiments, the targeted complement-activating molecule comprises a) a fusion protein comprising a target-binding domain derived from a Clone G heavy chain and b) a Clone G light chain or fragment thereof. In some embodiments, the targeted complement-activating molecule comprises a fusion protein comprising the sequence set forth as SEQ ID NO:126 or 127 and an antibody light chain comprising the sequence set forth as SEQ ID NO: 125. In some embodiments, the targeted complement-activating molecule comprises a) a fusion protein comprising a target-binding domain derived from a Clone G light chain and b) a Clone G heavy chain or fragment thereof.

In some embodiments, the targeted complement-activating molecule comprises a) a fusion protein comprising a target-binding domain derived from a 1A2 heavy chain and b) a 1A2 light chain or fragment thereof. In some embodiments, the targeted complement-activating molecule comprises a fusion protein comprising the sequence set forth as SEQ ID NO:130 or 131 and an antibody light chain comprising the sequence set forth as SEQ ID NO:129. In some embodiments, the targeted complement-activating molecule comprises a) a fusion protein comprising a target-binding domain derived from a 1A2 light chain and b) a 1A2 heavy chain or fragment thereof.

In some embodiments, the targeted complement-activating molecule comprises a) a fusion protein comprising a target-binding domain derived from a R5.004 heavy chain and b) a R5.004 light chain or fragment thereof. In some embodiments, the targeted complement-activating molecule comprises a fusion protein comprising the sequence set forth as SEQ ID NO:138 or 139 and an antibody light chain comprising the sequence set forth as SEQ ID NO: 137. In some embodiments, the targeted complement-activating molecule comprises a) a fusion protein comprising a target-binding domain derived from a R5.004 light chain and b) a R5.004 heavy chain or fragment thereof.

In some embodiments, the targeted complement-activating molecule comprises a) a fusion protein comprising a target-binding domain derived from a R5.016 heavy chain and b) a R5.016 light chain or fragment thereof. In some embodiments, the targeted complement-activating molecule comprises a fusion protein comprising the sequence set forth as SEQ ID NO:142 or 143 and an antibody light chain comprising the sequence set forth as SEQ ID NO: 141. In some embodiments, the targeted complement-activating molecule comprises a) a fusion protein comprising a target-binding domain derived from a R5.016 light chain and b) a R5.016 heavy chain or fragment thereof.

In some embodiments, the targeted complement-activating molecule comprises a) a fusion protein comprising a target-binding domain derived from a PGT121 heavy chain and b) a PGT121 light chain or fragment thereof. In some embodiments, the targeted complement-activating molecule comprises a fusion protein comprising the sequence set forth as SEQ ID NO:146 or 147 and an antibody light chain comprising the sequence set forth as SEQ ID NO: 145. In some embodiments, the targeted complement-activating molecule comprises a) a fusion protein comprising a target-binding domain derived from a PGT121 light chain and b) a PGT121 heavy chain or fragment thereof.

In some embodiments, the targeted complement-activating molecule comprises a) a fusion protein comprising a target-binding domain derived from a bebtelovimab heavy chain and b) a bebtelovimab light chain or fragment thereof. In some embodiments, the targeted complement-activating molecule comprises a fusion protein comprising the sequence set forth as SEQ ID NO:150 or 151 and an antibody light chain comprising the sequence set forth as SEQ ID NO: 149. In some embodiments, the targeted complement-activating molecule comprises a) a fusion protein comprising a target-binding domain derived from a bebtelovimab light chain and b) a bebtelovimab heavy chain or fragment thereof.

In some embodiments, the targeted complement-activating molecule comprises a) a fusion protein comprising a target-binding domain derived from an anti-SARS-CoV-2 M protein antibody heavy chain and b) a light chain from an anti-SARS-CoV-2 M protein antibody or fragment thereof. In some embodiments, the targeted complement-activating molecule comprises a) a fusion protein comprising a target-binding domain derived from an anti-SARS-CoV-2 M protein antibody light chain and b) a heavy chain from an anti-SARS-CoV-2 M protein antibody or fragment thereof.

In some embodiments, the targeted complement-activating molecule comprises a) a fusion protein comprising a target-binding domain derived from a hJF5 heavy chain and b) a hJF5 light chain or fragment thereof. In some embodiments, the targeted complement-activating molecule comprises a fusion protein comprising the sequence set forth as SEQ ID NO:134 or 135 and an antibody light chain comprising the sequence set forth as SEQ ID NO: 133. In some embodiments, the targeted complement-activating molecule comprises a) a fusion protein comprising a target-binding domain derived from a hJF5 light chain and b) a hJF5 heavy chain or fragment thereof.

### IV. POLYNUCLEOTIDES, VECTORS, AND HOST CELLS

Further provided herein are isolated polynucleotides that encode any of the presently disclosed targeted complement-activating molecules or a portion thereof (e.g., fusion protein, antibody heavy chain or fragment thereof, or antibody light chain or fragment thereof). In certain embodiments, the polynucleotide is codon-optimized for expression in a host cell. Once a coding sequence is known or identified, codon optimization can be performed using known techniques and tools, such as the GenScript® OptimumGene™ tool or the ThermoFisher Scientific® GeneArt GeneOptimizer™. Codon-optimized sequences include sequences that are partially codon optimized, having one or more codons optimized for expression in the host cell, and those that are fully codon-optimized. It will also be appreciated that polynucleotides encoding targeted complement-activating molecules and portions thereof may possess different nucleotide sequences while still encoding the same protein due to the degeneracy of the genetic code, splicing, etc.

In certain embodiments, a polynucleotide encoding a targeted complement-activating molecule or portion thereof may be comprised in a polynucleotide that includes other sequences and/or features. For example, a polynucleotide may include one or more sequences useful for control or expression of the encoding proteins, such as promoter sequence(s), polyadenylation sequence(s), sequence(s) encoding signal peptides, etc. The polynucleotide may comprise deoxyribonucleic acid (DNA) or ribonucleic acid (RNA).

Also provided are vectors comprising or containing a polynucleotide that encodes any of the presently disclosed targeted complement-activating molecules or a portion thereof. Any appropriate vector may be used, including viral vectors and plasmid vectors. In certain embodiments, a vector comprises a polynucleotide that encodes both a fusion protein and the corresponding antibody heavy chain or light chain that together make up a targeted complement-activating molecule. The sequence encoding the fusion protein and the sequence encoding the antibody heavy chain or light chain may be contained within a single open reading frame, in which case they may optionally be separated by a polynucleotide encoding a protease cleavage site and/or a polynucleotide encoding a self-cleaving peptide. Alternatively, the sequence encoding the fusion protein and the sequence encoding the antibody heavy chain or light chain may be contained within separate open reading frames on a single vector. In other embodiments, the sequence encoding the fusion protein and the sequence encoding the antibody heavy chain or light chain are present on two different vectors, such that a first vector encodes the fusion protein and a second vector encodes the antibody heavy chain or light chain.

In a further aspect, the present disclosure also provides a host cell comprising a polynucleotide or vector disclosed herein. Any appropriate cell into which such a polynucleotide or vector may be introduced may be used. Examples of such cells include eukaryotic cells, including yeast cells, animal cells, insect cells, mammalian cells, and plant cells, and prokaryotic cells, including bacterial cells such as E. coli. In some embodiments, the host cell is a mammalian cell. In some embodiments, the host cell is an immortalized mammalian cell line. Cells appropriate for use in producing and expressing polynucleotides and vectors are known in the art.

In some embodiments, the cell may be transfected with a polynucleotide or vector disclosed herein. The term “transfection” encompasses any method known to one of skill in the art for introducing nucleic acid molecules into cells. Such methods include, for example, electroporation, lipofection, nanoparticle-based transfection, virus-based transfection, etc. Host cells may be transfected stably or transiently.

In some embodiments, the host cell expresses the targeted complement-activating molecule or portion thereof encoded by the polynucleotide or vector. Such expression may include post-translational modifications such as removal of signal sequence, glycosylation, and other such modifications. In a related aspect, the present disclosure provides methods for producing targeted complement-activating molecules or portions thereof, which methods comprise culturing a host cell for a sufficient time under conditions allowing for expression of the molecules and isolating the molecules. Methods useful for isolating and purifying recombinantly produced proteins include, for example, obtaining supernatant from suitable host cells that secrete the proteins into culture medium, concentrating the medium, and purifying the protein by passing the concentrate through a suitable purification matrix or series of matrices. Methods for purification of proteins are well known in the art.

### V. PHARMACEUTICAL COMPOSITIONS

Also provided herein are compositions that comprise a therapeutic agent selected from any one or more of the presently disclosed targeted complement-activating molecules, polynucleotides, vectors, or host cells, singly or in any combination, and may also include other selected therapeutic agents. Such compositions may further comprise one or more pharmaceutically acceptable carriers, excipients, or diluents.

A pharmaceutically acceptable carrier is non-toxic, biocompatible and is selected so as not to detrimentally affect the biological activity of the therapeutic agent (and any other therapeutic agents combined therewith). Examples of pharmaceutically acceptable carriers for peptides are described in U.S. Pat. No. 5,211,657 to Yamada. The therapeutic agents described herein may be formulated into preparations in solid, semi solid, gel, liquid, or gaseous forms such as tablets, capsules, powders, granules, ointments, solutions, depositories, inhalants, and injections allowing for oral, parenteral, or surgical administration. Local administration of the compositions by coating medical devices and the like is also contemplated.

Suitable carriers for parenteral delivery via injectable, infusion or irrigation and topical delivery include distilled water, physiological phosphate buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or di-glycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay, or regulate) the delivery of the agent(s) or to enhance the delivery, uptake, stability, or pharmacokinetics of the therapeutic agent(s). Such a delivery vehicle may include, by way of non-limiting example, microparticles, microspheres, nanospheres or nanoparticles composed of proteins, liposomes, carbohydrates, synthetic organic compounds, inorganic compounds, polymeric or copolymeric hydrogels and polymeric micelles. Suitable hydrogel and micelle delivery systems include the PEO:PHB:PEO copolymers and copolymer/cyclodextrin complexes disclosed in WO 2004/009664 A2 and the PEO and PEO/cyclodextrin complexes disclosed in U.S. Patent Application Publication No. 2002/0019369 A1. Such hydrogels may be injected locally at the site of intended action, or subcutaneously or intramuscularly to form a sustained release depot.

Compositions of the present invention may be formulated for delivery by any appropriate method including, without limitation, oral, topical, transdermal, sublingual, buccal, subcutaneously, intra-muscularly, intravenously, intra-arterially or as an inhalant.

The compositions of the present invention may also include biocompatible excipients, such as dispersing or wetting agents, suspending agents, diluents, buffers, penetration enhancers, emulsifiers, binders, thickeners, flavoring agents (for oral administration).

Pharmaceutical compositions according to certain embodiments of the present invention are formulated so as to allow the active ingredients contained therein to be bioavailable upon administration of the composition to a patient. Compositions that will be administered to a subject may take the form of one or more dosage units, and a container of a herein described therapeutic agent may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain an effective amount of therapeutic agent or composition of the present disclosure, for treatment of a disease or condition of interest in accordance with teachings herein.

A composition may be in the form of a solid or liquid. In some embodiments, the carrier(s) are particulate, so that the compositions are, for example, in tablet or powder form. The carrier(s) may be liquid, with the compositions being, for example, an oral oil, injectable liquid, or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer, or the like. Such a solid composition will typically contain one or more inert fillers or diluents such as sucrose, corn starch, or cellulose. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the composition is in the form of a capsule, for example, a gelatin capsule, it may contain, in addition to materials of the above type, a liquid carrier such as polyethylene glycol or oil.

The composition may be in the form of a liquid, for example, an elixir, syrup, solution, emulsion, or suspension. The liquid may be for oral administration or for delivery by injection, as two examples. When intended for oral administration, preferred compositions contain, in addition to the present compounds, one or more of a sweetening agent, preservative, dye/colorant and flavor enhancer. In a composition intended to be administered by injection, one or more of a surfactant, preservative, wetting agent, dispersing agent, suspending agent, buffer, stabilizer, and isotonic agent may be included.

Liquid pharmaceutical compositions, whether they be solutions, suspensions or other like form, may include one or more of the following excipients: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred excipient. An injectable pharmaceutical composition is preferably sterile.

A liquid composition intended for either parenteral or oral administration should contain an amount of a therapeutic agent as described herein such that a suitable dosage will be obtained. The term “parenteral” includes subcutaneous, intravenous, intramuscular, intrasternal, or intra-arterial injection or infusion. Typically, the therapeutic agent is at least 0.01% of the composition. When intended for oral administration, this amount may be varied to be between about 0.1% and about 70% of the weight of the composition. Certain oral pharmaceutical compositions contain between about 4% and about 75% therapeutic agent.

The composition may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a composition for topical administration. If intended for transdermal administration, the composition may include a transdermal patch or iontophoresis device. The pharmaceutical composition may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

A composition may include various materials which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The composition in solid or liquid form may include an agent that binds to the therapeutic agent(s) of the disclosure and thereby assists in the delivery of the compound. Suitable agents that may act in this capacity include one or more proteins or a liposome.

The composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic system in order to deliver the active ingredient(s). Delivery of the aerosol includes the necessary container, activators, valves, sub-containers, and the like, which together may form a kit. One of ordinary skill in the art, without undue experimentation, may determine preferred aerosols.

It will be understood that compositions of the present disclosure also encompass carrier molecules for polynucleotides, as described herein (e.g., lipid nanoparticles, nanoscale delivery platforms, and the like).

The pharmaceutical compositions may be prepared by methodology well known in the pharmaceutical art. For example, a composition intended to be administered by injection can be prepared by combining a composition that comprises therapeutic agent as described herein and optionally, one or more of salts, buffers and/or stabilizers, with sterile, distilled water so as to form a solution. A surfactant may be added to facilitate the formation of a homogeneous solution or suspension. Surfactants are compounds that non-covalently interact with the composition so as to facilitate dissolution or homogeneous suspension in the aqueous delivery system.

### VI. METHODS AND USES

Further provided herein are methods for use of a targeted complement-activating molecule, polynucleotide, vector, host cell, or composition of the present disclosure in activating one or more complement pathways in a mammalian subject. In some embodiments, the complement classical pathway, complement lectin pathway, or complement alternative pathway are activated. In some embodiments, any two or all three of the complement pathways are activated. In some embodiments, the targeted complement-activating molecule, polynucleotide, vector, host cell, or composition of the present disclosure may be used to induce complement-dependent cell death (CDC), complement-dependent cell-mediated cytotoxicity (CDCC), or complement-dependent cellular phagocytosis (CDCP) of a target cell. Such methods comprise contacting a target cell with the targeted complement-activating molecule or a composition comprising the targeted complement-activating molecule, wherein said contacting results in complement deposition on the target cell, thereby leading to complement-mediated cell death.

Also provided herein are methods of treating cancer, autoimmune disease, or a microbial infection in a subject comprising administering a therapeutically effective amount of a targeted complement-activating molecule or composition comprising the targeted complement-activating molecule to the subject. In some embodiments, cancer is treated using a targeted complement-activating molecule comprising a targeting domain that binds a cancer antigen. In some embodiments, the cancer is a solid tumor cancer or a hematological cancer. For example, the cancer may be brain cancer, bladder cancer, breast cancer, cervical cancer, colorectal cancer, esophageal cancer, gastrointestinal cancer, liver cancer, kidney cancer, lymphoma, leukemia, lung cancer, melanoma, metastatic melanoma, mesothelioma, myeloma, neuroblastoma, ovarian cancer, prostate cancer, pancreatic cancer, renal cancer, skin cancer, or uterine cancer. In some embodiments, an autoimmune disease is treated using a targeted complement-activating molecule that comprises a targeting domain that binds a cell surface antigen on an immune cell that causes autoimmune disease. In some embodiments, the immune cell is a B cell or a T cell. In some embodiments, the autoimmune disease is rheumatoid arthritis, systemic lupus erythematosus, multiple sclerosis, autoimmune diabetes, autoimmune encephalitis, pemphigus vulgaris, vasculitis, Sjögren syndrome, or myasthenia gravis. In some embodiments, a microbial infection is treated using a targeted complement-activating molecule comprising a targeting domain that binds an antigen present of the surface of a microbial pathogen or on the surface of a cell infected with a microbial pathogen. In some embodiments, the infection is a bacterial infection, a viral infection, a fungal infection, or a parasitic infection. In some embodiments, the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species. In some embodiments, the viral pathogen is an Epstein-Barr virus, a Human Immunodeficiency Virus 1 (HIV-1), a Herpesvirus, an Influenza virus, a West Nile virus, or a Cytomegalovirus. In some embodiments, the fungal pathogen is Candida albicans or an Aspergillus species. In some embodiments, the parasitic pathogen is Schistosoma mansoni, Plasmodium falciparum, or Trypanosoma cruzei.

Provided herein is the use of a targeted complement-activating molecule, polynucleotide, vector, host cell, or composition of the present disclosure for treatment of cancer, autoimmune disease, or a microbial infection. In some embodiments, the targeted complement-activating molecule for use in treatment of cancer comprises a targeting domain that binds a cancer antigen. In some embodiments, the cancer is a solid tumor cancer or a hematological cancer. For example, the cancer may be brain cancer, bladder cancer, breast cancer, cervical cancer, colorectal cancer, esophageal cancer, gastrointestinal cancer, liver cancer, kidney cancer, lymphoma, leukemia, lung cancer, melanoma, metastatic melanoma, mesothelioma, myeloma, neuroblastoma, ovarian cancer, prostate cancer, pancreatic cancer, renal cancer, skin cancer, or uterine cancer. In some embodiments, the targeted complement-activating molecule for use in treatment of an autoimmune disease comprises a targeting domain that binds an autoimmune-related antigen. In some embodiments, the autoimmune disease is rheumatoid arthritis, systemic lupus erythematosus, multiple sclerosis, autoimmune diabetes, autoimmune encephalitis, pemphigus vulgaris, vasculitis, Sjogren syndrome, or myasthenia gravis. In some embodiments, the targeted complement-activating molecule for use in treatment of a microbial infection comprises a targeting domain that binds an antigen present of the surface of a microbial pathogen or on the surface of a cell infected with a microbial pathogen. In some embodiments, the infection is a bacterial infection, a viral infection, a fungal infection, or a parasitic infection. In some embodiments, the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species. In some embodiments, the viral pathogen is an Epstein-Barr virus, a Human Immunodeficiency Virus 1 (HIV-1), a Herpesvirus, an Influenza virus, a West Nile virus, or a Cytomegalovirus. In some embodiments, the fungal pathogen is Candida albicans or an Aspergillus species. In some embodiments, the parasitic pathogen is Schistosoma mansoni, Plasmodium falciparum, or Trypanosoma cruzei.

Provided herein is a targeted complement-activating molecule, polynucleotide, vector, host cell, or composition of the present disclosure for use in the manufacture of a medicament for treating cancer, autoimmune disease, or a microbial infection. In some embodiments, the medicament for treating cancer comprises a targeted complement-activating molecule comprising a targeting domain that binds a cancer antigen. In some embodiments, the cancer is a solid tumor cancer or a hematological cancer. For example, the cancer may be brain cancer, bladder cancer, breast cancer, cervical cancer, colorectal cancer, esophageal cancer, gastrointestinal cancer, liver cancer, kidney cancer, lymphoma, leukemia, lung cancer, melanoma, metastatic melanoma, mesothelioma, myeloma, neuroblastoma, ovarian cancer, prostate cancer, pancreatic cancer, renal cancer, skin cancer, or uterine cancer. In some embodiments, the medicament for treating an autoimmune disease comprises a targeted complement-activating molecule comprising a targeting domain that binds an autoimmune-related antigen. In some embodiments, the autoimmune disease is rheumatoid arthritis, systemic lupus erythematosus, multiple sclerosis, autoimmune diabetes, autoimmune encephalitis, pemphigus vulgaris, vasculitis, Sjogren syndrome, or myasthenia gravis. In some embodiments, the medicament for treating a microbial infection comprises a targeted complement-activating molecule comprising a targeting domain that binds an antigen present of the surface of a microbial pathogen or on the surface of a cell infected with a microbial pathogen. In some embodiments, the microbial infection is a bacterial infection, a viral infection, a fungal infection, or a parasitic infection. In some embodiments, the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species. In some embodiments, the viral pathogen is an Epstein-Barr virus, a Human Immunodeficiency Virus 1 (HIV-1), a Herpesvirus, an Influenza virus, a West Nile virus, or a Cytomegalovirus. In some embodiments, the fungal pathogen is Candida albicans or an Aspergillus species. In some embodiments, the parasitic pathogen is Schistosoma mansoni, Plasmodium falciparum, or Trypanosoma cruzei.

Administration of the targeted complement-activating molecules or compositions of the present disclosure may be by any appropriate route, including oral, topical, transdermal, sublingual, buccal, subcutaneously, intra-muscularly, intravenously, intra-arterially or as an inhalant. The targeted complement-activating molecules or compositions of the present disclosure are administered in a therapeutically effective amount, which amount will vary depending upon a variety of factors including the specific molecules employed, the metabolic stability and length of action of the molecules, the age, sex, body weight, general health, and diet of the subject, the mode and time of administration, the rate of excretion, any additional therapeutic agents administered to the subject in the same time frame, the severity of the particular disorder or disease, and the genetic and epigenetic makeup of the subject. In certain embodiments, the targeted complement-activating molecules or compositions may be administered to the subject 1, 2, 3, 4, 5, 6, 7, 8, 9, or 10 times, or more. Successive administration may be carried out at any interval, including about 6, about 12, about 24, about 36, about 48, about 74, about 96, or about 108 hours apart, or more.

In some embodiments, the targeted complement-activating molecules, polynucleotides, vectors, host cells, or compositions of the present disclosure are used in combination with other therapeutic agents. Such combination therapy may include administration of a single pharmaceutical dosage formulation that contains targeted complement-activating molecules or compositions of the present disclosure together with one or more additional therapeutic agents, or the targeted complement-activating molecules or compositions of the present disclosure and the additional therapeutic agents may each be administered as a separate dosage formulation. Where separate dosage formulations are used, the targeted complement-activating molecules or compositions of the present disclosure and the additional therapeutic agents may be administered at essentially the same time, i.e., concurrently, or at separate times, i.e., sequentially in any order. In some embodiments, a combination therapy may comprise administration of two or more different targeted complement-activating molecules of the present disclosure, or two or more compositions each comprising a different targeted complement-activating molecule of the present disclosure.

### VII. SEQUENCES

The sequences referred to within the present specification are summarized in Table 3.

TABLE 3

| SEQ | | |
|---|---|---|
| ID NO. | Description | Sequence |
| 1 | RTX_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 2 | RTX_LC | QIVLSQSPAILSASPGEKVTMTCRASSSVSYIHWFQQKPGS |
| | | SPKPWIYATSNLASGVPVRFSGSGSGTSYSLTISRVEAEDA |
| | | ATYYCQQWTSNPPTFGGGTKLEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGEC |
| 3 | RTX(H)^{ΔK}_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| 4 | RTX(H)-M2_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| | | GGGGSGGGGSGGGGQPCPYPMAPPNGHVSPVQAKYILKDSF |
| | | SIFCETGYELLQGHLPLKSFTAVCQKDGSWDRPMPACSIVD |
| | | CGPPDDLPSGRVEYITGPGVTTYKAVIQYSCEETFYTMKVN |
| | | DGKYVCEADGFWTSSKGEKSLPVCEPVCGLSARTTGGRIYG |
| | | GQKAKPGDFPWQVLILGGTTAAGALLYDNWVLTAAHAVYEQ |
| | | KHDASALDIRMGTLKRLSPHYTQAWSEAVFIHEGYTHDAGF |
| | | DNDIALIKLNNKVVINSNITPICLPRKEAESFMRTDDIGTA |
| | | SGWGLTQRGFLARNLMYVDIPIVDHQKCTAAYEKPPYPRGS |
| | | VTANMLCAGLESGGKDSCRGDSGGALVFLDSETERWFVGGI |
| | | VSWGSMNCGEAGQYGVYTKVINYIPWIENIISDE |
| 5 | RTX(H)^{ΔK}-M2_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGRIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 6 | M2-RTX(H)_HC | QPCPYPMAPPNGHVSPVQAKYILKDSFSIFCETGYELLQGH |
| | | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGRIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDFGGGGSGGGGSGGGGQVQLQQP |
| | | GAELVKPGASVKMSCKASGYTFTSYNMHWVKQTPGRGLEWI |
| | | GAIYPGNGDTSYNQKFKGKATLTADKSSSTAYMQLSSLTSE |
| | | DSAVYYCARSTYYGGDWYFNVWGAGTTVTVSAASTKGPSVF |
| | | PLAPSSKSTSGGTAALGCLVKDYFPEPVTVSWNSGALTSGV |
| | | HTFPAVLQSSGLYSLSSVVTVPSSSLGTQTYICNVNHKPSN |
| | | TKVDKKVEPKSCDKTHTCPPCPAPELLGGPSVFLFPPKPKD |
| | | TLMISRTPEVTCVVVDVSHEDPEVKFNWYVDGVEVHNAKTK |
| | | PREEQYNSTYRVVSVLTVLHQDWLNGKEYKCKVSNKALPAP |
| | | IEKTISKAKGQPREPQVYTLPPSRDELTKNQVSLTCLVKGF |
| | | YPSDIAVEWESNGQPENNYKTTPPVLDSDGSFFLYSKLTVD |
| | | KSRWQQGNVFSCSVMHEALHNHYTQKSLSLSPGK |
| 7 | RTX(L)-M2_LC | QIVLSQSPAILSASPGEKVTMTCRASSSVSYIHWFQQKPGS |
| | | SPKPWIYATSNLASGVPVRFSGSGSGTSYSLTISRVEAEDA |
| | | ATYYCQQWTSNPPTFGGGTKLEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGECGGGGSGGGGSGGGGQPCPYPMAPPNGHVSPVQA |
| | | KYILKDSFSIFCETGYELLQGHLPLKSFTAVCQKDGSWDRP |
| | | MPACSIVDCGPPDDLPSGRVEYITGPGVTTYKAVIQYSCEE |
| | | TFYTMKVNDGKYVCEADGFWTSSKGEKSLPVCEPVCGLSAR |
| | | TTGGRIYGGQKAKPGDFPWQVLILGGTTAAGALLYDNWVLT |
| | | AAHAVYEQKHDASALDIRMGTLKRLSPHYTQAWSEAVFIHE |
| | | GYTHDAGFDNDIALIKLNNKVVINSNITPICLPRKEAESFM |
| | | RTDDIGTASGWGLTQRGFLARNLMYVDIPIVDHQKCTAAYE |
| | | KPPYPRGSVTANMLCAGLESGGKDSCRGDSGGALVFLDSET |
| | | ERWFVGGIVSWGSMNCGEAGQYGVYTKVINYIPWIENIISD |
| | | F |
| 8 | M2-RTX(L)_LC | QPCPYPMAPPNGHVSPVQAKYILKDSFSIFCETGYELLQGH |
| | | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGRIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDFGGGGSGGGGSGGGGQIVLSQS |
| | | PAILSASPGEKVTMTCRASSSVSYIHWFQQKPGSSPKPWIY |
| | | ATSNLASGVPVRFSGSGSGTSYSLTISRVEAEDAATYYCQQ |
| | | WTSNPPTFGGGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGE |
| | | C |
| 9 | RTX(H)^{ΔK}- | QVQLQQPGAELVKPGASVKMSCKASGYTFTSYNMHWVKQTP |
| | M2^{R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 10 | RTX(H)^{ΔK}- | QVQLQQPGAELVKPGASVKMSCKASGYTFTSYNMHWVKQTP |
| | M2^{R444Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGQIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 11 | RTX(H)^{ΔK}- | QVQLQQPGAELVKPGASVKMSCKASGYTFTSYNMHWVKQTP |
| | M2^{S633A}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGRIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDAGGALVFLDSETERWFVGGIV |
| | | SWGSMNCGEAGQYGVYTKVINYIPWIENIISDF |
| 12 | RTX(H)^{ΔK}-M3_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGNECPELQPPVHGKIEPSQAKYFFKDQVL |
| | | VSCDTGYKVLKDNVEMDTFQIECLKDGTWSNKIPTCKIVDC |
| | | RAPGELEHGLITFSTRNNLTTYKSEIKYSCQEPYYKMLNNN |
| | | TGIYTCSAQGVWMNKVLGRSLPTCLPECGQPSRSLPSLVKR |
| | | IIGGRNAEPGLFPWQALIVVEDTSRVPNDKWEGSGALLSAS |
| | | WILTAAHVLRSQRRDTTVIPVSKEHVTVYLGLHDVRDKSGA |
| | | VNSSAARVVLHPDFNIQNYNHDIALVQLQEPVPLGPHVMPV |
| | | CLPRLEPEGPAPHMLGLVAGWGISNPNVTVDEIISSGTRTL |
| | | SDVLQYVKLPVVPHAECKTSYESRSGNYSVTENMFCAGYYE |
| | | GGKDTCLGDSGGAFVIFDDLSQRWVVQGLVSWGGPEECGSK |
| | | QVYGVYTKVSNYVDWVWEQMGLPQSVVEPQVER |
| 13 | M3-RTX(H)_HC | NECPELQPPVHGKIEPSQAKYFFKDQVLVSCDTGYKVLKDN |
| | | VEMDTFQIECLKDGTWSNKIPTCKIVDCRAPGELEHGLITF |
| | | STRNNLTTYKSEIKYSCQEPYYKMLNNNTGIYTCSAQGVWM |
| | | NKVLGRSLPTCLPECGQPSRSLPSLVKRIIGGRNAEPGLFP |
| | | WQALIVVEDTSRVPNDKWFGSGALLSASWILTAAHVLRSQR |
| | | RDTTVIPVSKEHVTVYLGLHDVRDKSGAVNSSAARVVLHPD |
| | | FNIQNYNHDIALVQLQEPVPLGPHVMPVCLPRLEPEGPAPH |
| | | MLGLVAGWGISNPNVTVDEIISSGTRTLSDVLQYVKLPVVP |
| | | HAECKTSYESRSGNYSVTENMFCAGYYEGGKDTCLGDSGGA |
| | | FVIFDDLSQRWVVQGLVSWGGPEECGSKQVYGVYTKVSNYV |
| | | DWVWEQMGLPQSVVEPQVERGGGGSGGGGSGGGGQVQLQQP |
| | | GAELVKPGASVKMSCKASGYTFTSYNMHWVKQTPGRGLEWI |
| | | GAIYPGNGDTSYNQKFKGKATLTADKSSSTAYMQLSSLTSE |
| | | DSAVYYCARSTYYGGDWYFNVWGAGTTVTVSAASTKGPSVF |
| | | PLAPSSKSTSGGTAALGCLVKDYFPEPVTVSWNSGALTSGV |
| | | HTFPAVLQSSGLYSLSSVVTVPSSSLGTQTYICNVNHKPSN |
| | | TKVDKKVEPKSCDKTHTCPPCPAPELLGGPSVFLFPPKPKD |
| | | TLMISRTPEVTCVVVDVSHEDPEVKFNWYVDGVEVHNAKTK |
| | | PREEQYNSTYRVVSVLTVLHQDWLNGKEYKCKVSNKALPAP |
| | | IEKTISKAKGQPREPQVYTLPPSRDELTKNQVSLTCLVKGF |
| | | YPSDIAVEWESNGQPENNYKTTPPVLDSDGSFFLYSKLTVD |
| | | KSRWQQGNVFSCSVMHEALHNHYTQKSLSLSPGK |
| 14 | RTX(L)-M3_LC | QIVLSQSPAILSASPGEKVTMTCRASSSVSYIHWFQQKPGS |
| | | SPKPWIYATSNLASGVPVRFSGSGSGTSYSLTISRVEAEDA |
| | | ATYYCQQWTSNPPTFGGGTKLEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGECGGGGSGGGGSGGGGNECPELQPPVHGKIEPSQA |
| | | KYFFKDQVLVSCDTGYKVLKDNVEMDTFQIECLKDGTWSNK |
| | | IPTCKIVDCRAPGELEHGLITFSTRNNLTTYKSEIKYSCQE |
| | | PYYKMLNNNTGIYTCSAQGVWMNKVLGRSLPTCLPECGQPS |
| | | RSLPSLVKRIIGGRNAEPGLFPWQALIVVEDTSRVPNDKWF |
| | | GSGALLSASWILTAAHVLRSQRRDTTVIPVSKEHVTVYLGL |
| | | HDVRDKSGAVNSSAARVVLHPDFNIQNYNHDIALVQLQEPV |
| | | PLGPHVMPVCLPRLEPEGPAPHMLGLVAGWGISNPNVTVDE |
| | | IISSGTRTLSDVLQYVKLPVVPHAECKTSYESRSGNYSVTE |
| | | NMFCAGYYEGGKDTCLGDSGGAFVIFDDLSQRWVVQGLVSW |
| | | GGPEECGSKQVYGVYTKVSNYVDWVWEQMGLPQSVVEPQVE |
| | | R |
| 15 | M3-RTX(L)_LC | NECPELQPPVHGKIEPSQAKYFFKDQVLVSCDTGYKVLKDN |
| | | VEMDTFQIECLKDGTWSNKIPTCKIVDCRAPGELEHGLITF |
| | | STRNNLTTYKSEIKYSCQEPYYKMLNNNTGIYTCSAQGVWM |
| | | NKVLGRSLPTCLPECGQPSRSLPSLVKRIIGGRNAEPGLFP |
| | | WQALIVVEDTSRVPNDKWFGSGALLSASWILTAAHVLRSQR |
| | | RDTTVIPVSKEHVTVYLGLHDVRDKSGAVNSSAARVVLHPD |
| | | FNIQNYNHDIALVQLQEPVPLGPHVMPVCLPRLEPEGPAPH |
| | | MLGLVAGWGISNPNVTVDEIISSGTRTLSDVLQYVKLPVVP |
| | | HAECKTSYESRSGNYSVTENMFCAGYYEGGKDTCLGDSGGA |
| | | FVIFDDLSQRWVVQGLVSWGGPEECGSKQVYGVYTKVSNYV |
| | | DWVWEQMGLPQSVVEPQVERGGGGSGGGGSGGGGQIVLSQS |
| | | PAILSASPGEKVTMTCRASSSVSYIHWFQQKPGSSPKPWIY |
| | | ATSNLASGVPVRFSGSGSGTSYSLTISRVEAEDAATYYCQQ |
| | | WTSNPPTFGGGTKLEIKRTVAAPSVFIFPPSDEQLKSGTAS |
| | | VVCLLNNFYPREAKVQWKVDNALQSGNSQESVTEQDSKDST |
| | | YSLSSTLTLSKADYEKHKVYACEVTHQGLSSPVTKSFNRGE |
| | | C |
| 16 | RTX(H)^{ΔK}- | QVQLQQPGAELVKPGASVKMSCKASGYTFTSYNMHWVKQTP |
| | M1^{R504Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGNECPELQPPVHGKIEPSQAKYFFKDQVL |
| | | VSCDTGYKVLKDNVEMDTFQIECLKDGTWSNKIPTCKIVDC |
| | | RAPGELEHGLITFSTRNNLTTYKSEIKYSCQEPYYKMLNNN |
| | | TGIYTCSAQGVWMNKVLGRSLPTCLPVCGLPKFSRKLMARI |
| | | FNGRPAQKGTTPWIAMLSHLNGQPFCGGSLLGSSWIVTAAH |
| | | CLHQSLDPEDPTLQDSDLLSPSDFKIILGKHWRLRSDENEQ |
| | | HLGVKHTTLHPQYDPNTFENDVALVELLESPVLNAFVMPIC |
| | | LPEGPQQEGAMVIVSGWGKQFLQRFPETLMEIEIPIVDHST |
| | | CQKAYAPLKKKVTRDMICAGEKEGGKDACAGDSGGPMVTLN |
| | | RERGQWYLVGTVSWGDDCGKKDRYGVYSYIHHNKDWIQRVT |
| | | GVRN |
| 17 | M1^{R504Q}- | NECPELQPPVHGKIEPSQAKYFFKDQVLVSCDTGYKVLKDN |
| | RTX(L)_LC | VEMDTFQIECLKDGTWSNKIPTCKIVDCRAPGELEHGLITF |
| | | STRNNLTTYKSEIKYSCQEPYYKMLNNNTGIYTCSAQGVWM |
| | | NKVLGRSLPTCLPVCGLPKFSRKLMARIFNGRPAQKGTTPW |
| | | IAMLSHLNGQPFCGGSLLGSSWIVTAAHCLHQSLDPEDPTL |
| | | QDSDLLSPSDFKIILGKHWRLRSDENEQHLGVKHTTLHPQY |
| | | DPNTFENDVALVELLESPVLNAFVMPICLPEGPQQEGAMVI |
| | | VSGWGKQFLQRFPETLMEIEIPIVDHSTCQKAYAPLKKKVT |
| | | RDMICAGEKEGGKDACAGDSGGPMVTLNRERGQWYLVGTVS |
| | | WGDDCGKKDRYGVYSYIHHNKDWIQRVTGVRNGGGGSGGGG |
| | | SGGGGQIVLSQSPAILSASPGEKVTMTCRASSSVSYIHWFQ |
| | | QKPGSSPKPWIYATSNLASGVPVRFSGSGSGTSYSLTISRV |
| | | EAEDAATYYCQQWTSNPPTFGGGTKLEIKRTVAAPSVFIFP |
| | | PSDEQLKSGTASVVCLLNNFYPREAKVQWKVDNALQSGNSQ |
| | | ESVTEQDSKDSTYSLSSTLTLSKADYEKHKVYACEVTHQGL |
| | | SSPVTKSFNRGEC |
| 18 | RTX(H)^{ΔK}-C1r_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 19 | RTX(H)^{ΔK}-C1s_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 20 | RTX^{N297G}_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 21 | RTX(H)^{N297G,ΔK}- | QVQLQQPGAELVKPGASVKMSCKASGYTFTSYNMHWVKQTP |
| | C1r_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 22 | RTX(H)^{N297G,ΔK}- | QVQLQQPGAELVKPGASVKMSCKASGYTFTSYNMHWVKQTP |
| | C1r_^{S6S4A}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDAGGVFAVRD |
| | | PNTDRWVATGIVSWGIGCSRGYGFYTKVLNYVDWIKKEMEE |
| | | ED |
| 23 | RTX(H)^{N297G,ΔK}- | QVQLQQPGAELVKPGASVKMSCKASGYTFTSYNMHWVKQTP |
| | C1s_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 24 | RTX(H)^{N297G,ΔK}- | QVQLQQPGAELVKPGASVKMSCKASGYTFTSYNMHWVKQTP |
| | C1s^{S632A}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDAGGAFAVQDPNDKTKF |
| | | YAAGLVSWGPQCGTYGLYTRVKNYVDWIMKTMQENSTPRED |
| 25 | RTX(H)^{ΔK}- | QVQLQQPGAELVKPGASVKMSCKASGYTFTSYNMHWVKQTP |
| | C2a_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGKIQIQRSGHLNLYLLLDCSQSVSENDFL |
| | | IFKESASLMVDRIFSFEINVSVAIITFASEPKVLMSVLNDN |
| | | SRDMTEVISSLENANYKDHENGTGTNTYAALNSVYLMMNNQ |
| | | MRLLGMETMAWQEIRHAIILLTDGKSNMGGSPKTAVDHIRE |
| | | ILNINQKRNDYLDIYAIGVGKLDVDWRELNELGSKKDGERH |
| | | AFILQDTKALHQVFEHMLDVSKLTDTICGVGNMSANASDQE |
| | | RTPWHVTIKPKSQETCRGALISDQWVLTAAHCFRDGNDHSL |
| | | WRVNVGDPKSQWGKEELIEKAVISPGFDVFAKKNQGILEFY |
| | | GDDIALLKLAQKVKMSTHARPIGLPCTMEANLALRRPQGST |
| | | CRDHENELLNKQSVPAHFVALNGSKLNINLKMGVEWTSCAE |
| | | VVSQEKTMFPNLTDVREVVTDQFLCSGTQEDESPCKGESGG |
| | | AVFLERRFRFFQVGLVSWGLYNPCLGSADKNSRKRAPRSKV |
| | | PPPRDFHINLFRMQPWLRQHLGDVLNFLPL |
| 26 | RTX(H)^{ΔK}-Bb_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGKIVLDPSGSMNIYLVLDGSDSIGASNET |
| | | GAKKCLVNLIEKVASYGVKPRYGLVTYATYPKIWVKVSEAD |
| | | SSNADWVTKQLNEINYEDHKLKSGTNTKKALQAVYSMMSWP |
| | | DDVPPEGWNRTRHVIILMTDGLHNMGGDPITVIDEIRDLLY |
| | | IGKDRKNPREDYLDVYVFGVGPLVNQVNINALASKKDNEQH |
| | | VFKVKDMENLEDVFYQMIDESQSLSLCGMVWEHRKGTDYHK |
| | | QPWQAKISVIRPSKGHESCMGAVVSEYFVLTAAHCFTVDDK |
| | | EHSIKVSVGGEKRDLEIEVVLFHPNYNINGKKEAGIPEFYD |
| | | YDVALIKLKNKLKYGQTIRPICLPCTEGTTRALRLPPTTTC |
| | | QQQKEELLPAQDIKALFVSEEEKKLTRKEVYIKNGDKKGSC |
| | | ERDAQYAPGYDKVKDISEVVTPRFLCTGGVSPYADPNTCRG |
| | | DSGGPLIVHKRSRFIQVGVISWGVVDVCKNQKRQKQVPAHA |
| | | RDFHINLFQVLPWLKEKLQDEDLGFL |
| 27 | RTX(H)^{ΔK}- | QVQLQQPGAELVKPGASVKMSCKASGYTFTSYNMHWVKQTP |
| | MatCFD_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGILGGREAEAHARPYMASVQLNGAHLCGG |
| | | VLVAEQWVLSAAHCLEDAADGKVQVLLGAHSLSQPEPSKRL |
| | | YDVLRAVPHPDSQPDTIDHDLLLLQLSEKATLGPAVRPLPW |
| | | QRVDRDVAPGTLCDVAGWGIVNHAGRRPDSLQHVLLPVLDR |
| | | ATCNRRTHHDGAITERLMCAESNRRDSCKGDSGGPLVCGGV |
| | | LEGWTSGSRVCGNRKKPGIYTRVASYAAWIDSVLA |
| 28 | MatCFD- | ILGGREAEAHARPYMASVQLNGAHLCGGVLVAEQWVLSAAH |
| | RTX(H)_HC | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDSGGPLVCGGVLEGVVTSGSRVCG |
| | | NRKKPGIYTRVASYAAWIDSVLAGGGGSGGGGSGGGGQVQL |
| | | QQPGAELVKPGASVKMSCKASGYTFTSYNMHWVKQTPGRGL |
| | | EWIGAIYPGNGDTSYNQKFKGKATLTADKSSSTAYMQLSSL |
| | | TSEDSAVYYCARSTYYGGDWYFNVWGAGTTVTVSAASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSCDKTHTCPPCPAPELLGGPSVFLFPPK |
| | | PKDTLMISRTPEVTCVVVDVSHEDPEVKFNWYVDGVEVHNA |
| | | KTKPREEQYNSTYRVVSVLTVLHQDWLNGKEYKCKVSNKAL |
| | | PAPIEKTISKAKGQPREPQVYTLPPSRDELTKNQVSLTCLV |
| | | KGFYPSDIAVEWESNGQPENNYKTTPPVLDSDGSFFLYSKL |
| | | TVDKSRWQQGNVFSCSVMHEALHNHYTQKSLSLSPGK |
| 29 | RTX(L)- | QIVLSQSPAILSASPGEKVTMTCRASSSVSYIHWFQQKPGS |
| | MatCFD_LC | SPKPWIYATSNLASGVPVRFSGSGSGTSYSLTISRVEAEDA |
| | | ATYYCQQWTSNPPTFGGGTKLEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVT |
| | | KSFNRGECGGGGSGGGGSGGGGILGGREAEAHARPYMASVQ |
| | | LNGAHLCGGVLVAEQWVLSAAHCLEDAADGKVQVLLGAHSL |
| | | SQPEPSKRLYDVLRAVPHPDSQPDTIDHDLLLLQLSEKATL |
| | | GPAVRPLPWQRVDRDVAPGTLCDVAGWGIVNHAGRRPDSLQ |
| | | HVLLPVLDRATCNRRTHHDGAITERLMCAESNRRDSCKGDS |
| | | GGPLVCGGVLEGVVTSGSRVCGNRKKPGIYTRVASYAAWID |
| | | SVLA |
| 30 | MatCFD- | ILGGREAEAHARPYMASVQLNGAHLCGGVLVAEQWVLSAAH |
| | RTX(L)_LC | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDSGGPLVCGGVLEGVVTSGSRVCG |
| | | NRKKPGIYTRVASYAAWIDSVLAGGGGSGGGGSGGGGQIVL |
| | | SQSPAILSASPGEKVTMTCRASSSVSYIHWFQQKPGSSPKP |
| | | WIYATSNLASGVPVRFSGSGSGTSYSLTISRVEAEDAATYY |
| | | CQQWTSNPPTFGGGTKLEIKRTVAAPSVFIFPPSDEQLKSG |
| | | TASVVCLLNNFYPREAKVQWKVDNALQSGNSQESVTEQDSK |
| | | DSTYSLSSTLTLSKADYEKHKVYACEVTHQGLSSPVTKSFN |
| | | RGEC |
| 31 | MatCFD^{S208A}- | ILGGREAEAHARPYMASVQLNGAHLCGGVLVAEQWVLSAAH |
| | RTX(H)_HC | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDAGGPLVCGGVLEGVVTSGSRVCG |
| | | NRKKPGIYTRVASYAAWIDSVLAGGGGSGGGGSGGGGQVQL |
| | | QQPGAELVKPGASVKMSCKASGYTFTSYNMHWVKQTPGRGL |
| | | EWIGAIYPGNGDTSYNQKFKGKATLTADKSSSTAYMQLSSL |
| | | TSEDSAVYYCARSTYYGGDWYFNVWGAGTTVTVSAASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSCDKTHTCPPCPAPELLGGPSVFLFPPK |
| | | PKDTLMISRTPEVTCVVVDVSHEDPEVKFNWYVDGVEVHNA |
| | | KTKPREEQYNSTYRVVSVLTVLHQDWLNGKEYKCKVSNKAL |
| | | PAPIEKTISKAKGQPREPQVYTLPPSRDELTKNQVSLTCLV |
| | | KGFYPSDIAVEWESNGQPENNYKTTPPVLDSDGSFFLYSKL |
| | | TVDKSRWQQGNVFSCSVMHEALHNHYTQKSLSLSPGK |
| 32 | ProCFD- | APPRGRILGGREAEAHARPYMASVQLNGAHLCGGVLVAEQW |
| | RTX(H)_HC | VLSAAHCLEDAADGKVQVLLGAHSLSQPEPSKRLYDVLRAV |
| | | PHPDSQPDTIDHDLLLLQLSEKATLGPAVRPLPWQRVDRDV |
| | | APGTLCDVAGWGIVNHAGRRPDSLQHVLLPVLDRATCNRRT |
| | | HHDGAITERLMCAESNRRDSCKGDSGGPLVCGGVLEGVVTS |
| | | GSRVCGNRKKPGIYTRVASYAAWIDSVLAGGGGSGGGGSGG |
| | | GGQVQLQQPGAELVKPGASVKMSCKASGYTFTSYNMHWVKQ |
| | | TPGRGLEWIGAIYPGNGDTSYNQKFKGKATLTADKSSSTAY |
| | | MQLSSLTSEDSAVYYCARSTYYGGDWYFNVWGAGTTVTVSA |
| | | ASTKGPSVFPLAPSSKSTSGGTAALGCLVKDYFPEPVTVSW |
| | | NSGALTSGVHTFPAVLQSSGLYSLSSVVTVPSSSLGTQTYI |
| | | CNVNHKPSNTKVDKKVEPKSCDKTHTCPPCPAPELLGGPSV |
| | | FLFPPKPKDTLMISRTPEVTCVVVDVSHEDPEVKFNWYVDG |
| | | VEVHNAKTKPREEQYNSTYRVVSVLTVLHQDWLNGKEYKCK |
| | | VSNKALPAPIEKTISKAKGQPREPQVYTLPPSRDELTKNQV |
| | | SLTCLVKGFYPSDIAVEWESNGQPENNYKTTPPVLDSDGSF |
| | | FLYSKLTVDKSRWQQGNVFSCSVMHEALHNHYTQKSLSLSP |
| | | GK |
| 33 | RTX(H)^{K121Q,ΔK}- | QVQLQQPGAELVKPGASVKMSCKASGYTFTSYNMHWVKQTP |
| | M2^{R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TQGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 34 | RTX(H)^{ΔK}- | QVQLQQPGAELVKPGASVKMSCKASGYTFTSYNMHWVKQTP |
| | M2^{K317Q,R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAQYILKDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 35 | RTX(H)^{ΔK}- | QVQLQQPGAELVKPGASVKMSCKASGYTFTSYNMHWVKQTP |
| | M2^{K321Q,R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILQDSFS |
| | | IFCETGYELLQGHLPLKSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 36 | RTX(H)^{ΔK}- | QVQLQQPGAELVKPGASVKMSCKASGYTFTSYNMHWVKQTP |
| | M2^{K342Q,R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLQSFTAVCQKDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 37 | RTX(H)^{ΔK}- | QVQLQQPGAELVKPGASVKMSCKASGYTFTSYNMHWVKQTP |
| | M2^{K350Q,R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQQDGSWDRPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 38 | RTX(H)^{ΔK}- | QVQLQQPGAELVKPGASVKMSCKASGYTFTSYNMHWVKQTP |
| | M2^{R356Q,R444K}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGQPCPYPMAPPNGHVSPVQAKYILKDSFS |
| | | IFCETGYELLQGHLPLKSFTAVCQKDGSWDQPMPACSIVDC |
| | | GPPDDLPSGRVEYITGPGVTTYKAVIQYSCEETFYTMKVND |
| | | GKYVCEADGFWTSSKGEKSLPVCEPVCGLSARTTGGKIYGG |
| | | QKAKPGDFPWQVLILGGTTAAGALLYDNWVLTAAHAVYEQK |
| | | HDASALDIRMGTLKRLSPHYTQAWSEAVFIHEGYTHDAGFD |
| | | NDIALIKLNNKVVINSNITPICLPRKEAESFMRTDDIGTAS |
| | | GWGLTQRGFLARNLMYVDIPIVDHQKCTAAYEKPPYPRGSV |
| | | TANMLCAGLESGGKDSCRGDSGGALVFLDSETERWFVGGIV |
| | | SWGSMNCGEAGQYGVYTKVINYIPWIENIISDF |
| 39 | RTX(H)^{N2970,ΔK}- | QVQLQQPGAELVKPGASVKMSCKASGYTFTSYNMHWVKQTP |
| | C1r^{K374Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIQD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 40 | RTX(H)^{N297G,ΔK}- | QVQLQQPGAELVKPGASVKMSCKASGYTFTSYNMHWVKQTP |
| | C1r^{R380Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPQNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 41 | RTX(H)^{N297G,ΔK}- | QVQLQQPGAELVKPGASVKMSCKASGYTFTSYNMHWVKQTP |
| | C1s^{K30SQ}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAQAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 42 | RTX(H)^{N297G,ΔK}- | QVQLQQPGAELVKPGASVKMSCKASGYTFTSYNMHWVKQTP |
| | C1s^{K310Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAQYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKE |
| | | YAAGLVSWGPQCGTYGLYTRVKNYVDWIMKTMQENSTPRED |
| 43 | RTX(H)^{N297G,ΔK}- | QVQLQQPGAELVKPGASVKMSCKASGYTFTSYNMHWVKQTP |
| | C1s^{R314Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFQDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 44 | RTX(H)^{N297G,ΔK}- | QVQLQQPGAELVKPGASVKMSCKASGYTFTSYNMHWVKQTP |
| | C1s^{R331Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGQVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKE |
| | | YAAGLVSWGPQCGTYGLYTRVKNYVDWIMKTMQENSTPRED |
| 45 | RTX(H)^{N297G,ΔK}- | QVQLQQPGAELVKPGASVKMSCKASGYTFTSYNMHWVKQTP |
| | C1s^{K346Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGQWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 46 | RTX(H)^{N297G,ΔK}- | QVQLQQPGAELVKPGASVKMSCKASGYTFTSYNMHWVKQTP |
| | C1s^{K351Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSQLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKE |
| | | YAAGLVSWGPQCGTYGLYTRVKNYVDWIMKTMQENSTPRED |
| 47 | RTX(H)^{N297G,ΔK}- | QVQLQQPGAELVKPGASVKMSCKASGYTFTSYNMHWVKQTP |
| | C1s^{K353Q}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLQCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 48 | RTX(H)^{N297G,ΔK}- | QVQLQQPGAELVKPGASVKMSCKASGYTFTSYNMHWVKQTP |
| | C1r^{H484W}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIWGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 49 | RTX(H)^{N297G,ΔK}- | QVQLQQPGAELVKPGASVKMSCKASGYTFTSYNMHWVKQTP |
| | C1r^{G485W}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHWRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 50 | RTX(H)^{N297G,ΔK}- | QVQLQQPGAELVKPGASVKMSCKASGYTFTSYNMHWVKQTP |
| | C1r^{R486W}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGWGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 51 | RTX(H)^{N297G,ΔK}- | QVQLQQPGAELVKPGASVKMSCKASGYTFTSYNMHWVKQTP |
| | C1s^{D456W}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFWNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 52 | RTX(H)^{N297G,ΔK}- | QVQLQQPGAELVKPGASVKMSCKASGYTFTSYNMHWVKQTP |
| | C1s^{N457W}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDWPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 53 | RTX(H)^{N297G,ΔK}- | QVQLQQPGAELVKPGASVKMSCKASGYTFTSYNMHWVKQTP |
| | C1s^{P458W}_HC | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNWWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 54 | RTX(H)^{N297G,ΔK}_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYGSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| 55 | RTX(H)^{K121Q}_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TQGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 56 | RTX(H)^{K121Q,ΔK}_HC | QVQLQQPGAELVKPGASVKMSCKASGYTFTSYNMHWVKQTP |
| | | GRGLEWIGAIYPGNGDTSYNQKFKGKATLTADKSSSTAYMQ |
| | | LSSLTSEDSAVYYCARSTYYGGDWYFNVWGAGTTVTVSAAS |
| | | TQGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| 57 | MASP-2 CCP1/2SP | QPCPYPMAPPNGHVSPVQAKYILKDSFSIFCETGYELLQGH |
| | | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGRIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDE |
| 58 | MASP-2^{R444K} | QPCPYPMAPPNGHVSPVQAKYILKDSFSIFCETGYELLQGH |
| | CCP1/2SP | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 59 | MASP-2^{R444Q} | QPCPYPMAPPNGHVSPVQAKYILKDSFSIFCETGYELLQGH |
| | CCP1/2SP | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGQIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 60 | MASP-2^{S633A} | QPCPYPMAPPNGHVSPVQAKYILKDSFSIFCETGYELLQGH |
| | CCP1/2SP | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDAGGALVFLDSETERWFVGGIVSWGSMNCGEAGQY |
| | | GVYTKVINYIPWIENIISDF |
| 61 | MASP-2^{K317Q,R444K} | QPCPYPMAPPNGHVSPVQAQYILKDSFSIFCETGYELLQGH |
| | CCP1/2SP | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 62 | MASP-2^{K321Q,R444K} | QPCPYPMAPPNGHVSPVQAKYILQDSFSIFCETGYELLQGH |
| | CCP1/2SP | LPLKSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESEMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 63 | MASP-2^{K342Q,R444K} | QPCPYPMAPPNGHVSPVQAKYILKDSFSIFCETGYELLQGH |
| | CCP1/2SP | LPLQSFTAVCQKDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 64 | MASP-2^{K350Q,R444K} | QPCPYPMAPPNGHVSPVQAKYILKDSFSIFCETGYELLQGH |
| | CCP1/2SP | LPLKSFTAVCQQDGSWDRPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 65 | MASP-2^{R356Q,R444K} | QPCPYPMAPPNGHVSPVQAKYILKDSFSIFCETGYELLQGH |
| | CCP1/2SP | LPLKSFTAVCQKDGSWDQPMPACSIVDCGPPDDLPSGRVEY |
| | | ITGPGVTTYKAVIQYSCEETFYTMKVNDGKYVCEADGFWTS |
| | | SKGEKSLPVCEPVCGLSARTTGGKIYGGQKAKPGDFPWQVL |
| | | ILGGTTAAGALLYDNWVLTAAHAVYEQKHDASALDIRMGTL |
| | | KRLSPHYTQAWSEAVFIHEGYTHDAGFDNDIALIKLNNKVV |
| | | INSNITPICLPRKEAESFMRTDDIGTASGWGLTQRGFLARN |
| | | LMYVDIPIVDHQKCTAAYEKPPYPRGSVTANMLCAGLESGG |
| | | KDSCRGDSGGALVFLDSETERWFVGGIVSWGSMNCGEAGQY |
| | | GVYTKVINYIPWIENIISDF |
| 66 | MASP-3 CCP1/2SP | NECPELQPPVHGKIEPSQAKYFFKDQVLVSCDTGYKVLKDN |
| | | VEMDTFQIECLKDGTWSNKIPTCKIVDCRAPGELEHGLITF |
| | | STRNNLTTYKSEIKYSCQEPYYKMLNNNTGIYTCSAQGVWM |
| | | NKVLGRSLPTCLPECGQPSRSLPSLVKRIIGGRNAEPGLFP |
| | | WQALIVVEDTSRVPNDKWFGSGALLSASWILTAAHVLRSQR |
| | | RDTTVIPVSKEHVTVYLGLHDVRDKSGAVNSSAARVVLHPD |
| | | FNIQNYNHDIALVQLQEPVPLGPHVMPVCLPRLEPEGPAPH |
| | | MLGLVAGWGISNPNVTVDEIISSGTRTLSDVLQYVKLPVVP |
| | | HAECKTSYESRSGNYSVTENMFCAGYYEGGKDTCLGDSGGA |
| | | FVIFDDLSQRWVVQGLVSWGGPEECGSKQVYGVYTKVSNYV |
| | | DWVWEQMGLPQSWEPQVER |
| 67 | MASP-1 CCP1/2SP | NECPELQPPVHGKIEPSQAKYFFKDQVLVSCDTGYKVLKDN |
| | | VEMDTFQIECLKDGTWSNKIPTCKIVDCRAPGELEHGLITF |
| | | STRNNLTTYKSEIKYSCQEPYYKMLNNNTGIYTCSAQGVWM |
| | | NKVLGRSLPTCLPVCGLPKFSRKLMARIFNGRPAQKGTTPW |
| | | IAMLSHLNGQPFCGGSLLGSSWIVTAAHCLHQSLDPEDPTL |
| | | RDSDLLSPSDFKIILGKHWRLRSDENEQHLGVKHTTLHPQY |
| | | DPNTFENDVALVELLESPVLNAFVMPICLPEGPQQEGAMVI |
| | | VSGWGKQFLQRFPETLMEIEIPIVDHSTCQKAYAPLKKKVT |
| | | RDMICAGEKEGGKDACAGDSGGPMVTLNRERGQWYLVGTVS |
| | | WGDDCGKKDRYGVYSYIHHNKDWIQRVTGVRN |
| 68 | MASP-1^{R504Q} | NECPELQPPVHGKIEPSQAKYFFKDQVLVSCDTGYKVLKDN |
| | CCP1/2SP | VEMDTFQIECLKDGTWSNKIPTCKIVDCRAPGELEHGLITF |
| | | STRNNLTTYKSEIKYSCQEPYYKMLNNNTGIYTCSAQGVWM |
| | | NKVLGRSLPTCLPVCGLPKFSRKLMARIFNGRPAQKGTTPW |
| | | IAMLSHLNGQPFCGGSLLGSSWIVTAAHCLHQSLDPEDPTL |
| | | QDSDLLSPSDFKIILGKHWRLRSDENEQHLGVKHTTLHPQY |
| | | DPNTFENDVALVELLESPVLNAFVMPICLPEGPQQEGAMVI |
| | | VSGWGKQFLQRFPETLMEIEIPIVDHSTCQKAYAPLKKKVT |
| | | RDMICAGEKEGGKDACAGDSGGPMVTLNRERGQWYLVGTVS |
| | | WGDDCGKKDRYGVYSYIHHNKDWIQRVTGVRN |
| 69 | C1r CCP1/2SP | IKCPQPKTLDEFTIIQNLQPQYQFRDYFIATCKQGYQLIEG |
| | | NQVLHSFTAVCQDDGTWHRAMPRCKIKDCGQPRNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIHGRGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDSGGVFAVRDPNTDRWVATGIVS |
| | | WGIGCSRGYGFYTKVLNYVDWIKKEMEEED |
| 70 | C1r^{K374Q} CCP1/2SP | IKCPQPKTLDEFTIIQNLQPQYQFRDYFIATCKQGYQLIEG |
| | | NQVLHSFTAVCQDDGTWHRAMPRCKIQDCGQPRNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIHGRGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDSGGVFAVRDPNTDRWVATGIVS |
| | | WGIGCSRGYGFYTKVLNYVDWIKKEMEEED |
| 71 | C1r^{R380Q} CCP1/2SP | IKCPQPKTLDEFTIIQNLQPQYQFRDYFIATCKQGYQLIEG |
| | | NQVLHSFTAVCQDDGTWHRAMPRCKIKDCGQPQNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIHGRGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDSGGVFAVRDPNTDRWVATGIVS |
| | | WGIGCSRGYGFYTKVLNYVDWIKKEMEEED |
| 72 | C1r^{H484W} CCP1/2SP | IKCPQPKTLDEFTIIQNLQPQYQFRDYFIATCKQGYQLIEG |
| | | NQVLHSFTAVCQDDGTWHRAMPRCKIKDCGQPRNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIWGRGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDSGGVFAVRDPNTDRWVATGIVS |
| | | WGIGCSRGYGFYTKVLNYVDWIKKEMEEED |
| 73 | C1r^{G485W} CCP1/2SP | IKCPQPKTLDEFTIIQNLQPQYQFRDYFIATCKQGYQLIEG |
| | | NQVLHSFTAVCQDDGTWHRAMPRCKIKDCGQPRNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIHWRGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDSGGVFAVRDPNTDRWVATGIVS |
| | | WGIGCSRGYGFYTKVLNYVDWIKKEMEEED |
| 74 | C1r^{R486W} CCP1/2SP | IKCPQPKTLDEFTIIQNLQPQYQFRDYFIATCKQGYQLIEG |
| | | NQVLHSFTAVCQDDGTWHRAMPRCKIKDCGQPRNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIHGWGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDSGGVFAVRDPNTDRWVATGIVS |
| | | WGIGCSRGYGFYTKVLNYVDWIKKEMEEED |
| 75 | C1r^{S654A} CCP1/2SP | IKCPQPKTLDEFTIIQNLQPQYQFRDYFIATCKQGYQLIEG |
| | | NQVLHSFTAVCQDDGTWHRAMPRCKIKDCGQPRNLPNGDFR |
| | | YTTTMGVNTYKARIQYYCHEPYYKMQTRAGSRESEQGVYTC |
| | | TAQGIWKNEQKGEKIPRCLPVCGKPVNPVEQRQRIIGGQKA |
| | | KMGNFPWQVFTNIHGRGGGALLGDRWILTAAHTLYPKEHEA |
| | | QSNASLDVFLGHTNVEELMKLGNHPIRRVSVHPDYRQDESY |
| | | NFEGDIALLELENSVTLGPNLLPICLPDNDTFYDLGLMGYV |
| | | SGFGVMEEKIAHDLRFVRLPVANPQACENWLRGKNRMDVFS |
| | | QNMFCAGHPSLKQDACQGDAGGVFAVRDPNTDRWVATGIVS |
| | | WGIGCSRGYGFYTKVLNYVDWIKKEMEEED |
| 76 | C1s CCP1/2SP | MPCPKEDTPNSVWEPAKAKYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 77 | C1s^{S632A} CCP1/2SP | MPCPKEDTPNSVWEPAKAKYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDAGGAFAVQDPNDKTKFYAAGLVSWGPQCG |
| | | TYGLYTRVKNYVDWIMKTMQENSTPRED |
| 78 | C1s^{K308Q} CCP1/2SP | MPCPKEDTPNSVWEPAQAKYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 79 | C1s^{K310Q} CCP1/2SP | MPCPKEDTPNSVWEPAKAQYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 80 | C1s^{R314Q} CCP1/2SP | MPCPKEDTPNSVWEPAKAKYVFQDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 81 | C1s^{R331Q} CCP1/2SP | MPCPKEDTPNSVWEPAKAKYVFRDVVQITCLDGFEVVEGQV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 82 | C1s^{K346Q} CCP1/2SP | MPCPKEDTPNSVWEPAKAKYVFRDVVQITCLDGFEVVEGRV |
| | | GATSFYSTCQSNGQWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 83 | C1s^{K351Q} CCP1/2SP | MPCPKEDTPNSVWEPAKAKYVFRDVVQITCLDGFEVVEGRV |
| | | GATSFYSTCQSNGKWSNSQLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 84 | C1s^{K353Q} CCP1/2SP | MPCPKEDTPNSVWEPAKAKYVFRDVVQITCLDGFEVVEGRV |
| | | GATSFYSTCQSNGKWSNSKLQCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 85 | C1s^{D456W} CCP1/2SP | MPCPKEDTPNSVWEPAKAKYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | WNPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 86 | C1s^{N457W} CCP1/2SP | MPCPKEDTPNSVWEPAKAKYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DWPWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 87 | C1s^{P458W} CCP1/2SP | MPCPKEDTPNSVWEPAKAKYVFRDVVQITCLDGFEVVEGRV |
| | | GATSFYSTCQSNGKWSNSKLKCQPVDCGIPESIENGKVEDP |
| | | ESTLFGSVIRYTCEEPYYYMENGGGGEYHCAGNGSWVNEVL |
| | | GPELPKCVPVCGVPREPFEEKQRIIGGSDADIKNFPWQVFF |
| | | DNWWAGGALINEYWVLTAAHVVEGNREPTMYVGSTSVQTSR |
| | | LAKSKMLTPEHVFIHPGWKLLEVPEGRTNFDNDIALVRLKD |
| | | PVKMGPTVSPICLPGTSSDYNLMDGDLGLISGWGRTEKRDR |
| | | AVRLKAARLPVAPLRKCKEVKVEKPTADAEAYVFTPNMICA |
| | | GGEKGMDSCKGDSGGAFAVQDPNDKTKFYAAGLVSWGPQCG |
| | | TYGLYTRVKNYVDWIMKTMQENSTPRED |
| 88 | C2a VWFA-SP | KIQIQRSGHLNLYLLLDCSQSVSENDFLIFKESASLMVDRI |
| | | FSFEINVSVAIITFASEPKVLMSVLNDNSRDMTEVISSLEN |
| | | ANYKDHENGTGTNTYAALNSVYLMMNNQMRLLGMETMAWQE |
| | | IRHAIILLTDGKSNMGGSPKTAVDHIREILNINQKRNDYLD |
| | | IYAIGVGKLDVDWRELNELGSKKDGERHAFILQDTKALHQV |
| | | FEHMLDVSKLTDTICGVGNMSANASDQERTPWHVTIKPKSQ |
| | | ETCRGALISDQWVLTAAHCFRDGNDHSLWRVNVGDPKSQWG |
| | | KEFLIEKAVISPGFDVFAKKNQGILEFYGDDIALLKLAQKV |
| | | KMSTHARPICLPCTMEANLALRRPQGSTCRDHENELLNKQS |
| | | VPAHFVALNGSKLNINLKMGVEWTSCAEVVSQEKTMFPNLT |
| | | DVREVVTDQFLCSGTQEDESPCKGESGGAVFLERRFRFFQV |
| | | GLVSWGLYNPCLGSADKNSRKRAPRSKVPPPRDFHINLFRM |
| | | QPWLRQHLGDVLNFLPL |
| 89 | Bb VWFA-SP | KIVLDPSGSMNIYLVLDGSDSIGASNFTGAKKCLVNLIEKV |
| | | ASYGVKPRYGLVTYATYPKIWVKVSEADSSNADWVTKQLNE |
| | | INYEDHKLKSGTNTKKALQAVYSMMSWPDDVPPEGWNRTRH |
| | | VIILMTDGLHNMGGDPITVIDEIRDLLYIGKDRKNPREDYL |
| | | DVYVFGVGPLVNQVNINALASKKDNEQHVFKVKDMENLEDV |
| | | FYQMIDESQSLSLCGMVWEHRKGTDYHKQPWQAKISVIRPS |
| | | KGHESCMGAVVSEYFVLTAAHCFTVDDKEHSIKVSVGGEKR |
| | | DLEIEVVLFHPNYNINGKKEAGIPEFYDYDVALIKLKNKLK |
| | | YGQTIRPICLPCTEGTTRALRLPPTTTCQQQKEELLPAQDI |
| | | KALFVSEEEKKLTRKEVYIKNGDKKGSCERDAQYAPGYDKV |
| | | KDISEVVTPRFLCTGGVSPYADPNTCRGDSGGPLIVHKRSR |
| | | FIQVGVISWGVVDVCKNQKRQKQVPAHARDFHINLFQVLPW |
| | | LKEKLQDEDLGFL |
| 90 | MatCFD | ILGGREAEAHARPYMASVQLNGAHLCGGVLVAEQWVLSAAH |
| | | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDSGGPLVCGGVLEGVVTSGSRVCG |
| | | NRKKPGIYTRVASYAAWIDSVLA |
| 91 | MatCFD^{S208A} | ILGGREAEAHARPYMASVQLNGAHLCGGVLVAEQWVLSAAH |
| | | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDAGGPLVCGGVLEGVVTSGSRVCG |
| | | NRKKPGIYTRVASYAAWIDSVLA |
| 92 | ProCFD | APPRGRILGGREAEAHARPYMASVQLNGAHLCGGVLVAEQW |
| | | VLSAAHCLEDAADGKVQVLLGAHSLSQPEPSKRLYDVLRAV |
| | | PHPDSQPDTIDHDLLLLQLSEKATLGPAVRPLPWQRVDRDV |
| | | APGTLCDVAGWGIVNHAGRRPDSLQHVLLPVLDRATCNRRT |
| | | HHDGAITERLMCAESNRRDSCKGDSGGPLVCGGVLEGVVTS |
| | | GSRVCGNRKKPGIYTRVASYAAWIDSVLA |
| 93 | ALM_HC | QVQLQESGPGLVRPSQTLSLTCTVSGFTFTDFYMNWVRQPP |
| | | GRGLEWIGFIRDKAKGYTTEYNPSVKGRVTMLVDTSKNQFS |
| | | LRLSSVTAADTAVYYCAREGHTAAPFDYWGQGSLVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 94 | ALM_LC | DIQMTQSPSSLSASVGDRVTITCKASQNIDKYLNWYQQKPG |
| | | KAPKLLIYNTNNLQTGVPSRFSGSGSGTDFTFTISSLQPED |
| | | IATYYCLQHISRPRTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 95 | DARA_HC | EVQLLESGGGLVQPGGSLRLSCAVSGFTFNSFAMSWVRQAP |
| | | GKGLEWVSAISGSGGGTYYADSVKGRFTISRDNSKNTLYLQ |
| | | MNSLRAEDTAVYFCAKDKILWFGEPVFDYWGQGTLVTVSSA |
| | | STKGPSVFPLAPSSKSTSGGTAALGCLVKDYFPEPVTVSWN |
| | | SGALTSGVHTFPAVLQSSGLYSLSSVVTVPSSSLGTQTYIC |
| | | NVNHKPSNTKVDKKVEPKSCDKTHTCPPCPAPELLGGPSVF |
| | | LFPPKPKDTLMISRTPEVTCVVVDVSHEDPEVKFNWYVDGV |
| | | EVHNAKTKPREEQYNSTYRVVSVLTVLHQDWLNGKEYKCKV |
| | | SNKALPAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVMHEALHNHYTQKSLSLSPG |
| | | K |
| 96 | DARA_LC | EIVLTQSPATLSLSPGERATLSCRASQSVSSYLAWYQQKPG |
| | | QAPRLLIYDASNRATGIPARFSGSGSGTDFTLTISSLEPED |
| | | FAVYYCQQRSNWPPTFGQGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 97 | MatCFD- | ILGGREAEAHARPYMASVQLNGAHLCGGVLVAEQWVLSAAH |
| | ALM(H)_HC | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDSGGPLVCGGVLEGVVTSGSRVCG |
| | | NRKKPGIYTRVASYAAWIDSVLAGGGGSGGGGSGGGGQVQL |
| | | QESGPGLVRPSQTLSLTCTVSGFTFTDFYMNWVRQPPGRGL |
| | | EWIGFIRDKAKGYTTEYNPSVKGRVTMLVDTSKNQFSLRLS |
| | | SVTAADTAVYYCAREGHTAAPFDYWGQGSLVTVSSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSCDKTHTCPPCPAPELLGGPSVFLFPPK |
| | | PKDTLMISRTPEVTCVVVDVSHEDPEVKFNWYVDGVEVHNA |
| | | KTKPREEQYNSTYRVVSVLTVLHQDWLNGKEYKCKVSNKAL |
| | | PAPIEKTISKAKGQPREPQVYTLPPSRDELTKNQVSLTCLV |
| | | KGFYPSDIAVEWESNGQPENNYKTTPPVLDSDGSFFLYSKL |
| | | TVDKSRWQQGNVFSCSVMHEALHNHYTQKSLSLSPGK |
| 98 | MatCFD- | ILGGREAEAHARPYMASVQLNGAHLCGGVLVAEQWVLSAAH |
| | DARA(H)_HC | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDSGGPLVCGGVLEGVVTSGSRVCG |
| | | NRKKPGIYTRVASYAAWIDSVLAGGGGSGGGGSGGGGEVQL |
| | | LESGGGLVQPGGSLRLSCAVSGFTFNSFAMSWVRQAPGKGL |
| | | EWVSAISGSGGGTYYADSVKGRFTISRDNSKNTLYLQMNSL |
| | | RAEDTAVYFCAKDKILWFGEPVFDYWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |
| 99 | Linker | GGGGS |
| 100 | Linker | GGGGSGGGGSGGGG |
| 101 | aN7_HC | EVQLQQSGAELVRPGALVKLSCKASGFNIKDFYMHWVKQRP |
| | | DQGLEWIGWIDPENDNTIYDPKFQGKASITADTSSNTAYLQ |
| | | LSSLTSEDTAVYYCAYRSPMITTGAMDFWGPGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 102 | aN7_LC | DIVLTQSPASLAVSLGQRATISGRASESVDNYGIGFMNWFQ |
| | | QKPGQPPKLLIYVASNQGSGVPARFSGSGSGTDFSLNIHPM |
| | | EEDDAAMYFCQQSKEVPFTFGSGTKLEIKRTVAAPSVFIFP |
| | | PSDEQLKSGTASVVCLLNNFYPREAKVQWKVDNALQSGNSQ |
| | | ESVTEQDSKDSTYSLSSTLTLSKADYEKHKVYACEVTHQGL |
| | | SSPVTKSFNRGEC |
| 103 | aN19_HC | EVQLVESGGGLVQPKGSLKLSCAASGETFNMYVMNWVRQAP |
| | | GKGLEWVARIRSKSYNFGTYYADSVKDRFTISRDDSQSVLY |
| | | LTMNNLKTEDTAMYYCVRLDDDFAYWGPGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |
| 104 | aN19_LC | DIVMTQAAPSVSVSLGESVSIYCRSNKSLLYSNGNTYLYWF |
| | | LQRPGQSPQLLIYRMSNLASGVPDRFSGSGSGTAFTLRISR |
| | | VEAEDVGVYYCLQHLEYPLTFGAGTKLELKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| 105 | aN5_HC | EVQLQQSGAELVRPGALVKLSCKASGFNIKDYYMHWVRQRP |
| | | EQGLEWIGWIDPENGNTIYDPKFQGKASITADTSSNTAYLQ |
| | | LSSLTSEDTAVYYCAYRSTMITTGAMDYWGQGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGK |
| 106 | aN5_LC | DIVLTQSPASLAVSLGQRATISGRASESVDNYGISFMNWFQ |
| | | QKPGQSPKLLIYAASNQGSGVPARFSGSGSGTDFSLNIHPM |
| | | EEDDTAMYFCQQSKEVPYTFGGGTKLEIKRTVAAPSVFIFP |
| | | PSDEQLKSGTASVVCLLNNFYPREAKVQWKVDNALQSGNSQ |
| | | ESVTEQDSKDSTYSLSSTLTLSKADYEKHKVYACEVTHQGL |
| | | SSPVTKSFNRGEC |
| 107 | aN7(H)^{ΔK}-C1r_HC | EVQLQQSGAELVRPGALVKLSCKASGFNIKDFYMHWVKQRP |
| | | DQGLEWIGWIDPENDNTIYDPKFQGKASITADTSSNTAYLQ |
| | | LSSLTSEDTAVYYCAYRSPMITTGAMDFWGPGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 108 | aN19(H)^{ΔK}- | EVQLVESGGGLVQPKGSLKLSCAASGFTFNMYVMNWVRQAP |
| | C1r_HC | GKGLEWVARIRSKSYNFGTYYADSVKDRFTISRDDSQSVLY |
| | | LTMNNLKTEDTAMYYCVRLDDDFAYWGPGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGGGGG |
| | | SGGGGSGGGGIKCPQPKTLDEFTIIQNLQPQYQFRDYFIAT |
| | | CKQGYQLIEGNQVLHSFTAVCQDDGTWHRAMPRCKIKDCGQ |
| | | PRNLPNGDFRYTTTMGVNTYKARIQYYCHEPYYKMQTRAGS |
| | | RESEQGVYTCTAQGIWKNEQKGEKIPRCLPVCGKPVNPVEQ |
| | | RQRIIGGQKAKMGNFPWQVFTNIHGRGGGALLGDRWILTAA |
| | | HTLYPKEHEAQSNASLDVFLGHTNVEELMKLGNHPIRRVSV |
| | | HPDYRQDESYNFEGDIALLELENSVTLGPNLLPICLPDNDT |
| | | FYDLGLMGYVSGFGVMEEKIAHDLRFVRLPVANPQACENWL |
| | | RGKNRMDVFSQNMFCAGHPSLKQDACQGDSGGVFAVRDPNT |
| | | DRWVATGIVSWGIGCSRGYGFYTKVLNYVDWIKKEMEEED |
| 109 | aN5(H)^{ΔK}-C1r_HC | EVQLQQSGAELVRPGALVKLSCKASGFNIKDYYMHWVRQRP |
| | | EQGLEWIGWIDPENGNTIYDPKFQGKASITADTSSNTAYLQ |
| | | LSSLTSEDTAVYYCAYRSTMITTGAMDYWGQGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGIKCPQPKTLDEFTIIQNLQPQYQFRDYF |
| | | IATCKQGYQLIEGNQVLHSFTAVCQDDGTWHRAMPRCKIKD |
| | | CGQPRNLPNGDFRYTTTMGVNTYKARIQYYCHEPYYKMQTR |
| | | AGSRESEQGVYTCTAQGIWKNEQKGEKIPRCLPVCGKPVNP |
| | | VEQRQRIIGGQKAKMGNFPWQVFTNIHGRGGGALLGDRWIL |
| | | TAAHTLYPKEHEAQSNASLDVFLGHTNVEELMKLGNHPIRR |
| | | VSVHPDYRQDESYNFEGDIALLELENSVTLGPNLLPICLPD |
| | | NDTFYDLGLMGYVSGFGVMEEKIAHDLRFVRLPVANPQACE |
| | | NWLRGKNRMDVFSQNMFCAGHPSLKQDACQGDSGGVFAVRD |
| | | PNTDRWVATGIVSWGIGCSRGYGFYTKVLNYVDWIKKEMEE |
| | | ED |
| 110 | aN7(H)^{ΔK}-C1s_HC | EVQLQQSGAELVRPGALVKLSCKASGFNIKDFYMHWVKQRP |
| | | DQGLEWIGWIDPENDNTIYDPKFQGKASITADTSSNTAYLQ |
| | | LSSLTSEDTAVYYCAYRSPMITTGAMDFWGPGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 111 | aN19(H)^{ΔK}- | EVQLVESGGGLVQPKGSLKLSCAASGETFNMYVMNWVRQAP |
| | C1s_HC | GKGLEWVARIRSKSYNFGTYYADSVKDRFTISRDDSQSVLY |
| | | LTMNNLKTEDTAMYYCVRLDDDFAYWGPGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGGGGG |
| | | SGGGGSGGGGMPCPKEDTPNSVWEPAKAKYVFRDVVQITCL |
| | | DGFEVVEGRVGATSFYSTCQSNGKWSNSKLKCQPVDCGIPE |
| | | SIENGKVEDPESTLFGSVIRYTCEEPYYYMENGGGGEYHCA |
| | | GNGSWVNEVLGPELPKCVPVCGVPREPFEEKQRIIGGSDAD |
| | | IKNFPWQVFFDNPWAGGALINEYWVLTAAHVVEGNREPTMY |
| | | VGSTSVQTSRLAKSKMLTPEHVFIHPGWKLLEVPEGRTNFD |
| | | NDIALVRLKDPVKMGPTVSPICLPGTSSDYNLMDGDLGLIS |
| | | GWGRTEKRDRAVRLKAARLPVAPLRKCKEVKVEKPTADAEA |
| | | YVFTPNMICAGGEKGMDSCKGDSGGAFAVQDPNDKTKFYAA |
| | | GLVSWGPQCGTYGLYTRVKNYVDWIMKTMQENSTPRED |
| 112 | aN5(H)^{ΔK}-C1s_HC | EVQLQQSGAELVRPGALVKLSCKASGFNIKDYYMHWVRQRP |
| | | EQGLEWIGWIDPENGNTIYDPKFQGKASITADTSSNTAYLQ |
| | | LSSLTSEDTAVYYCAYRSTMITTGAMDYWGQGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPGG |
| | | GGGSGGGGSGGGGMPCPKEDTPNSVWEPAKAKYVFRDVVQI |
| | | TCLDGFEVVEGRVGATSFYSTCQSNGKWSNSKLKCQPVDCG |
| | | IPESIENGKVEDPESTLFGSVIRYTCEEPYYYMENGGGGEY |
| | | HCAGNGSWVNEVLGPELPKCVPVCGVPREPFEEKQRIIGGS |
| | | DADIKNFPWQVFFDNPWAGGALINEYWVLTAAHVVEGNREP |
| | | TMYVGSTSVQTSRLAKSKMLTPEHVFIHPGWKLLEVPEGRT |
| | | NFDNDIALVRLKDPVKMGPTVSPICLPGTSSDYNLMDGDLG |
| | | LISGWGRTEKRDRAVRLKAARLPVAPLRKCKEVKVEKPTAD |
| | | AEAYVFTPNMICAGGEKGMDSCKGDSGGAFAVQDPNDKTKF |
| | | YAAGLVSWGPQCGTYGLYTRVKNYVDWIMKTMQENSTPRED |
| 113 | aN7(H)^{ΔK}_HC | EVQLQQSGAELVRPGALVKLSCKASGFNIKDFYMHWVKQRP |
| | | DQGLEWIGWIDPENDNTIYDPKFQGKASITADTSSNTAYLQ |
| | | LSSLTSEDTAVYYCAYRSPMITTGAMDFWGPGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| 114 | aN19(H)^{ΔK}_HC | EVQLVESGGGLVQPKGSLKLSCAASGETFNMYVMNWVRQAP |
| | | GKGLEWVARIRSKSYNFGTYYADSVKDRFTISRDDSQSVLY |
| | | LTMNNLKTEDTAMYYCVRLDDDFAYWGPGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPG |
| 115 | aN5(H)^{ΔK}_HC | EVQLQQSGAELVRPGALVKLSCKASGFNIKDYYMHWVRQRP |
| | | EQGLEWIGWIDPENGNTIYDPKFQGKASITADTSSNTAYLQ |
| | | LSSLTSEDTAVYYCAYRSTMITTGAMDYWGQGTSVTVSSAS |
| | | TKGPSVFPLAPSSKSTSGGTAALGCLVKDYFPEPVTVSWNS |
| | | GALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| 116 | aN19(H)^{ΔK}- | EVQLVESGGGLVQPKGSLKLSCAASGFTFNMYVMNWVRQAP |
| | M2^{R444K}_HC | GKGLEWVARIRSKSYNFGTYYADSVKDRFTISRDDSQSVLY |
| | | LTMNNLKTEDTAMYYCVRLDDDFAYWGPGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGGGGG |
| | | SGGGGSGGGGQPCPYPMAPPNGHVSPVQAKYILKDSFSIFC |
| | | ETGYELLQGHLPLKSFTAVCQKDGSWDRPMPACSIVDCGPP |
| | | DDLPSGRVEYITGPGVTTYKAVIQYSCEETFYTMKVNDGKY |
| | | VCEADGFWTSSKGEKSLPVCEPVCGLSARTTGGKIYGGQKA |
| | | KPGDFPWQVLILGGTTAAGALLYDNWVLTAAHAVYEQKHDA |
| | | SALDIRMGTLKRLSPHYTQAWSEAVFIHEGYTHDAGFDNDI |
| | | ALIKLNNKvvINSNITPICLPRKEAESFMRTDDIGTASGWG |
| | | LTQRGFLARNLMYVDIPIVDHQKCTAAYEKPPYPRGSVTAN |
| | | MLCAGLESGGKDSCRGDSGGALVFLDSETERWFVGGIVSWG |
| | | SMNCGEAGQYGVYTKVINYIPWIENIISDF |
| 117 | aN19(H)^{ΔK}-M3_HC | EVQLVESGGGLVQPKGSLKLSCAASGETFNMYVMNWVRQAP |
| | | GKGLEWVARIRSKSYNFGTYYADSVKDRFTISRDDSQSVLY |
| | | LTMNNLKTEDTAMYYCVRLDDDFAYWGPGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGGGGG |
| | | SGGGGSGGGGNECPELQPPVHGKIEPSQAKYFFKDQVLVSC |
| | | DTGYKVLKDNVEMDTFQIECLKDGTWSNKIPTCKIVDCRAP |
| | | GELEHGLITFSTRNNLTTYKSEIKYSCQEPYYKMLNNNTGI |
| | | YTCSAQGVWMNKVLGRSLPTCLPECGQPSRSLPSLVKRIIG |
| | | GRNAEPGLFPWQALIVVEDTSRVPNDKWFGSGALLSASWIL |
| | | TAAHVLRSQRRDTTVIPVSKEHVTVYLGLHDVRDKSGAVNS |
| | | SAARvvLHPDFNIQNYNHDIALVQLQEPVPLGPHVMPVCLP |
| | | RLEPEGPAPHMLGLVAGWGISNPNVTVDEIISSGTRTLSDV |
| | | LQYVKLPVVPHAECKTSYESRSGNYSVTENMFCAGYYEGGK |
| | | DTCLGDSGGAFVIFDDLSQRWVVQGLVSWGGPEECGSKQVY |
| | | GVYTKVSNYVDWVWEQMGLPQSWEPQVER |
| 118 | MatCFD- | ILGGREAEAHARPYMASVQLNGAHLCGGVLVAEQWVLSAAH |
| | aN19(H)_HC | CLEDAADGKVQVLLGAHSLSQPEPSKRLYDVLRAVPHPDSQ |
| | | PDTIDHDLLLLQLSEKATLGPAVRPLPWQRVDRDVAPGTLC |
| | | DVAGWGIVNHAGRRPDSLQHVLLPVLDRATCNRRTHHDGAI |
| | | TERLMCAESNRRDSCKGDSGGPLVCGGVLEGvvTSGSRVCG |
| | | NRKKPGIYTRVASYAAWIDSVLAGGGGSGGGGSGGGGEVQL |
| | | VESGGGLVQPKGSLKLSCAASGETFNMYVMNWVRQAPGKGL |
| | | EWVARIRSKSYNFGTYYADSVKDRFTISRDDSQSVLYLTMN |
| | | NLKTEDTAMYYCVRLDDDFAYWGPGTLVTVSSASTKGPSVF |
| | | PLAPSSKSTSGGTAALGCLVKDYFPEPVTVSWNSGALTSGV |
| | | HTFPAVLQSSGLYSLSSVVTVPSSSLGTQTYICNVNHKPSN |
| | | TKVDKKVEPKSCDKTHTCPPCPAPELLGGPSVFLFPPKPKD |
| | | TLMISRTPEVTCVVVDVSHEDPEVKFNWYVDGVEVHNAKTK |
| | | PREEQYNSTYRVVSVLTVLHQDWLNGKEYKCKVSNKALPAP |
| | | IEKTISKAKGQPREPQVYTLPPSRDELTKNQVSLTCLVKGF |
| | | YPSDIAVEWESNGQPENNYKTTPPVLDSDGSFFLYSKLTVD |
| | | KSRWQQGNVFSCSVMHEALHNHYTQKSLSLSPGK |
| 119 | ProCFD- | APPRGRILGGREAEAHARPYMASVQLNGAHLCGGVLVAEQW |
| | aN19(H)_HC | VLSAAHCLEDAADGKVQVLLGAHSLSQPEPSKRLYDVLRAV |
| | | PHPDSQPDTIDHDLLLLQLSEKATLGPAVRPLPWQRVDRDV |
| | | APGTLCDVAGWGIVNHAGRRPDSLQHVLLPVLDRATCNRRT |
| | | HHDGAITERLMCAESNRRDSCKGDSGGPLVCGGVLEGVVTS |
| | | GSRVCGNRKKPGIYTRVASYAAWIDSVLAGGGGSGGGGSGG |
| | | GGEVQLVESGGGLVQPKGSLKLSCAASGETFNMYVMNWVRQ |
| | | APGKGLEWVARIRSKSYNFGTYYADSVKDRFTISRDDSQSV |
| | | LYLTMNNLKTEDTAMYYCVRLDDDFAYWGPGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 120 | RX1MI005_HC | EVMLVESGGGLVKPGGSLKLSCAASGFTFSSYTMSWVRQTP |
| | | EKRLEWVATISGGGGNTYYSDSVKGRFTISRDNAKNTLYLQ |
| | | MSSLRSEDTALYYCARHDYGSFDYWGQGTTLTVSSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSCDKTHTCPPCPAPELLGGPSVFLFPPK |
| | | PKDTLMISRTPEVTCVVVDVSHEDPEVKFNWYVDGVEVHNA |
| | | KTKPREEQYNSTYRVVSVLTVLHQDWLNGKEYKCKVSNKAL |
| | | PAPIEKTISKAKGQPREPQVYTLPPSRDELTKNQVSLTCLV |
| | | KGFYPSDIAVEWESNGQPENNYKTTPPVLDSDGSFFLYSKL |
| | | TVDKSRWQQGNVFSCSVMHEALHNHYTQKSLSLSPGK |
| 121 | RX1MI005_LC | DIKMTQSPSSMYASLGERVTITCKASQDINRYLSWFQQKPG |
| | | KSPKTLIYRANRLVDGVPSRFSGSGSGQDYSLTISSLEYED |
| | | MGIYYCLQYDEFPFTFGSGTKLEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 122 | RX1MI005(H)^{ΔK}- | EVMLVESGGGLVKPGGSLKLSCAASGFTFSSYTMSWVRQTP |
| | C1r_HC | EKRLEWVATISGGGGNTYYSDSVKGRFTISRDNAKNTLYLQ |
| | | MSSLRSEDTALYYCARHDYGSFDYWGQGTTLTVSSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSCDKTHTCPPCPAPELLGGPSVFLFPPK |
| | | PKDTLMISRTPEVTCVVVDVSHEDPEVKFNWYVDGVEVHNA |
| | | KTKPREEQYNSTYRVVSVLTVLHQDWLNGKEYKCKVSNKAL |
| | | PAPIEKTISKAKGQPREPQVYTLPPSRDELTKNQVSLTCLV |
| | | KGFYPSDIAVEWESNGQPENNYKTTPPVLDSDGSFFLYSKL |
| | | TVDKSRWQQGNVFSCSVMHEALHNHYTQKSLSLSPGGGGGS |
| | | GGGGSGGGGIKCPQPKTLDEFTIIQNLQPQYQFRDYFIATC |
| | | KQGYQLIEGNQVLHSFTAVCQDDGTWHRAMPRCKIKDCGQP |
| | | RNLPNGDFRYTTTMGVNTYKARIQYYCHEPYYKMQTRAGSR |
| | | ESEQGVYTCTAQGIWKNEQKGEKIPRCLPVCGKPVNPVEQR |
| | | QRIIGGQKAKMGNFPWQVFTNIHGRGGGALLGDRWILTAAH |
| | | TLYPKEHEAQSNASLDVFLGHTNVEELMKLGNHPIRRVSVH |
| | | PDYRQDESYNFEGDIALLELENSVTLGPNLLPICLPDNDTF |
| | | YDLGLMGYVSGFGVMEEKIAHDLRFVRLPVANPQACENWLR |
| | | GKNRMDVFSQNMFCAGHPSLKQDACQGDSGGVFAVRDPNTD |
| | | RWVATGIVSWGIGCSRGYGFYTKVLNYVDWIKKEMEEED |
| 123 | RX1MI005(H)^{ΔK}- | EVMLVESGGGLVKPGGSLKLSCAASGFTFSSYTMSWVRQTP |
| | C1s_HC | EKRLEWVATISGGGGNTYYSDSVKGRETISRDNAKNTLYLQ |
| | | MSSLRSEDTALYYCARHDYGSFDYWGQGTTLTVSSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSCDKTHTCPPCPAPELLGGPSVFLFPPK |
| | | PKDTLMISRTPEVTCVVVDVSHEDPEVKFNWYVDGVEVHNA |
| | | KTKPREEQYNSTYRVVSVLTVLHQDWLNGKEYKCKVSNKAL |
| | | PAPIEKTISKAKGQPREPQVYTLPPSRDELTKNQVSLTCLV |
| | | KGFYPSDIAVEWESNGQPENNYKTTPPVLDSDGSFFLYSKL |
| | | TVDKSRWQQGNVFSCSVMHEALHNHYTQKSLSLSPGGGGGS |
| | | GGGGSGGGGMPCPKEDTPNSVWEPAKAKYVFRDVVQITCLD |
| | | GFEVVEGRVGATSFYSTCQSNGKWSNSKLKCQPVDCGIPES |
| | | IENGKVEDPESTLFGSVIRYTCEEPYYYMENGGGGEYHCAG |
| | | NGSWVNEVLGPELPKCVPVCGVPREPFEEKQRIIGGSDADI |
| | | KNFPWQVFFDNPWAGGALINEYWVLTAAHVVEGNREPTMYV |
| | | GSTSVQTSRLAKSKMLTPEHVFIHPGWKLLEVPEGRTNFDN |
| | | DIALVRLKDPVKMGPTVSPICLPGTSSDYNLMDGDLGLISG |
| | | WGRTEKRDRAVRLKAARLPVAPLRKCKEVKVEKPTADAEAY |
| | | VFTPNMICAGGEKGMDSCKGDSGGAFAVQDPNDKTKFYAAG |
| | | LVSWGPQCGTYGLYTRVKNYVDWIMKTMQENSTPRED |
| 124 | C1.G_HC | EVQLQQSGPELVKPGASVKMSCKASGYTFTDYYMKWVKQSH |
| | | GKSLEWIGDINPNNGDTFYNQKFKGKATLTVDKSSSTAYMQ |
| | | LNSLTSEDSAVYYCARGDYWGQGTTLTVSSASTKGPSVFPL |
| | | APSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALPAPIE |
| | | KTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGK |
| 125 | C1.G_LC | DVVMTQTPLTLSVTIGQPASISCKSSQSLLDSDGKTYLNWL |
| | | LQRPGQSPKRLIYLVSKLDSGVPDRFTGSGSGTDFTLKISR |
| | | VEAEDLGVYYCWQGTHFPWTFGGGTKLEIKRTVAAPSVFIF |
| | | PPSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| 126 | C1.G(H)^{ΔK}-C1r_HC | EVQLQQSGPELVKPGASVKMSCKASGYTFTDYYMKWVKQSH |
| | | GKSLEWIGDINPNNGDTFYNQKFKGKATLTVDKSSSTAYMQ |
| | | LNSLTSEDSAVYYCARGDYWGQGTTLTVSSASTKGPSVFPL |
| | | APSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALPAPIE |
| | | KTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGGGGGSGGGGS |
| | | GGGGIKCPQPKTLDEFTIIQNLQPQYQFRDYFIATCKQGYQ |
| | | LIEGNQVLHSFTAVCQDDGTWHRAMPRCKIKDCGQPRNLPN |
| | | GDFRYTTTMGVNTYKARIQYYCHEPYYKMQTRAGSRESEQG |
| | | VYTCTAQGIWKNEQKGEKIPRCLPVCGKPVNPVEQRQRIIG |
| | | GQKAKMGNFPWQVFTNIHGRGGGALLGDRWILTAAHTLYPK |
| | | EHEAQSNASLDVFLGHTNVEELMKLGNHPIRRVSVHPDYRQ |
| | | DESYNEEGDIALLELENSVTLGPNLLPICLPDNDTFYDLGL |
| | | MGYVSGFGVMEEKIAHDLRFVRLPVANPQACENWLRGKNRM |
| | | DVFSQNMFCAGHPSLKQDACQGDSGGVFAVRDPNTDRWVAT |
| | | GIVSWGIGCSRGYGFYTKVLNYVDWIKKEMEEED |
| 127 | C1.G(H)^{ΔK}-C1s_HC | EVQLQQSGPELVKPGASVKMSCKASGYTFTDYYMKWVKQSH |
| | | GKSLEWIGDINPNNGDTFYNQKFKGKATLTVDKSSSTAYMQ |
| | | LNSLTSEDSAVYYCARGDYWGQGTTLTVSSASTKGPSVFPL |
| | | APSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALPAPIE |
| | | KTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGGGGGSGGGGS |
| | | GGGGMPCPKEDTPNSVWEPAKAKYVFRDVVQITCLDGFEVV |
| | | EGRVGATSFYSTCQSNGKWSNSKLKCQPVDCGIPESIENGK |
| | | VEDPESTLFGSVIRYTCEEPYYYMENGGGGEYHCAGNGSWV |
| | | NEVLGPELPKCVPVCGVPREPFEEKQRIIGGSDADIKNFPW |
| | | QVFFDNPWAGGALINEYWVLTAAHVVEGNREPTMYVGSTSV |
| | | QTSRLAKSKMLTPEHVFIHPGWKLLEVPEGRTNFDNDIALV |
| | | RLKDPVKMGPTVSPICLPGTSSDYNLMDGDLGLISGWGRTE |
| | | KRDRAVRLKAARLPVAPLRKCKEVKVEKPTADAEAYVFTPN |
| | | MICAGGEKGMDSCKGDSGGAFAVQDPNDKTKFYAAGLVSWG |
| | | PQCGTYGLYTRVKNYVDWIMKTMQENSTPRED |
| 128 | 1A2_HC | QVRLQESGPSLVKPSQTLSLTCTVSGFGLTTYSIEWVRQAP |
| | | GKALEWVGAVNNNGRTFYNPALKSRLSITRDTSKSQVSLSL |
| | | SSVTTEDTAVYYCVRTWDVWGRGLLVTVSSASTKGPSVFPL |
| | | APSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALPAPIE |
| | | KTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGK |
| 129 | 1A2_LC | QAVLTQPSSVSRSLGQSVSITCSGSSSNIGSWNYVDWFQVI |
| | | PGSAPRTLITAATSRTSGVPDRFSGSRSGNTATLTITSLQA |
| | | EDEADYYCAAWDRSNSKIFGSGTRLTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 130 | 1A2(H)^{ΔK}-C1r_HC | QVRLQESGPSLVKPSQTLSLTCTVSGFGLTTYSIEWVRQAP |
| | | GKALEWVGAVNNNGRTFYNPALKSRLSITRDTSKSQVSLSL |
| | | SSVTTEDTAVYYCVRTWDVWGRGLLVTVSSASTKGPSVFPL |
| | | APSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALPAPIE |
| | | KTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGGGGGSGGGGS |
| | | GGGGIKCPQPKTLDEFTIIQNLQPQYQFRDYFIATCKQGYQ |
| | | LIEGNQVLHSFTAVCQDDGTWHRAMPRCKIKDCGQPRNLPN |
| | | GDFRYTTTMGVNTYKARIQYYCHEPYYKMQTRAGSRESEQG |
| | | VYTCTAQGIWKNEQKGEKIPRCLPVCGKPVNPVEQRQRIIG |
| | | GQKAKMGNFPWQVFTNIHGRGGGALLGDRWILTAAHTLYPK |
| | | EHEAQSNASLDVFLGHTNVEELMKLGNHPIRRVSVHPDYRQ |
| | | DESYNFEGDIALLELENSVTLGPNLLPICLPDNDTFYDLGL |
| | | MGYVSGFGVMEEKIAHDLRFVRLPVANPQACENWLRGKNRM |
| | | DVFSQNMFCAGHPSLKQDACQGDSGGVFAVRDPNTDRWVAT |
| | | GIVSWGIGCSRGYGFYTKVLNYVDWIKKEMEEED |
| 131 | 1A2(H)^{ΔK}-C1s_HC | QVRLQESGPSLVKPSQTLSLTCTVSGFGLTTYSIEWVRQAP |
| | | GKALEWVGAVNNNGRTFYNPALKSRLSITRDTSKSQVSLSL |
| | | SSVTTEDTAVYYCVRTWDVWGRGLLVTVSSASTKGPSVFPL |
| | | APSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSCDKTHTCPPCPAPELLGGPSVFLFPPKPKDTL |
| | | MISRTPEVTCVVVDVSHEDPEVKFNWYVDGVEVHNAKTKPR |
| | | EEQYNSTYRVVSVLTVLHQDWLNGKEYKCKVSNKALPAPIE |
| | | KTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGGGGGSGGGGS |
| | | GGGGMPCPKEDTPNSVWEPAKAKYVFRDVVQITCLDGFEVV |
| | | EGRVGATSFYSTCQSNGKWSNSKLKCQPVDCGIPESIENGK |
| | | VEDPESTLFGSVIRYTCEEPYYYMENGGGGEYHCAGNGSWV |
| | | NEVLGPELPKCVPVCGVPREPFEEKQRIIGGSDADIKNFPW |
| | | QVFFDNPWAGGALINEYWVLTAAHVVEGNREPTMYVGSTSV |
| | | QTSRLAKSKMLTPEHVFIHPGWKLLEVPEGRTNFDNDIALV |
| | | RLKDPVKMGPTVSPICLPGTSSDYNLMDGDLGLISGWGRTE |
| | | KRDRAVRLKAARLPVAPLRKCKEVKVEKPTADAEAYVFTPN |
| | | MICAGGEKGMDSCKGDSGGAFAVQDPNDKTKFYAAGLVSWG |
| | | PQCGTYGLYTRVKNYVDWIMKTMQENSTPRED |
| 132 | hJF5_HC | EVQLVESGGGLVQPGGSLKLSCAASGFDFSRYWMSWVRQAP |
| | | GKGLEWVAEINPDSSKINYMPSLKDRFTISRDNAKNSLYLQ |
| | | MNSLRAEDTAVYYCARPRGYYAMDFWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGK |
| 133 | hJF5_LC | DVVMTQSPSSLSASVGDRVTITCKASQDVSTAVAWYQQKPG |
| | | KAPKPLIYSASYQYTGVPSRFSGSGSGTDFTFTITSLQPED |
| | | IAIYYCQQHYSIPWTFGGGTKVEIKRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 134 | hJF5(H)^{ΔK}-C1r_HC | EVQLVESGGGLVQPGGSLKLSCAASGFDFSRYWMSWVRQAP |
| | | GKGLEWVAEINPDSSKINYMPSLKDRFTISRDNAKNSLYLQ |
| | | MNSLRAEDTAVYYCARPRGYYAMDFWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGGGGG |
| | | SGGGGSGGGGIKCPQPKTLDEFTIIQNLQPQYQFRDYFIAT |
| | | CKQGYQLIEGNQVLHSFTAVCQDDGTWHRAMPRCKIKDCGQ |
| | | PRNLPNGDFRYTTTMGVNTYKARIQYYCHEPYYKMQTRAGS |
| | | RESEQGVYTCTAQGIWKNEQKGEKIPRCLPVCGKPVNPVEQ |
| | | RQRIIGGQKAKMGNFPWQVFTNIHGRGGGALLGDRWILTAA |
| | | HTLYPKEHEAQSNASLDVFLGHTNVEELMKLGNHPIRRVSV |
| | | HPDYRQDESYNFEGDIALLELENSVTLGPNLLPICLPDNDT |
| | | FYDLGLMGYVSGFGVMEEKIAHDLRFVRLPVANPQACENWL |
| | | RGKNRMDVFSQNMFCAGHPSLKQDACQGDSGGVFAVRDPNT |
| | | DRWVATGIVSWGIGCSRGYGFYTKVLNYVDWIKKEMEEED |
| 135 | hJF5(H)^{ΔK}-C1s_HC | EVQLVESGGGLVQPGGSLKLSCAASGFDFSRYWMSWVRQAP |
| | | GKGLEWVAEINPDSSKINYMPSLKDRFTISRDNAKNSLYLQ |
| | | MNSLRAEDTAVYYCARPRGYYAMDFWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPELLGGPSVFLFPP |
| | | KPKDTLMISRTPEVTCVVVDVSHEDPEVKFNWYVDGVEVHN |
| | | AKTKPREEQYNSTYRVVSVLTVLHQDWLNGKEYKCKVSNKA |
| | | LPAPIEKTISKAKGQPREPQVYTLPPSRDELTKNQVSLTCL |
| | | VKGFYPSDIAVEWESNGQPENNYKTTPPVLDSDGSFFLYSK |
| | | LTVDKSRWQQGNVFSCSVMHEALHNHYTQKSLSLSPGGGGG |
| | | SGGGGSGGGGMPCPKEDTPNSVWEPAKAKYVFRDVVQITCL |
| | | DGFEVVEGRVGATSFYSTCQSNGKWSNSKLKCQPVDCGIPE |
| | | SIENGKVEDPESTLFGSVIRYTCEEPYYYMENGGGGEYHCA |
| | | GNGSWVNEVLGPELPKCVPVCGVPREPFEEKQRIIGGSDAD |
| | | IKNFPWQVFFDNPWAGGALINEYWVLTAAHVVEGNREPTMY |
| | | VGSTSVQTSRLAKSKMLTPEHVFIHPGWKLLEVPEGRTNFD |
| | | NDIALVRLKDPVKMGPTVSPICLPGTSSDYNLMDGDLGLIS |
| | | GWGRTEKRDRAVRLKAARLPVAPLRKCKEVKVEKPTADAEA |
| | | YVFTPNMICAGGEKGMDSCKGDSGGAFAVQDPNDKTKFYAA |
| | | GLVSWGPQCGTYGLYTRVKNYVDWIMKTMQENSTPRED |
| 136 | R5.004_HC | EVQLVQSGAEVKKPGSSVKVSCKASGGTFSNYAINWVRQAP |
| | | GQGLEWMGGIIPIFATTNYAQKFQGRVTITADESTSTAYME |
| | | LSSLRSEDTAVYFCARDKHSWSYAFDIWGQGTMVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 137 | R5.004_LC | QSVLTQPPSASGTPGLRVTISCSGSSSNIGSNTVNWYQHLP |
| | | GTAPKLLIHSNNQRPSGVPDRFSGSKSGTSASLAISGLQSE |
| | | DEADYYCAAWDDSLNGWVFGGGTKLTVLGQPKAAPSVTLFP |
| | | PSSEELQANKATLVCLISDFYPGAVTVAWKADSSPVKAGVE |
| | | TTTPSKQSNNKYAASSYLSLTPEQWKSHRSYSCQVTHEGST |
| | | VEKTVAPTECS |
| 138 | R5.004(H)^{ΔK}- | EVQLVQSGAEVKKPGSSVKVSCKASGGTFSNYAINWVRQAP |
| | C1r_HC | GQGLEWMGGIIPIFATTNYAQKFQGRVTITADESTSTAYME |
| | | LSSLRSEDTAVYFCARDKHSWSYAFDIWGQGTMVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGGG |
| | | GGSGGGGSGGGGIKCPQPKTLDEFTIIQNLQPQYQFRDYFI |
| | | ATCKQGYQLIEGNQVLHSFTAVCQDDGTWHRAMPRCKIKDC |
| | | GQPRNLPNGDFRYTTTMGVNTYKARIQYYCHEPYYKMQTRA |
| | | GSRESEQGVYTCTAQGIWKNEQKGEKIPRCLPVCGKPVNPV |
| | | EQRQRIIGGQKAKMGNFPWQVFTNIHGRGGGALLGDRWILT |
| | | AAHTLYPKEHEAQSNASLDVFLGHTNVEELMKLGNHPIRRV |
| | | SVHPDYRQDESYNFEGDIALLELENSVTLGPNLLPICLPDN |
| | | DTFYDLGLMGYVSGFGVMEEKIAHDLRFVRLPVANPQACEN |
| | | WLRGKNRMDVFSQNMFCAGHPSLKQDACQGDSGGVFAVRDP |
| | | NTDRWVATGIVSWGIGCSRGYGFYTKVLNYVDWIKKEMEEE |
| | | D |
| 139 | R5.004(H)^{ΔK}- | EVQLVQSGAEVKKPGSSVKVSCKASGGTFSNYAINWVRQAP |
| | C1s_HC | GQGLEWMGGIIPIFATTNYAQKFQGRVTITADESTSTAYME |
| | | LSSLRSEDTAVYFCARDKHSWSYAFDIWGQGTMVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNV |
| | | NHKPSNTKVDKKVEPKSCDKTHTCPPCPAPELLGGPSVFLF |
| | | PPKPKDTLMISRTPEVTCVVVDVSHEDPEVKFNWYVDGVEV |
| | | HNAKTKPREEQYNSTYRVVSVLTVLHQDWLNGKEYKCKVSN |
| | | KALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGGG |
| | | GGSGGGGSGGGGMPCPKEDTPNSVWEPAKAKYVFRDVVQIT |
| | | CLDGFEVVEGRVGATSFYSTCQSNGKWSNSKLKCQPVDCGI |
| | | PESIENGKVEDPESTLFGSVIRYTCEEPYYYMENGGGGEYH |
| | | CAGNGSWVNEVLGPELPKCVPVCGVPREPFEEKQRIIGGSD |
| | | ADIKNFPWQVFFDNPWAGGALINEYWVLTAAHVVEGNREPT |
| | | MYVGSTSVQTSRLAKSKMLTPEHVFIHPGWKLLEVPEGRTN |
| | | FDNDIALVRLKDPVKMGPTVSPICLPGTSSDYNLMDGDLGL |
| | | ISGWGRTEKRDRAVRLKAARLPVAPLRKCKEVKVEKPTADA |
| | | EAYVFTPNMICAGGEKGMDSCKGDSGGAFAVQDPNDKTKFY |
| | | AAGLVSWGPQCGTYGLYTRVKNYVDWIMKTMQENSTPRED |
| 140 | R5.016_HC | QVQLVQSGAEVKKPGASVRVSCKASGYTFTSYGISWVRQAP |
| | | GQGLEWMGWISGYDGNTNYAQKLQGRVTMTTDTSTSTAYME |
| | | LRSLRSDDTAVYYCARDGPQVGDFDWQVYYYYGMDVWGQGT |
| | | TVTVSSASTKGPSVFPLAPSSKSTSGGTAALGCLVKDYFPE |
| | | PVTVSWNSGALTSGVHTFPAVLQSSGLYSLSSVVTVPSSSL |
| | | GTQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPGK |
| 141 | R5.016_LC | AIRMTQSPSTLSASVGDRVTITCRASQSINTWLAWYQQKPG |
| | | KAPNLLISKASSLESGVPSRFSGSGSGTEFTLTISSLQPDD |
| | | FATYFCQQYNSYLYTFGQGTKVEIRRTVAAPSVFIFPPSDE |
| | | QLKSGTASVVCLLNNFYPREAKVQWKVDNALQSGNSQESVT |
| | | EQDSKDSTYSLSSTLTLSKADYEKHKVYACEVTHQGLSSPV |
| | | TKSFNRGEC |
| 142 | R5.016(H)^{ΔK}- | QVQLVQSGAEVKKPGASVRVSCKASGYTFTSYGISWVRQAP |
| | C1r_HC | GQGLEWMGWISGYDGNTNYAQKLQGRVTMTTDTSTSTAYME |
| | | LRSLRSDDTAVYYCARDGPQVGDFDWQVYYYYGMDVWGQGT |
| | | TVTVSSASTKGPSVFPLAPSSKSTSGGTAALGCLVKDYFPE |
| | | PVTVSWNSGALTSGVHTFPAVLQSSGLYSLSSVVTVPSSSL |
| | | GTQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPGGGGGSGGGGSGGGGIKCPQPKTLDEFTIIQNLQP |
| | | QYQFRDYFIATCKQGYQLIEGNQVLHSFTAVCQDDGTWHRA |
| | | MPRCKIKDCGQPRNLPNGDFRYTTTMGVNTYKARIQYYCHE |
| | | PYYKMQTRAGSRESEQGVYTCTAQGIWKNEQKGEKIPRCLP |
| | | VCGKPVNPVEQRQRIIGGQKAKMGNFPWQVFTNIHGRGGGA |
| | | LLGDRWILTAAHTLYPKEHEAQSNASLDVFLGHTNVEELMK |
| | | LGNHPIRRVSVHPDYRQDESYNFEGDIALLELENSVTLGPN |
| | | LLPICLPDNDTFYDLGLMGYVSGFGVMEEKIAHDLRFVRLP |
| | | VANPQACENWLRGKNRMDVFSQNMFCAGHPSLKQDACQGDS |
| | | GGVFAVRDPNTDRWVATGIVSWGIGCSRGYGFYTKVLNYVD |
| | | WIKKEMEEED |
| 143 | R5.016(H)^{ΔK}- | QVQLVQSGAEVKKPGASVRVSCKASGYTFTSYGISWVRQAP |
| | C1s_HC | GQGLEWMGWISGYDGNTNYAQKLQGRVTMTTDTSTSTAYME |
| | | LRSLRSDDTAVYYCARDGPQVGDFDWQVYYYYGMDVWGQGT |
| | | TVTVSSASTKGPSVFPLAPSSKSTSGGTAALGCLVKDYFPE |
| | | PVTVSWNSGALTSGVHTFPAVLQSSGLYSLSSVVTVPSSSL |
| | | GTQTYICNVNHKPSNTKVDKKVEPKSCDKTHTCPPCPAPEL |
| | | LGGPSVFLFPPKPKDTLMISRTPEVTCVVVDVSHEDPEVKF |
| | | NWYVDGVEVHNAKTKPREEQYNSTYRVVSVLTVLHQDWLNG |
| | | KEYKCKVSNKALPAPIEKTISKAKGQPREPQVYTLPPSRDE |
| | | LTKNQVSLTCLVKGFYPSDIAVEWESNGQPENNYKTTPPVL |
| | | DSDGSFFLYSKLTVDKSRWQQGNVFSCSVMHEALHNHYTQK |
| | | SLSLSPGGGGGSGGGGSGGGGMPCPKEDTPNSVWEPAKAKY |
| | | VFRDVVQITCLDGFEVVEGRVGATSFYSTCQSNGKWSNSKL |
| | | KCQPVDCGIPESIENGKVEDPESTLFGSVIRYTCEEPYYYM |
| | | ENGGGGEYHCAGNGSWVNEVLGPELPKCVPVCGVPREPFEE |
| | | KQRIIGGSDADIKNFPWQVFFDNPWAGGALINEYWVLTAAH |
| | | VVEGNREPTMYVGSTSVQTSRLAKSKMLTPEHVFIHPGWKL |
| | | LEVPEGRTNFDNDIALVRLKDPVKMGPTVSPICLPGTSSDY |
| | | NLMDGDLGLISGWGRTEKRDRAVRLKAARLPVAPLRKCKEV |
| | | KVEKPTADAEAYVFTPNMICAGGEKGMDSCKGDSGGAFAVQ |
| | | DPNDKTKFYAAGLVSWGPQCGTYGLYTRVKNYVDWIMKTMQ |
| | | ENSTPRED |
| 144 | PGT121_HC | QMQLQESGPGLVKPSETLSLTCSVSGASISDSYWSWIRRSP |
| | | GKGLEWIGYVHKSGDTNYSPSLKSRVNLSLDTSKNQVSLSL |
| | | VAATAADSGKYYCARTLHGRRIYGIVAFNEWFTYFYMDVWG |
| | | NGTQVTVSSASTKGPSVFPLAPSSKSTSGGTAALGCLVKDY |
| | | FPEPVTVSWNSGALTSGVHTFPAVLQSSGLYSLSSVVTVPS |
| | | SSLGTQTYICNVNHKPSNTKVDKKVEPKSCDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLHQDW |
| | | LNGKEYKCKVSNKALPAPIEKTISKAKGQPREPQVYTLPPS |
| | | RDELTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGK |
| 145 | PGT121_LC | SDISVAPGETARISCGEKSLGSRAVQWYQHRAGQAPSLIIY |
| | | NNQDRPSGIPERFSGSPDSPFGTTATLTITSVEAGDEADYY |
| | | CHIWDSRVPTKWVFGGGTTLTVLGQPKAAPSVTLFPPSSEE |
| | | LQANKATLVCLISDFYPGAVTVAWKADSSPVKAGVETTTPS |
| | | KQSNNKYAASSYLSLTPEQWKSHRSYSCQVTHEGSTVEKTV |
| | | APTECS |
| 146 | PGT121(H)^{ΔK}- | QMQLQESGPGLVKPSETLSLTCSVSGASISDSYWSWIRRSP |
| | C1r_HC | GKGLEWIGYVHKSGDTNYSPSLKSRVNLSLDTSKNQVSLSL |
| | | VAATAADSGKYYCARTLHGRRIYGIVAFNEWFTYFYMDVWG |
| | | NGTQVTVSSASTKGPSVFPLAPSSKSTSGGTAALGCLVKDY |
| | | FPEPVTVSWNSGALTSGVHTFPAVLQSSGLYSLSSVVTVPS |
| | | SSLGTQTYICNVNHKPSNTKVDKKVEPKSCDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLHQDW |
| | | LNGKEYKCKVSNKALPAPIEKTISKAKGQPREPQVYTLPPS |
| | | RDELTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGGGGGSGGGGSGGGGIKCPQPKTLDEFTIIQN |
| | | LQPQYQFRDYFIATCKQGYQLIEGNQVLHSFTAVCQDDGTW |
| | | HRAMPRCKIKDCGQPRNLPNGDFRYTTTMGVNTYKARIQYY |
| | | CHEPYYKMQTRAGSRESEQGVYTCTAQGIWKNEQKGEKIPR |
| | | CLPVCGKPVNPVEQRQRIIGGQKAKMGNFPWQVFTNIHGRG |
| | | GGALLGDRWILTAAHTLYPKEHEAQSNASLDVFLGHTNVEE |
| | | LMKLGNHPIRRVSVHPDYRQDESYNFEGDIALLELENSVTL |
| | | GPNLLPICLPDNDTFYDLGLMGYVSGFGVMEEKIAHDLRFV |
| | | RLPVANPQACENWLRGKNRMDVFSQNMFCAGHPSLKQDACQ |
| | | GDSGGVFAVRDPNTDRWVATGIVSWGIGCSRGYGFYTKVLN |
| | | YVDWIKKEMEEED |
| 147 | PGT121(H)^{ΔK}- | QMQLQESGPGLVKPSETLSLTCSVSGASISDSYWSWIRRSP |
| | C1s_HC | GKGLEWIGYVHKSGDTNYSPSLKSRVNLSLDTSKNQVSLSL |
| | | VAATAADSGKYYCARTLHGRRIYGIVAFNEWFTYFYMDVWG |
| | | NGTQVTVSSASTKGPSVFPLAPSSKSTSGGTAALGCLVKDY |
| | | FPEPVTVSWNSGALTSGVHTFPAVLQSSGLYSLSSVVTVPS |
| | | SSLGTQTYICNVNHKPSNTKVDKKVEPKSCDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLHQDW |
| | | LNGKEYKCKVSNKALPAPIEKTISKAKGQPREPQVYTLPPS |
| | | RDELTKNQVSLTCLVKGFYPSDIAVEWESNGQPENNYKTTP |
| | | PVLDSDGSFFLYSKLTVDKSRWQQGNVFSCSVMHEALHNHY |
| | | TQKSLSLSPGGGGGSGGGGSGGGGMPCPKEDTPNSVWEPAK |
| | | AKYVFRDVVQITCLDGFEVVEGRVGATSFYSTCQSNGKWSN |
| | | SKLKCQPVDCGIPESIENGKVEDPESTLFGSVIRYTCEEPY |
| | | YYMENGGGGEYHCAGNGSWVNEVLGPELPKCVPVCGVPREP |
| | | FEEKQRIIGGSDADIKNFPWQVFFDNPWAGGALINEYWVLT |
| | | AAHVVEGNREPTMYVGSTSVQTSRLAKSKMLTPEHVFIHPG |
| | | WKLLEVPEGRTNFDNDIALVRLKDPVKMGPTVSPICLPGTS |
| | | SDYNLMDGDLGLISGWGRTEKRDRAVRLKAARLPVAPLRKC |
| | | KEVKVEKPTADAEAYVFTPNMICAGGEKGMDSCKGDSGGAF |
| | | AVQDPNDKTKFYAAGLVSWGPQCGTYGLYTRVKNYVDWIMK |
| | | TMQENSTPRED |
| 148 | bebtelovimab_HC | QITLKESGPTLVKPTQTLTLTCTFSGFSLSISGVGVGWLRQ |
| | | PPGKALEWLALIYWDDDKRYSPSLKSRLTISKDTSKNQVVL |
| | | KMTNIDPVDTATYYCAHHSISTIFDHWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGK |
| 149 | bebtelovimab_LC | QSALTQPASVSGSPGQSITISCTATSSDVGDYNYVSWYQQH |
| | | PGKAPKLMIFEVSDRPSGISNRFSGSKSGNTASLTISGLQA |
| | | EDEADYYCSSYTTSSAVFGGGTKLTVLGQPKAAPSVTLFPP |
| | | SSEELQANKATLVCLISDFYPGAVTVAWKADSSPVKAGVET |
| | | TTPSKQSNNKYAASSYLSLTPEQWKSHRSYSCQVTHEGSTV |
| | | EKTVAPTECS |
| 150 | bebtelovimab(H)^{ΔK}- | QITLKESGPTLVKPTQTLTLTCTFSGFSLSISGVGVGWLRQ |
| | C1r_HC | PPGKALEWLALIYWDDDKRYSPSLKSRLTISKDTSKNQVVL |
| | | KMTNIDPVDTATYYCAHHSISTIFDHWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGGGG |
| | | GSGGGGSGGGGIKCPQPKTLDEFTIIQNLQPQYQFRDYFIA |
| | | TCKQGYQLIEGNQVLHSFTAVCQDDGTWHRAMPRCKIKDCG |
| | | QPRNLPNGDFRYTTTMGVNTYKARIQYYCHEPYYKMQTRAG |
| | | SRESEQGVYTCTAQGIWKNEQKGEKIPRCLPVCGKPVNPVE |
| | | QRQRIIGGQKAKMGNFPWQVFTNIHGRGGGALLGDRWILTA |
| | | AHTLYPKEHEAQSNASLDVFLGHTNVEELMKLGNHPIRRVS |
| | | VHPDYRQDESYNFEGDIALLELENSVTLGPNLLPICLPDND |
| | | TFYDLGLMGYVSGFGVMEEKIAHDLRFVRLPVANPQACENW |
| | | LRGKNRMDVFSQNMFCAGHPSLKQDACQGDSGGVFAVRDPN |
| | | TDRWVATGIVSWGIGCSRGYGFYTKVLNYVDWIKKEMEEED |
| 151 | bebtelovimab(H)^{ΔK}- | QITLKESGPTLVKPTQTLTLTCTFSGFSLSISGVGVGWLRQ |
| | C1s_HC | PPGKALEWLALIYWDDDKRYSPSLKSRLTISKDTSKNQVVL |
| | | KMTNIDPVDTATYYCAHHSISTIFDHWGQGTLVTVSSASTK |
| | | GPSVFPLAPSSKSTSGGTAALGCLVKDYFPEPVTVSWNSGA |
| | | LTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLMISRTPEVTCVVVDVSHEDPEVKFNWYVDGVEVH |
| | | NAKTKPREEQYNSTYRVVSVLTVLHQDWLNGKEYKCKVSNK |
| | | ALPAPIEKTISKAKGQPREPQVYTLPPSRDELTKNQVSLTC |
| | | LVKGFYPSDIAVEWESNGQPENNYKTTPPVLDSDGSFFLYS |
| | | KLTVDKSRWQQGNVFSCSVMHEALHNHYTQKSLSLSPGGGG |
| | | GSGGGGSGGGGMPCPKEDTPNSVWEPAKAKYVFRDVVQITC |
| | | LDGFEVVEGRVGATSFYSTCQSNGKWSNSKLKCQPVDCGIP |
| | | ESIENGKVEDPESTLFGSVIRYTCEEPYYYMENGGGGEYHC |
| | | AGNGSWVNEVLGPELPKCVPVCGVPREPFEEKQRIIGGSDA |
| | | DIKNFPWQVFFDNPWAGGALINEYWVLTAAHVVEGNREPTM |
| | | YVGSTSVQTSRLAKSKMLTPEHVFIHPGWKLLEVPEGRTNF |
| | | DNDIALVRLKDPVKMGPTVSPICLPGTSSDYNLMDGDLGLI |
| | | SGWGRTEKRDRAVRLKAARLPVAPLRKCKEVKVEKPTADAE |
| | | AYVFTPNMICAGGEKGMDSCKGDSGGAFAVQDPNDKTKFYA |
| | | AGLVSWGPQCGTYGLYTRVKNYVDWIMKTMQENSTPRED |

### VIII. EXAMPLES

### Example 1

### Preparation of Rituximab-Derived Targeted Complement-Activating Molecules

MASP fusions

The C-terminal catalytic segment of MASP-1, MASP-2 and MASP-3, the CCP1-CCP2-SP domains, were fused with anti-CD20 antibody rituximab (RTX; IgG1 isotype), with various configurations, and the fusion proteins were expressed using the expression vector pCAG. The vector is a modified version of pD2610-v1 (ATUM; originally from (Miyazaki et al., 1989)) and contains the characteristic CMV and chicken beta-actin hybrid promoter, and a kanamycin resistance marker.

The fusion positions were either at the C- or N-terminus of the antibody's heavy chain (HC-CCP1/2SP or CCP1/2SP-HC, respectively) or at the C- or N-terminus of the antibody's light chain (LC-CCP1/2SP or CCP1/2SP-LC, respectively), which resulted in the following various constructs: M3-RTX(H) (SEQ ID NO:13), RTX(L)-M3 (SEQ ID NO:14), M3-RTX(L) (SEQ ID NO:15), RTX(H)-M2 (SEQ ID NO:4), M2-RTX(H) (SEQ ID NO:6), RTX(L)-M2 (SEQ ID NO:7), and M2-RTX(L) (SEQ ID NO:8). The MASP-1 fusions include fusions with a single substitution in the serine protease domain as compared to the wild-type serine protease sequence that was intended to improve stability, and one MASP-1 fusion incorporates a rituximab heavy chain with a deletion of the lysine (K) from the C-terminus: RTX(H)^{ΔK}-M1^{R504Q} (SEQ ID NO:16), and M1^{R504Q}-RTX(L) (SEQ ID NO:17). Additionally, mutant forms of the constructs RTX(H)-M2 and RTX(H)-M3 were generated which were altered by a deletion of the single amino acid, lysine (K), from the C-terminus of rituximab's heavy chain, resulting in constructs RTX(H)^{ΔK}-M2 (SEQ ID NO:5) and RTX(H)^{ΔK}-M3 (SEQ ID NO:12). A catalytically inactive version of the MASP-2 fusion was generated by introducing a single substitution into the serine protease domain: RTX(H)^{ΔK}-M2^{S633A} (SEQ ID NO:11). A constitutive zymogen form of the MASP-2 fusion was also generated: RTX(H)^{ΔK}-M2^{R444Q} (SEQ ID NO:10), as was a MASP-2 fusion with slower activation kinetics than wild-type: RTX(H)^{ΔK}-M2^{R444K} (SEQ ID NO:9).

Additional molecules were produced with one or two single amino acid substitutions in the MASP-2 serine protease domain: RTX(H)^{ΔK}-M2^{R444K} (SEQ ID NO:9), RTX(H)^{ΔK,K121Q}-M2^{R444K} (SEQ ID NO:33), RTX(H)^{ΔK}-M2^{K317Q,R444K} (SEQ ID NO:34), RTX(H)^{ΔK}-M2^{K321Q,R444K} (SEQ ID NO:35), RTX(H)^{ΔK}-M2^{K342Q,R444K} (SEQ ID NO:36), RTX(H)^{ΔK}-M2^{K350Q,R444K} (SEQ ID NO:37), and RTX(H)^{ΔK}-M2^{R356Q,R444K} (SEQ ID NO:38), as part of an effort to identify molecules with increased stability/reduced degradation.

C1r and C1s Fusions

The C1r and C1s serine protease effector domains were fused to RTX and expressed similar to the MASP fusions; the C-terminal catalytic fragment of C1r and C1s (CCP1-CCP2-SP) was fused with RTX at the C-terminus of the antibody's heavy chain (HC).

Three constructs of each complement component were generated, including a “wild-type” form, an aglycosylated form having a single substitution in the antibody's Fc region, and a catalytically inactive fusion having a single substitution in the serine protease domain: RTX(H)^{ΔK}-C1r (SEQ ID NO:18), RTX(H)^{N297G,ΔK}-C1r (SEQ ID NO:21), RTX(H)^{N297G,ΔK}-C1r^{S654A} (SEQ ID NO:22), RTX(H)^{ΔK}-C1s (SEQ ID NO:19), RTX(H)^{N297G,ΔK}-C1s (SEQ ID NO:23), and RTX(H)^{N297,ΔK}-C1s^{S632A} (SEQ ID NO:24).

Additional molecules were produced with one of several single amino acid substitutions in the serine protease domain: RTX(H)^{N297G,ΔK}-C1r^{K374Q} (SEQ ID NO:39), RTX(H)^{N297G,ΔK}-C1r^{R380Q} (SEQ ID NO:40), RTX(H)^{N297G,ΔK}-C1s^{K308Q} (SEQ ID NO:41), RTX(H)^{N297G,ΔK}-C1 s^{K310Q} (SEQ ID NO:42), RTX(H)^{N297G,ΔK}-C1s^{R314Q} (SEQ ID NO:43), RTX(H)^{N297G,ΔK}-C1s^{R331Q} (SEQ ID NO:44), RTX(H)^{N297G,ΔK}-C1s^{K346Q} (SEQ ID NO:45), RTX(H)^{N297G,ΔK}-C1s^{K351Q} (SEQ ID NO:46), RTX(H)^{N297G,ΔK}-C1s^{K353Q} (SEQ ID NO:47), RTX(H)^{N297G,ΔK}-C1r^{H484W} (SEQ ID NO:48), RTX(H)^{N297G,ΔK}-C1r^{G485W} (SEQ ID NO:49), RTX(H)^{N297G,ΔK}-C1r^{R486W} (SEQ ID NO:50), RTX(H)^{N297G,ΔK}-C1s^{D456W} (SEQ ID NO:51), RTX(H)^{N297G,ΔK}-C1s^{N457W} (SEQ ID NO:52), and RTX(H)^{N297G,ΔK}-C1s^{P458W} (SEQ ID NO:53), as part of an effort to identify molecules with increased stability/reduced degradation.

Fusions with other Complement Components

The catalytic segment of each of complement factors C2 (C2a), B (Bb) and D was fused to RTX and expressed by using the expression vector pCAG.

Various configurations of the pro- and mature CFD fusions were generated. CFD was fused either to the C- or N-terminus of the antibody's heavy or light chain, resulting in the following constructs: RTX(H)^{ΔK}-MatCFD (SEQ ID NO:27), MatCFD-RTX(H) (SEQ ID NO:28), RTX(L)-MatCFD (SEQ ID NO:29), MatCFD-RTX(L) (SEQ ID NO:30)- and ProCFD-RTX(H) (SEQ ID NO:32). A catalytically inactive version having a single point mutation in the serine protease domain was also generated: MatCFD^{S208A}-RTX(H) (SEQ ID NO:31).

One configuration was generated for the C2a and Bb fusions, having the catalytic segment fused to the C-terminus of the antibody's heavy chain: RTX(H)^{ΔK}-C2a (SEQ ID NO:25) and RTX(H)^{ΔK}-Bb (SEQ ID NO:26).

The various rituximab fusion configurations are described in detail in Table 1.

TABLE 1

| | Recom- | | |
|---|---|---|---|
| | binant | | |
| | proteins | Abbreviations | Description |
| Ab- | rituximab- | RTX(H)-M2 | CCP1/2SP domains of |
| MASP-2 | MASP-2 | | MASP-2 are fused |
| fusions | (HC- | | to the C-terminus |
| | CCP12SP) | | of the heavy chain |
| | | | of rituximab. |
| | rituximab- | RTX(H)^{ΔK}-M2 | CCP1/2SP domains of |
| | MASP-2 | | MASP-2 are fused |
| | (HCΔK- | | to the C-terminus |
| | CCP12SP) | | of the heavy chain |
| | | | of rituximab. |
| | | | Mutation: Deletion of |
| | | | the amino acid lysine |
| | | | (K) in Ab’s HC. |
| | rituximab- | RTX(H)^{ΔK}- | CCP1/2SP domains of |
| | MASP-2 | M2^{R444K} | MASP-2 are fused to |
| | (HCΔK- | | the C-terminus |
| | CCP12SP- | | of the heavy chain |
| | R444K) | | of rituximab. |
| | | | Mutations: Deletion |
| | | | of the amino acid lysine |
| | | | (K) in Ab’s HC. |
| | | | Single residue substitution |
| | | | mutant arginine (R) to |
| | | | lysine (K) on MASP-2. |
| | rituximab- | RTX(H)^{ΔK}- | CCP1/2SP domains of |
| | MASP-2 | M2^{R444Q} | MASP-2 are fused to |
| | (HCΔK- | | the C-terminus |
| | CCP12SP- | | of the heavy chain |
| | R444Q) | | of rituximab. |
| | | | Mutations: Deletion of |
| | | | the amino acid lysine |
| | | | (K) in Ab’s HC. |
| | | | Single residue substitution |
| | | | mutant arginine (R) to |
| | | | glutamine (Q) on MASP-2. |
| | rituximab- | RTX(H)^{ΔK}- | CCP1/2SP domains of |
| | MASP-2 | M2^{S633A} | MASP-2 are fused |
| | (HCΔK- | | to the C-terminus |
| | CCP12SP- | | of the heavy chain |
| | S633A) | | of rituximab. |
| | | | Mutations: Deletion of the |
| | | | amino acid lysine |
| | | | (K) in Ab’s HC. |
| | | | Single residue substitution |
| | | | mutant serine (S) to |
| | | | alanine (A) on MASP-2. |
| | MASP-2- | M2-RTX(H) | CCP1/2SP domains of |
| | rituximab | | MASP-2 are fused to |
| | (CCP12SP- | | the N-terminus of the |
| | HC) | | heavy chain of rituximab. |
| | rituximab- | RTX(L)-M2 | CCP1/2SP domains of |
| | MASP-2 | | MASP-2 are fused |
| | (LC- | | to the C-terminus |
| | CCP12SP) | | of the light chain |
| | | | of rituximab. |
| | MASP-2- | M2-RTX(L) | CCP1/2SP domains of |
| | rituximab | | MASP-2 are fused to |
| | (CCP12SP- | | the N-terminus of the |
| | LC) | | light chain of rituximab. |
| Ab- | rituximab- | RTX(H)^{ΔK}-M3 | CCP1/2SP domains of |
| MASP-3 | MASP-3 | | MASP-3 are fused |
| fusions | (HCΔK- | | to the C-terminus of the |
| | CCP12SP) | | heavy chain of rituximab. |
| | | | Mutation: Deletion of the |
| | | | amino acid lysine (K) |
| | | | in Ab’s HC. |
| | MASP-3- | M3-RTX(H) | CCP1/2SP domains of |
| | rituximab | | MASP-3 are fused to |
| | (CCP12SP- | | the N-terminus of the |
| | HC) | | heavy chain of rituximab. |
| | rituximab- | RTX(L)-M3 | CCP1/2SP domains of |
| | MASP-3 | | MASP-3 are fused to |
| | (LC- | | the C-terminus of the |
| | CCP12SP) | | light chain of rituximab. |
| | MASP-3- | M3-RTX(L) | CCP1/2SP domains of |
| | rituximab | | MASP-3 are fused to |
| | (CCP12SP- | | the N-terminus of the |
| | LC) | | light chain of rituximab. |
| Ab- | rituximab- | RTX(H)^{ΔK}- | CCP1/2SP domains of |
| MASP-1 | MASP-1 | M1^{R504Q} | MASP-1 are fused to |
| fusions | (HCΔK- | | the C-terminus of the |
| | CCP12SP- | | heavy chain of rituximab. |
| | R504Q) | | Mutations: Deletion of |
| | | | the amino acid lysine (K) |
| | | | in Ab’s HC. |
| | | | Single residue substitution |
| | | | mutant arginine |
| | | | (R) to glutamine |
| | | | (Q) on MASP-1. |
| | MASP-1 | M1^{R504Q}- | CCP1/2SP domains of |
| | (CCP12SP- | RTX(L) | MASP-1 are fused to the |
| | R504Q))- | | N-terminus of the |
| | rituximab | | light chain of rituximab. |
| | (LC) | | Mutations: Single residue |
| | | | substitution mutant |
| | | | arginine (R) to glutamine |
| | | | (Q) on MASP-1. |
| Ab-C1r | rituximab- | RTX(H)^{ΔK}-C1r | CCP1/2SP domains of |
| fusions | C1r | | C1r are fused to the |
| | (HCΔK- | | C-terminus of the heavy |
| | CCP12SP) | | chain of rituximab. |
| | | | Mutations: Deletion of |
| | | | the amino acid lysine |
| | | | (K) in Ab’s HC. |
| | rituximab- | RTX(H)^{ΔK,N297G}- | CCP1/2SP domains of |
| | C1r | C1r | C1r are fused to the |
| | (HCΔK- | | C-terminus of the |
| | N297G- | | heavy chain of rituximab. |
| | CCP12SP) | | Mutations: Deletion of |
| | | | the amino acid lysine |
| | | | (K) in Ab’s HC. |
| | | | Single residue substitution |
| | | | mutant asparagine |
| | | | (N) to glycine |
| | | | (G) on Ab’s Fc region. |
| | rituximab- | RTX(H)^{ΔK,N297G}- | CCP1/2SP domains of |
| | C1r | C1r^{S654A} | C1r are fused to |
| | (HCΔK- | | the C-terminus of the |
| | N297G- | | heavy chain of rituximab. |
| | CCP12SP- | | Mutations: Deletion of |
| | S654A) | | the amino acid lysine |
| | | | (K) in Ab’s HC. |
| | | | Single residue substitution |
| | | | mutant asparagine (N) |
| | | | to glycine (G) on Ab’s |
| | | | Fc region and single |
| | | | residue substitution |
| | | | mutant serine (S) to |
| | | | alanine (A) on C1r. |
| Ab-C1s | rituximab- | RTX(H)^{ΔK}-C1s | CCP1/2SP domains of |
| fusions | C1s | | C1s are fused to |
| | (HCΔK- | | the C-terminus of the |
| | CCP12SP) | | heavy chain of rituximab. |
| | | | Mutations: Deletion of |
| | | | the amino acid lysine |
| | | | (K) in Ab’s HC. |
| | rituximab- | RTX(H)^{ΔK,N297G}- | CCP1/2SP domains of |
| | C1s | C1s | C1s are fused to the |
| | (HCΔK- | | C-terminus of the |
| | N297G- | | heavy chain of rituximab. |
| | CCP12SP) | | Mutations: Deletion of |
| | | | the amino acid lysine |
| | | | (K) in Ab’s HC. |
| | | | Single residue substitution |
| | | | mutant asparagine |
| | | | (N) to glycine |
| | | | (G) on Ab’s Fc region. |
| | rituximab- | RTX(H)^{ΔK,N297G}- | CCP1/2SP domains of C1s |
| | C1s | C1s^{S632A} | are fused to the |
| | (HCΔK- | | C-terminus of the |
| | N297G- | | heavy chain of rituximab. |
| | CCP12SP- | | Mutations: Deletion of the |
| | S632A) | | amino acid lysine |
| | | | (K) in Ab’s HC. |
| | | | Single residue substitution |
| | | | mutant asparagine |
| | | | (N) to glycine |
| | | | (G) on Ab’s Fc region and |
| | | | single residue substitution |
| | | | mutant serine (S) to |
| | | | alanine (A) on C1s. |
| Ab-CFD | proCFD- | ProCFD- | SP domain of the pro-form |
| fusions | RTX(HC) | RTX(H) | of CFD is fused to the N- |
| | | | terminus of the heavy |
| | | | chain of rituximab. |
| | pro-CFD- | ProCFD- | SP domain of the pro-form |
| | RTX(HC- | RTX(H)^{N297G} | of CFD is fused to the N- |
| | N297G) | | terminus of the heavy |
| | | | chain of rituximab. |
| | | | Mutations: Single residue |
| | | | substitution mutant |
| | | | asparagine |
| | | | (N) to glycine (G) |
| | | | on Ab’s Fc region. |
| | MatCFD- | MatCFD- | SP domain of mature CFD is |
| | RTX(HC) | RTX(H) | fused to the |
| | | | N-terminus of the |
| | | | heavy chain of rituximab. |
| | MatCFD- | MatCFD- | SP domain of mature CFD |
| | RTX(HC- | RTX(H)^{N297G} | is fused to the N-terminus |
| | N297G) | | of the heavy |
| | | | chain of rituximab. |
| | | | Mutations: Single residue |
| | | | substitution mutant |
| | | | asparagine |
| | | | (N) to glycine (G) |
| | | | on Ab’s Fc region. |
| | MatCFD- | MatCFD^{S208A}- | SP domain of mature CFD |
| | RTX | RTX(H) | is fused to the |
| | (S208A- | | N-terminus of the |
| | HC) | | heavy chain of rituximab. |
| | | | Mutations: Single residue |
| | | | substitution mutant serine |
| | | | (S) to alanine (A) on CFD. |
| | MatCFD- | MatCFD^{S208A}- | SP domain of mature CFD |
| | RTX | RTX(H)^{N297G} | is fused to the N-terminus |
| | (S208A- | | of the heavy chain |
| | HC- | | of rituximab. |
| | N297G) | | Mutations: Single residue |
| | | | substitution mutant |
| | | | serine (S) to |
| | | | alanine (A) on CFD and |
| | | | single residue |
| | | | substitution mutant |
| | | | asparagine (N) to glycine |
| | | | (G) on Ab’s Fc region |
| | RTX- | RTX(H)^{ΔK}- | SP domain of mature |
| | matCFD | matCFD | CFD is fused to the |
| | (HCΔK) | | C-terminus of the |
| | | | heavy chain of rituximab. |
| | | | Mutations: Deletion of the |
| | | | amino acid lysine (K) |
| | | | in Ab’s HC. |
| | RTX- | RTX(L)- | SP domain of mature CFD is |
| | matCFD | matCFD | fused to the C-terminus of |
| | (LC) | | the light chain of rituximab. |
| | MatCFD- | MatCFD- | SP domain of mature CFD is |
| | RTX(LC) | RTX(L) | fused to the N-terminus of |
| | | | the light chain of rituximab. |
| Ab-C2a | rituximab- | RTX(H)^{ΔK}-C2a | vWFA-SP domains of C2a |
| fusions | C2a | | are fused to the |
| | (HCΔK) | | C-terminus of |
| | | | the heavy chain of rituximab. |
| | | | Mutations: Deletion of the |
| | | | amino acid lysine (K) |
| | | | in Ab’s HC. |
| Ab-Bb | rituximab- | RTX(H)^{ΔK}-Bb | vWFA-SP domains of Bb |
| fusions | Bb | | are fused to the |
| | (HCΔK) | | C-terminus of the |
| | | | heavy chain of rituximab. |
| | | | Mutations: Deletion of the |
| | | | amino acid lysine (K) |
| | | | in Ab’s HC |

Plasmid Preparation and Cloning

In order to generate the constructs of the recombinant fusion proteins, the following steps were performed: (i) plasmid vector digestion (ii) cloning of inserts (iii) transformation into E. coli competent cells.

The vector pCAG was linearized for cloning by digestion with the restriction enzyme SapI (NEB). For the digestion in a 50 μL reaction, 3 μg of the vector, 2 μL of the endonuclease and 5 μL of CutSmart Buffer (NEB) were used. The restriction digestion was performed at 37° C. for approximately two hours.

To produce MASP-3, MASP-2, MASP-1, C1r, C1s, CFD, C2a and Bb fusions, the following constructs were made:

(i) rituximab's HC

(ii) rituximab's LC and

(iii) the catalytic segment of the complement proteins fused either to:
a) the C-terminus of RTX HC,
b) the N-terminus of RTX HC,
c) the C-terminus of RTX LC
d) the N-terminus of RTX LC.

PCR of inserts was carried out with specific primers, 5× Phusion HF Buffer (NEB), dNTPs, and Phusion DNA polymerase using the following thermocycling conditions:

Initial denaturation: at 98° C. for 30 sec

Denaturation: at 98° C. for 10 sec

Annealing: at 60° C. for 30 sec

Extension: at 72° C. for 40 sec

Additional extension: at 72° C. for 5 min.

The PCR was performed in 30 cycles. Amplification of the inserts was followed by gel electrophoresis to confirm the PCR reactions and the products were purified using NucleoSpin Purification Kit (Macherey-Nagel). Next, a 5 μL cloning reaction with the linearized vector, the purified inserts, and 5× In-Fusion HD Enzyme Premix (Takara Bio) was performed at 50° C. for 15 minutes.

The expression constructs used for making the recombinant fusion proteins were transformed into competent E. coli cells (One Shot Machl T1 Phage-Resistant Chemically Component E. coli, Invitrogen) and selected on Kanamycin LB agar plates. 10 ng of the plasmid was added into a vial of competent cells and the cells were incubated on ice for 30 minutes. The mixture was then heat-shocked at 42° C. for 30 seconds followed by incubation on ice for 2 minutes. 250 μL of S.O.C. medium was added to the vial and the cells were incubated at 37° C. for 1 hour at 225 rpm in a shaking incubator. 100 μL of this culture was plated on a kanamycin containing plate to grow overnight at 37° C. From the plate, single transformed colonies were selected and propagated in their resistant antibiotic (25 μg/mL) and 2.5 mL LB overnight in a 37° C. shaker. The next day, plasmid DNA was extracted from part of the cultures using the Qiagen plasmid miniprep kit; the other part of the 1 mL culture was stored in 4° C. for future use. The plasmid DNA was sequenced to ensure the success of the cloning and the selected clone was cultured in a 37° C. shaker for 1 hour and inoculated in 120 mL broth containing Kanamycin (Teknova). After overnight culture at 37° C., plasmid DNA were extracted using the Qiagen plasmid maxiprep kit.

Protein Expression and Purification

Expi293 cells were cultured in Expi293 medium (Gibco) and prepared for transfection with a cell density of 2.9×10⁶ cells/mL. Concentration of the purified plasmid DNA was measured, and the DNA was prepared in a ratio of 1:2 of HC:LC for transfection. For a transfection of 500 mL, a total amount of 500 μg DNA was used. DNA was suspended into OptiMEM (Gibco) and mixed with Lipofectamine (Invitrogen). After 20 minutes incubation at room temperature, the mixture of DNA and Lipofectamine was added to the cells. The transfected cells were cultured in a 37° C. shaker at 125 rpm for 4-5 days. After approximately 18 hours of transfection, enhancers (ExpiFectamine 293 Transfection Kit; Gibco), were added to boost cell viability and protein expression. For a total volume of 500 mL of culture, 2.5 mL of Enhancer-1 and 25 mL of Enhancer-2 were used.

For most of the recombinant fusion proteins, the transfected cells were cultured at 37° C. A lower temperature (at 32.5° C.) during culture was tested for the fusion proteins rituximab-MASP-3 (LC-CCP12SP) and MASP-3-rituximab (CCP12SP-LC) to see if the lower temperature would reduce aggregation of the proteins.

After 4-5 days of culture at 37° C., the transfected cells were removed and the culture supernatant was filtered using Sartoclear Dynamics lab kit (Sartorius). All the fusion proteins were purified with Protein A sepharose. After filtration of the supernatant, Protein A sepharose was added and, in order to maximize protein binding, the mixture was incubated on a rotator for 2 hours at room temperature. The mixtures were then loaded onto chromatography columns (Bio-Rad). After washing 2.5 times with 19 mL 1× PBS (Gibco), the proteins were eluted with 15 mL of pH 3 Glycine Buffer (Teknova) and neutralized with 3 mL of pH 9 Tris-HCl buffer (Teknova). The purification was completed by concentration and buffer-exchange to PBS or Histidine-Buffer (20 mM Histidine, 150 mM NaCl) (storage buffer) using centrifugal filter units (MilliporeSigma Amicon Ultra centrifugal Units).

The expression yield (mg/mL) of each protein is shown in Table 2.

TABLE 2

| | | | Exp. |
|---|---|---|---|
| | | | Yield |
| Fusion protein | Abbreviation | M.W. | (mg/L) |
| Rituximab-MASP-2 (HC-CCP12SP) | RTX(H)-M2 | 231.2 | 5.5 |
| Rituximab-MASP-2(HCΔK- | RTX(H)^{ΔK}-M2 | 231.0 | 2.2 |
| CCP12SP) | | | |
| Rituximab-MASP-2 (HCΔK- | RTX(H)^{ΔK}-M2^{R444K} | 230.9 | 1.4 |
| CCP12SP-R444K) | | | |
| Rituximab-MASP-2 (HCΔK- | RTX(H)^{ΔK}-M2^{R444Q} | 230.9 | 0.5 |
| CCP12SP-R444Q) | | | |
| Rituximab-MASP-2 (HCΔK- | RTX(H)^{ΔK}-M2^{S633A} | 230.9 | 2.3 |
| CCP12SP-S633A) | | | |
| MASP-2-Rituximab (CCP12SP-HC) | M2-RTX(H) | 231.2 | 0.8 |
| Rituximab-MASP-2 (LC-CCP12SP) | RTX(L)-M2 | 231.2 | 2.5 |
| MASP-2-Rituximab (CCP12SP-LC) | M2-RTX(L) | 231.2 | 0.8 |
| Rituximab-MASP-3(HCΔK- | RTX(H)^{ΔK}-M3 | 241.2 | 163.8 |
| CCP12SP) | | | |
| MASP-3-Rituximab (CCP12SP-HC) | M3-RTX(H) | 241.5 | 143.0 |
| Rituximab-MASP-3 (LC-CCP12SP) | RTX(L)-M3 | 241.5 | 52.0 |
| MASP-3-Rituximab (CCP12SP-LC) | M3-RTX(L) | 241.5 | 48.0 |
| Rituximab-MASP-1 (HCΔK- | RTX(H)^{ΔK}-M1^{R504Q} | 235.9 | 2.5 |
| CCP12SP-R504Q) | | | |
| MASP-1(CCP12SP-R504Q)- | M1^{R504Q}-RTX(L) | 236.2 | 1.1 |
| Rituximab (LC) | | | |
| Rituximab-C1r (HCΔK-CCP12SP) | RTX(H)^{ΔK}-C1r | 236.7 | 81.7 |
| Rituximab-C1r(HCΔK-N297G- | RTX(H)^{ΔK,N297G}- | 236.6 | 96.2 |
| CCP12SP) | C1r | | |
| Rituximab-C1r(HCΔK-N297G- | RTX(H)^{ΔK,N297G}- | 236.6 | 65.6 |
| CCP12SP-S654A) | C1r^{S654A} | | |
| Rituximab-C1s (HCΔK-CCP12SP) | RTX(H)^{ΔK}-C1s | 236.6 | 123.8 |
| Rituximab-C1s(HCΔK-N297G- | RTX(H)^{ΔK,N297G}- | 236.5 | 91.7 |
| CCP12SP) | C1s | | |
| Rituximab-C1s(HCΔK-N297G- | RTX(H)^{ΔK,N297G}- | 236.5 | 116.1 |
| CCP12SP-S632A) | C1s^{S632A} | | |
| ProCFD-RTX(HC) | ProCFD-RTX(H) | 196.3 | 3.4 |
| Pro-CFD-RTX(HC-N297G) | ProCFD- | 196.2 | 1.3 |
| | RTX(H)^{N297G} | | |
| MatCFD-RTX(HC) | MatCFD-RTX(H) | 195.0 | 11.7 |
| MatCFD-RTX(HC-N297G) | MatCFD- | 194.9 | 8.0 |
| | RTX(H)^{N297G} | | |
| MatCFD-RTX (S208A-HC) | MatCFD^{S208A}- | 195.0 | 23.1 |
| | RTX(H) | | |
| MatCFD-RTX (S208A-HC-N297G) | MatCFD^{S208A}- | 194.9 | 31.7 |
| | RTX(H)^{N297G} | | |
| RTX-MatCFD (HCΔK) | RTX(H)^{ΔK}- | 194.8 | 99.8 |
| | MatCFD | | |
| RTX-MatCFD (LC) | RTX(L)-MatCFD | 195.0 | 77.7 |
| MatCFD-RTX(LC) | MatCFD-RTX(L) | 195.0 | 15.1 |
| Rituximab-C2a (HCΔK) | RTX(H)^{ΔK}-C2a | 260.8 | 61.2 |
| Rituximab-Bb (HCΔK) | RTX(H)^{ΔK}-Bb | 260.0 | 18.1 |

### Example 2

### Analysis of Rituximab-Derived Expressed Proteins

Protein integrity

SDS-PAGE was performed to assess the protein integrity. Polyacrylamide gel with a gradient concentration of 4-12% (NuPAGE Bis-Tris gel, Invitrogen) was used to separate subunits of each protein, and polypeptide sizes were estimated using molecular weight marker (SeeBlue Plus 2, Invitrogen). SDS-PAGE was performed under both reducing and non-reducing conditions. After the gel was run in running buffer (MOPS SDS Running Buffer, NuPage) at 120V for 40 minutes, it was stained with staining solution (SimplyBlue SafeStain, Invitrogen) on a shaking rotator for 1 hour followed by de-staining overnight.

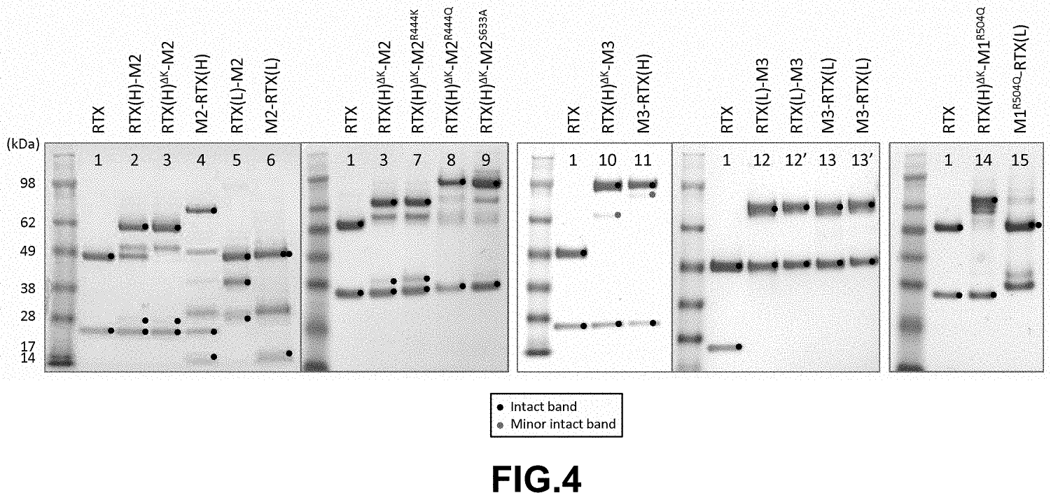

It was observed that all the Ab-MASP-2 fusions were degraded to some extent and in active form, apart from RTX(H)^{ΔK}-M2^{S633A} and RTX(H)^{ΔK}-M2^{R444Q}, which due to mutation are in an inactive or zymogen form. Conversely to MASP-2, the MASP-3 fusions were generated in the zymogen form and no degradation product was detected. See FIG. 4. Furthermore, SDS-PAGE analysis was performed for the Ab-MASP-3 fusion proteins which were cultured in lower temperature during expression. For comparison, the Ab-MASP-3 proteins that were cultured at 37° C., were included. Culture at lower temperature was performed in order to reduce aggregates that are formed during expression. The SDS-PAGE analysis was performed with reducing and non-reducing conditions and revealed no degradation of the MASP-3 fusion proteins and no difference among the proteins with different culture conditions (data not shown).

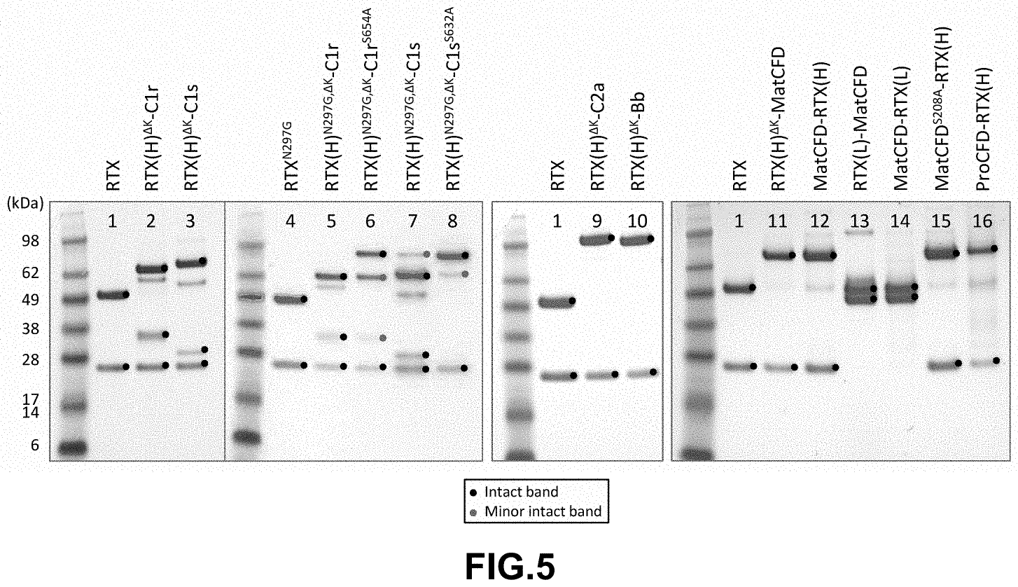

The Ab-C1r and Ab-C1s fusions were also found to have minor degradation products, whereas this was not the case for the Ab-C2a, Ab-Bb, or Ab-CFD fusions, which showed an intact protein band upon SDS-PAGE analysis. See FIG. 5.

Activation of MASP-3 Serine Protease Activity

The SDS-PAGE analysis of the Ab-MASP-3 fusions showed that the proteins are in the zymogen form, while no degradation product was detected. See FIG. 4.

Activation of RTX-M3 proteins was performed with the use of a truncated MASP-2 (CCP1/2SP) which was followed by SDS-PAGE analysis with reducing conditions to verify the conversion of the MASP-3 fusion protein from zymogen to active cleaved form. The activation of MASP-3 fusion was performed based to the published report by Oroszlan et al., 2017. Zymogen RTX-M3 (2 μM) was diluted in 140 mM NaCl, 10 mM HEPES, pH 7.4, 0.1 mM EDTA buffer and was incubated at 37° C. alone (negative control) or with the addition of MASP-2 (CCP1/2SP) (91 nM) in various timepoints (0, 10, 20, 40, 60, 90, 120, 150 and 190 minutes). The samples were removed in each timepoint at place on −20° C. to stop the reaction. Samples were analyzed by SDS-PAGE under reducing conditions.

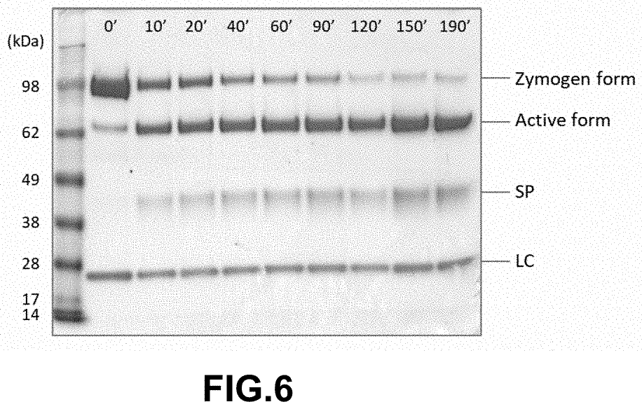

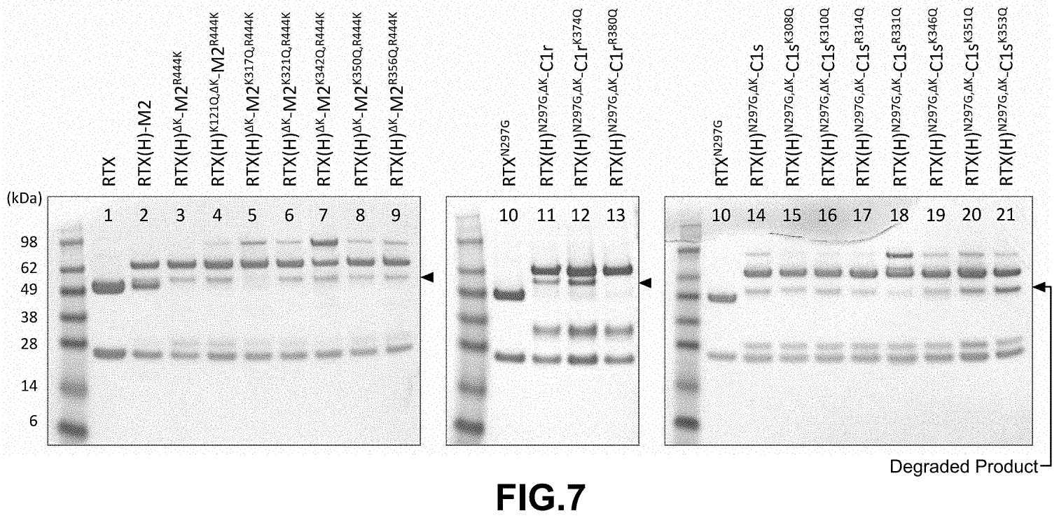

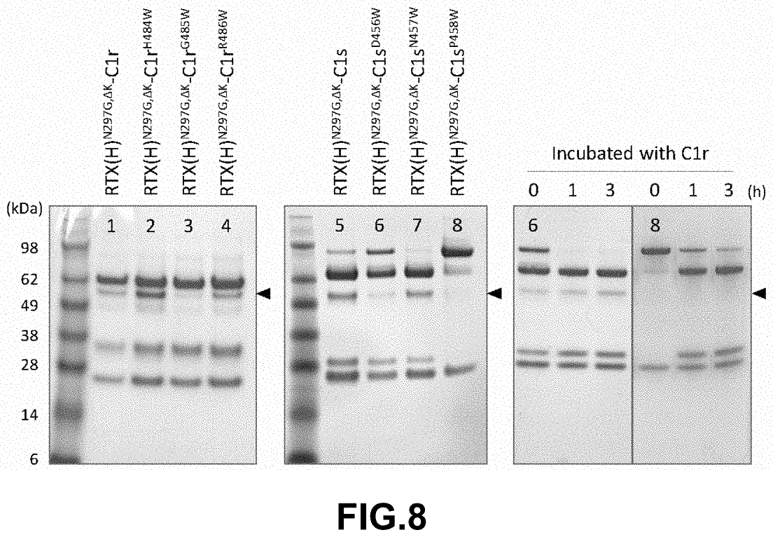

The assay demonstrated that the recombinant MASP-3 fusion can be activated by another serine protease, thereby becoming functional. A zymogen RTX-M3 fusion polypeptide runs at about 92 kDa, while the active form gives two bands, the RTX-M3(CCP1/2) and the cleaved SP domain. See FIG. 6.

Aggregation

Size exclusion chromatography was performed to assess aggregation of the recombinant MASP-3 fusion proteins using ÄKTA (GE Healthcare). 200 μg of the sample diluted in His-Buffer (20 mM Histidine, 150 mM NaCl) was injected and was run through a column (Superdex 200 Increase 5/150 GL column), to separate the proteins by size. The proteins that were analyzed were rituximab heavy chain fusions rituximab-MASP-3 (RTX(H)^{ΔK}-M3) and MASP-3-rituximab (M3-RTX(H)), and rituximab light chain fusions rituximab-MASP-3 (RTX(L)-M3) and MASP-3-rituximab (M3-RTX(L)). The latter two proteins were expressed at two different temperatures (37 and 32.5° C.).

The analysis showed more than 10% aggregation in RTX(H)^{ΔK}-M3 and M3-RTX(H) cultured at 37° C. Analysis of RTX(L)-M3 and M3-RTX(L) cultured at 37° C. or at 32.5° C. showed about a two-fold higher aggregation level and about a three-fold decrease in expression yield at the lower culture temperature (data not shown).

### Example 3

### Binding of Rituximab-Derived Targeted Complement-Activating Molecules to Targets

Flow Cytometry

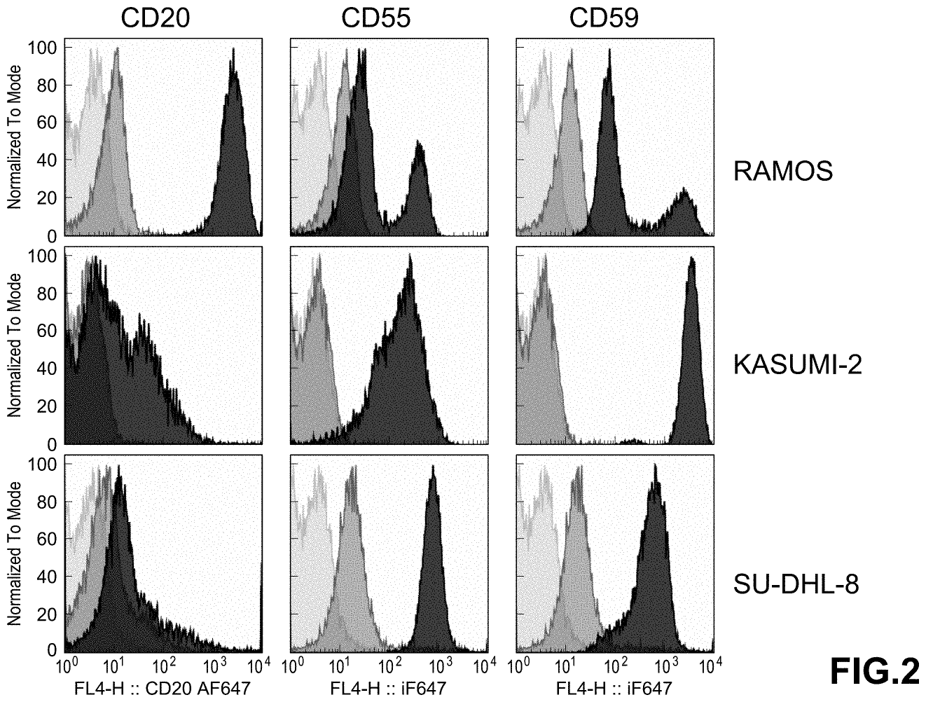

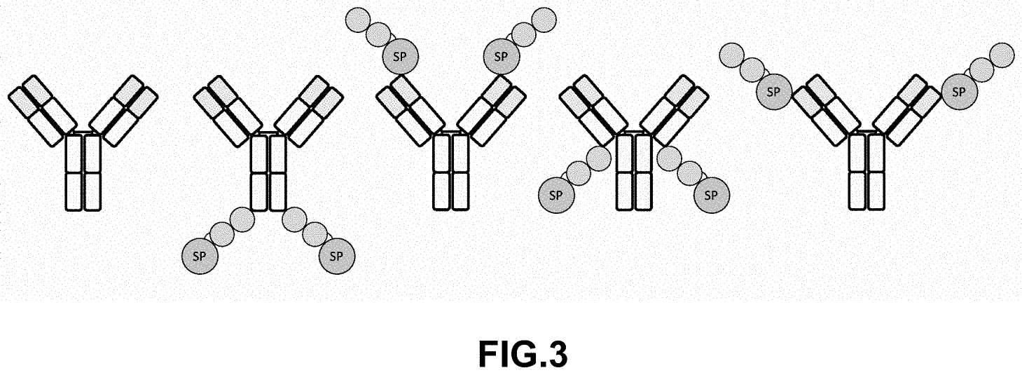

Binding of certain targeted complement-activating molecules to CD20 expressed on a cell surface was assessed using flow cytometry. First, expression levels of CD20 were examined on three cell lines, the human Burkitt lymphoma line Ramos (ATCC), the large cell lymphoma line SU-DHL-8 (ATCC) and the acute lymphoblastic leukemia line Kasumi-2 (DSMZ). All cell lines were maintained at 37° C. in RPMI 1640 medium [-] L-Glutamine supplemented with 10% heat inactivated FBS and GlutaMax (Gibco). About a half million cells were harvested and resuspended in FACS buffer (PBS with 2% FBS and 0.05% Sodium azide). To prevent non-specific binding, 5 μL of blocking solution (FcR binding inhibitor, eBioscience) was added to 100 μL of cell suspension, followed by 15 minutes incubation at room temperature. A primary antibody targeted against CD20 (rituximab) was added to the cell suspension. After 20 minutes incubation on ice, the cells were washed twice and were resuspended in FACS buffer containing the secondary antibody, an anti-human IgG Fc Ab conjugated with Alexa Fluor 647 (Clone HP6017, Biolegend). The cells were incubated on ice for 20 minutes and then were washed three times and resuspended in FACS buffer. Finally, the stained cell samples were analyzed on FACSCalibur. Results are shown in FIG. 2, left column.

The human Burkitt lymphoma line Ramos (ATCC) was selected for assay of CD20 binding by targeted complement-activating molecules comprising a rituximab binding domain. Cells were maintained at 37° C. in RPMI 1640 medium [-] L-Glutamine supplemented with 10% heat inactivated FBS and GlutaMax (Gibco). About a half million cells were harvested and resuspended in FACS buffer (PBS with 2% FBS and 0.05% Sodium azide). To prevent non-specific binding, 5 μL of blocking solution (Fc binding inhibitor, eBioscience) was added to 100 μL of cell suspension, followed by 15 minutes incubation at room temperature. Primary antibodies targeted against CD20 were added to the cell suspension. These antibodies included rituximab (anti-CD20 mAb) and the recombinant targeted complement-activating molecule, M2-RTX(H), RTX(L)-M2, M2-RTX(L), RTX(H)^{ΔK}-M3, M3-RTX(H), RTX(L)-M3, M3-RTX(L), RTX(H)^{ΔK}-MatCFD, MatCFD-RTX(H), RTX(L)-MatCFD, and MatCFD-RTX(L), as well as catalytically inactive construct RTX(H)^{ΔK}-M2^{S633A}. After 20 minutes incubation on ice, the cells were washed twice and were resuspended in FACS buffer containing the secondary antibody, an anti-human IgG Fc Ab conjugated with Alexa Fluor 647 (Clone HP6017, Biolegend). The cells were incubated on ice for 20 minutes and then were washed three times and resuspended in FACS buffer. Finally, the stained cell samples were analyzed on FACSCalibur.

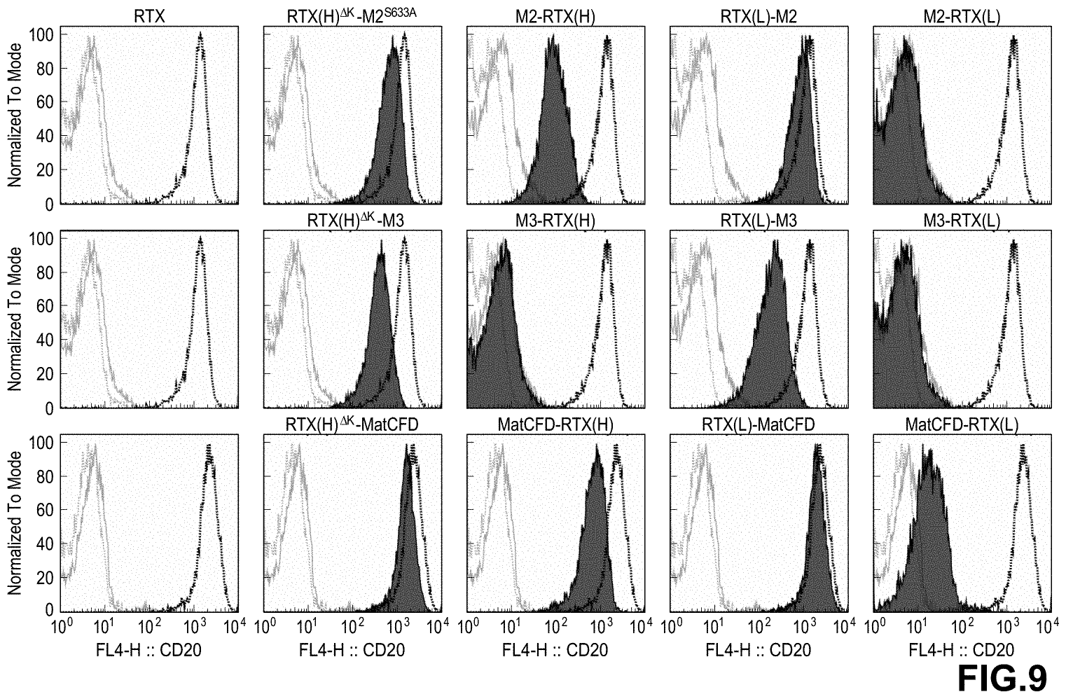

The FACS analysis showed that all the C-terminus protein fusions bind to CD20 on the cell surface, whereas of the N-terminus configurations, only M2-RTX(H) and MatCFD-RTX(H) bound CD20 on the cell surface. See FIG. 9.

Biolayer Interferometry

Biolayer interferometry was carried out to analyze binding kinetics of the recombinant targeted complement-activating molecules against the target CD20 (Acro Biosystems), using the Octet RED96 system (ForteBio Inc.). Anti-hIgG Fc (AHC) (ForteBio Inc.) was used to load the recombinant targeted complement-activating molecule RTX(H)^{ΔK}-C1r, RTX(H)^{ΔK}-C1s, ProCFD-RTX(H), RTX(H)^{ΔK}-M2^{R444K}, or RTX(H)^{ΔK}-M3 which was diluted in Kinetic Buffer (PBS, 0.02% Tween 20, 0.1% BSA, 0.05% DDM, 0.01% CHS) at a concentration of 69 nM and was added into one column of the assay plate. Various concentrations of CD20 were tested. The antigen was diluted in Kinetic Buffer with a 2-fold serial dilution, with starting concentration of 200 nM and was added into one column. To dissociate the proteins from the biosensors in order to load the next test sample, a regeneration step is required. For that purpose, Regeneration Buffer (10 mM Glycine pH 1.6) was added into one column of the assay plate and for neutralization Kinetic Buffer was added into another column. The binding kinetics of the proteins were analyzed by using Octet CFR Software (ForteBio Inc.).

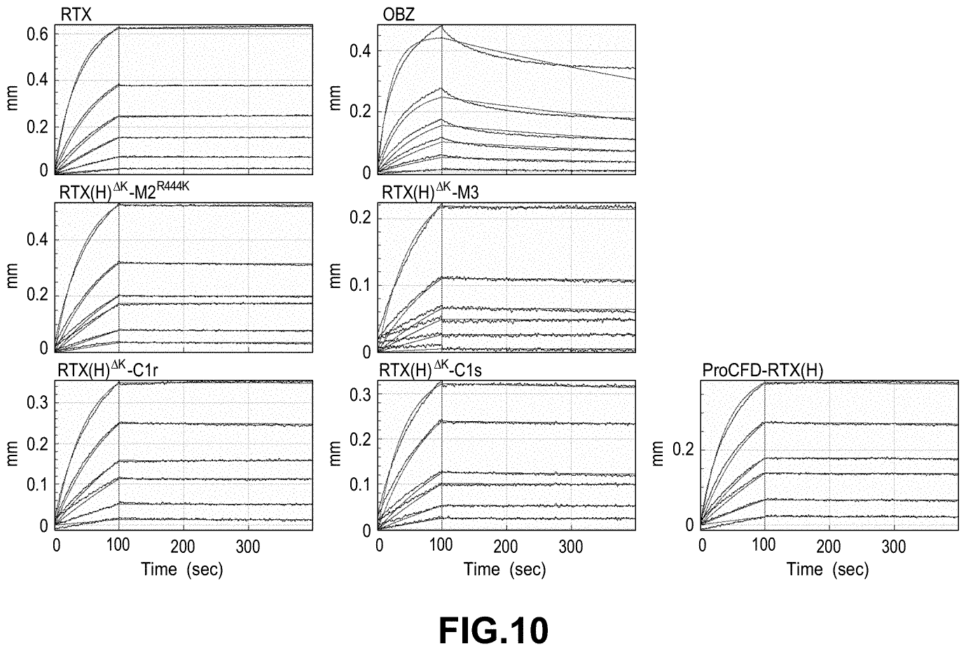

All the targeted complement-activating molecules tested were shown to bind to the antigen CD20 in dose-response manner. The calculated equilibrium constants (K_{D}) are as follows: RTX <1.00E-12M, OBZ 5.03E-09 M, RTX(H)^{ΔK}-M3 8.03E-10M, RTX^{ΔK}-M2^{R444K} 2.89E-10M, RTX(H)^{ΔK}-C1r 6.30E-11M, RTX(H)^{ΔK}-C1s 7.31E-10M, and ProCFD-RTX(H) 3.20E-10M. See FIG. 10 and Table 4.

TABLE 4

| Sample | K_{D} (M) | Kₒₙ (1/MS) | K_{dis} (1/s) | Full R² |
|---|---|---|---|---|
| RTX | <1.00E−12 | 1.40E+05 | <1.00E−07 | 0.9993 |
| OBZ | 5.03E−09 | 2.46E+05 | 1.23E−03 | 0.9923 |
| RTX(H)^{ΔK}-M2^{R444K} | 2.89E−10 | 1.17E+05 | 3.89E−05 | 0.9987 |
| RTX(H)^{ΔK}-M3 | 8.03E−10 | 9.52E+04 | 7.64E−05 | 0.9942 |
| RTX(H)^{ΔK}-C1r | 6.30E−11 | 1.05E+05 | 6.61E−06 | 0.9986 |
| RTX(H)^{ΔK}-C1s | 7.31E−10 | 1.21E+05 | 8.86E−05 | 0.9972 |
| ProCFD-RTX(H) | 3.2E−10 | 1.26E+05 | 4.03E−05 | 0.9983 |

### Example 4

### Activity of Rituximab-Derived Targeted Complement-Activating Molecules

Serine Protease Activity

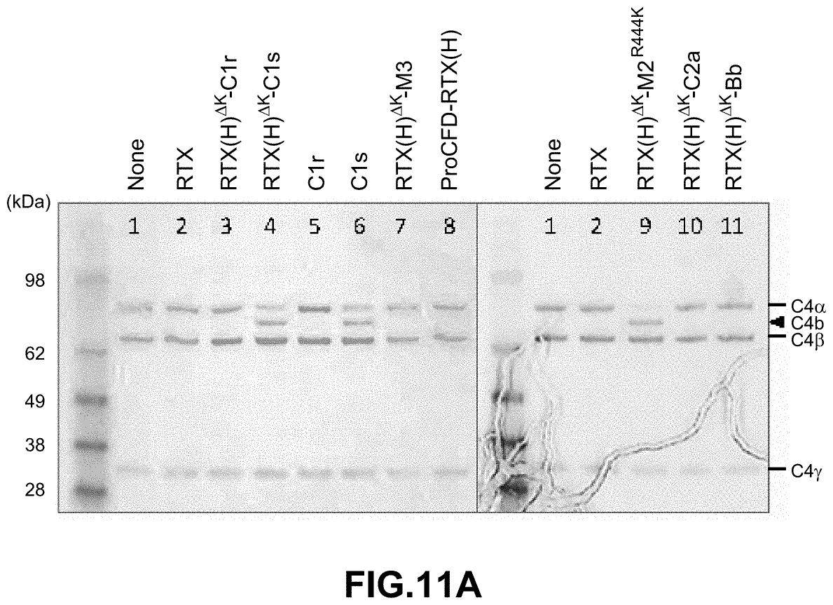

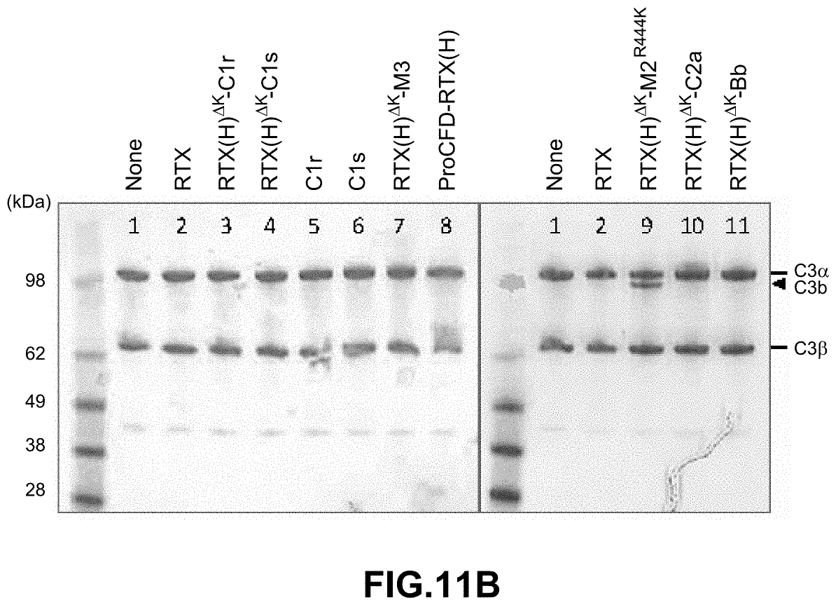

A substrate cleavage activation assay of various targeted complement-activating molecules was performed in which the complement components C4 and C3 were used as substrates. The substrates were diluted in PBS (1×), pH 7.4, and incubated at 37° C. alone or with the addition of one of RTX(H)^{ΔK}-C1r, RTX(H)^{ΔK}-C1s, RTX(H)^{ΔK}-M3, proCFD-RTX(H), RTX(H)^{ΔK}-M2^{R444K}, RTX(H)^{ΔK}-C2a or RTX(H)^{ΔK}-Bb in an enzyme/substrate ratio of 1:20. The samples were removed after three hours and analyzed by SDS-PAGE under reducing conditions to detect the cleavage of either C4 or C3. Results are shown in FIGS. 11A (C4 substrate) and 11B (C3 substrate).

Enzyme activity of the Ab-protease fusions was measured in a microtiter plate based on cleavage of a synthetic fluorogenic or chromogenic peptide substrate. Recombinant fusion proteins were incubated in assay buffer (for fluorometric assay, 20 mM HEPES pH 7.4, 140 mM NaCl, 0.1% Tween 20; for colorimetric assay, 50 mM Tris pH 7.5, 1 M NaCl) at room temperature with an appropriate synthetic peptide substrate (5-200 μM depending on the enzymes). Changes in fluorescence or absorbance were monitored for 20 min, and the enzyme activity was calculated from initial rates of the changes and expressed as RU/min/μmol of enzyme catalytic site to allow comparison to purified enzyme controls. Results are shown in Table 5.

TABLE 5

| | | |
|---|---|---|
| | | Activity relative to |
| | Cleavage activity | an enzyme control |
| Protein sample | [RU/min/μmol] | [%] |

MASP-2 assay

| | | |
|---|---|---|
| RTX(H)-M2 | 1.82E+11 | 25.3 |
| RTX(H)^{ΔK}-M2 | 1.42E+11 | 19.8 |
| | 9.08E+10 | 17.0 |
| | 1.19E+11 | 16.2 |
| RTX(H)^{ΔK}-M2^{R444K} | 2.62E+11 | 35.7 |
| RTX(H)^{ΔK}-M2^{R444Q} | n.d. | 0 |
| RTX(H)^{ΔK}-M2^{S633A} | n.d. | 0 |
| M2-RTX(H) | 4.84E+11 | 67.5 |
| | 2.75E+11 | 51.6 |
| RTX(L)-M2 | 1.15E+11 | 21.6 |
| M2-RTX(L) | 3.54E+11 | 66.3 |
| MASP2(CCP12SP) | 7.17E+11 | 100 |
| | 5.34E+11 | |
| | 7.35E+11 | |

MASP-3 assay

| | | |
|---|---|---|
| RTX(H)^{ΔK}-M3 | 1.84E+13 | 5.3 |
| | 8.65E+12 | 22.7 |
| RTX(H)^{ΔK}-M3 | 3.97E+13 | 104 |
| (activated by M2) | 3.12E+13 | 82.0 |
| | 2.89E+13 | 76.0 |
| M3-RTX(H) | 1.25E+13 | 33.0 |
| RTX(L)-M3 | 2.42E+13 | 8.0 |
| M3-RTX(L) | 1.54E+13 | 5.1 |
| MASP3(SP) | 3.48E+14 | 100 |
| | 3.81E+13 | |
| | 3.04E+14 | |

MASP-1 assay

| | | |
|---|---|---|
| RTX(H)^{ΔK}-M1^{R504Q} | n.d. | 0 |
| M1^{R504Q}-RTX(L) | n.d. | 0 |
| MASP1(CCP2SP) | 2.65E+14 | 100 |

C1r assay

| | | |
|---|---|---|
| RTX(H)^{ΔK}-C1r | 3.69E+12 | 284 |
| RTX(H)^{ΔK}-C1r^{S654A} | n.d. | 0 |
| C1r(CCP12SP) | 1.30E+12 | 100 |

C1s assay

| | | |
|---|---|---|
| RTX(H)^{ΔK}-C1s | 1.37E+14 | 278 |
| RTX(H)^{ΔK}-C1s^{S632A} | n.d. | 0 |
| C1s(CCP12SP) | 4.91E+13 | 100 |

CFD assay

| | | |
|---|---|---|
| RTX(H)^{ΔK}-MatCFD | n.d. | 0 |
| MatCFD-RTX(H) | 9.40E−03 | 115 |
| RTX(L)-MatCFD | n.d. | 0 |
| MatCFD-RTX(L) | 9.79E−03 | 119 |
| MatCFD^{S208A}-RTX(H) | n.d. | 0 |
| ProCFD-RTX(H) | 4.04E−04 | 12.7 |
| MatCFD | 8.21E−03 | 100 |

### C4 Deposition Assays

In the lectin pathway of the complement system, MASP-2, a serine protease, activates the complement cascade by cleaving the proteins C4 and C2. The activation of C4 leads to C4b deposition onto the surface of the target cell. This cleavage activity is shared with the C1 complex of the classical pathway. Therefore, functional activity of the MASP-2, C1r, and C1s proteins was studied through a C4 deposition assay. In the absence of MASP-2, the lectin pathway is not functional, as was shown in plasma from MASP-2-depleted human serum (Møller-Kristensen et al., 2007) and in plasma from MASP-2 knockout mice (Schwaeble et al., 2011). Hence, in order to prevent deposition from serine proteases in the plasma itself, the plasma that was used were collected from MASP-2 knockout mice. Activity in normal human serum was tested as well, and those tested include serum-only control, aglycosylated antibody domain (to prevent C1q binding to the Fc region and initiating classical pathway activity), and negative controls with catalytically inactive molecules.

For mouse plasma assays, Nunc Maxisorp microtiter ELISA plates were coated with 100 μL of coating buffer (15 mM Na₂CO₃, 35 mM NaHCO₃) containing mannan (50 μg/mL; Sigma-Aldrich, M7504) and/or the recombinant targeted complement-activating molecules (215 nM) and incubated at 4° C. overnight. The next day, 250 μL of PBS buffer containing 1% BSA (Sigma-Aldrich, A3294) was added to each well and incubated at room temperature for 2 hours to block the remaining protein binding surface. The plates were washed 3 times with PBS containing 0.05% Tween 20 (wash buffer). Hirudin mouse plasma was diluted with PBS (no calcium, no magnesium) and added to the wells. The plates were incubated for 15 minutes at 4° C. and washed three times.

To assess the deposition of C4 on the mannan surface, C4b was detected with a rat anti-C4 monoclonal antibody (16D2, Santa Cruz Biotechnology). 100 μL/well, diluted in wash buffer at a final concentration of 0.2 μg/mL, were added and the plates were agitated for 30 minutes at 37° C. at 200 rpm. The plates were washed three times and 100 μL of a secondary antibody were added. The secondary antibody that was used was a goat anti-rat IgG(H+L) (Cat #3051-05, Southern Biotech) conjugated with an alkaline phosphatase (AP) which was diluted in wash buffer at a final concentration of 0.043 μg/mL. The plates were incubated for 30 minutes at room temperature. Finally, 100 μL of a colorimetric substrate TMB (1-step Ultra TMB-ELIS, Thermo-Scientific, 34029) were added to the plates. The reaction was stopped by adding 100 μL of 0.1 N sulfuric acid (BDH7230-1) and the absorbance was measured at 450 nm using a plate reader.

For human serum assays, Nunc Maxisorp microtiter ELISA plates were coated with 100 μL of coating buffer (15 mM Na₂CO₃, 35 mM NaHCO₃) containing the recombinant fusion proteins (69 nM) and incubated at 4° C. overnight. The next day, 250 μL of PBS buffer containing 1% BSA (Sigma-Aldrich, A3294) was added to each well and incubated at room temperature for 2 hours to block the remaining protein binding surface. The plates were washed 3 times with PBS containing 0.05% Tween 20 (wash buffer). Normal Human Serum (NETS) was diluted with PBS (no calcium, no magnesium) and added to the wells. The plates were incubated for 10 minutes at 4° C. and washed three times.

To assess the deposition of C4 on the mannan surface, C4b was detected with a C4c polyclonal rabbit anti-human (Q0369, Dako). 100 μL/well, diluted in wash buffer at a final concentration of 0.88 μg/mL, were added and the plates were agitated for 30 minutes at 37° C. at 200 rpm. The plates were washed three times and 100 μL of a secondary antibody were added. The secondary antibody that was used was a goat anti-rabbit IgG (H+L) (Lot #G0710-V488D, Southern Biotech) conjugated with an alkaline phosphatase (AP) which was diluted in wash buffer at a final concentration of 0.043 μg/mL. The plates were incubated for 30 minutes at room temperature. Finally, 100 μL of a colorimetric substrate TMB (1-step Ultra TMB-ELIS, Thermo-Scientific, 34029) were added to the plates. The reaction was stopped by adding 100 μL of 0.1 N sulfuric acid (BDH7230-1) and the absorbance was measured at 450 nm using a plate reader.

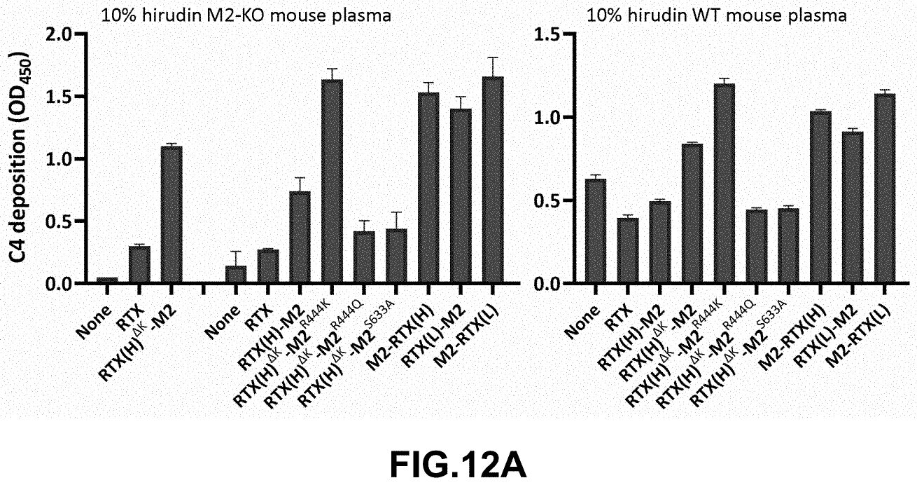

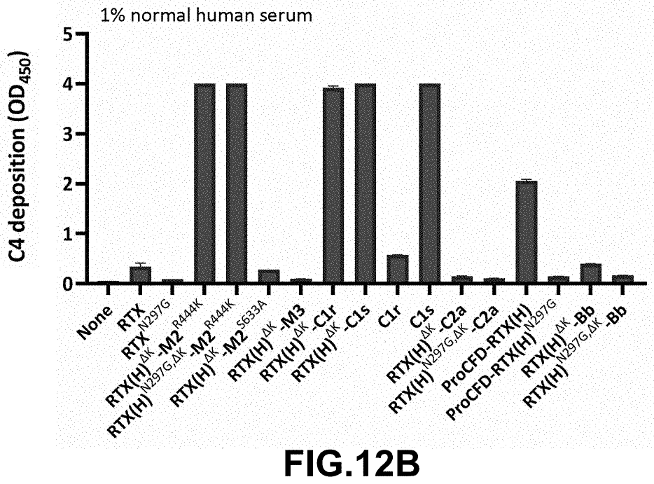

Results are shown in FIGS. 12A and 12B.

C3 Deposition Assays

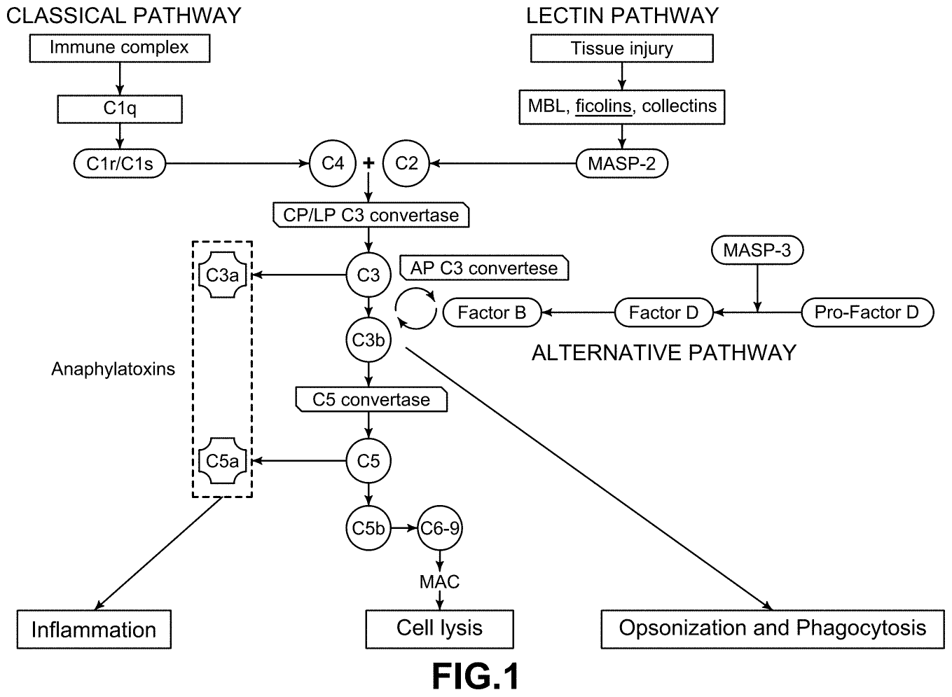

The role of MASP-3 in the alternative pathway of the complement system was revealed recently and it was shown that active MASP-3 converts pro-CFD to mature CFD (Dobó et al., 2016). Activated complement factor D (CFD) is the serine protease that cleaves Factor B (FB) in the pro-convertase C3bB of the alternative pathway resulting in the formation of the C3 convertase C3bBb. The C3 convertase cleaves C3 and generates C3b molecules, which bind covalently onto the cell surface. Formation of the C4bC2a convertase involves the participation of complement components of the classical and lectin pathways. The complement component C2 undergoes cleavage by MASP-1, MASP-2, C1r and C1s ensuing binding to C4b, that results to the C3 convertase of the classical and lectin pathway. The C3 convertase subsequently cleaves C3 and generates C3b molecules, which bound covalently onto the surface (FIG. 1). Therefore, the functional activity of all these serine proteases described above can be assessed with a C3 deposition assay. To prevent deposition of C3 due to MASP-3 activity from the plasma (Takahashi et al., 2008), plasma from MASP-1/3 knockout mice was used.

For mouse plasma assays, Nunc Maxisorp microtiter enzyme-linked immunosorbent assay plates were coated with 100 μL zymosan (10 μg/mL) and/or the recombinant targeted complement-activating molecules (215 nM) suspended in coating buffer (15 mM Na₂CO₃, 35 mM NaHCO₃) followed by overnight incubation at 4° C. The next day, 250 μL of 1% BSA (Sigma-Aldrich, A3294) in PBS buffer were added to each well and the plate was incubated for 2 hours at room temperature, followed by washes with wash buffer. Hirudin mouse plasma was diluted with MgEGTA buffer (10 mM EGTA, 5 mM MgCl₂, 5 mM Barbital, 145 mM NaCl [pH 7.4]) and added to the wells. The plates were incubated for 50 minutes at 37° C. and washed three times.

For C3b detection on the surface, a rabbit anti-human C3c antibody (Dako, Lot #B298875) was used. This C3c antibody is able to recognize C3b. 100 μL/well ((2.4 μg/mL)) diluted in wash buffer were added and the plates were incubated for 30 minutes at 37° C. at 200 rpm. The plates were washed three times and 100 μL of a secondary antibody Goat Anti-Rabbit (Southern Biotech) conjugated with an alkaline phosphatase (AP) and diluted in wash buffer (0.043 μg/mL) was added to the plates. The plates were incubated for 30 minutes at room temperature. For determination of C3 deposition, 100 μL of TMB (1-step Ultra TMB-ELIS, Thermo-Scientific, 34029) were added to the plates. The reaction was stopped by adding 100 μL of 0.1 N sulfuric acid (BDH7230-1) and the absorbance was measured at 450 nm using a plate reader.

For human serum assays, Nunc Maxisorp microtiter enzyme-linked immunosorbent assay plates were coated with the recombinant fusion proteins (250 nM) suspended in coating buffer (15 mM Na₂CO₃, 35 mM NaHCO₃) followed by overnight incubation at 4° C. The next day, 250 μL of 1% BSA (Sigma-Aldrich, A3294) in PBS buffer were added to each well and the plate was incubated for 2 hours at room temperature, followed by washes with wash buffer. NHS was diluted with MgEGTA buffer (10 mM EGTA, 5 mM MgCl₂, 5 mM Barbital, 145 mM NaCl [pH 7.4]) and added to the wells. The plates were incubated for 25 minutes at 37° C. and washed three times. C3b detection was carried out as for the mouse plasma assays.

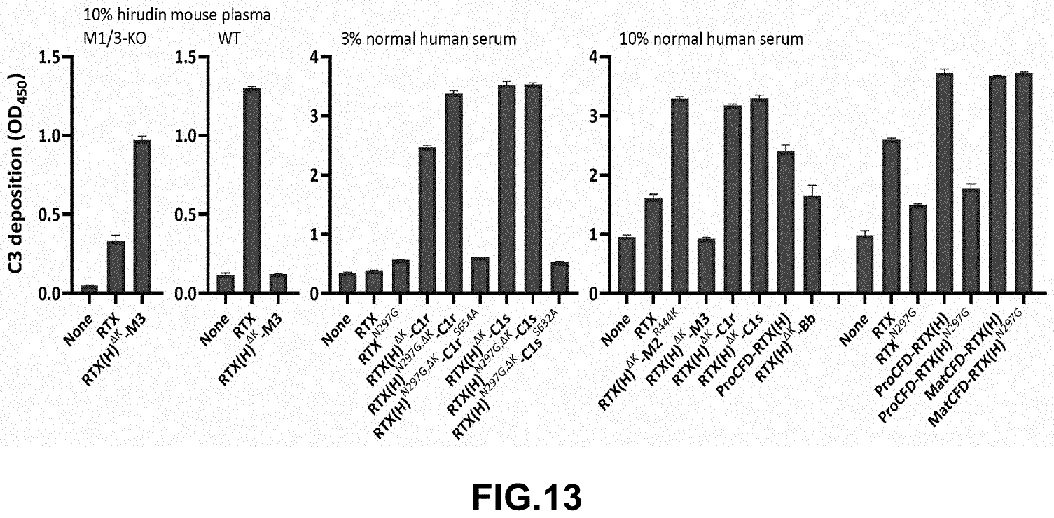

Results are shown in FIG. 13.

Complement Deposition on Target Cells

The targeted complement-activating molecules consist of two domains; the target-binding domain, which binds to the target via the variable regions (Fv) of the Fab fragment of an antibody, and the serine protease effector domain from a complement-activating serine protease. A complement component deposition assay on CD20 positive cells was performed to assess the effect of the targeted complement-activating molecule as a whole. The acute lymphoblastic leukemia line Kasumi-2 (purchased from DSMZ) was used to examine complement deposition on the cell surface after treatment with targeted complement-activating molecules ProCFD-RTX(H) and MatCFD-RTX(H). Final concentration of 12.5 nM and 1.4 nM of the proteins were diluted in CDC Assay Buffer (RPMI 1640 Medium [-] L-Glutamine, 5% heat-inactivated FBS, GlutaMax and 25 mM HEPES). RTX and the aglycosylated forms were included as controls. Normal Human Serum (NETS) was also diluted into the Assay Buffer to obtain a final concentration at 15%. The cells were resuspended into the Assay Buffer to a final concentration of 300,000 cells/mL and were transferred to a 6-well assay plate followed by addition of the diluted proteins and the human serum. The assay plates were incubated at 37° C. in a humidified incubator for 2 hours. After treatment the cells were resuspended into FACS buffer (PBS with 2% FBS and 0.05% sodium azide), blocked to prevent non-specific binding with blocking solution (Human TruStain FcX, Biolegend) (5 μL/100 μL), and were stained with primary antibodies against the complement components C3 or C5b-9 (MAC). The primary antibodies (5 μg/mL) that were used are the rabbit anti-human C3c (A0062, Dako) and the monoclonal mouse anti-human C5b-9 (M0777, Dako). After 20 minutes incubation on ice, the cells were washed twice and were resuspended in FACS buffer containing the secondary antibody (5 μL/100 μL), APC anti-rabbit IgG (F0111, R&D Systems) and PE anti-mouse IgG (405307, BioLegend) recognizing the C3 and C5b-9 antibodies, respectively. The cells were incubated on ice for 20 minutes and then were washed three times and resuspended in FACS buffer. Finally, the stained cell samples were analyzed on FACSCalibur.

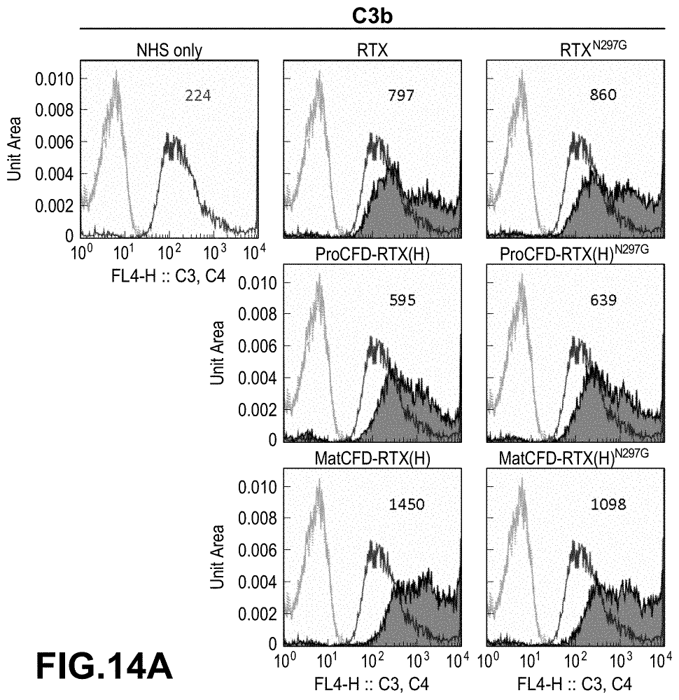

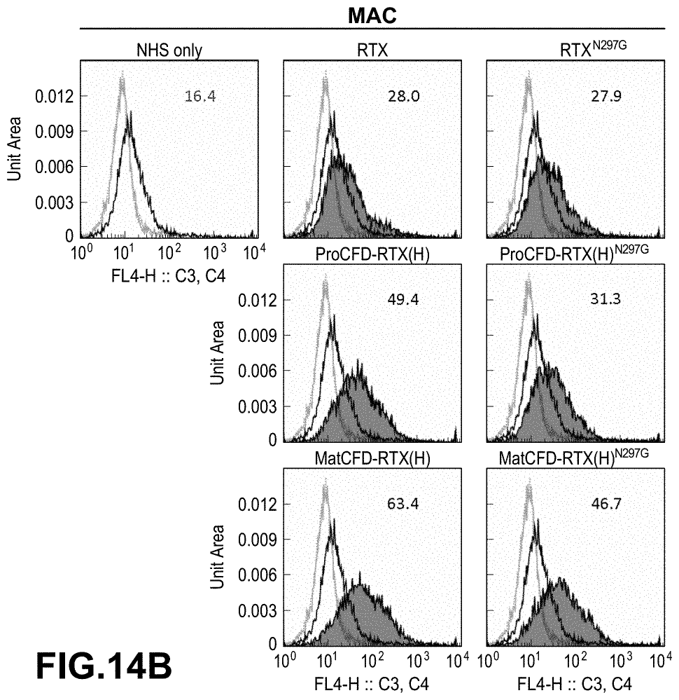

CFD fusions, especially MatCFD fusions, induced significantly higher deposition of C3 and MAC on target cells as compared to serum only or RTX treatment. Results are shown in FIGS. 14A (C3 deposition) and 14B (MAC deposition).

### Example 5

### Cytotoxicity of Rituximab-Derived Targeted Complement-Activating Molecules

CDC Assays

The Ramos B cell line expresses high levels of CD20, to which the antibody rituximab binds. Increased density of the antigen plays a role in the initiation of the complement cascade through antibody binding that initiates the classical pathway. Likewise, the active C1r and C1s serine proteases are activators of the classical pathway. The catalytic domains of MASP-1 and MASP-2 activate the lectin pathway and the catalytic domains of MASP-3 and CFD activate the alternative pathway. C2 is an activator of the classical and lectin pathways, and Factor B activates the alternative pathway.

All three pathways of the complement system lead to the formation of MAC on the surface of the target cell, followed by lysis of the cell. It is expected that activating more than one complement pathway would lead to enhanced complement-dependent cytotoxicity (CDC). Therefore, the targeted complement-activating molecules were tested for the ability to increase levels of CDC, which could result from activation of any two or more of the classical, lectin, and alternative complement pathways.

Complement-dependent cytotoxicity (CDC) assays were performed using a CytoTox-Glo Cytotoxicity assay kit (Promega). Serial dilutions of rituximab and targeted complement-activating molecules (highest concentration: 12.5 nM) were prepared with Assay Buffer (RPMI 1640, 5% heat-inactivated FBS, GlutaMax, 25 mM HEPES). Normal Human Serum (NETS) was also diluted into the Assay Buffer to obtain a final concentration of 15%. CD20+ cells were resuspended into the Assay Buffer to a final concentration of 10,000 cells/well and were transferred to the 96-well assay plate followed by addition of the diluted proteins and the human serum. The assay plates were incubated at 37° C. in a humidified incubator for 2 hours. After cooling down at room temperature for 15 minutes, the CytoTox-Glo reagent (Promega) was added and incubated for additional 10 minutes. Finally, the luminescence was measured using a microplate luminometer (Luminoskan Lab systems).

In some cases, CDC assays were performed using propidium iodide. Serial dilutions of rituximab and targeted complement-activating molecules were prepared with Assay Buffer (Opti-MEM cell culture medium, Gibco). Normal Human Serum (NETS) was also diluted into Assay Buffer to obtain a final concentration at 10%. CD20+ cells were washed with PBS, resuspended with the Assay Buffer to a final concentration of 150,000 cells/well and transferred to the 96-well assay plate followed by addition of the diluted proteins and the human serum. The assay plates were incubated at 37° C. in a humidified incubator for 2 hours. After incubation, 5 μL of propidium iodide (Invitrogen, cat #00-6990-50) were added and the stained cells were immediately analyzed by flow cytometry using a FACSCalibur.

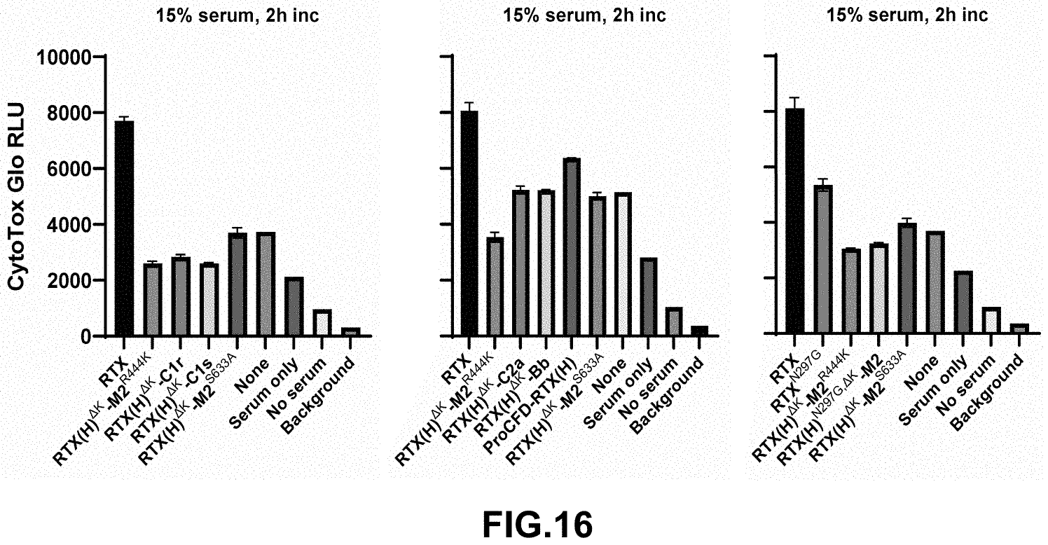

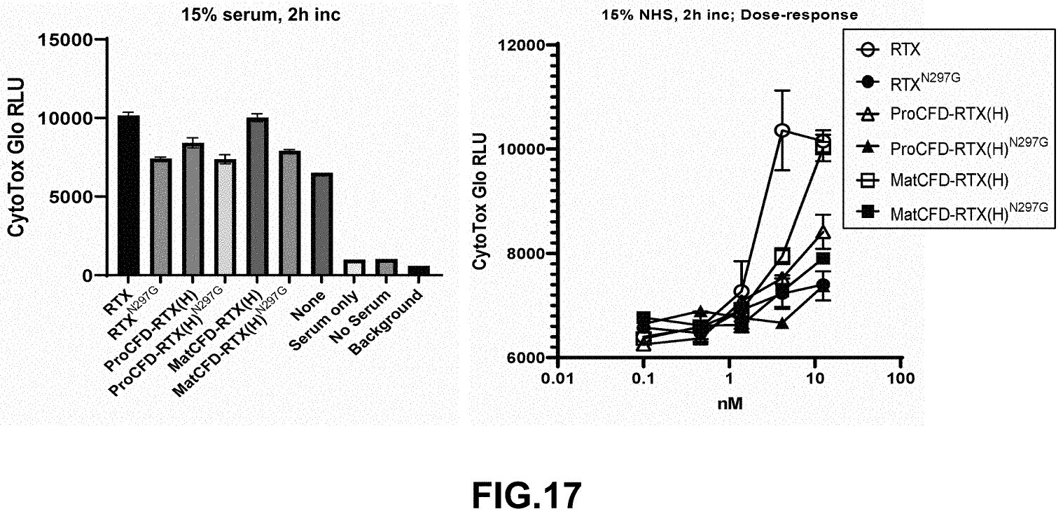

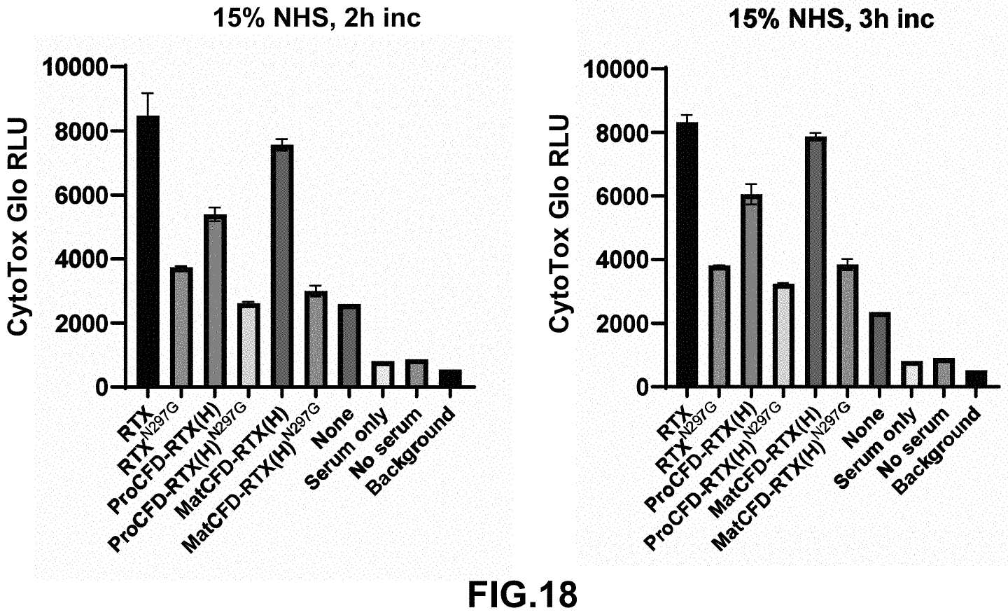

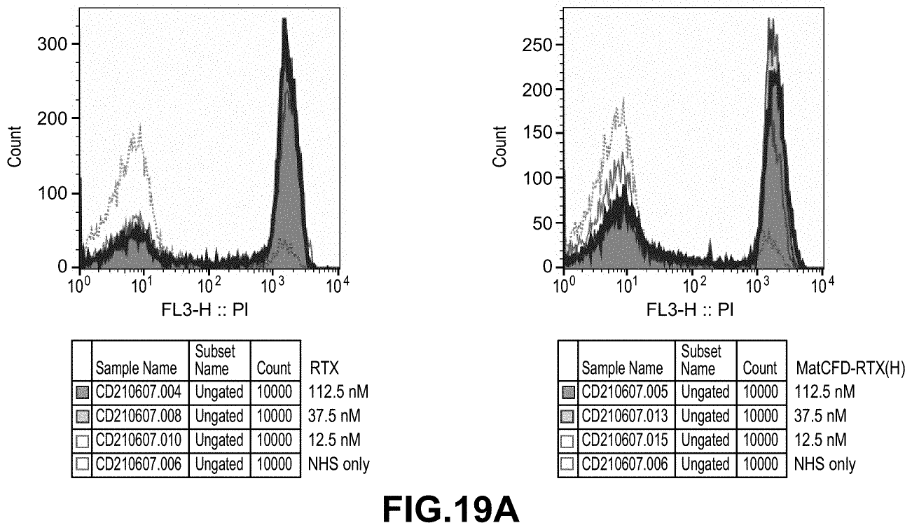

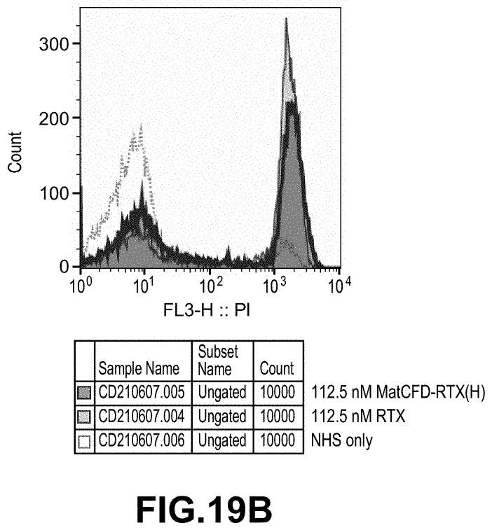

Results of CytoTox-Glo assays are shown in FIGS. 16-18. Results of propidium iodide assays are shown in FIG. 19.

Inhibition of CD55 and CD59

In order to determine the possible impact of complement regulatory proteins (CRPs) in the CDC assays, additional assays were carried out using CRP inhibitors. Either antibodies against CRP CD55 (clone BRIC 216, Sigma-Aldrich), CRP CD59 (clone BRIX 229, IBGRL), or both were used to inhibit the activity of CD55 and/or CD59 during the assay.

Dilutions of rituximab (RTX), modified rituximab (RTX^{N297G}), targeted complement-activating molecule MatCFD-RTX, and targeted complement-activating molecule MatCFD-RTX^{N297G} were prepared with Assay Buffer to a final concentration of 337.5 nM. Anti-CD55 antibody was prepared with Assay Buffer to a final concentration 10 μg/mL and anti-CD59 to a final concentration of 2 μg/mL. Normal Human Serum (NHS) was also diluted into Assay Buffer to obtain a final concentration of 15%. Ramos cells were resuspended with Assay Buffer to a final concentration of 300,000 cells/well and transferred to the 96-well assay plate followed by addition of the anti-CRPs, the diluted RTX and targeted complement-activating molecules and the human serum. The assay plate was incubated at 37° C. in a humidified incubator for 2 hours. After incubation, 5μL of propidium iodide (Invitrogen, cat #00-6990-50) were added and cells were immediately analyzed by flow cytometry using FACSCalibur. Samples with no anti-CRP antibodies were also run as controls (no inh).

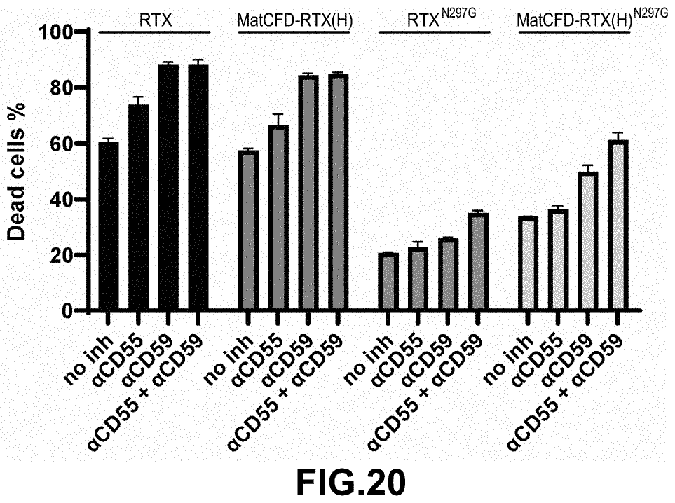

MatCFD-RTX^{N297G} induced significantly higher CDC on Ramos cells as compared to RTX^{N297G} when one or both CRPs were inhibited. Glycosylated RTX had already high CDC, thereby no further enhancement was observed with MatCFD-RTX(H). Results are shown in FIG. 20.

### Example 6

### Production of Alemtuzumab and Daratumumab Fusion Proteins

Mature complement factor D (MatCFD) was fused with anti-CD52 antibody alemtuzumab (ALM) or with anti-CD38 antibody daratumumab (DARA), and the fusion proteins were expressed using the expression vector pCAG. The vector is a modified version of pD2610-v1 (ATUM; originally from (Miyazaki et al., 1989)) and contains the characteristic CMV and chicken beta-actin hybrid promoter, and a kanamycin resistance marker.

The MatCFD domains were fused to the N-terminus of the antibody's heavy chain and resulted in the following constructs: MatCFD-ALM(H) (SEQ ID NO:97) and MatCFD-DARA(H) (SEQ ID NO:98).

Plasmid preparation, cloning, protein expression, and purification was carried out as described in Example 1.

### Example 7

### Binding of Alemtuzumab-Derived and Daratumumab-Derived Targeted Complement-Activating Molecules to Targets

Flow Cytometry

Binding of targeted complement-activating molecules having an alemtuzumab-derived binding domain to CD52 expressed on a cell surface and binding of targeted complement-activating molecules having a daratumumab-derived binding domain to CD38 expressed on a cell surface were assessed using flow cytometry. About 500,000 cells of human B cell lymphoma line HT (ATCC) were harvested and resuspended in FACS buffer. To prevent non-specific binding, 5 μl of blocking solution was added to 100 μl of cell suspension, which was then incubated 15 minutes at room temperature. Antibodies alemtuzumab (targeting CD52) and daratumumab (targeting CD38) or one of targeted complement-activating molecules MatCFD-ALM(H) or MatCFD-DARA(H) were added to the cell suspension and incubated 20 minutes on ice. The cells were then washed twice and resuspended in FACS buffer containing secondary antibody (mouse anti-human IgG1 conjugated with Alexa Fluor 647). The cells were incubated on ice for 20 minutes, then washed three times and resuspended in FACS buffer. The stained cell samples were analyzed by FACS (FACSCalibur).

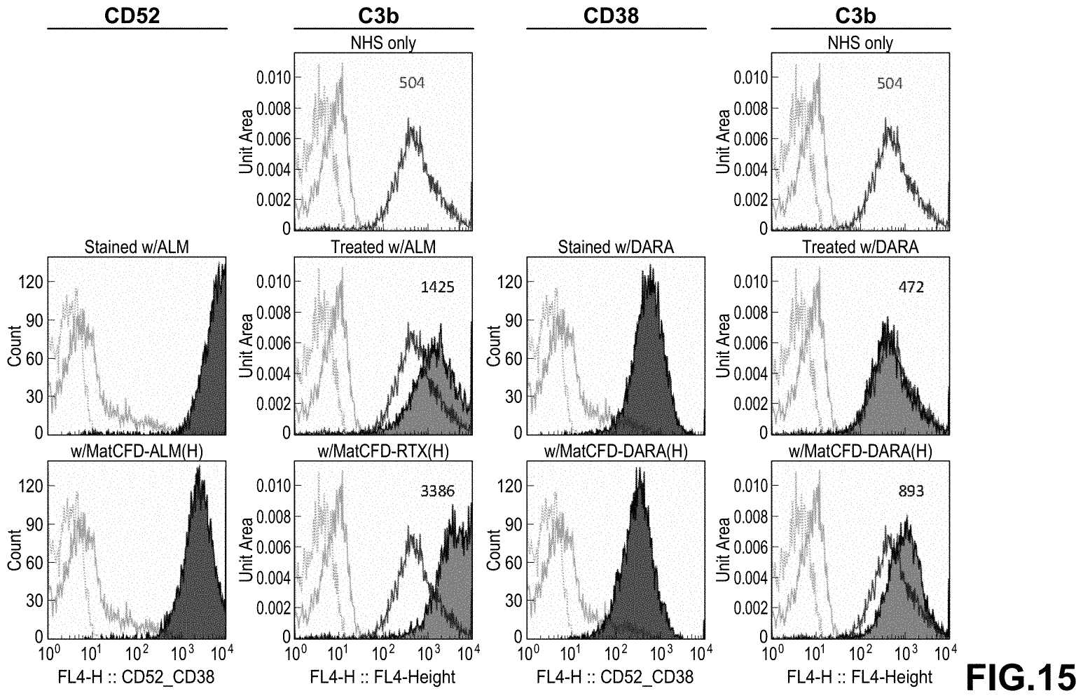

The FACS analysis showed that both ALM and targeted complement-activating molecule MatCFD-ALM(H) bind to CD52 on the surface of HT cells, and both DARA and MatCFD-DARA(H) bind to CD38 on the surface of HT cells. See FIG. 15, columns headed “CD52” and “CD38.”

### Example 8

### Activity of Alemtuzumab-Derived and Daratumumab-Derived Targeted Complement-Activating Molecules

C3 Deposition

Complement activation by targeted complement-activating molecules MatCFD-ALM(H) and MatCFD-DARA(H) were assessed by measurement of C3b deposition on HT target cells. Normal human serum (NETS) was diluted into Assay Buffer to obtain a final concentration of 15%. HT cells were resuspended into Assay Buffer to a final concentration of 300,000 cells/ml and were transferred to a 6-well assay plate. The diluted proteins and NETS were added to the wells. Plates were incubated at 37° C. in a humidified incubator for two hours. The cells were then resuspended into FACS buffer, blocked to prevent non-specific binding, and stained with primary antibody (rabbit anti-human C3c). After 20 minutes incubation in ice, the cells were washed twice and resuspended in FACS buffer containing secondary antibody (APC anti-rabbit IgG). The cells were incubated a further 20 minutes on ice, then washed three times and resuspended in FACS buffer. The stained cell samples were analyzed by FACS (FACSCalibur).

The FACS analysis showed that C3b deposition resulted from treatment with MatCFD-ALM(H) or MatCFD-DARA(H). See FIG. 15, columns labeled “C3b”.

### Example 9

### Preparation of Anti-fHbP-Derived Targeted Complement-Activating Molecules

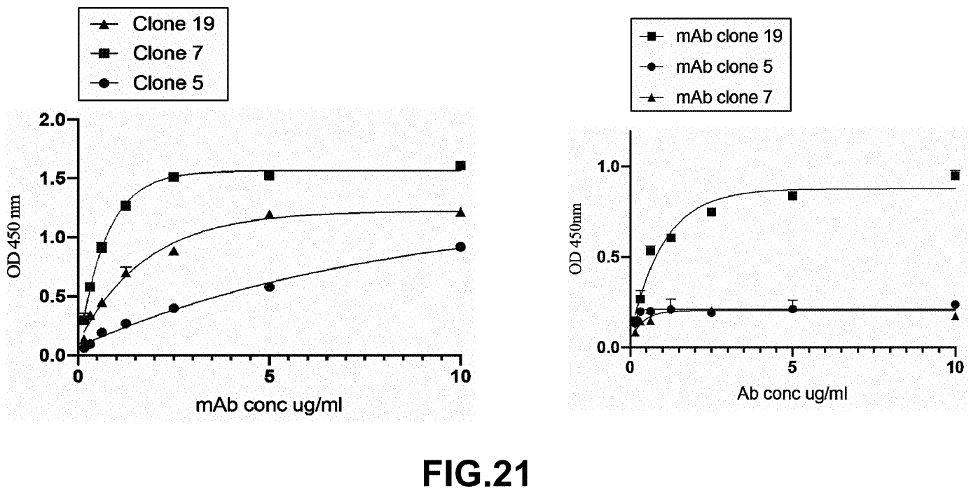

Monoclonal antibodies to factor H binding protein (fHbP) of Neisseria meningitidis (N. meningitidis) were produced in mice and isolated using hybridoma technique. Three different mouse monoclonal antibodies were identified: anti-fHbP clone 5 (aN5), anti-fHbP clone 7 (aN7), and anti-fHbP clone 19 (aN19). The binding of each of these three antibodies to fHbP on the surface of an ELISA plate was tested. All three antibodies showed binding to fHbP under these conditions. See FIG. 21, left panel. The binding of each of the three antibodies to N. meningitidis on the surface of an ELISA plate was then tested. Clone 19 showed binding to N. meningitidis under these conditions. See FIG. 21, right panel.

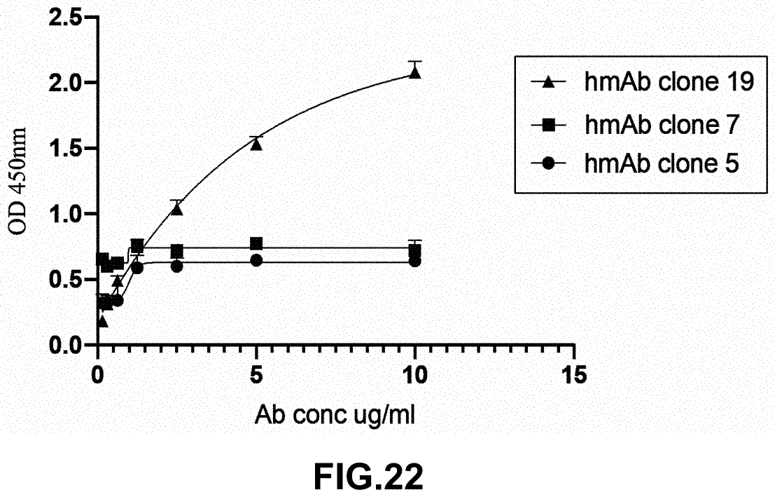

Each of clones 5, 7, and 19 was sequenced and expressed as a recombinant mouse-human chimera. Binding of the chimeric versions to N. meningitidis on the surface of an ELISA plate was tested. Clone 19 showed binding to N. meningitidis under these conditions. See FIG. 22.

The C1r and C1s serine protease effector domains were fused to one of three monoclonal antibodies that bind Neisseria meningitidis factor H binding protein (fHbP). The fusion proteins were expressed using the expression vector pCAG similarly to the proteins described in Example 1. The C-terminal catalytic fragment of C1r and C1s (CCP1-CCP2-SP) was fused with anti-fHbP clone 5 (aN5), clone 7 (aN7), or clone 19 (aN19) at the C-terminus of the antibody's heavy chain (HC), which was altered by a deletion of the single amino acid lysine (K) from the C-terminus. This process resulted in the following constructs: aN7(H)^{ΔK}-C1r (SEQ ID NO:107), aN19(H)^{ΔK}-C1r (SEQ ID NO:108), aN5(H)^{ΔK}-C1r (SEQ ID NO:109), aN7(H)^{ΔK}-C1s (SEQ ID NO:110), aN19(H)^{ΔK}-C1s (SEQ ID NO:111), aN5(H)^{ΔK}-C1s (SEQ ID NO:112).

The following additional clone 19-derived targeted complement-activating molecules were produced: aN19(H)^{ΔK}-M2^{R444K} (^{SE}Q ID NO:116), aN19(H)^{ΔK}-M3 (SEQ ID NO:117), MatCFD-aN19(H) (SEQ ID NO:118), ProCFD-aN19(H) (SEQ ID NO:119).

Plasmid preparation, cloning, protein expression, and purification was carried out as described in Example 1.

### Example 10

### Binding of Anti-fHbP-Derived Targeted Complement-Activating Molecules to N. meningitidis

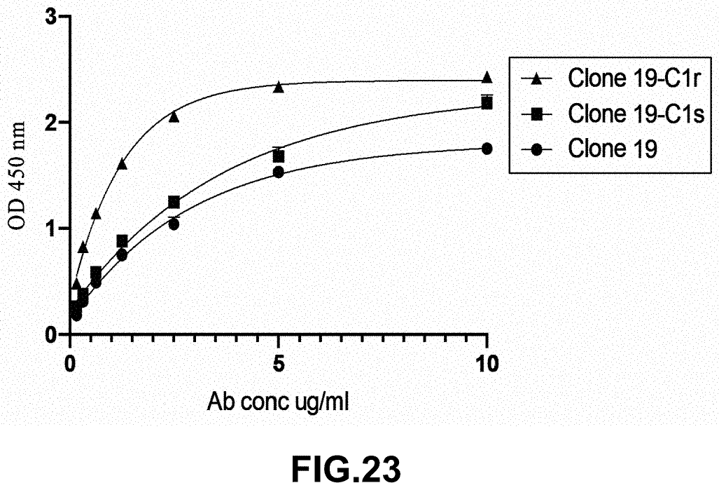

Binding to N. meningitidis was tested for each of the targeted complement-activating molecules comprising a clone 19 binding domain. Targeted complement-activating molecules aN19(H)^{ΔK}-C1r (also referred to as clone19-C1r) and aN19(H)^{ΔK}-C1s (also referred to as clone19-C1s) were tested, along with monoclonal antibody clone 19. All three molecules showed binding to N. meningitidis. See FIG. 23.

### Example 11

### Activity of Anti-fHbP-Derived Targeted Complement-Activating Molecules

Complement Deposition Activity

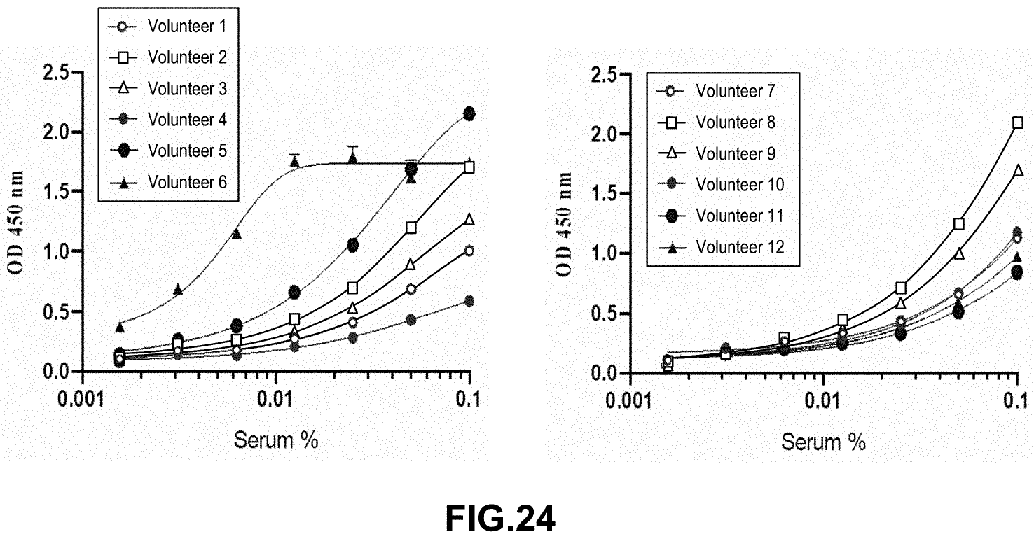

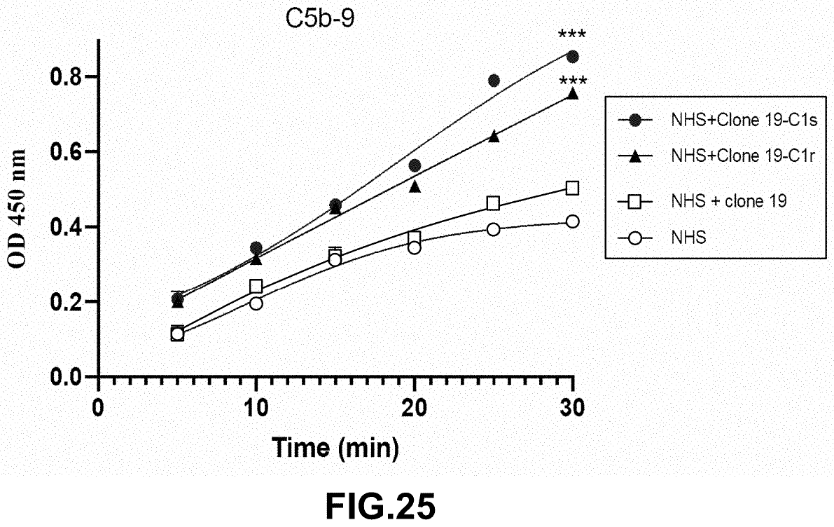

Serum samples from twelve individuals were assessed for fHbP antibody titer. Results are shown in FIG. 24. Deposition of C5b-9 (MAC) by targeted complement-activating molecules clone19-C1s and clone19-C1r was assayed using serum from individual “6,” which showed the lowest titer of fHbP antibodies. Maxisorp polystyrene microtiter plates were coated with 100 μL of 10 μg/mL mannan or an immune complex in carbonate buffer (15 mM Na₂CO₃, 35 mM NaHCO₃, pH 9.6). One percent BSA (w/v) in TBS buffer (10 mM Tris-HCl, 140 mM NaCl, pH7.4) was used to block the residual binding sites of ELISA plates for 2 hours. ELISA plates were then washed with TBS containing 0.05% (v/v) Tween 20 and 5 mM CaCl₂. Human serum from individual “NL” was diluted in BBS and added to the plates, which were then incubated for 1 h at 37° C. Deposition of C5b-9 was detected using anti C5b-9 (Abcam), followed by peroxidase-conjugated goat anti-rabbit IgG. After 1 hour, wells were washed and 100 uL of 1-Step Ultra TMB Solution (Thermo fisher scientific) was added to each well and incubated for 5 min at room temperature. The reaction was stopped by the addition of 2M H2504 and the optical density at 450 nm was immediately measured. C5b-9 deposition by monoclonal antibody clone 19 and by serum alone were also measured as controls. Both clone19-C1s and clone19-C1r targeted complement-activating molecules showed enhanced C5b-9 deposition as compared to the controls. See FIG. 25.

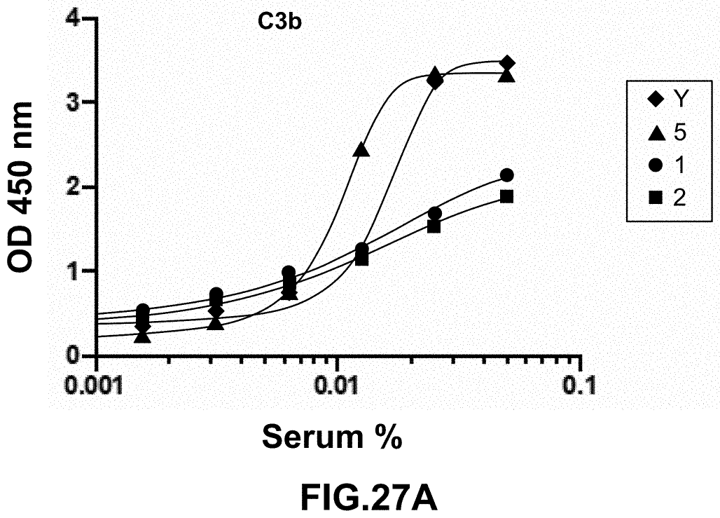

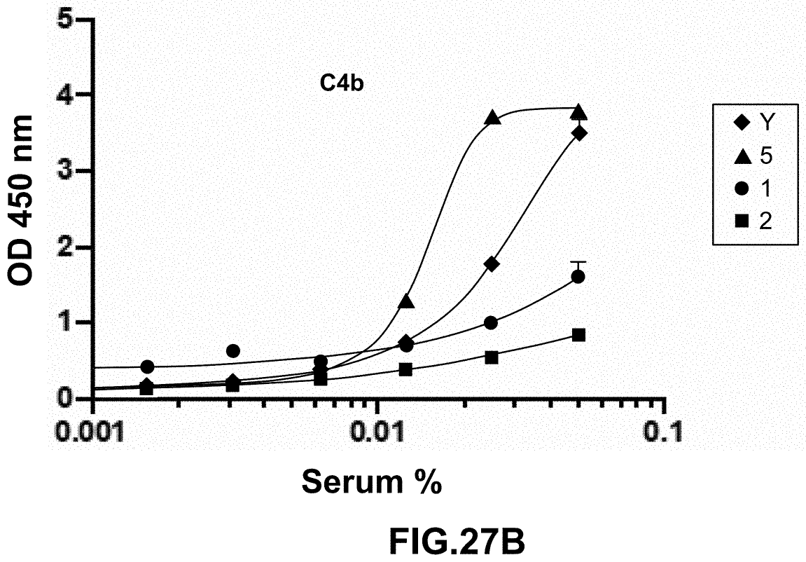

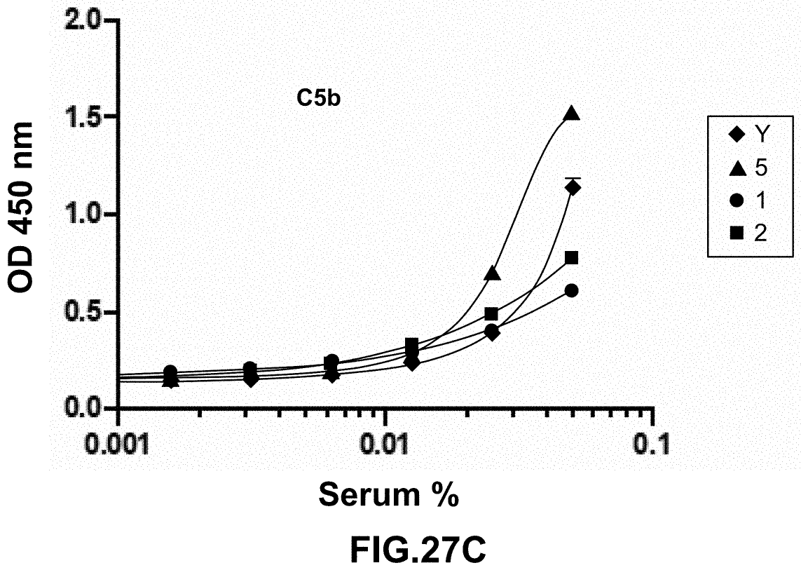

Deposition of C3b, C4b, and C5b by targeted complement-activating molecule clone19-C1r was assessed using serum from individuals “1”, “2”, “5”, and “Y”. Complement component deposition was assayed as described above for C5b, using varied serum concentrations. Deposition of C3b and C4b were assayed similarly, using rabbit anti-C3c (Dako) or rabbit anti-C4c (Dako), respectively, as detection antibody. Results are shown in FIG. 27A (C3b deposition), FIG. 27B (C4b deposition), and FIG. 27C (C5b deposition).

Additional targeted complement-activating molecules were prepared that comprise a Clone 19 binding domain and a domain from MASP-2, MASP-3, or Factor D. These targeted complement-activating molecules were named aN19(H)^{ΔK}-M2^{R444K} (also referred to as anti-fHbp-MASP-2), aN19(H)^{ΔK}-M3 (also referred to as anti-fHbp-MASP-3), and MatCFD-aN19(H) (also referred to as anti-fHbp-fD), respectively. These targeted complement-activating molecules were assayed for C3b deposition on the surface of N. meningiditis bacteria, along with the clone19-C1r (also referred to as anti-fHbp-C1r) and clone19-C1s (also referred to as anti-fHbp-C1s) targeted complement-activating molecules.

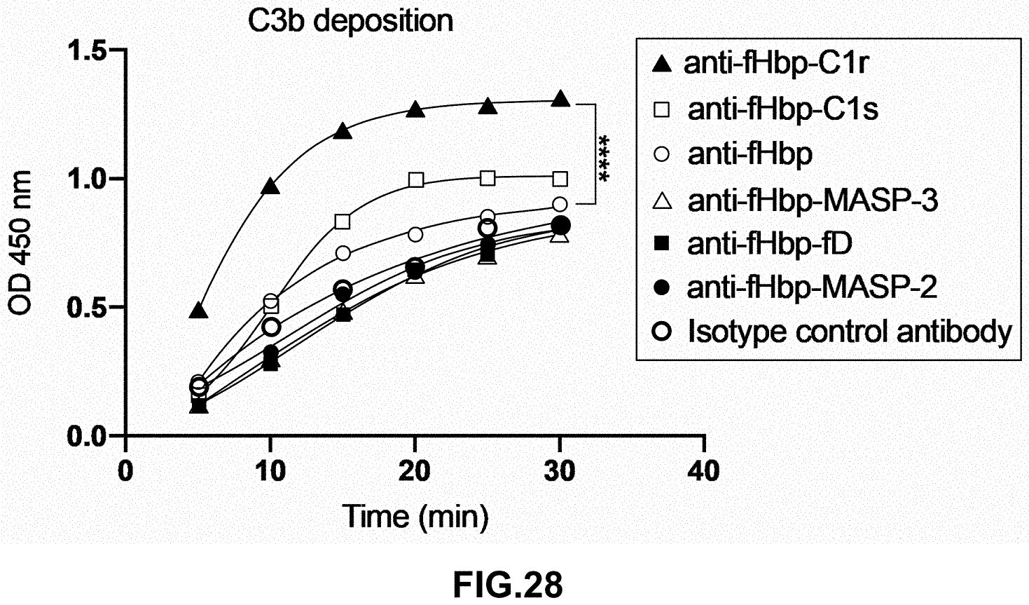

Maxisorp polystyrene microtiter ELISA plates were coated with formalin-fixed N. meningitidis bacteria (OD₆₀₀=0.6) in carbonate buffer (15 mM Na₂CO₃, 35 mM NaHCO₃, pH 9.6). The next day, wells were blocked with 5% skimmed milk in TBS buffer (10 mM Tris-HCl, 140 mM NaCl, pH7.4) for 2 hours then washed with TBS buffer containing 0.05% (v/v) Tween 20 and 5 mM CaCl₂. 1% NHS or 5% mouse serum containing 150 nM of monospecific antibodies diluted in BBS⁺⁺ buffer (4 mM barbital, 145 mM NaCl, 2 mM CaCl₂, 1 mM MgCl₂, pH 7.4) and added to the plate and incubated for 5, 10, 15, 20 and 25 minutes at room temperature then washed. Deposition of C3b was detected using rabbit anti-C3c (Dako) followed by peroxidase-conjugated goat anti-rabbit IgG. After 1 hour, wells were washed and 100 μL of 1-Step Ultra TMB Solution (Thermo Fisher Scientific) was then added to each well and incubated for 5 minutes at room temperature. The reaction was stopped by the addition of 2M H₂SO₄ and the optical density at 450 nm was immediately measured. Results are shown in FIG. 28. The difference in C3b deposition between Clone 19 (i.e., anti-fHbp) and anti-fHbp-C1r was observed to be significant.

Serum Bactericidal Activity

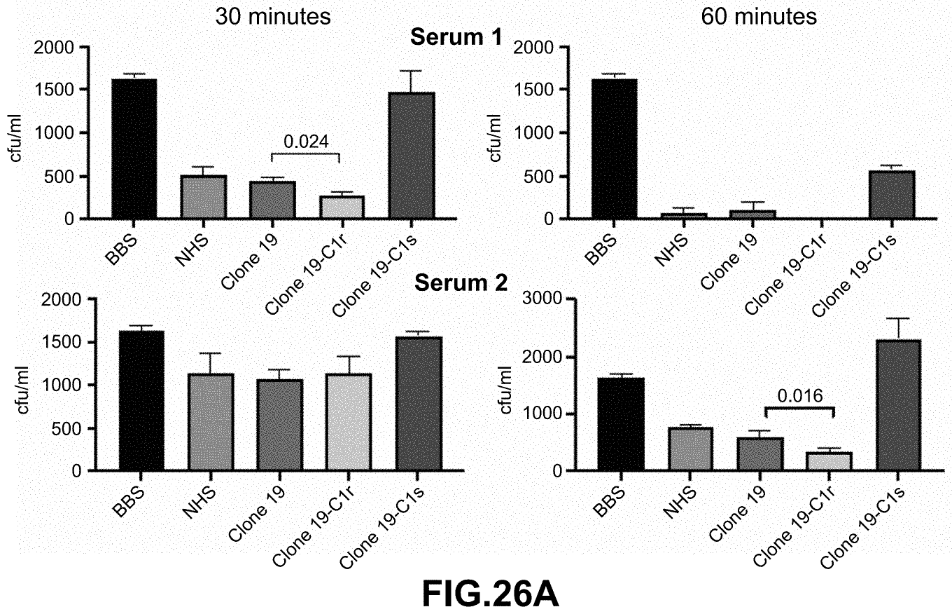

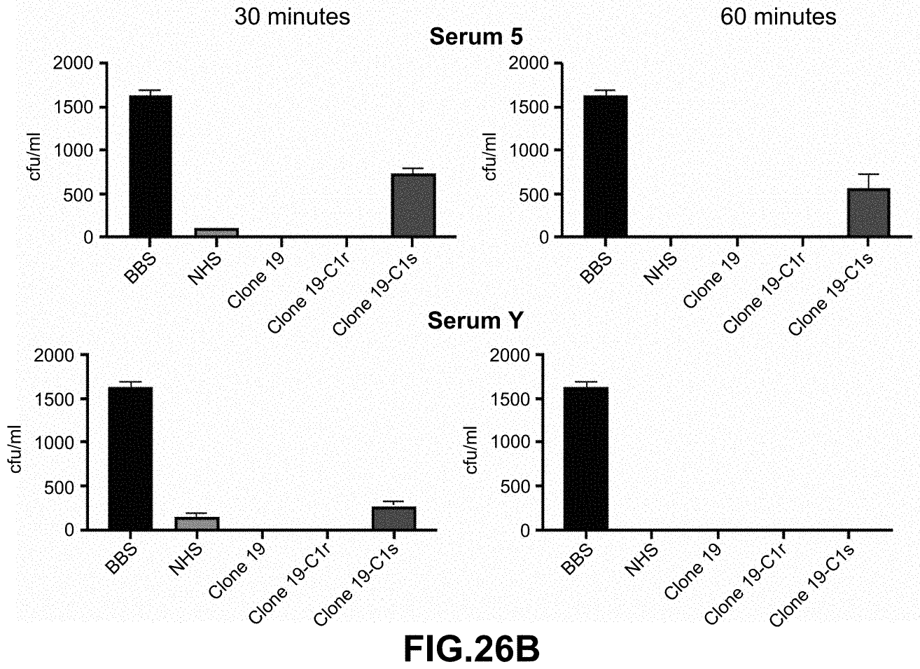

Targeted complement-activating molecules clone19-C1s and clone19-C1r were assessed for serum bactericidal activity. Neisseria meningitidis serotype B (MC58) were grown on a blood agar plate at 37° C. and 5% CO₂. Next day, cells were scraped and suspended in BBS (4 mM barbital, 145 mM NaCl, 2 mM CaCl₂, 1 mM MgCl₂, pH 7.4). 1500 cells were incubated with 25% normal human serum from individual “1” (FIG. 26A, top row), individual “2” (FIG. 26A, bottom row), individual “5” (FIG. 26B, top row), or individual “Y” (FIG. 26B, bottom row) with or without 10 ug of anti-fHbp clone 19, clone19-C1s, or clone19-C1r. At predetermined time points (30 and 60 minutes), samples were taken and plated on blood agar plates for overnight at 37° C. and 5% CO₂. Serum bactericidal activity was calculated by measuring the decrease in the viable bacterial count recovered after incubation with NHS compared to the original bacterial count at zero time point and heat inactivated serum. Targeted complement-activating molecule clone19-C1r showed a significant reduction in viable bacterial count as compared to controls or to clone19 monoclonal antibody in the Serum 1 assay at the 30-minute timepoint and in the Serum 2 assay at the 60-minute time point. See FIGS. 26A and 26B.

### Example 12

### In Vivo Study of Anti-fHbP-Derived Targeted Complement-Activating Molecules in a Mouse Model of N. meningitides Infection

The effect of the anti-fHbP-derived targeted complement-activating molecules in a mouse model of N. meningitides infection was studied. 12-week-old female C57BL/6 wild-type mice (Charles River Laboratory) were used in this study. Mice were injected intraperitoneally (i.p.) with iron dextran (400 mg/kg; Sigma-Aldrich) 12 hours before infection. The next day, mice were injected i.p with 100 μL of passaged N. meningitidis B-MC58 suspension containing 5×10⁶ cfu in PBS and with iron dextran (400 mg/kg). Monoclonal antibodies or targeted complement-activating molecules were injected i.p. 18 hours before infection. Mice treated with an isotype control antibody served as a control. Each group consisted of 12 mice. The inoculum dose was confirmed by viable count after plating on blood agar with 5% (vol/vol). Mice were monitored for progression of clinical signs and euthanized when they became lethargic. Blood samples were obtained at pre-determined time points, and viable counts were calculated after serial dilution in PBS and plating out on blood agar plates.

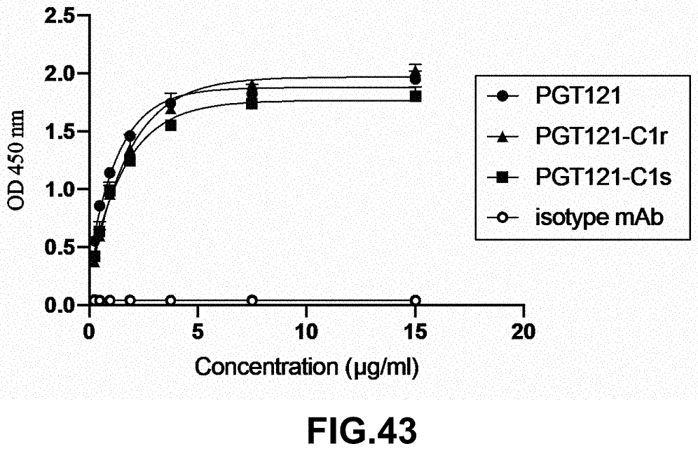

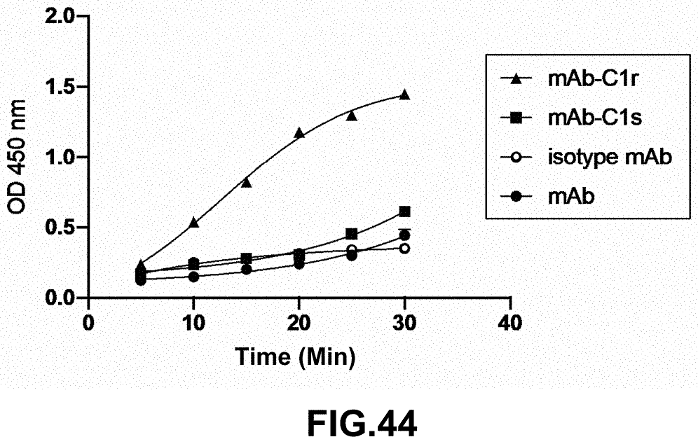

Mice were treated with monoclonal antibody Clone 19 or with targeted complement-activating molecules Clone 19-C1r or Clone 19-C1s. Bacterial load in blood samples collected at 8 hours and 24 hours after infection is shown in FIG. 43. Bacterial load at both time points was significantly lower in mice treated with Clone 19-C1r as compared to mice treated with antibody Clone 19. Survival of mice after infection is shown in FIG. 44. Survival, i.e., mice not requiring euthanization, was significantly improved for mice treated with Clone 19-C1r as compared to mice treated with antibody Clone 19. For both FIG. 43 and FIG. 44, *p<0.05 and **p<0.01 using the Mantel-Coxlog-rank test.

### Example 13

### Preparation of Anti-PspA-Derived Targeted Complement-Activating Molecules

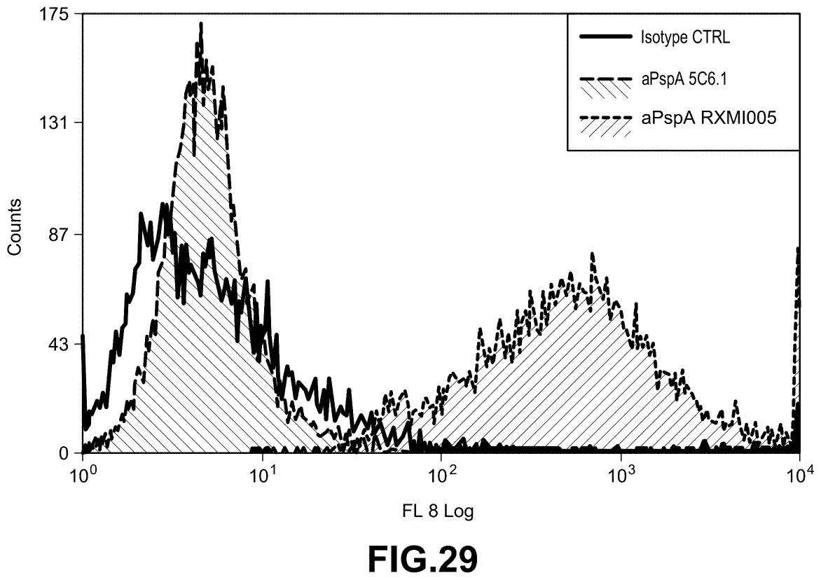

Monoclonal antibodies to pneumococcal surface protein A (PspA) of Streptococcus pneumoniae were produced using mouse hybridomas kindly provided by Dr. David Briles and Dr. W. Edward Swords at the University of Alabama at Birmingham. Anti-PspA antibodies 5C6.1 and RX1MI005 were described in Vaccine (2013); 32(1):39-47 and mSphere (2019) 4:e00589-19, respectively. The binding of each of these antibodies to S. pneumoniae was tested. S. pneumoniae strain D39 was incubated with either 5C6.1 or RX1MI005 at a concentration of 10 μg/mL for 30 minutes at room temperature, then washed and incubated with Alexa Fluor goat anti-human IgG for 30 minutes. Binding was measured by FACS analysis. Results are shown in FIG. 29. Antibody RX1MI005 was observed to bind better than 5C6.1, and was therefore selected for further use.

Antibody RX1MI005 was sequenced, and the sequence used to create targeted complement-activating molecules comprising an RX1MI005 binding domain and a fragment of either C1r or C1s. The C-terminal catalytic fragment of C1r or C1s was fused with anti-PspA antibody RX1MI005 at the C-terminus of the antibody's heavy chain (HC), which was altered by a deletion of the single amino acid lysine (K) from the C-terminus. This process resulted in the following constructs: RX1MI005(H)^{ΔK}-C1r_HC (SEQ ID NO:122) and RX1MI005(H)^{ΔK}-C1s_HC (SEQ ID NO:123).

Plasmid preparation, cloning, protein expression, and purification was carried out as described in Example 1.

### Example 14

### Binding of Anti-PspA-Derived Targeted Complement-Activating Molecules to S. pneumoniae

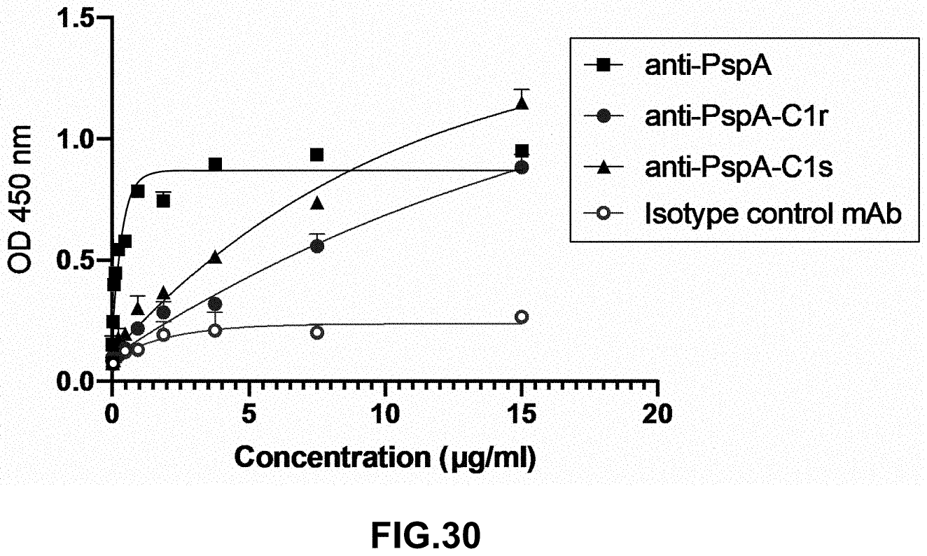

Binding to S. pneumoniae was tested for each of the targeted complement-activating molecules comprising a RX1MI005 binding domain. Targeted complement-activating molecules RX1MI005(H)^{ΔK}-C1r (also referred to as anti-PspA-C1r or MI005-C1r) and RX1MI005(H)^{ΔK}-C1s (also referred to as anti-PspA-C1s or MI005-C1s) were tested, along with monoclonal antibody RX1MI005. An ELISA plate was coated with S. pneumoniae strain D39 in coating buffer and blocked with 5% skimmed milk. Serial dilutions of antibody or targeted complement-activating molecules were added to the plate and incubated for 30 minutes at room temperature then washed. Bound antibodies and targeted complement-activating molecules were detected using HRP conjugated anti-human IgG. An unrelated isotype antibody was included as a control. All three molecules showed binding to S. pneumoniae. See FIG. 30.

### Example 15

### Activity of Anti-PspA-Derived Targeted Complement-Activating Molecules

Complement Deposition Activity

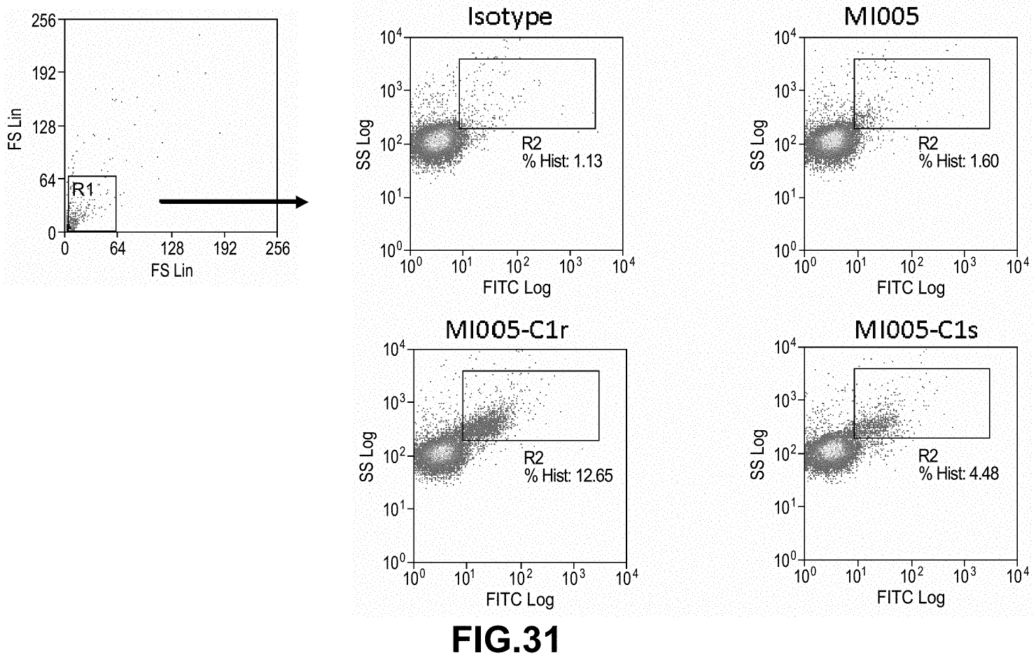

Deposition of C3b by anti-PspA antibody RX1MI005 and RX1MI005-derived targeted complement-activating molecules on the surface of S. pneumoniae was assessed. S. pneumoniae bacteria were washed twice with TBS buffer and resuspended in BBS⁺⁺ buffer (4 mM barbital, 145 mM NaCl, 2 mM CaCl₂, 1 mM MgCl₂, pH 7.4) buffer to a final concentration of 10⁶ cfu/mL. The bacterial suspension (100 μL) was opsonized with 1% (vol/vol) NHS or 5% (vol/vol) wild-type mouse serum for 15 minutes at room temperature with antibody or targeted complement-activating molecules. Nonopsonized bacteria served as a negative control. After opsonization, the bacterial samples were washed twice with TBS buffer, and bound C3b was detected using FITC-conjugated rabbit anti-human C3c (Dako). Fluorescence intensity was measured with a FACSCalibur cell analyzer (BD Biosciences). Results are shown in FIG. 31. Complement C3b deposition on the surface of S. pneumoniae was observed to be enhanced when the MI005-Cr and MI005-C1s targeted complement-activating molecules were present, as compared to the RX1MI005 antibody or the isotype control antibody.

### Example 16

### Preparation of Targeted Complement-Activating Molecules Derived from Antibodies to C. albicans

Monoclonal antibody 1A2 binds to a fungal mannan epitope on the surface of Candida albicans. This antibody was described in PCT patent application publication WO 2014/174293. The sequence of antibody 1A2 used to create targeted complement-activating molecules comprising a 1A2 binding domain and a fragment of either C1r or C1s. The C-terminal catalytic fragment of C1r or C1s was fused with antibody 1A2 at the C-terminus of the antibody's heavy chain (HC), which was altered by a deletion of the single amino acid lysine (K) from the C-terminus. This process resulted in the following constructs: 1A2(H)^{ΔK}-C1r_HC (SEQ ID NO:130) and 1A2(H)^{ΔK}-C1s_HC (SEQ ID NO:131).

Plasmid preparation, cloning, protein expression, and purification was carried out as described in Example 1.

### Example 17

### Binding to C. albicans of Targeted Complement-Activating Molecules Derived from Antibodies to C. albicans

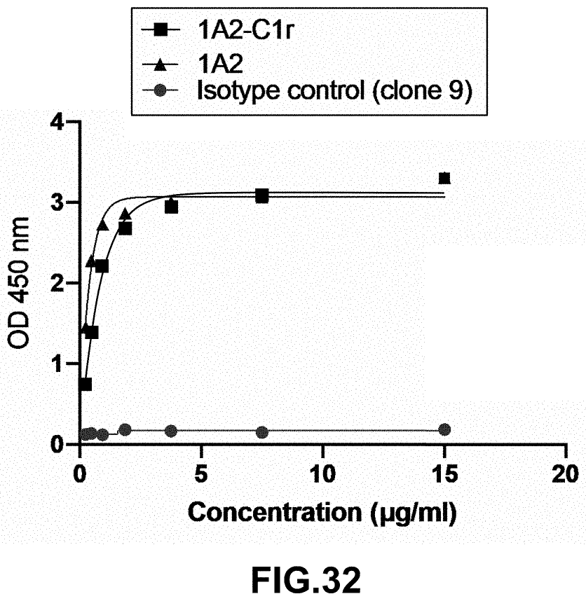

Binding to C albicans was tested for targeted complement-activating molecule 1A2(H)^{ΔK}-C1r (also referred to as 1A2-C1r) and monoclonal antibody 1A2. An ELISA plate was coated with C. albicans in coating buffer and blocked with 5% skimmed milk. Serial dilutions of antibody 1A2 and the targeted complement-activating molecule were added to the plate and incubated for 30 minutes at room temperature, then washed. Bound antibodies were detected using HRP conjugated anti-human IgG. Both antibody 1A2 and targeted complement-activating molecule 1A2-C1r showed binding to C. albicans. An unrelated isotype antibody was used as a control. See FIG. 32.

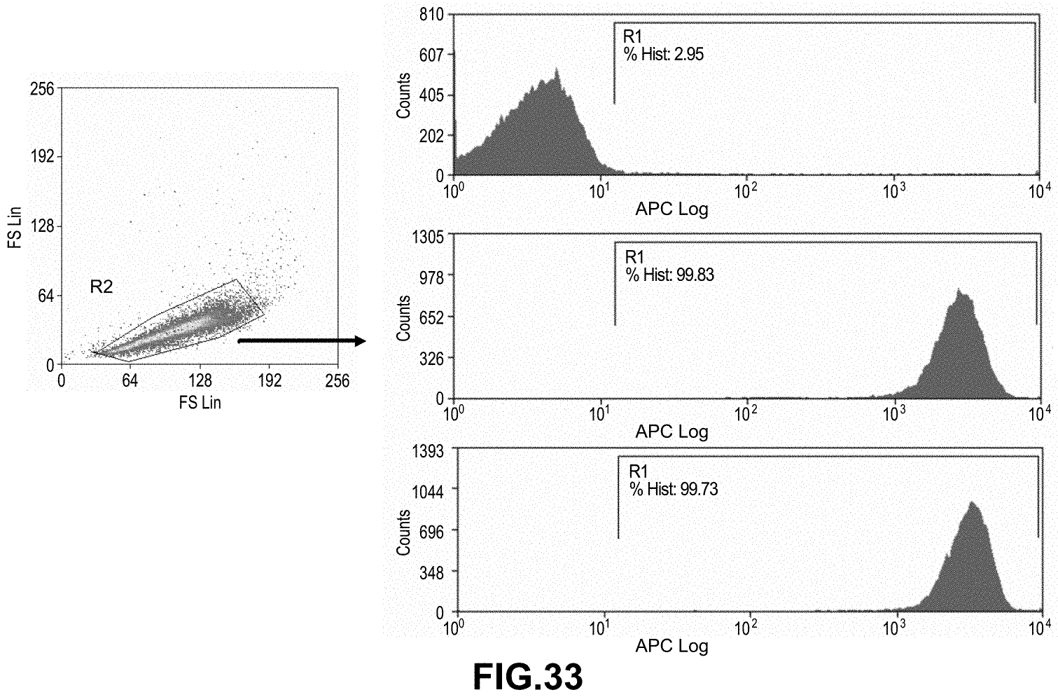

Binding to C. albicans was also tested using an alternative assay. Fungal cells were incubated with antibody 1A2 or targeted complement-activating molecule 1A2-C1r for 30 minutes at room temperature, then washed and incubated with Alexa Fluor goat anti-human IgG for 30 minutes. Binding was measured by FACS analysis. An unrelated isotype antibody was used as a control. Both antibody 1A2 and targeted complement-activating molecule 1A2-C1r showed binding to C. albicans. See FIG. 33.

### Example 18

### Activity of Targeted Complement-Activating Molecules Derived from Antibodies to C. albicans

Complement Deposition Activity

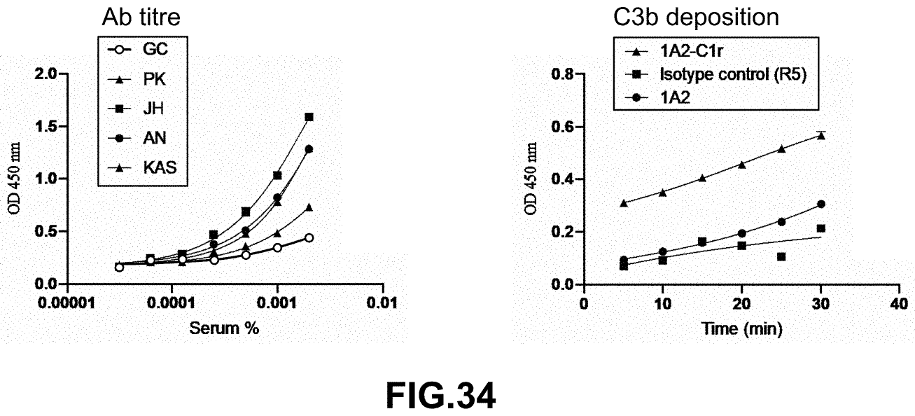

Deposition of C3b by antibody 1A2 and targeted complement-activating molecule 1A2-C1r on the surface of C. albicans was assessed. First, a human serum with minimal natural antibodies to C. albicans was identified by screening sera from five different individuals. ELISA plates were coated with C. albicans and incubated with serum from each of the individuals. Antibodies against C. albicans were detected using horseradish peroxidase (HRP)-conjugated anti-human IgG antibody. The serum having the lowest measured titer of C. albicans antibodies, indicated as “GC”, was used in a C3b deposition assay. Results are shown in FIG. 34, left panel.

For the C3b deposition assay, Maxisorp polystyrene microtiter ELISA plates were coated with formalin-fixed C. albicans in carbonate buffer (15 mM Na₂CO₃, 35 mM NaHCO₃, pH 9.6). The next day, wells were blocked with 5% skimmed milk in TBS buffer (10 mM Tris-HCl, 140 mM NaCl, pH7.4) for 2 hours then washed with TBS buffer containing 0.05% (v/v) Tween 20 and 5 mM CaCl₂. 1% NHS serum “GC” containing 150 nM of antibodies or targeted complement-activating molecules diluted in BBS⁺⁺ buffer (4 mM barbital, 145 mM NaCl, 2 mM CaCl₂, 1 mM MgCl₂, pH 7.4) and added to the plate and incubated for 5, 10, 15, 20 and 25 minutes at room temperature then washed. Deposition of C3b, detected using rabbit anti-C3c (Dako) followed by peroxidase-conjugated goat anti-rabbit IgG. After 1 hour, wells were washed and 100 μL of 1-Step Ultra TMB Solution (Thermo Fisher Scientific) was then added to each well and incubated for 5 minutes at room temperature. The reaction was stopped by the addition of 2M H₂SO₄ and the optical density at 450 nm was immediately measured. Results are shown in FIG. 34, right panel. The level of C3b deposition was significantly enhanced when targeted complement-activating molecule 1A2-C1r was used, as compared to antibody IA2 or an isotype control.

### Example 19

### Preparation of Anti-Fnbp-Derived Targeted Complement-Activating Molecules

Monoclonal antibodies to fibronectin binding protein (Fnbp) of Staphylococcus aureus were produced in mice and isolated using hybridoma technique. Mouse spleen cells were mixed with NS0 myeloma cells in a ratio 1:4 in RPMI SFM (Sigma) and pelleted at 1200×g for 5 minutes. After centrifugation, the supernatant was completely removed. Splenocytes and NS0 cells were then fused together by addition of 0.8 mL of polyethylene glycol 1500 (Roche) through a period of 1 minute with gentle stirring. After that, 10 mL of RPMI-SFM was added stepwise with gentle stirring over a period of 5 minutes. Fused cells were then pelleted and re-suspended into 50 mL of RPMI medium supplemented with 15% FCS (Sigma), 200 u/mL Penicillin/Streptomycin (Sigma), 1 mM pyruvic acid (Sigma), 0.05 μM β-mercaptoethanol (Sigma), 0.5 μg/mL hydrocortisone (Sigma) and 0.4 mM L-glutamine (Sigma). Hybridoma cells were finally plated into 96 well plates and incubated at 37° C. and 5% CO₂. As a negative control, NS0 myeloma cells were added to the last two rows of each plate. Next day, hypoxanthine and azaserine (Sigma) were added to each well at a final concentration of 100 μM hypoxanthine and 5.7 μM azaserine. The hybridomas were fed every 3 days by removing 100 μL of the old medium and replacing it with fresh RPMI medium containing 15% FCS. When the hybridomas reached 30-50% confluence, supernatant samples were taken for screening using ELISA. Positive clones were selected and transferred into 24 well plates and finally into 25 cm² flasks.

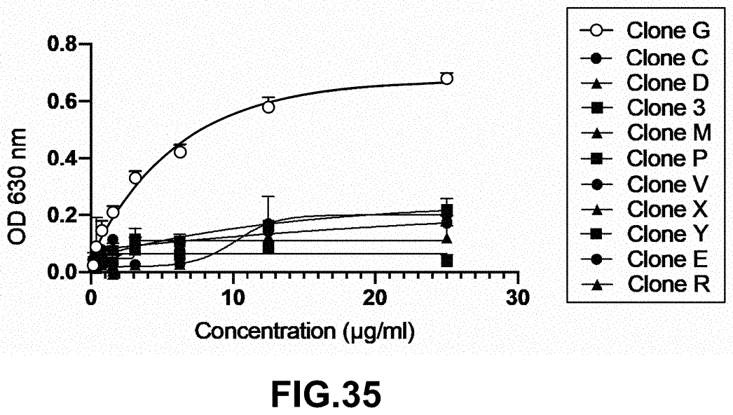

Eleven candidate antibodies were identified and screened for binding to S. aureus. Maxisorp polystyrene microtiter ELISA plates were coated with formalin-fixed S. aureus (OD600=0.6) in carbonate buffer (15 mM Na₂CO₃, 35 mM NaHCO₃, pH 9.6). The next day, wells were blocked with 5% skimmed milk in TBS buffer (10 mM Tris-HCl, 140 mM NaCl, pH7.4) for 2 hours then washed with TBS buffer containing 0.05% (v/v) Tween 20 and 5 mM CaCl₂. Candidate antibodies were serially diluted in TBS buffer, added to the plate and incubated for 1 hour at room temperature then washed. Binding of antibodies was detected using peroxidase-conjugated rabbit anti-human IgG. After 1 hour, wells were washed and 100 μL of 1-Step Ultra TMB Solution (Thermo Fisher Scientific) was then added to each well and incubated for 5 minutes at room temperature. The reaction was stopped by the addition of 2M H₂SO₄ and the optical density at 450 nm was immediately measured. Antibody Clone G was identified as showing the best binding to S. aureus. See FIG. 35.

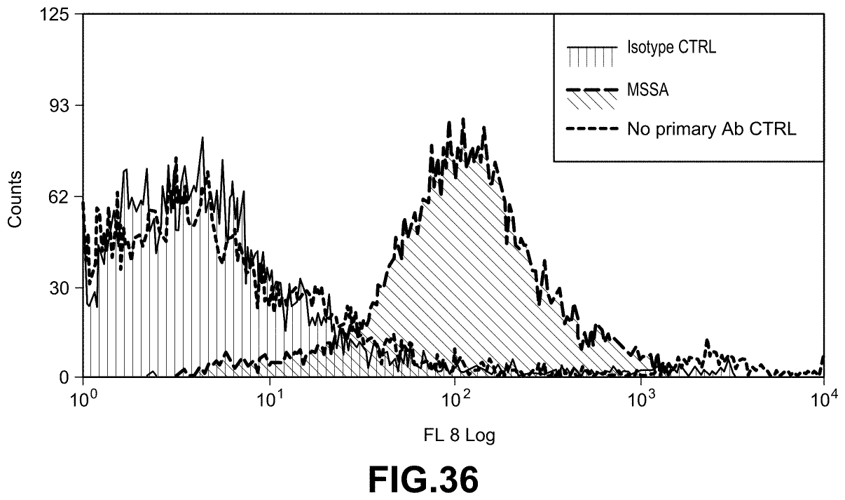

Antibody Clone G was tested for binding to S. aureus strain MSSA. S. aureus MSSA bacteria were washed twice with TBS buffer and resuspended in BBS⁺⁺ buffer (4 mM barbital, 145 mM NaCl, 2 mM CaCl₂, 1 mM MgCl₂, pH 7.4) buffer to a final concentration of 10⁶ cfu/mL. Bacterial suspension (100 μL) was incubated with 150 nM of antibody Clone G for 30 minutes at room temperature. Bacteria opsonized with an isotype control antibody were used as a negative control. After incubation, the bacterial samples were washed twice with TBS buffer, and bound antibodies were detected using FITC-conjugated rabbit anti-human IgG. Fluorescence intensity was measured with a FACSCalibur cell analyzer (BD Biosciences). Results are shown in FIG. 36. Clone G was observed to bind to the S. aureus MSSA strain.

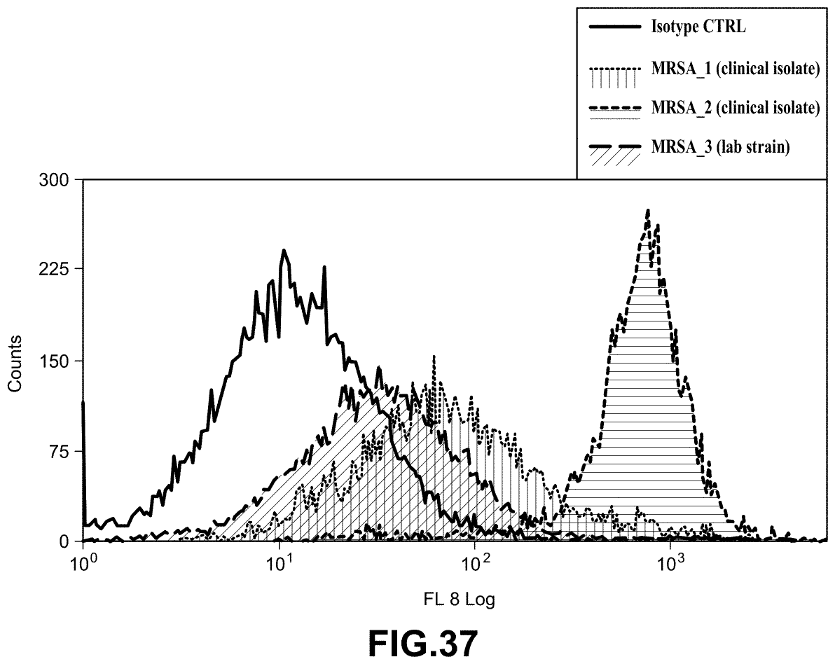

Antibody Clone G was also tested for binding to several different isolates of S. aureus strain MRSA. S. aureus MRSA bacteria from one of two clinical isolates and a lab strain isolate were washed twice with TBS buffer and resuspended in BBS++ buffer (4 mM barbital, 145 mM NaCl, 2 mM CaCl2, 1 mM MgCl2, pH 7.4) buffer to a final concentration of 106 cfu/mL. Bacterial suspension (100 μL) was incubated with 150 nM of antibody Clone G for 30 minutes at room temperature. Bacteria opsonized with an isotype control antibody were used as a negative control. After incubation, the bacterial samples were washed twice with TBS buffer, and bound antibodies were detected using FITC-conjugated rabbit anti-human IgG. Fluorescence intensity was measured with a FACSCalibur cell analyzer (BD Biosciences). Results are shown in FIG. 37. Clone G was observed to bind to all three isolates of the S. aureus MRSA strain.

Antibody Clone G was sequenced, and the sequence used to create targeted complement-activating molecules comprising a Clone G binding domain and a fragment of either C1r or C1s. The C-terminal catalytic fragment of C1r or C1s was fused with anti-Fnbp antibody Clone G at the C-terminus of the antibody's heavy chain (HC), which was altered by a deletion of the single amino acid lysine (K) from the C-terminus. This process resulted in the following constructs: C1.G(H)^{ΔK}-C1r_HC (SEQ ID NO:126) and C1.G(H)^{ΔK}-C1s_HC (SEQ ID NO:127).

Plasmid preparation, cloning, protein expression, and purification was carried out as described in Example 1.

### Example 20

### Binding of Anti-Fnbp-Derived Targeted Complement-Activating Molecules

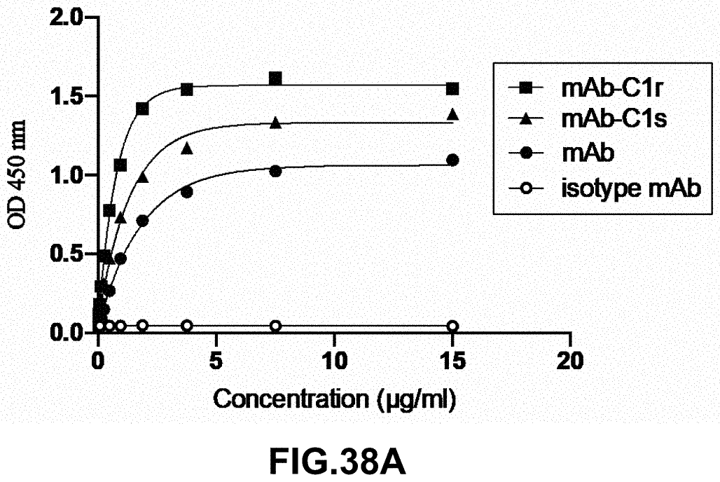

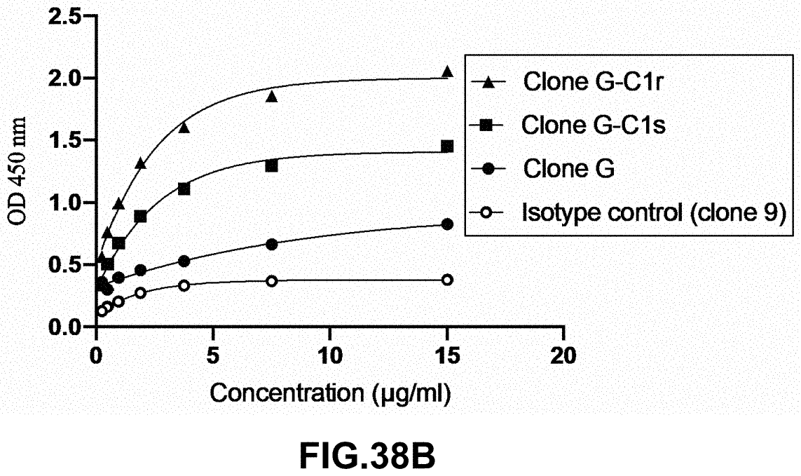

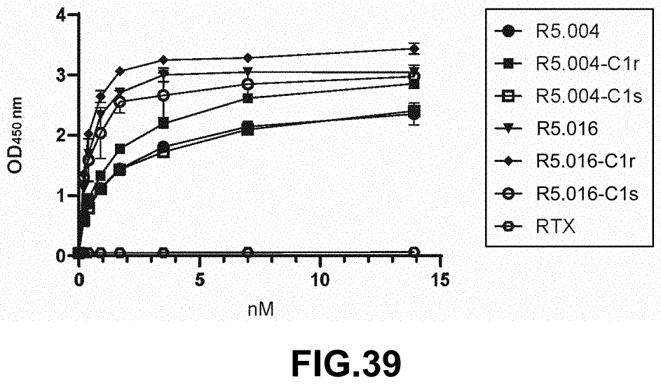

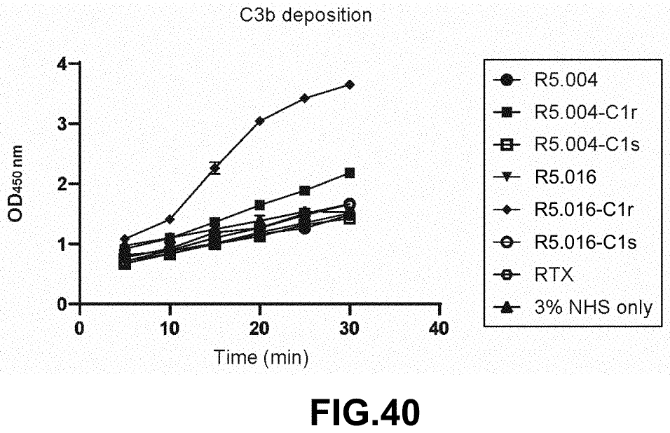

Binding to Fnbp was tested for each of the targeted complement-activating molecules comprising a Clone G binding domain. Targeted complement-activating molecules C1.G(H)^{ΔK}-C1r (also referred to as Clone G-C1r) and C1.G(H)^{ΔK}-C1s (also referred to as Clone G-C1s) were tested, along with monoclonal antibody Clone G. Maxisorp polystyrene microtiter ELISA plates were coated with 2 μg/mL of recombinant S. aureus FnbpB in coating buffer. The next day, wells were blocked with 5% skimmed milk in PBS for two hours, then washed with PBS buffer containing 0.05% (v/v) Tween 20. Two-fold serial dilutions of antibodies and targeted complement-activating molecules were prepared in PBS buffer starting from 15 μg/mL. Samples of 100 μL were transferred to the ELISA plate and were incubated at room temperature. After one hour, the plate was washed and 100 μL of HRP-conjugated goat anti-human IgG detection antibody was added to the plate, followed by 30 minutes incubation at room temperature. The plate was washed and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured. See FIG. 38A.

Binding to S. aureus was also tested for each of the targeted complement-activating molecules comprising a Clone G binding domain. Targeted complement-activating molecules Clone G-C1r and Clone G-C1s were tested, along with monoclonal antibody Clone G. An ELISA plate was coated with S. aureus in coating buffer and blocked with 5% skimmed milk. Serial dilutions of antibody or targeted complement-activating molecules were added to the plate and incubated for 30 minutes at room temperature then washed. Bound antibodies and targeted complement-activating molecules were detected using HRP conjugated anti-human IgG. An unrelated isotype antibody was included as a control. All three molecules showed binding to S. aureus. See FIG. 38B.

### Example 21

### Activity of Anti-Fnbp-Derived Targeted Complement-Activating Molecules

Complement Deposition Activity

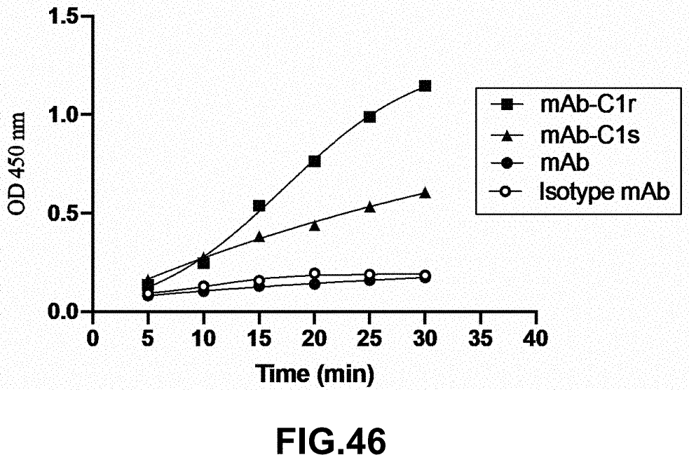

Deposition of C3b by antibody Cone G and targeted complement-activating molecules Clone G-C1r and Clone G-C1s on a Fnbp-coated surface was assessed. Maxisorp polystyrene microtiter ELISA plates were coated with 2 μg/mL of recombinant FnbpB in coating buffer. The next day, wells were blocked with 5% skimmed milk in PBS for two hours, then washed with PBS buffer containing 0.05% (v/v) Tween 20. NHS containing 7.5 μg of antibody or targeted complement-activating molecules was diluted in BBS⁺⁺ buffer (4 mM barbital, 145 mM NaCl, 2 mM CaCl₂, 1 mM MgCl₂, pH 7.4) to a concentration of 2.5%, added to the plate, and incubated for 5, 10, 15, 20 and 25 minutes at room temperature then washed three times. C3b deposition was detected by using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured. Results are shown in FIG. 46.

### Example 22

### Preparation of Anti-PfRH5-Derived Targeted Complement-Activating Molecules

Antibodies to Plasmodium falciparum antigen reticulocyte binding protein homologue 5 (PfRH5) were described by Alanine et al. (Cell (2019) 178:216-228). The sequences of anti-PfRH5 antibodies R5.004 and R5.016 were used to create targeted complement-activating molecules comprising and antibody binding domain and a fragment of either C1r or C1s. The C-terminal catalytic fragment of C1r or C1s was fused with the antibody at the C-terminus of the antibody's heavy chain (HC), which was altered by a deletion of the single amino acid lysine (K) from the C-terminus. This process resulted in the following constructs: R5.004(H)^{ΔK}-C1r_HC (SEQ ID NO:138), R5.004(H)^{ΔK}-C1s_HC (SEQ ID NO:139), R5.016(H)^{ΔK}-C1r_HC (SEQ ID NO:142) and R5.016(H)^{ΔK}-C1s_HC (SEQ ID NO:143).

Plasmid preparation, cloning, protein expression, and purification was carried out as described in Example 1.

### Example 23

### Binding of Anti-PfRH5-Derived Targeted Complement-Activating Molecules to PfRH5

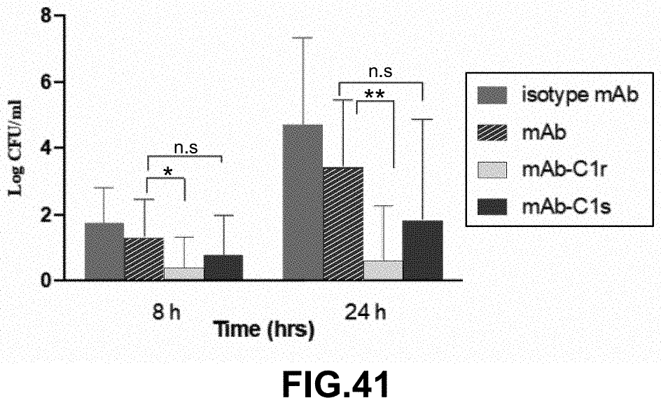

Binding to P. falciparum was tested for each of the targeted complement-activating molecules comprising an anti-PfRH5 binding domain. Maxisorp polystyrene microtiter ELISA plates were coated with 50 μL per well of cell supernatant from cells transfected with PfRH5. The next day, the wells were blocked with 1% BSA in PBS (1×) for two hours, then washed with PBS buffer containing 0.05% (v/v) Tween 20. Two-fold serial dilutions of antibodies and targeted complement-activating molecules were prepared in buffer containing 0.1% BSA in PBS (1×) with the highest concentration being 13.9 nM. Samples of 100 μL each were transferred to the ELISA plate and incubated at room temperature. After 1 hour, the plate was washed and 100 μL of HRP-conjugated goat anti-human IgG detection antibody was added to the plate followed by 30 minutes incubation at room temperature. The plate was washed and 100 μL of 1-step Ultra TMB Solution (Thermo Fisher Scientific) was added to each well and incubated for two minutes at room temperature. The reaction was stopped by the addition of 2M H₂SO₄ and the optical density at 450 nm was immediately measured. An unrelated antibody (RTX) was used as a control. Results are shown in FIG. 41. Both antibodies and all targeted-complement-activating molecules tested showed binding to PfRH5.

### Example 24

### Activity of Anti-PfRH5-Derived Targeted Complement-Activating Molecules

Complement Deposition Activity

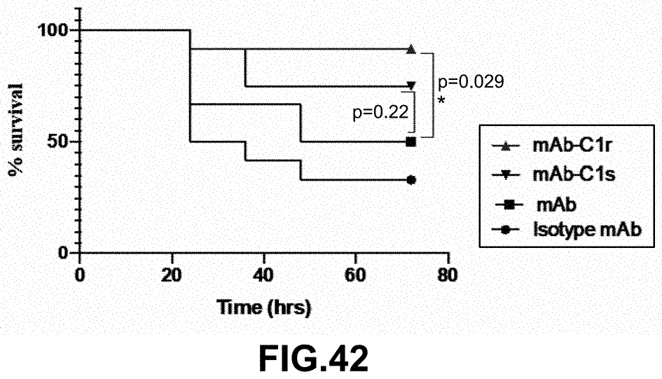

Deposition of C3b by antibodies R5.004, R5.016 and targeted complement-activating molecules R5.004(H)^{ΔK}-C1r (also referred to as R5.004-C1r), R5.004(H)^{ΔK}-C1s (also referred to as R5.004-C1s), R5.016(H)^{ΔK}-C1r (also referred to as R5.016-C1r) and R5.016(H)^{ΔK}-C1s (also referred to as R5.016-C1s) on a PfRH5-coated surface was assessed. Maxisorp polystyrene microtiter ELISA plates were coated with 50 μL per well of cell supernatant from cells transfected with PfRH5. The next day, the wells were blocked with 1% BSA in PBS (1×) for two hours, then washed with PBS buffer containing 0.05% (v/v) Tween 20. Normal human serum (NETS) containing 13.9 nM of antibody or targeted complement-activating molecules was diluted in BBS⁺⁺ buffer (4 mM barbital, 145 mM CaCl, 2 mM CaCl₂, 1 mM MgCl₂, pH 7.4) to a concentration of 3%, and added to the wells. The plate was incubated for either 5, 10, 15, 20, or 25 minutes at room temperature, then washed three times. C3b deposition was detected using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured. An unrelated antibody (RTX) was used as a control. Results are shown in FIG. 42.

### Example 25

### Preparation of Anti-GP120-Derived Targeted Complement-Activating Molecules

Antibodies to HIV-1 envelope glycoprotein GP120 were described by Julien et al. (PLoS Pathog (2013) 9:e1003342). The sequence of anti-GP120 antibody PGT121 was used to create targeted complement-activating molecules comprising and antibody binding domain and a fragment of either C1r or C1s. The C-terminal catalytic fragment of C1r or C1s was fused with the antibody at the C-terminus of the antibody's heavy chain (HC), which was altered by a deletion of the single amino acid lysine (K) from the C-terminus. This process resulted in the following constructs: PGT121(H)^{ΔK}-C1r_HC (SEQ ID NO:146) and PGT121(H)^{ΔK}-C1s_HC (SEQ ID NO:147).

Plasmid preparation, cloning, protein expression, and purification was carried out as described in Example 1.

### Example 26

### Binding of Anti-GP120-Derived Targeted Complement-Activating Molecules to GP120

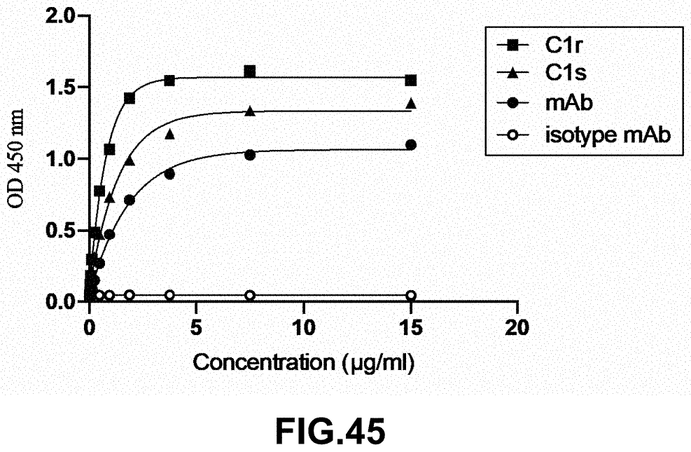

Binding to GP120 was tested for each of the targeted complement-activating molecules PGT121(H)^{ΔK}-C1r (also referred to as PGT121-C1r) and PGT121(H)^{ΔK}-C1s (also referred to as PGT121-C1s), along with monoclonal antibody PGT121. An unrelated isotype antibody was used as a control. Maxisorp polystyrene microtiter ELISA plates were coated with 2 μg/mL of recombinant GP120 in coating buffer. The next day, wells were blocked with 5% skimmed milk in PBS for two hours, then washed with PBS buffer containing 0.05% (v/v) Tween 20. Two-fold serial dilutions of antibodies and targeted complement-activating molecules were prepared in PBS buffer starting from 15 μg/mL. Samples of 100 μL were transferred to the ELISA plate and were incubated at room temperature. After one hour, the plate was washed and 100 μL of HRP-conjugated goat anti-human IgG detection antibody was added to the plate and incubated 30 minutes at room temperature. The plate was washed and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured. Results are shown in FIG. 45. Antibody PGT121 and both targeted complement-activating molecules tested showed binding to GP120.

### Example 27

### Activity of Anti-GP120-Derived Targeted Complement-Activating Molecules

Complement Deposition Activity

Deposition of C3b by antibody PGT121 and targeted complement-activating molecules PGT121-C1r and PGT121-C1s on a GP120-coated surface was assayed. Maxisorp polystyrene microtiter ELISA plates were coated with 2 μg/mL of recombinant GP120 in coating buffer. The next day, the wells were blocked with 5% skimmed milk in PBS for two hours, then washed with PBS buffer containing 0.05% (v/v) Tween 20. NHS containing 7.5 μg of antibodies or targeted complement-activating molecules was diluted in BBS⁺⁺ buffer (4 mM barbital, 145 mM NaCl, 2 mM CaCl₂, 1 mM MgCl₂, pH 7.4) to a concentration of 2.5%, added to the plate, and incubated for 5, 10, 15, 20 and 25, and 25 minutes at room temperature then washed three times. C3b deposition was detected by using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured. An unrelated isotype antibody was used as a control. Results are shown in FIG. 46.

### Example 28

### Preparation of Anti-S Protein-Derived Targeted Complement-Activating Molecules

Antibody to SARS-CoV-2 S protein (also referred to as spike protein) was described by Westendorf et al. (Cell Rep (2022) 39:110812). The sequence of anti-S protein antibody bebtelovimab was used to create targeted complement-activating molecules comprising an antibody binding domain and a fragment of either C1r or C1s. The C-terminal catalytic fragment of C1r or C1s was fused with the antibody at the C-terminus of the antibody's heavy chain (HC), which was altered by a deletion of the single amino acid lysine (K) from the C-terminus. This process resulted in the following constructs: bebtelovimab(H)^{ΔK}-C1r_HC (SEQ ID NO:150) and bebtelovimab(H)^{ΔK}-C1s_HC (SEQ ID NO:151).

Plasmid preparation, cloning, protein expression, and purification was carried out as described in Example 1.

### Example 29

### Binding of Anti-S Protein-Derived Targeted Complement-Activating Molecules

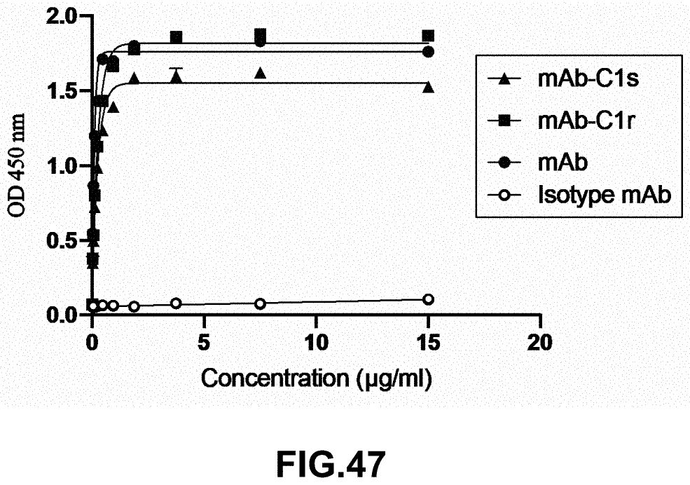

Binding to S protein was tested for each of the targeted complement-activating molecules bebtelovimab(H)^{ΔK}-C1r (also referred to as bebtelovimab-C1r) and bebtelovimab(H)^{ΔK}-C1s (also referred to as bebtelovimab-C1s), along with monoclonal antibody bebtelovimab. An unrelated isotype antibody was used as a control. Maxisorp polystyrene microtiter ELISA plates were coated with 2 μg/mL of recombinant SARS-CoV-2 S protein in coating buffer. The next day, wells were blocked with 5% skimmed milk in PBS for two hours, then washed with PBS buffer containing 0.05% (v/v) Tween 20. Two-fold serial dilutions of antibodies and targeted complement-activating molecules were prepared in PBS buffer starting from 15 μg/mL. Samples of 100 μL were transferred to the ELISA plate and were incubated at room temperature. After one hour, the plate was washed and 100 μL of HRP-conjugated goat anti-human IgG detection antibody was added to the plate and incubated 30 minutes at room temperature. The plate was washed and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured. Results are shown in FIG. 47. Bebtelovimab and both targeted complement-activating molecules tested showed binding to S protein.

### Example 30

### Activity of Anti-S Protein-Derived Targeted Complement-Activating Molecules

Complement Deposition Activity

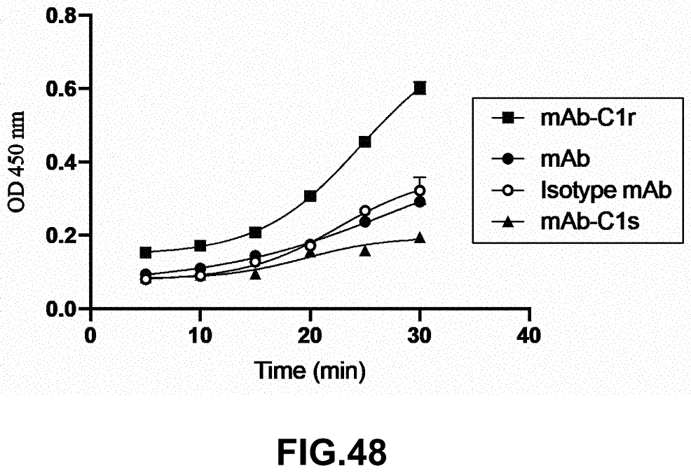

Deposition of C3b by antibody bebtelovimab and targeted complement-activating molecules bebtelovimab-C1r and bebtelovimab-C1s on an S protein-coated surface was assayed. Maxisorp polystyrene microtiter ELISA plates were coated with 2 μg/mL of recombinant S protein (R&D Systems) in coating buffer. The next day, the wells were blocked with 5% skimmed milk in PBS for two hours, then washed with PBS buffer containing 0.05% (v/v) Tween 20. NHS containing 7.5 μg of antibodies or targeted complement-activating molecules was diluted in BBS⁺⁺ buffer (4 mM barbital, 145 mM NaCl, 2 mM CaCl₂, 1 mM MgCl₂, pH 7.4) to a concentration of 2.5%, added to the plate, and incubated for 5, 10, 15, 20, 25, and 30 minutes at room temperature then washed three times. C3b deposition was detected by using rabbit anti-human C3c (Dako) followed by peroxidase-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured. An unrelated isotype antibody was used as a control. Results are shown in FIG. 48.

### Example 31

### Preparation of Anti-M Protein-Derived Targeted Complement-Activating Molecules

Antibodies (nanobody formats) to SARS-CoV-2 M protein (also referred to as membrane protein) were described by Hammel and Zenhausern (see Antib. Rep. (2020) 3(4):e230). The sequences of anti-M protein antibodies RB572 and RB574 were kindly provided by the Geneva Antibody Facility, University of Geneva, and used to create targeted complement-activating molecules comprising an antibody binding domain from either RB572 or RB574 and a fragment of either C1r or C1s. The C-terminal catalytic fragment of C1r or C1s was fused with the antibody at the C-terminus of the antibody's heavy chain (HC), resulting in targeted complement-activating molecules RB572-C1r and RB574-C1r.

Plasmid preparation, cloning, protein expression, and purification was carried out as described in Example 1.

### Example 32

### Binding of Anti-M Protein-Derived Targeted Complement-Activating Molecules

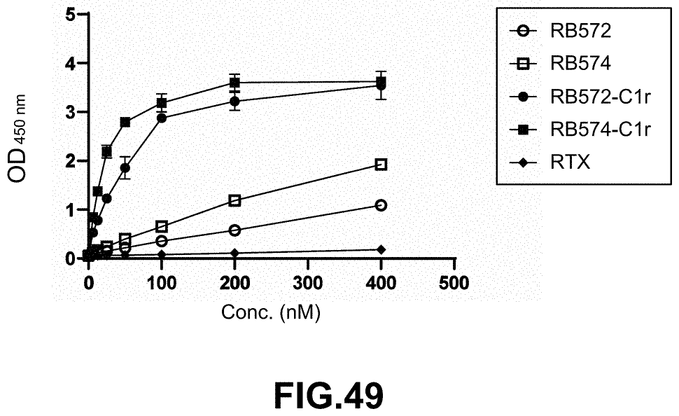

Binding to M protein was tested for each of the targeted complement-activating molecules RB572-C1r and RB574-C1r, along with antibodies RB572 and RB574. An unrelated isotype antibody (RTX) was used as a control. Maxisorp polystyrene microtiter ELISA plates were coated with 2 μg/mL of recombinant SARS-CoV-2 M protein (Trenzyme) in coating buffer. The next day, wells were blocked with 5% skimmed milk in PBS for two hours, then washed with PBS buffer containing 0.05% (v/v) Tween 20. Two-fold serial dilutions of antibodies and targeted complement-activating molecules were prepared in PBS buffer starting from 1400 nM. Samples of 100 μL were transferred to the ELISA plate and were incubated at room temperature. After one hour, the plate was washed and 100 μL of HRP-conjugated goat anti-human IgG detection antibody was added to the plate and incubated 30 minutes at room temperature. The plate was washed and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured. Results are shown in FIG. 49. RB572, RB574, and both targeted complement-activating molecules tested showed binding to M protein.

### Example 33

### Activity of Anti-M Protein-Derived Targeted Complement-Activating Molecules

Complement Deposition Activity

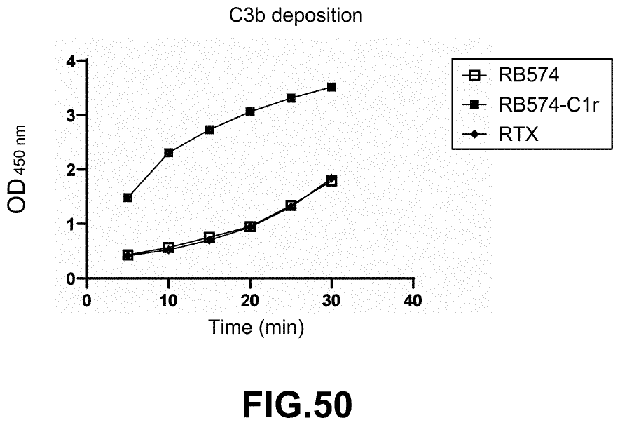

Deposition of C3b by antibody RB574 and targeted complement-activating molecule RB574-C1r on an M protein-coated surface was assayed. Maxisorp polystyrene microtiter ELISA plates were coated with 2 μg/mL of recombinant M protein in coating buffer. The next day, the wells were blocked with 1% BSA in PBS for two hours, then washed with PBS buffer containing 0.05% (v/v) Tween 20. NHS containing 200 nM of antibodies or targeted complement-activating molecules was diluted in BBS⁺⁺ buffer (4 mM barbital, 145 mM NaCl, 2 mM CaCl₂, 1 mM MgCl₂, pH 7.4) to a concentration of 3%, added to the plate, and incubated for 5, 10, 15, 20, 25, and 30 minutes at room temperature then washed three times. C3b deposition was detected by using rabbit anti-human C3c (Dako) followed by HRP-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured. An unrelated isotype antibody (RTX) was used as a control. Results are shown in FIG. 50.

### Example 34

### Preparation of Anti-Aspergillus Antibody-Derived Targeted Complement-Activating Molecules

Antibody to Aspergillus species was described by Davies et al. (Theranostics (2017) 7(14):3398). The sequence of anti-Aspergillus antibody hJF5 was used to create targeted complement-activating molecules comprising an antibody binding domain and a fragment of either C1r or C1s. The C-terminal catalytic fragment of C1r or C1s was fused with the antibody at the C-terminus of the antibody's heavy chain (HC), which was altered by a deletion of the single amino acid lysine (K) from the C-terminus. This process resulted in the following constructs: hJF5(H)^{ΔK}-C1r_HC (SEQ ID NO:134) and hJF5(H)^{ΔK}-C1s_HC (SEQ ID NO:135).

Plasmid preparation, cloning, protein expression, and purification was carried out as described in Example 1.

### Example 35

### Binding of Anti-Aspergillus Antibody-Derived Targeted Complement-Activating Molecules

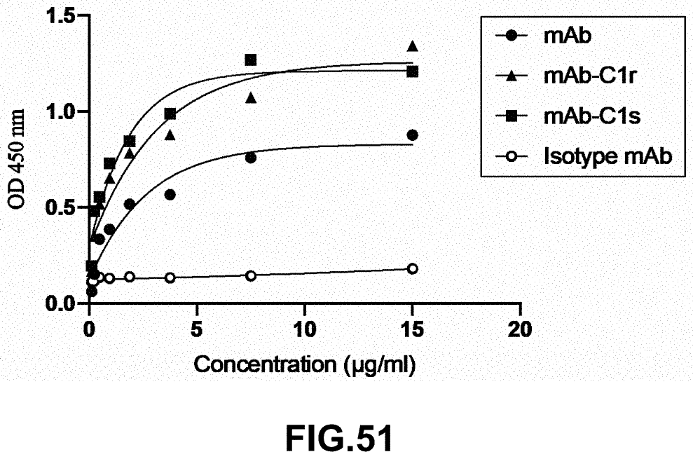

Binding to Aspergillus was tested for targeted complement-activating molecules hJF5(H)^{ΔK}-C1r (also referred to as hJF5-C1r) and hJF5(H)^{ΔK}-C1s (also referred to as hJF5-C1s), along with monoclonal antibody hJF5. Aspergillus fumigatus spores were subcultured on Sabouraud dextrose agar at 37° C. for 5-7 days then harvested using sterile physiological saline (PBS) containing 0.05% Tween-20. The fungus suspension was centrifuged at 3000 g for 10 minutes then washed with sterile PBS to remove any remaining detergent. After washing, cells were re-suspended in coating buffer and passed through a cell strainer (40 um) to give a homogenous suspension. The optical density at 550 nm, OD₅₅₀ was adjusted to be 0.5, then ELISA plates were coated with the fungal suspension. Maxisorp polystyrene microtiter ELISA plates were coated with Aspergillus fumigatus cells in coating buffer. The next day, wells were blocked with 5% skimmed milk in PBS for two hours, then washed with PBS buffer containing 0.05% (v/v) Tween 20. Two-fold serial dilutions of antibodies and targeted complement-activating molecules were prepared in PBS buffer starting from 15 μg/mL. Samples of 100 μL were transferred to the ELISA plate and were incubated at room temperature. After one hour, the plate was washed and 100 μL of HRP-conjugated goat anti-human IgG detection antibody was added to the plate and incubated 30 minutes at room temperature. The plate was washed and 100 μL of 1-Step Ultra TMB Solution (Thermo Fisher Scientific) was added to each well and incubated for two minutes at room temperature. The reaction was stopped by the addition of 2M H₂SO₄ and the optical density at 450 nm was immediately measured. Results are shown in FIG. 51. hJF5 antibody and both targeted complement-activating molecules tested showed binding to Aspergillus mannoprotein.

### Example 36

### Activity of Anti-Aspergillus Antibody-Derived Targeted Complement-Activating Molecules

Complement Deposition Activity

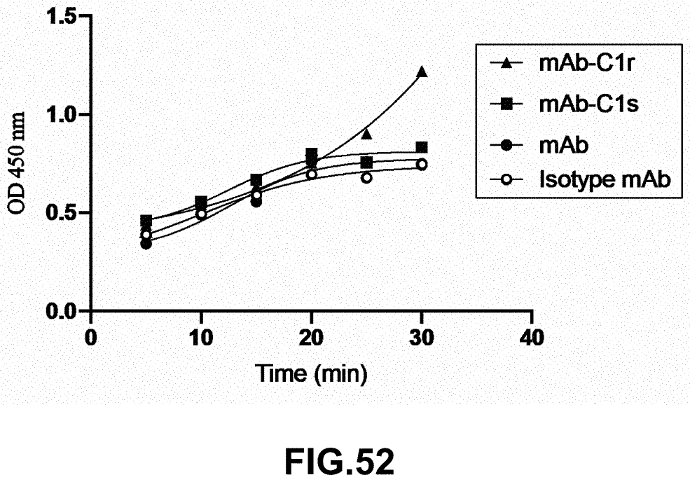

Deposition of C3b by antibody hJF5 and targeted complement-activating molecules hJF5-C1r and hJF5-C1s on an Aspergillus-coated surface was assayed. Aspergillus cells were prepared as described in Example 35. Maxisorp polystyrene microtiter ELISA plates were coated with Aspergillus cells in coating buffer. The next day, the wells were blocked with 5% skimmed milk in PBS for two hours, then washed with PBS buffer containing 0.05% (v/v) Tween 20. NHS containing 7.5 μg of antibodies or targeted complement-activating molecules was diluted in BBS⁺⁺ buffer (4 mM barbital, 145 mM NaCl, 2 mM CaCl₂, 1 mM MgCl₂, pH 7.4) to a concentration of 2.5%, added to the plate, and incubated for 5, 10, 15, 20, 25, and 30 minutes at room temperature then washed three times. C3b deposition was detected by using rabbit anti-human C3c (Dako) followed by HRP-conjugated goat anti-rabbit IgG (Southern Biotech). After one hour, the plate was washed three times and 100 μL of 1-Step Ultra TMB Solution (Thermo Fisher Scientific) was added to each well at room temperature. The reaction was stopped by the addition of 2M H₂SO₄ and the optical density at 450 nm was immediately measured. An unrelated isotype antibody was used as a control. Results are shown in FIG. 52.

### IX. OTHER EMBODIMENTS

All publications, patent applications, and patents mentioned in this specification are herein incorporated by reference.

While certain embodiments of the invention have been illustrated and described, it will be appreciated that various changes can be made therein without departing from the spirit and scope of the invention. Although the invention has been described in connection with specific embodiments, it should be understood that the invention as claimed should not be unduly limited to such specific embodiments. Indeed, various modifications of the specific embodiments described that are obvious to those skilled in the fields of medicine, immunology, pharmacology, or related fields are intended to be within the scope of the invention.

Accordingly, the following numbered paragraphs describing specific embodiments are provided for clarity, but should not be construed to limit the claims.

1. A targeted complement-activating molecule comprising:

(a) a target-binding domain; and

(b) a complement-activating serine protease effector domain.

2. The molecule of paragraph 1, wherein the complement-activating serine protease effector domain comprises MASP-1 or a fragment thereof, MASP-2 or a fragment thereof, MASP-3 or a fragment thereof, C1r or a fragment thereof, C1s or a fragment thereof, factor D or a fragment thereof, C2a or a fragment thereof, or factor Bb or a fragment thereof.

3. The molecule of paragraph 1 or paragraph 2, wherein the complement-activating serine protease effector domain is catalytically active.

4. The molecule of paragraph 1 or paragraph 2, wherein the complement-activating serine protease effector domain is in a zymogen form.

5. The molecule of any of paragraphs 1-4, wherein the target-binding domain binds to an antigen present on a cell.

6. The molecule of any of paragraphs 1-5, wherein the target-binding domain binds to CD20, CD38, or CD52.

7. The molecule of any of paragraphs 1-4, wherein the target-binding domain binds to an antigen present on a microbial pathogen.

8. The molecule of paragraph 7, wherein the target-binding domain binds to an antigen present on a bacterial pathogen, a viral pathogen, a fungal pathogen, or a parasitic pathogen.

9. The molecule of paragraph 8, wherein the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species.

10. The molecule of paragraph 9, wherein the bacterial pathogen is Neisseria meningitidis.

11. The molecule of paragraph 8, wherein the viral pathogen is an Epstein-Barr virus, a Human Immunodeficiency Virus 1 (HIV-1), a Herpesvirus, an Influenza virus, a West Nile virus, a Cytomegalovirus, or a Coronavirus.

12. The molecule of paragraph 8, wherein the fungal pathogen is Candida albicans or an Aspergillus species.

13. The molecule of paragraph 8, wherein the parasitic pathogen is Schistosoma mansoni, Plasmodium falciparum, or Trypanosoma cruzei.

14. The molecule any of paragraphs 1-13, wherein the target-binding domain comprises an antibody or an antigen-binding fragment thereof.

15. The molecule of paragraph 14, wherein the target-binding domain comprises an anti-CD20 antibody or an antigen-binding fragment thereof, an anti-CD38 antibody or an antigen-binding fragment thereof, or an anti-CD52 antibody or an antigen-binding fragment thereof.

16. The molecule of paragraph 15, wherein the target-binding domain comprises rituximab or an antigen-binding fragment thereof, alemtuzumab or an antigen-binding fragment thereof, or daratumumab or an antigen-binding fragment thereof.

17. The molecule of paragraph 14, wherein the target-binding domain comprises an antibody that binds an antigen present on a microbial pathogen.

18. The molecule of paragraph 17, wherein the target-binding domain comprises an anti-Neisseria antibody or an antigen-binding fragment thereof.

19. The molecule of paragraph 18, wherein the target-binding domain comprises an anti-fHbP antibody or an antigen-binding fragment thereof.

20. The molecule of paragraph 19, wherein the target-binding domain comprises anti-fHbP antibody clone 19, or an antigen-binding fragment thereof.

21. The molecule of paragraph 17, wherein the target-binding domain comprises an anti-Streptococcus antibody or an antigen-binding fragment thereof.

22. The molecule of paragraph 21, wherein the target-binding domain comprises an anti-PspA antibody or an antigen-binding fragment thereof.

23. The molecule of paragraph 22, wherein the target-binding domain comprises anti-PspA antibody RX1MI005 or an antigen-binding fragment thereof.

24. The molecule of paragraph 17, wherein the target-binding domain comprises an anti-Staphylococcus antibody or an antigen-binding fragment thereof.

25. The molecule of paragraph 24, wherein the target-binding domain comprises an anti-Fnbp antibody or an antigen-binding fragment thereof.

26. The molecule of paragraph 25, wherein the target-binding domain comprises anti-Fnbp antibody clone G or an antigen-binding fragment thereof.

27. The molecule of paragraph 17, wherein the target-binding domain comprises an anti-Candida antibody or an antigen-binding fragment thereof.

28. The molecule of paragraph 27, wherein the target-binding domain comprises an anti-fungal mannan antibody or an antigen-binding fragment thereof.

29. The molecule of paragraph 28, wherein the target-binding domain comprises anti-fungal mannan antibody 1A2 or an antigen-binding fragment thereof.

30. The molecule of paragraph 17, wherein the target-binding domain comprises an anti-Plasmodium antibody or an antigen-binding fragment thereof.

31. The molecule of paragraph 30, wherein the target-binding domain comprises an anti-PfRH5 antibody or an antigen-binding fragment thereof.

32. The molecule of paragraph 31, wherein the target-binding domain comprises anti-PfHR5 antibody R5.004 or an antigen-binding fragment thereof.

33. The molecule of paragraph 31, wherein the target-binding domain comprises anti-PfHR5 antibody R5.016 or an antigen-binding fragment thereof.

34. The molecule of paragraph 17, wherein the target-binding domain comprises an anti-HIV-1 antibody or an antigen-binding fragment thereof.

35. The molecule of paragraph 34, wherein the target-binding domain comprises an anti-GP120 antibody or an antigen-binding fragment thereof.

36. The molecule of paragraph 35, wherein the target-binding domain comprises anti-GP120 antibody PGT121 or an antigen-binding fragment thereof.

37. The molecule of paragraph 17, wherein the target-binding domain comprises an anti-SARS-CoV-2 antibody or an antigen-binding fragment thereof.

38. The molecule of paragraph 37, wherein the target-binding domain comprises an anti-S protein antibody or an antigen-binding fragment thereof.

39. The molecule of paragraph 38, wherein the target-binding domain comprises anti-S protein antibody bebtelovimab or an antigen-binding fragment thereof.

40. The molecule of paragraph 37, wherein the target-binding domain comprises an anti-M protein antibody or an antigen-binding fragment thereof.

41. The molecule of paragraph 40, wherein the target-binding domain comprises anti-M protein antibody RB572 or RB574.

42. The molecule of paragraph 17, wherein the target-binding domain comprises an anti-Aspergillus antibody or an antigen-binding fragment thereof.

43. The molecule of paragraph 42, wherein the target-binding domain comprises anti-Aspergillus antibody hJF5 or an antigen-binding fragment thereof.

44. The molecule of any of paragraphs 1-43, wherein the complement-activating serine protease effector domain comprises one or more mutations relative to a wild-type serine protease and/or the target-binding domain comprises one or more mutations relative to a wild-type antibody.

45. The molecule of paragraph 44, wherein the one or more mutations inhibit protein degradation.

46. The molecule of paragraph 44, wherein the one or more mutations confer resistance to serine protease inhibition by C1 inhibitor or other serpins.

47. The molecule of paragraph 44, wherein the one or more mutations inhibit glycosylation of the molecule at one or more amino acid residues.

48. The molecule of any one of paragraphs 14-47, wherein the target-binding domain comprises an antibody heavy chain or fragment thereof and an antibody light chain or fragment thereof.

49. The molecule of paragraph 48, wherein the molecule comprises:

a) a fusion protein comprising:
i) the N-terminus of the complement-activating serine protease effector domain fused to the C-terminus of the antibody heavy chain or fragment thereof; or
ii) the C-terminus of the complement-activating serine protease effector domain fused to the N-terminus of the antibody heavy chain or fragment thereof; and

an antibody light chain or fragment thereof; or

b) a fusion protein comprising:
i) the N-terminus of the complement-activating serine protease effector domain fused to the C-terminus of the antibody light chain or fragment thereof; or
ii) the C-terminus of the complement-activating serine protease effector domain fused to the N-terminus of the antibody light chain or fragment thereof; and

an antibody heavy chain or fragment thereof.

50. The molecule of any one of paragraphs 14-47, wherein the molecule comprises a fusion protein comprising:

a) the N-terminus of the complement-activating serine protease effector domain fused to the C-terminus of a single-chain antibody or fragment thereof or single-domain antibody or fragment thereof; or

b) the C-terminus of the complement-activating serine protease effector domain fused to the N-terminus of a single-chain antibody or fragment thereof or single-domain antibody or fragment thereof.

51. The molecule of any one of paragraphs 1-50, wherein the target-binding domain and the serine protease effector domain are connected by a linker.

52. The molecule of any one of paragraphs 48-51, wherein the target-binding domain comprises rituximab or an antigen-binding fragment thereof and the serine protease effector domain comprises factor D or a fragment thereof.

53. The molecule of any one of paragraphs 48-51, wherein the target-binding domain comprises rituximab or an antigen-binding fragment thereof and the serine protease effector domain comprises C1r or a fragment thereof.

54. The molecule of any one of paragraphs 48-51, wherein the target-binding domain comprises rituximab or an antigen-binding fragment thereof and the serine protease effector domain comprises C1s or a fragment thereof.

55. The molecule of any one of paragraphs 48-51, wherein the target-binding domain comprises rituximab or an antigen-binding fragment thereof and the serine protease effector domain comprises MASP-2 or a fragment thereof.

56. The molecule of any one of paragraphs 48-51, wherein the target-binding domain comprises rituximab or an antigen-binding fragment thereof and the serine protease effector domain comprises MASP-3 or a fragment thereof.

57. The molecule of any one of paragraphs 48-51, wherein the target-binding domain comprises rituximab or an antigen-binding fragment thereof and the serine protease effector domain comprises MASP-1 or a fragment thereof.

58. The molecule of any one of paragraphs 48-51, wherein the target-binding domain comprises rituximab or an antigen-binding fragment thereof and the serine protease effector domain comprises C2a or a fragment thereof.

59. The molecule of any one of paragraphs 48-51, wherein the target-binding domain comprises rituximab or an antigen-binding fragment thereof and the serine protease effector domain comprises factor Bb or a fragment thereof.

60. The molecule of any one of paragraphs 48-51, wherein the target-binding domain comprises alemtuzumab or an antigen-binding fragment thereof and the serine protease effector domain comprises factor D or a fragment thereof.

61. The molecule of any one of paragraphs 48-51, wherein the target-binding domain comprises alemtuzumab or an antigen-binding fragment thereof and the serine protease effector domain comprises C1r or a fragment thereof.

62. The molecule of any one of paragraphs 48-51, wherein the target-binding domain comprises alemtuzumab or an antigen-binding fragment thereof and the serine protease effector domain comprises C1s or a fragment thereof.

63. The molecule of any one of paragraphs 48-51, wherein the target-binding domain comprises alemtuzumab or an antigen-binding fragment thereof and the serine protease effector domain comprises MASP-2 or a fragment thereof.

64. The molecule of any one of paragraphs 48-51, wherein the target-binding domain comprises alemtuzumab or an antigen-binding fragment thereof and the serine protease effector domain comprises MASP-3 or a fragment thereof.

65. The molecule of any one of paragraphs 48-51, wherein the target-binding domain comprises alemtuzumab or an antigen-binding fragment thereof and the serine protease effector domain comprises MASP-1 or a fragment thereof.

66. The molecule of any one of paragraphs 48-51, wherein the target-binding domain comprises alemtuzumab or an antigen-binding fragment thereof and the serine protease effector domain comprises C2a or a fragment thereof.

67. The molecule of any one of paragraphs 48-51, wherein the target-binding domain comprises alemtuzumab or an antigen-binding fragment thereof and the serine protease effector domain comprises factor Bb or a fragment thereof.

68. The molecule of any one of paragraphs 48-51, wherein the target-binding domain comprises daratumumab or an antigen-binding fragment thereof and the serine protease effector domain comprises factor D or a fragment thereof.

69. The molecule of any one of paragraphs 48-51, wherein the target-binding domain comprises daratumumab or an antigen-binding fragment thereof and the serine protease effector domain comprises C1r or a fragment thereof.

70. The molecule of any one of paragraphs 48-51, wherein the target-binding domain comprises daratumumab or an antigen-binding fragment thereof and the serine protease effector domain comprises C1s or a fragment thereof.

71. The molecule of any one of paragraphs 48-51, wherein the target-binding domain comprises daratumumab or an antigen-binding fragment thereof and the serine protease effector domain comprises MASP-2 or a fragment thereof.

72. The molecule of any one of paragraphs 48-51, wherein the target-binding domain comprises daratumumab or an antigen-binding fragment thereof and the serine protease effector domain comprises MASP-3 or a fragment thereof.

73. The molecule of any one of paragraphs 48-51, wherein the target-binding domain comprises daratumumab or an antigen-binding fragment thereof and the serine protease effector domain comprises MASP-1 or a fragment thereof.

74. The molecule of any one of paragraphs 48-51, wherein the target-binding domain comprises daratumumab or an antigen-binding fragment thereof and the serine protease effector domain comprises C2a or a fragment thereof.

75. The molecule of any one of paragraphs 48-51, wherein the target-binding domain comprises daratumumab or an antigen-binding fragment thereof and the serine protease effector domain comprises factor Bb or a fragment thereof.

76. The molecule of any one of paragraphs 48-51, wherein the target-binding domain comprises anti-fHbP antibody clone 19 or an antigen-binding fragment thereof and the serine protease effector domain comprises factor D or a fragment thereof.

77. The molecule of any one of paragraphs 48-51, wherein the target-binding domain comprises anti-fHbP antibody clone 19 or an antigen-binding fragment thereof and the serine protease effector domain comprises C1r or a fragment thereof.

78. The molecule of any one of paragraphs 48-51, wherein the target-binding domain comprises anti-fHbP antibody clone 19 or an antigen-binding fragment thereof and the serine protease effector domain comprises C1s or a fragment thereof.

79. The molecule of any one of paragraphs 48-51, wherein the target-binding domain comprises anti-fHbP antibody clone 19 or an antigen-binding fragment thereof and the serine protease effector domain comprises MASP-2 or a fragment thereof.

80. The molecule of any one of paragraphs 48-51, wherein the target-binding domain comprises anti-fHbP antibody clone 19 or an antigen-binding fragment thereof and the serine protease effector domain comprises MASP-3 or a fragment thereof.

81. The molecule of any one of paragraphs 48-51, wherein the target-binding domain comprises anti-fHbP antibody clone 19 or an antigen-binding fragment thereof and the serine protease effector domain comprises MASP-1 or a fragment thereof.

82. The molecule of any one of paragraphs 48-51, wherein the target-binding domain comprises anti-fHbP antibody clone 19 or an antigen-binding fragment thereof and the serine protease effector domain comprises C2a or a fragment thereof.

83. The molecule of any one of paragraphs 48-51, wherein the target-binding domain comprises anti-fHbP antibody clone 19 or an antigen-binding fragment thereof and the serine protease effector domain comprises factor Bb or a fragment thereof.

84. The molecule of any one of paragraphs 48-51, wherein the target-binding domain comprises anti-PspA antibody RX1MI005 or an antigen-binding fragment thereof and the serine protease effector domain comprises factor D or a fragment thereof.

85. The molecule of any one of paragraphs 48-51, wherein the target-binding domain comprises anti-PspA antibody RX1MI005 or an antigen-binding fragment thereof and the serine protease effector domain comprises C1r or a fragment thereof.

86. The molecule of any one of paragraphs 48-51, wherein the target-binding domain comprises anti-PspA antibody RX1MI005 or an antigen-binding fragment thereof and the serine protease effector domain comprises C1s or a fragment thereof.

87. The molecule of any one of paragraphs 48-51, wherein the target-binding domain comprises anti-PspA antibody RX1MI005 or an antigen-binding fragment thereof and the serine protease effector domain comprises MASP-2 or a fragment thereof.

88. The molecule of any one of paragraphs 48-51, wherein the target-binding domain comprises anti-PspA antibody RX1MI005 or an antigen-binding fragment thereof and the serine protease effector domain comprises MASP-3 or a fragment thereof.

89. The molecule of any one of paragraphs 48-51, wherein the target-binding domain comprises anti-PspA antibody RX1MI005 or an antigen-binding fragment thereof and the serine protease effector domain comprises MASP-1 or a fragment thereof.

90. The molecule of any one of paragraphs 48-51, wherein the target-binding domain comprises anti-PspA antibody RX1MI005 or an antigen-binding fragment thereof and the serine protease effector domain comprises C2a or a fragment thereof.

91. The molecule of any one of paragraphs 48-51, wherein the target-binding domain comprises anti-PspA antibody RX1MI005 or an antigen-binding fragment thereof and the serine protease effector domain comprises factor Bb or a fragment thereof.

92. The molecule of any one of paragraphs 48-51, wherein the target-binding domain comprises anti-Fnbp antibody clone G or an antigen-binding fragment thereof and the serine protease effector domain comprises factor D or a fragment thereof.

93. The molecule of any one of paragraphs 48-51, wherein the target-binding domain comprises anti-Fnbp antibody clone G or an antigen-binding fragment thereof and the serine protease effector domain comprises C1r or a fragment thereof.

94. The molecule of any one of paragraphs 48-51, wherein the target-binding domain comprises anti-Fnbp antibody clone G or an antigen-binding fragment thereof and the serine protease effector domain comprises C1s or a fragment thereof.

95. The molecule of any one of paragraphs 48-51, wherein the target-binding domain comprises anti-Fnbp antibody clone G or an antigen-binding fragment thereof and the serine protease effector domain comprises MASP-2 or a fragment thereof.

96. The molecule of any one of paragraphs 48-51, wherein the target-binding domain comprises anti-Fnbp antibody clone G or an antigen-binding fragment thereof and the serine protease effector domain comprises MASP-3 or a fragment thereof.

97. The molecule of any one of paragraphs 48-51, wherein the target-binding domain comprises anti-Fnbp antibody clone G or an antigen-binding fragment thereof and the serine protease effector domain comprises MASP-1 or a fragment thereof.

98. The molecule of any one of paragraphs 48-51, wherein the target-binding domain comprises anti-Fnbp antibody clone G or an antigen-binding fragment thereof and the serine protease effector domain comprises C2a or a fragment thereof.

99. The molecule of any one of paragraphs 48-51, wherein the target-binding domain comprises anti-Fnbp antibody clone G or an antigen-binding fragment thereof and the serine protease effector domain comprises factor Bb or a fragment thereof.

100. The molecule of any one of paragraphs 48-51, wherein the target-binding domain comprises anti-Candida antibody 1A2 or an antigen-binding fragment thereof and the serine protease effector domain comprises factor D or a fragment thereof.

101. The molecule of any one of paragraphs 48-51, wherein the target-binding domain comprises anti-Candida antibody 1A2 or an antigen-binding fragment thereof and the serine protease effector domain comprises C1r or a fragment thereof.

102. The molecule of any one of paragraphs 48-51, wherein the target-binding domain comprises anti-Candida antibody 1A2 or an antigen-binding fragment thereof and the serine protease effector domain comprises C1s or a fragment thereof.

103. The molecule of any one of paragraphs 48-51, wherein the target-binding domain comprises anti-Candida antibody 1A2 or an antigen-binding fragment thereof and the serine protease effector domain comprises MASP-2 or a fragment thereof.

104. The molecule of any one of paragraphs 48-51, wherein the target-binding domain comprises anti-Candida antibody 1A2 or an antigen-binding fragment thereof and the serine protease effector domain comprises MASP-3 or a fragment thereof.

105. The molecule of any one of paragraphs 48-51, wherein the target-binding domain comprises anti-Candida antibody 1A2 or an antigen-binding fragment thereof and the serine protease effector domain comprises MASP-1 or a fragment thereof.

106. The molecule of any one of paragraphs 48-51, wherein the target-binding domain comprises anti-Candida antibody 1A2 or an antigen-binding fragment thereof and the serine protease effector domain comprises C2a or a fragment thereof.

107. The molecule of any one of paragraphs 48-51, wherein the target-binding domain comprises anti-Candida antibody 1A2 or an antigen-binding fragment thereof and the serine protease effector domain comprises factor Bb or a fragment thereof.

108. The molecule of any one of paragraphs 48-51, wherein the target-binding domain comprises anti-PfRH5 antibody R5.004 or an antigen-binding fragment thereof and the serine protease effector domain comprises factor D or a fragment thereof.

109. The molecule of any one of paragraphs 48-51, wherein the target-binding domain comprises anti-PfRH5 antibody R5.004 or an antigen-binding fragment thereof and the serine protease effector domain comprises C1r or a fragment thereof.

110. The molecule of any one of paragraphs 48-51, wherein the target-binding domain comprises anti-PfRH5 antibody R5.004 or an antigen-binding fragment thereof and the serine protease effector domain comprises C1s or a fragment thereof.

111. The molecule of any one of paragraphs 48-51, wherein the target-binding domain comprises anti-PfRH5 antibody R5.004 or an antigen-binding fragment thereof and the serine protease effector domain comprises MASP-2 or a fragment thereof.

112. The molecule of any one of paragraphs 48-51, wherein the target-binding domain comprises anti-PfRH5 antibody R5.004 or an antigen-binding fragment thereof and the serine protease effector domain comprises MASP-3 or a fragment thereof.

113. The molecule of any one of paragraphs 48-51, wherein the target-binding domain comprises anti-PfRH5 antibody R5.004 or an antigen-binding fragment thereof and the serine protease effector domain comprises MASP-1 or a fragment thereof.

114. The molecule of any one of paragraphs 48-51, wherein the target-binding domain comprises anti-PfRH5 antibody R5.004 or an antigen-binding fragment thereof and the serine protease effector domain comprises C2a or a fragment thereof.

115. The molecule of any one of paragraphs 48-51, wherein the target-binding domain comprises anti-PfRH5 antibody R5.004 or an antigen-binding fragment thereof and the serine protease effector domain comprises factor Bb or a fragment thereof.

116. The molecule of any one of paragraphs 48-51, wherein the target-binding domain comprises anti-PfRH5 antibody R5.016 or an antigen-binding fragment thereof and the serine protease effector domain comprises factor D or a fragment thereof.

117. The molecule of any one of paragraphs 48-51, wherein the target-binding domain comprises anti-PfRH5 antibody R5.016 or an antigen-binding fragment thereof and the serine protease effector domain comprises C1r or a fragment thereof.

118. The molecule of any one of paragraphs 48-51, wherein the target-binding domain comprises anti-PfRH5 antibody R5.016 or an antigen-binding fragment thereof and the serine protease effector domain comprises C1s or a fragment thereof.

119. The molecule of any one of paragraphs 48-51, wherein the target-binding domain comprises anti-PfRH5 antibody R5.016 or an antigen-binding fragment thereof and the serine protease effector domain comprises MASP-2 or a fragment thereof.

120. The molecule of any one of paragraphs 48-51, wherein the target-binding domain comprises anti-PfRH5 antibody R5.016 or an antigen-binding fragment thereof and the serine protease effector domain comprises MASP-3 or a fragment thereof.

121. The molecule of any one of paragraphs 48-51, wherein the target-binding domain comprises anti-PfRH5 antibody R5.016 or an antigen-binding fragment thereof and the serine protease effector domain comprises MASP-1 or a fragment thereof.

122. The molecule of any one of paragraphs 48-51, wherein the target-binding domain comprises anti-PfRH5 antibody R5.016 or an antigen-binding fragment thereof and the serine protease effector domain comprises C2a or a fragment thereof.

123. The molecule of any one of paragraphs 48-51, wherein the target-binding domain comprises anti-PfRH5 antibody R5.016 or an antigen-binding fragment thereof and the serine protease effector domain comprises factor Bb or a fragment thereof.

124. The molecule of any one of paragraphs 48-51, wherein the target-binding domain comprises anti-GP120 antibody PGT121 or an antigen-binding fragment thereof and the serine protease effector domain comprises factor D or a fragment thereof.

125. The molecule of any one of paragraphs 48-51, wherein the target-binding domain comprises anti-GP120 antibody PGT121 or an antigen-binding fragment thereof and the serine protease effector domain comprises C1r or a fragment thereof.

126. The molecule of any one of paragraphs 48-51, wherein the target-binding domain comprises anti-GP120 antibody PGT121 or an antigen-binding fragment thereof and the serine protease effector domain comprises C1s or a fragment thereof.

127. The molecule of any one of paragraphs 48-51, wherein the target-binding domain comprises anti-GP120 antibody PGT121 or an antigen-binding fragment thereof and the serine protease effector domain comprises MASP-2 or a fragment thereof.

128. The molecule of any one of paragraphs 48-51, wherein the target-binding domain comprises anti-GP120 antibody PGT121 or an antigen-binding fragment thereof and the serine protease effector domain comprises MASP-3 or a fragment thereof.

129. The molecule of any one of paragraphs 48-51, wherein the target-binding domain comprises anti-GP120 antibody PGT121 or an antigen-binding fragment thereof and the serine protease effector domain comprises MASP-1 or a fragment thereof.

130. The molecule of any one of paragraphs 48-51, wherein the target-binding domain comprises anti-GP120 antibody PGT121 or an antigen-binding fragment thereof and the serine protease effector domain comprises C2a or a fragment thereof.

131. The molecule of any one of paragraphs 48-51, wherein the target-binding domain comprises anti-GP120 antibody PGT121 or an antigen-binding fragment thereof and the serine protease effector domain comprises factor Bb or a fragment thereof.

132. The molecule of any one of paragraphs 48-51, wherein the target-binding domain comprises anti-SARS-CoV-2 S protein antibody bebtelovimab or an antigen-binding fragment thereof and the serine protease effector domain comprises factor D or a fragment thereof.

133. The molecule of any one of paragraphs 48-51, wherein the target-binding domain comprises anti-SARS-CoV-2 S protein antibody bebtelovimab or an antigen-binding fragment thereof and the serine protease effector domain comprises C1r or a fragment thereof.

134. The molecule of any one of paragraphs 48-51, wherein the target-binding domain comprises anti-SARS-CoV-2 S protein antibody bebtelovimab or an antigen-binding fragment thereof and the serine protease effector domain comprises C1s or a fragment thereof.

135. The molecule of any one of paragraphs 48-51, wherein the target-binding domain comprises anti-SARS-CoV-2 S protein antibody bebtelovimab or an antigen-binding fragment thereof and the serine protease effector domain comprises MASP-2 or a fragment thereof.

136. The molecule of any one of paragraphs 48-51, wherein the target-binding domain comprises anti-SARS-CoV-2 S protein antibody bebtelovimab or an antigen-binding fragment thereof and the serine protease effector domain comprises MASP-3 or a fragment thereof.

137. The molecule of any one of paragraphs 48-51, wherein the target-binding domain comprises anti-SARS-CoV-2 S protein antibody bebtelovimab or an antigen-binding fragment thereof and the serine protease effector domain comprises MASP-1 or a fragment thereof.

138. The molecule of any one of paragraphs 48-51, wherein the target-binding domain comprises anti-SARS-CoV-2 S protein antibody bebtelovimab or an antigen-binding fragment thereof and the serine protease effector domain comprises C2a or a fragment thereof.

139. The molecule of any one of paragraphs 48-51, wherein the target-binding domain comprises anti-SARS-CoV-2 S protein antibody bebtelovimab or an antigen-binding fragment thereof and the serine protease effector domain comprises factor Bb or a fragment thereof.

140. The molecule of any one of paragraphs 48-51, wherein the target-binding domain comprises anti-SARS-CoV-2 M protein antibody RB572 or RB574 or an antigen-binding fragment thereof and the serine protease effector domain comprises factor D or a fragment thereof.

141. The molecule of any one of paragraphs 48-51, wherein the target-binding domain comprises anti-SARS-CoV-2 M protein antibody RB572 or RB574 or an antigen-binding fragment thereof and the serine protease effector domain comprises C1r or a fragment thereof.

142. The molecule of any one of paragraphs 48-51, wherein the target-binding domain comprises anti-SARS-CoV-2 M protein antibody RB572 or RB574 or an antigen-binding fragment thereof and the serine protease effector domain comprises C1s or a fragment thereof.

143. The molecule of any one of paragraphs 48-51, wherein the target-binding domain comprises anti-SARS-CoV-2 M protein antibody RB572 or RB574 or an antigen-binding fragment thereof and the serine protease effector domain comprises MASP-2 or a fragment thereof.

144. The molecule of any one of paragraphs 48-51, wherein the target-binding domain comprises anti-SARS-CoV-2 M protein antibody RB572 or RB574 or an antigen-binding fragment thereof and the serine protease effector domain comprises MASP-3 or a fragment thereof.

145. The molecule of any one of paragraphs 48-51, wherein the target-binding domain comprises anti-SARS-CoV-2 M protein antibody RB572 or RB574 or an antigen-binding fragment thereof and the serine protease effector domain comprises MASP-1 or a fragment thereof.

146. The molecule of any one of paragraphs 48-51, wherein the target-binding domain comprises anti-SARS-CoV-2 M protein antibody RB572 or RB574 or an antigen-binding fragment thereof and the serine protease effector domain comprises C2a or a fragment thereof.

147. The molecule of any one of paragraphs 48-51, wherein the target-binding domain comprises anti-SARS-CoV-2 M protein antibody RB572 or RB574 or an antigen-binding fragment thereof and the serine protease effector domain comprises factor Bb or a fragment thereof.

148. The molecule of any one of paragraphs 48-51, wherein the target-binding domain comprises anti-Aspergillus antibody hJF5 or an antigen-binding fragment thereof and the serine protease effector domain comprises factor D or a fragment thereof.

149. The molecule of any one of paragraphs 48-51, wherein the target-binding domain comprises anti-Aspergillus antibody hJF5 or an antigen-binding fragment thereof and the serine protease effector domain comprises C1r or a fragment thereof.

150. The molecule of any one of paragraphs 48-51, wherein the target-binding domain comprises anti-Aspergillus antibody hJF5 or an antigen-binding fragment thereof and the serine protease effector domain comprises C1s or a fragment thereof.

151. The molecule of any one of paragraphs 48-51, wherein the target-binding domain comprises anti-Aspergillus antibody hJF5 or an antigen-binding fragment thereof and the serine protease effector domain comprises MASP-2 or a fragment thereof.

152. The molecule of any one of paragraphs 48-51, wherein the target-binding domain comprises anti-Aspergillus antibody hJF5 or an antigen-binding fragment thereof and the serine protease effector domain comprises MASP-3 or a fragment thereof.

153. The molecule of any one of paragraphs 48-51, wherein the target-binding domain comprises anti-Aspergillus antibody hJF5 or an antigen-binding fragment thereof and the serine protease effector domain comprises MASP-1 or a fragment thereof.

154. The molecule of any one of paragraphs 48-51, wherein the target-binding domain comprises anti-Aspergillus antibody hJF5 or an antigen-binding fragment thereof and the serine protease effector domain comprises C2a or a fragment thereof.

155. The molecule of any one of paragraphs 48-51, wherein the target-binding domain comprises anti-Aspergillus antibody hJF5 or an antigen-binding fragment thereof and the serine protease effector domain comprises factor Bb or a fragment thereof.

156. The molecule of any one of paragraphs 48-51, wherein the target-binding domain comprises a heavy chain as set forth in any one of SEQ ID NOs:1, 3, 20, and 54-56.

157. The molecule of any one of paragraphs 48-51, wherein the target-binding domain comprises a light chain as set forth in SEQ ID NO:2.

158. The molecule of any one of paragraphs 48-51, wherein the target-binding domain comprises a heavy chain as set forth in SEQ ID NO:93.

159. The molecule of any one of paragraphs 48-51, wherein the target-binding domain comprises a light chain as set forth in SEQ ID NO:94.

160. The molecule of any one of paragraphs 48-51, wherein the target-binding domain comprises a heavy chain as set forth in SEQ ID NO:95.

161. The molecule of any one of paragraphs 48-51, wherein the target-binding domain comprises a light chain as set forth in SEQ ID NO:96.

162. The molecule of any one of paragraphs 48-51, wherein the target-binding domain comprises a heavy chain as set forth in SEQ ID NO:103.

163. The molecule of any one of paragraphs 48-51, wherein the target-binding domain comprises a light chain as set forth in SEQ ID NO:104.

164. The molecule of any one of paragraphs 48-51, wherein the target-binding domain comprises a heavy chain as set forth in SEQ ID NO:120.

165. The molecule of any one of paragraphs 48-51, wherein the target-binding domain comprises a light chain as set forth in SEQ ID NO:121.

166. The molecule of any one of paragraphs 48-51, wherein the target-binding domain comprises a heavy chain as set forth in SEQ ID NO:124.

167. The molecule of any one of paragraphs 48-51, wherein the target-binding domain comprises a light chain as set forth in SEQ ID NO:125.

168. The molecule of any one of paragraphs 48-51, wherein the target-binding domain comprises a heavy chain as set forth in SEQ ID NO:128.

169. The molecule of any one of paragraphs 48-51, wherein the target-binding domain comprises a light chain as set forth in SEQ ID NO:129.

170. The molecule of any one of paragraphs 48-51, wherein the target-binding domain comprises a heavy chain as set forth in SEQ ID NO:136.

171. The molecule of any one of paragraphs 48-51, wherein the target-binding domain comprises a light chain as set forth in SEQ ID NO:137.

172. The molecule of any one of paragraphs 48-51, wherein the target-binding domain comprises a heavy chain as set forth in SEQ ID NO:140.

173. The molecule of any one of paragraphs 48-51, wherein the target-binding domain comprises a light chain as set forth in SEQ ID NO:141.

174. The molecule of any one of paragraphs 48-51, wherein the target-binding domain comprises a heavy chain as set forth in SEQ ID NO:144.

175. The molecule of any one of paragraphs 48-51, wherein the target-binding domain comprises a light chain as set forth in SEQ ID NO:145.

176. The molecule of any one of paragraphs 48-51, wherein the target-binding domain comprises a heavy chain as set forth in SEQ ID NO:148.

177. The molecule of any one of paragraphs 48-51, wherein the target-binding domain comprises a light chain as set forth in SEQ ID NO:149.

178. The molecule of any one of paragraphs 48-51, wherein the target-binding domain comprises a heavy chain as set forth in SEQ ID NO:132.

179. The molecule of any one of paragraphs 48-51, wherein the target-binding domain comprises a light chain as set forth in SEQ ID NO:133.

180. The molecule of any one of paragraphs 48-51, wherein the target-binding domain comprises a heavy chain as set forth in any one of SEQ ID NOs:1, 3, 20, and 54-56 and a light chain as set forth in SEQ ID NO:2.

181. The molecule of any one of paragraphs 48-51, wherein the target-binding domain comprises a heavy chain as set forth in SEQ ID NO:93 or 97 and a light chain as set forth in SEQ ID NO:94.

182. The molecule of any one of paragraphs 48-51, wherein the target-binding domain comprises a heavy chain as set forth in SEQ ID NO:95 or 98 and a light chain as set forth in SEQ ID NO:96.

183. The molecule of any one of paragraphs 48-51, wherein the target-binding domain comprises a heavy chain as set forth in any one of SEQ ID NOs:103, 114, 116, 117, 118, and 119.

184. The molecule of any one of paragraphs 48-51, wherein the target-binding domain comprises a light chain as set forth in SEQ ID NO:104.

185. The molecule of any one of paragraphs 48-51, wherein the target-binding domain comprises a heavy chain as set forth in any one of SEQ ID NOs:103, 114, 116, 117, 118, and 119 and a light chain as set forth in SEQ ID NO:104.

186. The molecule of any one of paragraphs 48-51, wherein the target-binding domain comprises a heavy chain as set forth in SEQ ID NO:122 or 123.

187. The molecule of any one of paragraphs 48-51, wherein the target-binding domain comprises a heavy chain as set forth in SEQ ID NO: 122 or 123 and a light chain as set forth in SEQ ID NO:121.

188. The molecule of any one of paragraphs 48-51, wherein the target-binding domain comprises a heavy chain as set forth in SEQ ID NO:126 or 127.

189. The molecule of any one of paragraphs 48-51, wherein the target-binding domain comprises a heavy chain as set forth in SEQ ID NO:126 or 127 and a light chain as set forth in SEQ ID NO:125.

190. The molecule of any one of paragraphs 48-51, wherein the target-binding domain comprises a heavy chain as set forth in SEQ ID NO:130 or 131.

191. The molecule of any one of paragraphs 48-51, wherein the target-binding domain comprises a heavy chain as set forth in SEQ ID NO:130 or 131 and a light chain as set forth in SEQ ID NO:129.

192. The molecule of any one of paragraphs 48-51, wherein the target-binding domain comprises a heavy chain as set forth in SEQ ID NO:138 or 139.

193. The molecule of any one of paragraphs 48-51, wherein the target-binding domain comprises a heavy chain as set forth in SEQ ID NO:138 or 139 and a light chain as set forth in SEQ ID NO:137.

194. The molecule of any one of paragraphs 48-51, wherein the target-binding domain comprises a heavy chain as set forth in SEQ ID NO:142 or 143.

195. The molecule of any one of paragraphs 48-51, wherein the target-binding domain comprises a heavy chain as set forth in SEQ ID NO:142 or 143 and a light chain as set forth in SEQ ID NO:141.

196. The molecule of any one of paragraphs 48-51, wherein the target-binding domain comprises a heavy chain as set forth in SEQ ID NO:146 or 147.

197. The molecule of any one of paragraphs 48-51, wherein the target-binding domain comprises a heavy chain as set forth in SEQ ID NO:146 or 147 and a light chain as set forth in SEQ ID NO:145.

198. The molecule of any one of paragraphs 48-51, wherein the target-binding domain comprises a heavy chain as set forth in SEQ ID NO:150 or 151.

199. The molecule of any one of paragraphs 48-51, wherein the target-binding domain comprises a heavy chain as set forth in SEQ ID NO:150 or 151 and a light chain as set forth in SEQ ID NO:149.

200. The molecule of any one of paragraphs 48-51, wherein the target-binding domain comprises a heavy chain as set forth in SEQ ID NO:134 or 135.

201. The molecule of any one of paragraphs 48-51, wherein the target-binding domain comprises a heavy chain as set forth in SEQ ID NO:134 or 135 and a light chain as set forth in SEQ ID NO:133.

202. The molecule of any one of paragraphs 1-48, wherein the serine protease effector domain comprises an amino acid sequence set forth in any one of SEQ ID NOs:57, 58, and 61-65.

203. The molecule of any one of paragraphs 1-48, wherein the serine protease effector domain comprises an amino acid sequence set forth in SEQ ID NO:66.

204. The molecule of any one of paragraphs 1-48, wherein the serine protease effector domain comprises an amino acid sequence set forth in SEQ ID NO:67 or SEQ ID NO:68.

205. The molecule of any one of paragraphs 1-48, wherein the serine protease effector domain comprises an amino acid sequence set forth in any one of SEQ ID NOs:69-74.

206. The molecule of any one of paragraphs 1-48, wherein the serine protease effector domain comprises an amino acid sequence set forth in any one of SEQ ID NO:76 and 78-87.

207. The molecule of any one of paragraphs 1-48, wherein the serine protease effector domain comprises an amino acid sequence set forth in SEQ ID NO:88.

208. The molecule of any one of paragraphs 1-48, wherein the serine protease effector domain comprises an amino acid sequence set forth in SEQ ID NO:89.

209. The molecule of any one of paragraphs 1-48, wherein the serine protease effector domain comprises an amino acid sequence set forth in SEQ ID NO:90 or 92.

210. The molecule of paragraph 49, wherein the fusion protein comprises an amino acid sequence set forth in any one of SEQ ID NOs:4-6, 9, and 33-38.

211. The molecule of paragraph 210, further comprising a light chain as set forth in SEQ ID NO:2.

212. The molecule of paragraph 49, wherein the fusion protein comprises an amino acid sequence set forth in any SEQ ID NO:7 or SEQ ID NO:8.

213. The molecule of paragraph 212, further comprising a heavy chain as set forth in any one of SEQ ID NOs:1, 3, 20, and 54-56.

214. The molecule of paragraph 49, wherein the fusion protein comprises an amino acid sequence set forth in SEQ ID NO:12 or SEQ ID NO:13.

215. The molecule of paragraph 214, further comprising a light chain as set forth in SEQ ID NO:2.

216. The molecule of paragraph 49, wherein the fusion protein comprises an amino acid sequence set forth in SEQ ID NO:14 or SEQ ID NO:15.

217. The molecule of paragraph 216, further comprising a heavy chain as set forth in any one of SEQ ID NOs: 1, 3, 20, and 54-56.

218. The molecule of paragraph 49, wherein the fusion protein comprises an amino acid sequence set forth in SEQ ID NO:16.

219. The molecule of paragraph 218, further comprising a light chain as set forth in SEQ ID NO:2.

220. The molecule of paragraph 49, wherein the fusion protein comprises an amino acid sequence set forth in SEQ ID NO:17.

221. The molecule of paragraph 220, further comprising a heavy chain as set forth in any one of SEQ ID NOs: 1, 3, 20, and 54-56.

222. The molecule of paragraph 49, wherein the fusion protein comprises an amino acid sequence set forth in any one of SEQ ID NOs:18, 21, 39-40, or 48-50.

223. The molecule of paragraph 222, further comprising a light chain as set forth in SEQ ID NO:2.

224. The molecule of paragraph 49, wherein the fusion protein comprises an amino acid sequence set forth in any one of SEQ ID NOs:19, 23, 41-47, or 51-53.

225. The molecule of paragraph 224, further comprising a light chain as set forth in SEQ ID NO:2.

226. The molecule of paragraph 49, wherein the fusion protein comprises an amino acid sequence set forth in SEQ ID NO:25.

227. The molecule of paragraph 226, further comprising a light chain as set forth in SEQ ID NO:2.

228. The molecule of paragraph 49, wherein the fusion protein comprises an amino acid sequence set forth in SEQ ID NO:26.

229. The molecule of paragraph 228, further comprising a light chain as set forth in SEQ ID NO:2.

230. The molecule of paragraph 49, wherein the fusion protein comprises an amino acid sequence set forth in any one of SEQ ID NOs:27, 28, 31, and 32.

231. The molecule of paragraph 230, further comprising a light chain as set forth in SEQ ID NO:2.

232. The molecule of paragraph 49, wherein the fusion protein comprises an amino acid sequence set forth in SEQ ID NO:29 or SEQ ID NO:30.

233. The molecule of paragraph 232, further comprising a heavy chain as set forth in any one of SEQ ID NOs: 1, 3, 20, and 54-56.

234. The molecule of paragraph 49, wherein the fusion protein comprises an amino acid sequence set forth in SEQ ID NO:97.

235. The molecule of paragraph 234, further comprising a light chain as set forth in SEQ ID NO:94.

236. The molecule of paragraph 49, wherein the fusion protein comprises an amino acid sequence set forth in SEQ ID NO:98.

237. The molecule of paragraph 236, further comprising a light chain as set forth in SEQ ID NO:96.

238. The molecule of paragraph 49, wherein the fusion protein comprises an amino acid sequence set forth in any one of SEQ ID NOs:108, 111, 116, 117, 119, and 119.

239. The molecule of paragraph 238, further comprising a light chain as set forth in SEQ ID NO:104.

240. The molecule of paragraph 49, wherein the fusion protein comprises an amino acid sequence set forth in SEQ ID NO:122 or 123.

241. The molecule of paragraph 240, further comprising a light chain as set forth in SEQ ID NO:121.

242. The molecule of paragraph 49, wherein the fusion protein comprises an amino acid sequence set forth in SEQ ID NO:126 or 127.

243. The molecule of paragraph 242, further comprising a light chain as set forth in SEQ ID NO:125.

244. The molecule of paragraph 49, wherein the fusion protein comprises an amino acid sequence set forth in SEQ ID NO:130 or 131.

245. The molecule of paragraph 244, further comprising a light chain as set forth in SEQ ID NO:129.

246. The molecule of paragraph 49, wherein the fusion protein comprises an amino acid sequence set forth in SEQ ID NO:138 or 139.

247. The molecule of paragraph 246, further comprising a light chain as set forth in SEQ ID NO:137.

248. The molecule of paragraph 49, wherein the fusion protein comprises an amino acid sequence set forth in SEQ ID NO:142 or 143.

249. The molecule of paragraph 248, further comprising a light chain as set forth in SEQ ID NO:141.

250. The molecule of paragraph 49, wherein the fusion protein comprises an amino acid sequence set forth in SEQ ID NO:146 or 147.

251. The molecule of paragraph 250, further comprising a light chain as set forth in SEQ ID NO:145.

252. The molecule of paragraph 49, wherein the fusion protein comprises an amino acid sequence set forth in SEQ ID NO:150 or 151.

253. The molecule of paragraph 252, further comprising a light chain as set forth in SEQ ID NO:149.

254. The molecule of paragraph 49, wherein the fusion protein comprises an amino acid sequence set forth in SEQ ID NO:134 or 135.

255. The molecule of paragraph 254, further comprising a light chain as set forth in SEQ ID NO:133.

256. The molecule of any one of paragraphs 1-255, wherein the molecule binds to a target with an affinity between 1 pM and 1 μM.

257. The molecule of any one of paragraphs 1-255, wherein the molecule binds to a target on a cell surface with an affinity between 1 pM and 1 μM.

258. The molecule of any one of paragraphs 1-257, wherein the molecule has a serine protease activity that is at least 70% of the serine protease activity of the serine protease domain alone.

259. The molecule of any one of paragraphs 1-257, wherein the molecule has a serine protease activity that is at least 80% of the serine protease activity of the serine protease domain alone.

260. The molecule of any one of paragraphs 1-257, wherein the molecule has a serine protease activity that is at least 90% of the serine protease activity of the serine protease domain alone.

261. The molecule of any one of paragraphs 1-260, wherein the molecule binds to a target on a cell surface and activates a complement pathway when administered to a mammalian subject.

262. The molecule of any one of paragraphs 1-261, wherein the molecule induces complement dependent cytotoxicity (CDC), complement-dependent cell-mediated cytotoxicity (CDCC), and/or complement-dependent cellular phagocytosis (CDCP).

263. A polynucleotide encoding the molecule of any one of paragraphs 1-262.

264. A polynucleotide encoding the fusion protein of any one of paragraphs 26, 27, and 210-253.

265. A cloning vector or expression cassette comprising the polynucleotide of paragraphs 263 or 264.

266. A cloning vector or expression cassette comprising a first polynucleotide encoding the fusion protein of any one of paragraphs 26, 27, and 210-253 and a second polynucleotide; wherein the second polynucleotide encodes an antibody heavy chain or fragment thereof if the fusion protein comprises an antibody light chain or fragment thereof, and the second polynucleotide encodes an antibody light chain or fragment thereof if the fusion protein comprises an antibody heavy chain or fragment thereof.

267. A first cloning vector or expression cassette comprising a first polynucleotide encoding the fusion protein of any one of paragraphs 26, 27, and 210-253 and a second cloning vector or expression cassette comprising a second polynucleotide; wherein the second polynucleotide encodes an antibody heavy chain or fragment thereof if the fusion protein comprises an antibody light chain or fragment thereof, and the second polynucleotide encodes an antibody light chain or fragment thereof if the fusion protein comprises an antibody heavy chain or fragment thereof.

268. A host cell expressing the molecule of any one of paragraphs 1-262, or comprising the cloning vector(s) or expression cassette(s) of any one of paragraphs 265-267.

268. A method of producing a molecule comprising:

(a) a target-binding domain; and

(b) a complement-activating serine protease effector domain; the method comprising culturing the host cell of paragraph 266 under conditions allowing for expression of the molecule and isolating the molecule.

270. The use of the molecule of any one of paragraphs 1-262 to activate at least one complement pathway in a mammalian subject.

271. The use of paragraph 270, wherein the activation of the at least one complement pathway comprises:

a) activation of the complement classical pathway;

b) activation of the complement lectin pathway;

c) activation of the complement alternative pathway; or

d) two or more of (a)-(c).

272. The use of the molecule of any one of paragraphs 1-262 to induce complement dependent cell death (CDC), complement-dependent cell-mediated cytotoxicity (CDCC), or complement-dependent cellular phagocytosis (CDCP) in a target cell.

273. The use of the molecule of any one of paragraphs 1-262 to treat cancer.

274. The use of the molecule of any one of paragraphs 1-262 to treat autoimmune disease.

275. The use of the molecule of any one of paragraphs 1-262 to treat a microbial infection in a mammalian subject.

276. The use of paragraph 275, wherein the infection is a bacterial infection, a viral infection, a fungal infection, or a parasitic infection.

277. A composition comprising the molecule of any one of paragraphs 1-262 and one or more excipients.

278. A method of activating at least one complement pathway in a mammalian subject by administering the molecule of any one of paragraphs 1-262 or the composition of paragraph 239.

279. The method of paragraph 278, wherein the activation of the at least one complement pathway comprises:

a) activation of the complement classical pathway;

b) activation of the complement lectin pathway;

c) activation of the complement alternative pathway; or

d) two or more of (a)-(c).

280. A method of inducing complement dependent cell death (CDC) in a target cell, comprising contacting the target cell with the molecule of any one of paragraphs 1-262 or the composition of paragraph 277, wherein said contacting results in complement deposition on the target cell, thereby leading to complement-mediated cell death.

281. A method of inducing complement-dependent cell-mediated cytotoxicity (CDCC) or complement-dependent cellular phagocytosis (CDCP) toward a target cell, comprising contacting the target cell with the molecule of any one of paragraphs 1-262 or the composition of paragraph 277, wherein said contacting results in complement deposition on the target cell, thereby leading to complement-mediated cell death.

282. A method of treating cancer, comprising administering the molecule of any one of paragraphs 1-262 or the composition of paragraph 277 to a mammalian subject in need thereof.

283. The method of paragraph 282, wherein the cancer is a solid tumor cancer.

284. The method of paragraph 282 wherein the cancer is a hematological cancer.

285. A method of treating an autoimmune disease, comprising administering the molecule of any one of paragraphs 1-262 or the composition of paragraph 277 to a mammalian subject in need thereof.

286. A method of treating a microbial infection in a mammalian subject, comprising administering the molecule of any one of paragraphs 1-262 or the composition of paragraph 277 to the subject.

287. The method of paragraph 286, wherein the infection is a bacterial infection, a viral infection, a fungal infection, or a parasitic infection.

288. The method of paragraph 287, wherein the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species.

289. The method of paragraph 287, wherein the bacterial pathogen is Neisseria meningitidis.

290. The method of paragraph 287, wherein the bacterial pathogen is Streptococcus pneumoniae.

291. The method of paragraph 287, wherein the bacterial pathogen is Staphylococcus aureus.

292. The method of paragraph 287, wherein the viral pathogen is an Epstein-Barr virus, a Human Immunodeficiency Virus 1 (HIV-1), a Herpesvirus, an Influenza virus, a West Nile virus, a Cytomegalovirus, or a Coronavirus.

293. The method of paragraph 287, wherein the viral pathogen is HIV-1.

294. The method of paragraph 287, wherein the viral pathogen is SARS-CoV-2.

295. The method of paragraph 287, wherein the fungal pathogen is Candida albicans or an Aspergillus species.

296. The method of paragraph 287, wherein the fungal pathogen is Candida albicans.

297. The method of paragraph 287, wherein the parasitic pathogen is Schistosoma mansoni, Plasmodium falciparum, or Trypanosoma cruzei.

298. The method of paragraph 287, wherein the parasitic pathogen is Plasmodium falciparum.

299. The targeted complement-activating molecule of any one of paragraphs 1-262 or the composition of paragraph 277 for use in the manufacture of a medicament for treating cancer, autoimmune disease, or a microbial infection.

300. The composition of paragraph 277 for use in treating cancer, autoimmune disease, or a microbial infection.

1. A targeted complement-activating molecule comprising:
(a) a target-binding domain; and
(b) a complement-activating serine protease effector domain.

2. The molecule of claim 1, wherein the complement-activating serine protease effector domain comprises MASP-1 or a fragment thereof, MASP-2 or a fragment thereof, MASP-3 or a fragment thereof, C1r or a fragment thereof, C1s or a fragment thereof, factor D or a fragment thereof, C2a or a fragment thereof, or factor Bb or a fragment thereof.

3. The molecule of claim 1, wherein the complement-activating serine protease effector domain is catalytically active.

4. The molecule of claim 1, wherein the complement-activating serine protease effector domain is in a zymogen form.

5. The molecule of claim 1, wherein the target-binding domain binds to an antigen present on a cell.

6. The molecule of claim 1, wherein the target-binding domain binds to CD20, CD38, or CD52.

7. The molecule of claim 1, wherein the target-binding domain binds to an antigen present on a microbial pathogen.

8. The molecule of claim 7, wherein the target-binding domain binds to an antigen present on a bacterial pathogen, a viral pathogen, a fungal pathogen, or a parasitic pathogen.

9. The molecule of claim 8, wherein the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species.

10. The molecule of claim 9, wherein the bacterial pathogen is Neisseria meningitidis.

11. The molecule of claim 8, wherein the viral pathogen is an Epstein-Barr virus, a Human Immunodeficiency Virus 1 (HIV-1), a Herpesvirus, an Influenza virus, a West Nile virus, a Cytomegalovirus, or a Coronavirus.

12. The molecule of claim 8, wherein the fungal pathogen is Candida albicans or an Aspergillus species.

13. The molecule of claim 8, wherein the parasitic pathogen is Schistosoma mansoni, Plasmodium falciparum, or Trypanosoma cruzei.

14. The molecule of claim 1, wherein the target-binding domain comprises an antibody or an antigen-binding fragment thereof.

15. The molecule of claim 14, wherein the target-binding domain comprises an anti-CD20 antibody or an antigen-binding fragment thereof, an anti-CD38 antibody or an antigen-binding fragment thereof, or an anti-CD52 antibody or an antigen-binding fragment thereof.

16. The molecule of claim 15, wherein the target-binding domain comprises rituximab or an antigen-binding fragment thereof, alemtuzumab or an antigen-binding fragment thereof, or daratumumab or an antigen-binding fragment thereof.

17. The molecule of claim 14, wherein the target-binding domain comprises an antibody that binds an antigen present on a microbial pathogen.

18. The molecule of claim 17, wherein the target-binding domain comprises an anti-Neisseria antibody or an antigen-binding fragment thereof, an anti-Streptococcus antibody or antigen-binding fragment thereof, an anti-Staphylococcus antibody or antigen-binding fragment thereof, and anti-Candida antibody or antigen-binding fragment thereof, an anti-Plasmodium antibody or antigen-binding fragment thereof, an anti-HIV-1 antibody or antigen-binding fragment thereof, an anti-SARS-CoV-2 antibody or antigen-binding fragment thereof, or an anti-Aspergillus antibody or antigen-binding fragment thereof.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. The molecule of claim 1, wherein the complement-activating serine protease effector domain comprises one or more mutations relative to a wild-type serine protease and/or the target-binding domain comprises one or more mutations relative to a wild-type antibody.

45. The molecule of claim 44, wherein the one or more mutations inhibit protein degradation.

46. The molecule of claim 44, wherein the one or more mutations confer resistance to serine protease inhibition by C1 inhibitor or other serpins.

47. The molecule of claim 44, wherein the one or more mutations inhibit glycosylation of the molecule at one or more amino acid residues.

48. The molecule of claim 14, wherein the target-binding domain comprises an antibody heavy chain or fragment thereof and an antibody light chain or fragment thereof.

49. The molecule of claim 48, wherein the molecule comprises:
a) a fusion protein comprising:
i) the N-terminus of the complement-activating serine protease effector domain fused to the C-terminus of the antibody heavy chain or fragment thereof; or
ii) the C-terminus of the complement-activating serine protease effector domain fused to the N-terminus of the antibody heavy chain or fragment thereof; and
an antibody light chain or fragment thereof; or
b) a fusion protein comprising:
i) the N-terminus of the complement-activating serine protease effector domain fused to the C-terminus of the antibody light chain or fragment thereof; or
ii) the C-terminus of the complement-activating serine protease effector domain fused to the N-terminus of the antibody light chain or fragment thereof; and
an antibody heavy chain or fragment thereof.

50. The molecule of claim 14, wherein the molecule comprises a fusion protein comprising:
a) the N-terminus of the complement-activating serine protease effector domain fused to the C-terminus of a single-chain antibody or fragment thereof or single-domain antibody or fragment thereof; or
b) the C-terminus of the complement-activating serine protease effector domain fused to the N-terminus of a single-chain antibody or fragment thereof or single-domain antibody or fragment thereof.

51. The molecule of claim 1, wherein the target-binding domain and the serine protease effector domain are connected by a linker.

52. The molecule of claim 48, wherein the target-binding domain comprises rituximab or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

53. (canceled)

54. (canceled)

55. (canceled)

56. (canceled)

57. (canceled)

58. (canceled)

59. (canceled)

60. The molecule of claim 48, wherein the target-binding domain comprises alemtuzumab or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

61. (canceled)

62. (canceled)

63. (canceled)

64. (canceled)

65. (canceled)

66. (canceled)

67. (canceled)

68. The molecule of claim 48, wherein the target-binding domain comprises daratumumab or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

69. (canceled)

70. (canceled)

71. (canceled)

72. (canceled)

73. (canceled)

74. (canceled)

75. (canceled)

76. The molecule of claim 48, wherein the target-binding domain comprises anti-fHbP antibody clone 19 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

77. (canceled)

78. (canceled)

79. (canceled)

80. (canceled)

81. (canceled)

82. (canceled)

83. (canceled)

84. The molecule of claim 48, wherein the target-binding domain comprises anti-PspA antibody RX1MI005 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

85. (canceled)

86. (canceled)

87. (canceled)

88. (canceled)

89. (canceled)

90. (canceled)

91. (canceled)

92. The molecule of claim 48, wherein the target-binding domain comprises anti-Fnbp antibody clone G or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

93. (canceled)

94. (canceled)

95. (canceled)

96. (canceled)

97. (canceled)

98. (canceled)

99. (canceled)

100. The molecule of claim 48, wherein the target-binding domain comprises anti-Candida antibody 1A2 or an antigen-binding fragment thereof and the serine protease effector domain comprises factor D or a fragment thereof, C1s or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

101. (canceled)

102. (canceled)

103. (canceled)

104. (canceled)

105. (canceled)

106. (canceled)

107. (canceled)

108. The molecule of claim 48, wherein the target-binding domain comprises anti-PfRH5 antibody R5.004 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

109. (canceled)

110. (canceled)

111. (canceled)

112. (canceled)

113. (canceled)

114. (canceled)

115. (canceled)

116. The molecule of claim 48, wherein the target-binding domain comprises anti-PfRH5 antibody R5.016 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

117. (canceled)

118. (canceled)

119. (canceled)

120. (canceled)

121. (canceled)

122. (canceled)

123. (canceled)

124. The molecule of claim 48, wherein the target-binding domain comprises anti-GP120 antibody PGT121 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

125. (canceled)

126. (canceled)

127. (canceled)

128. (canceled)

129. (canceled)

130. (canceled)

131. (canceled)

132. The molecule of claim 48, wherein the target-binding domain comprises anti-SARS-CoV-2 S protein antibody bebtelovimab or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

133. (canceled)

134. (canceled)

135. (canceled)

136. (canceled)

137. (canceled)

138. (canceled)

139. (canceled)

140. The molecule of claim 48, wherein the target-binding domain comprises anti-SARS-CoV-2 M protein antibody RB572 or RB574 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, or Bb or a fragment thereof.

141. (canceled)

142. (canceled)

143. (canceled)

144. (canceled)

145. (canceled)

146. (canceled)

147. (canceled)

148. The molecule of claim 48, wherein the target-binding domain comprises anti-Aspergillus antibody hJF5 or an antigen-binding fragment thereof and the serine protease effector domain comprises factor D or a fragment thereof, C1r or a fragment thereof, C1s or a fragment thereof, MASP-2 or a fragment thereof, MASP-3 or a fragment thereof, MASP-1 or a fragment thereof, C2a or a fragment thereof, Bb or a fragment thereof.

149. (canceled)

150. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)

174. (canceled)

175. (canceled)

176. (canceled)

178. (canceled)

179. (canceled)

180. The molecule of claim 48, wherein the target-binding domain comprises a heavy chain as set forth in any one of SEQ ID NOs:1, 3, 20, and 54-56 and a light chain as set forth in SEQ ID NO:2.

181. The molecule of claim 48, wherein the target-binding domain comprises a heavy chain as set forth in SEQ ID NO:93 or 97 and a light chain as set forth in SEQ ID NO:94.

182. The molecule of claim 48, wherein the target-binding domain comprises a heavy chain as set forth in SEQ ID NO:95 or 98 and a light chain as set forth in SEQ ID NO:96.

183. (canceled)

184. (canceled)

185. The molecule of claim 48, wherein the target-binding domain comprises a heavy chain as set forth in any one of SEQ ID NOs:103, 114, 116, 117, 118, and 119 and a light chain as set forth in SEQ ID NO:104.

186. (canceled)

187. The molecule of claim 48, wherein the target-binding domain comprises a heavy chain as set forth in SEQ ID NO: 122 or 123 and a light chain as set forth in SEQ ID NO:121.

188. (canceled)

189. The molecule of claim 48, wherein the target-binding domain comprises a heavy chain as set forth in SEQ ID NO:126 or 127 and a light chain as set forth in SEQ ID NO:125.

190. (canceled)

191. The molecule of claim 48, wherein the target-binding domain comprises a heavy chain as set forth in SEQ ID NO:130 or 131 and a light chain as set forth in SEQ ID NO:129.

192. (canceled)

193. The molecule of claim 48, wherein the target-binding domain comprises a heavy chain as set forth in SEQ ID NO:138 or 139 and a light chain as set forth in SEQ ID NO:137.

194. (canceled)

195. The molecule of claim 48, wherein the target-binding domain comprises a heavy chain as set forth in SEQ ID NO:142 or 143 and a light chain as set forth in SEQ ID NO:141.

196. (canceled)

197. The molecule of claim 48, wherein the target-binding domain comprises a heavy chain as set forth in SEQ ID NO:146 or 147 and a light chain as set forth in SEQ ID NO:145.

198. (canceled)

199. The molecule of claim 48, wherein the target-binding domain comprises a heavy chain as set forth in SEQ ID NO:150 or 151 and a light chain as set forth in SEQ ID NO:149.

200. (canceled)

201. The molecule of claim 48, wherein the target-binding domain comprises a heavy chain as set forth in SEQ ID NO:134 or 135 and a light chain as set forth in SEQ ID NO:133.

202. The molecule of claim 1, wherein the serine protease effector domain comprises an amino acid sequence set forth in any one of SEQ ID NOs:57, 58, 61-74, 76, 78-90, and 92.

203. (canceled)

204. (canceled)

205. (canceled)

206. (canceled)

207. (canceled)

208. (canceled)

209. (canceled)

210. The molecule of claim 49, wherein the fusion protein comprises an amino acid sequence set forth in any one of SEQ ID NOs:4-6, 9, and 33-38.

211. The molecule of claim 210, further comprising a light chain as set forth in SEQ ID NO:2.

212. The molecule of claim 49, wherein the fusion protein comprises an amino acid sequence set forth in any SEQ ID NO:7 or SEQ ID NO:8.

213. The molecule of claim 212, further comprising a heavy chain as set forth in any one of SEQ ID NOs:1, 3, 20, and 54-56.

214. The molecule of claim 49, wherein the fusion protein comprises an amino acid sequence set forth in SEQ ID NO:12 or SEQ ID NO:13.

215. The molecule of claim 214, further comprising a light chain as set forth in SEQ ID NO:2.

216. The molecule of claim 49, wherein the fusion protein comprises an amino acid sequence set forth in SEQ ID NO:14 or SEQ ID NO:15.

217. The molecule of claim 216, further comprising a heavy chain as set forth in any one of SEQ ID NOs: 1, 3, 20, and 54-56.

218. The molecule of claim 49, wherein the fusion protein comprises an amino acid sequence set forth in SEQ ID NO:16.

219. The molecule of claim 218, further comprising a light chain as set forth in SEQ ID NO:2.

220. The molecule of claim 49, wherein the fusion protein comprises an amino acid sequence set forth in SEQ ID NO:17.

221. The molecule of claim 220, further comprising a heavy chain as set forth in any one of SEQ ID NOs: 1, 3, 20, and 54-56.

222. The molecule of claim 49, wherein the fusion protein comprises an amino acid sequence set forth in any one of SEQ ID NOs:18, 21, 39-40, or 48-50.

223. The molecule of claim 222, further comprising a light chain as set forth in SEQ ID NO:2.

224. The molecule of claim 49, wherein the fusion protein comprises an amino acid sequence set forth in any one of SEQ ID NOs:19, 23, 41-47, or 51-53.

225. The molecule of claim 224, further comprising a light chain as set forth in SEQ ID NO:2.

226. The molecule of claim 49, wherein the fusion protein comprises an amino acid sequence set forth in SEQ ID NO:25.

227. The molecule of claim 226, further comprising a light chain as set forth in SEQ ID NO:2.

228. The molecule of claim 49, wherein the fusion protein comprises an amino acid sequence set forth in SEQ ID NO:26.

229. The molecule of claim 228, further comprising a light chain as set forth in SEQ ID NO:2.

230. The molecule of claim 49, wherein the fusion protein comprises an amino acid sequence set forth in any one of SEQ ID NOs:27, 28, 31, and 32.

231. The molecule of claim 230, further comprising a light chain as set forth in SEQ ID NO:2.

232. The molecule of claim 49, wherein the fusion protein comprises an amino acid sequence set forth in SEQ ID NO:29 or SEQ ID NO:30.

233. The molecule of claim 232, further comprising a heavy chain as set forth in any one of SEQ ID NOs: 1, 3, 20, and 54-56.

234. The molecule of claim 49, wherein the fusion protein comprises an amino acid sequence set forth in SEQ ID NO:97.

235. The molecule of claim 234, further comprising a light chain as set forth in SEQ ID NO:94.

236. The molecule of claim 49, wherein the fusion protein comprises an amino acid sequence set forth in SEQ ID NO:98.

237. The molecule of claim 236, further comprising a light chain as set forth in SEQ ID NO:96.

238. The molecule of claim 49, wherein the fusion protein comprises an amino acid sequence set forth in any one of SEQ ID NOs:108, 111, 116, 117, 119, and 119.

239. The molecule of claim 238, further comprising a light chain as set forth in SEQ ID NO:104.

240. The molecule of claim 49, wherein the fusion protein comprises an amino acid sequence set forth in SEQ ID NO:122 or 123.

241. The molecule of claim 240, further comprising a light chain as set forth in SEQ ID NO:121.

242. The molecule of claim 49, wherein the fusion protein comprises an amino acid sequence set forth in SEQ ID NO:126 or 127.

243. The molecule of claim 242, further comprising a light chain as set forth in SEQ ID NO:125.

244. The molecule of claim 49, wherein the fusion protein comprises an amino acid sequence set forth in SEQ ID NO:130 or 131.

245. The molecule of claim 244, further comprising a light chain as set forth in SEQ ID NO:129.

246. The molecule of claim 49, wherein the fusion protein comprises an amino acid sequence set forth in SEQ ID NO:138 or 139.

247. The molecule of claim 246, further comprising a light chain as set forth in SEQ ID NO:137.

248. The molecule of claim 49, wherein the fusion protein comprises an amino acid sequence set forth in SEQ ID NO:142 or 143.

249. The molecule of claim 248, further comprising a light chain as set forth in SEQ ID NO:141.

250. The molecule of claim 49, wherein the fusion protein comprises an amino acid sequence set forth in SEQ ID NO:146 or 147.

251. The molecule of claim 250, further comprising a light chain as set forth in SEQ ID NO:145.

252. The molecule of claim 49, wherein the fusion protein comprises an amino acid sequence set forth in SEQ ID NO:150 or 151.

253. The molecule of claim 252, further comprising a light chain as set forth in SEQ ID NO:149.

254. The molecule of claim 49, wherein the fusion protein comprises an amino acid sequence set forth in SEQ ID NO:134 or 135.

255. The molecule of claim 254, further comprising a light chain as set forth in SEQ ID NO:133.

256. The molecule of claim 1, wherein the molecule binds to a target with an affinity between 1 pM and 1 μM.

257. The molecule of claim 1, wherein the molecule binds to a target on a cell surface with an affinity between 1 pM and 1 μM.

258. The molecule of claim 1, wherein the molecule has a serine protease activity that is at least 70% of the serine protease activity of the serine protease domain alone.

259. The molecule of claim 1, wherein the molecule has a serine protease activity that is at least 80% of the serine protease activity of the serine protease domain alone.

260. The molecule of claim 1, wherein the molecule has a serine protease activity that is at least 90% of the serine protease activity of the serine protease domain alone.

261. The molecule of claim 1, wherein the molecule binds to a target on a cell surface and activates a complement pathway when administered to a mammalian subject.

262. The molecule of claim 1, wherein the molecule induces complement dependent cytotoxicity (CDC), complement-dependent cell-mediated cytotoxicity (CDCC), and/or complement-dependent cellular phagocytosis (CDCP).

263. A polynucleotide encoding the molecule of claim 1.

264. A polynucleotide encoding the fusion protein of claim 48.

265. A cloning vector or expression cassette comprising the polynucleotide of claim 263, 264, or 301.

266. A cloning vector or expression cassette comprising a first polynucleotide encoding the fusion protein of claim 48 and a second polynucleotide; wherein the second polynucleotide encodes an antibody heavy chain or fragment thereof if the fusion protein comprises an antibody light chain or fragment thereof, and the second polynucleotide encodes an antibody light chain or fragment thereof if the fusion protein comprises an antibody heavy chain or fragment thereof.

267. A first cloning vector or expression cassette comprising a first polynucleotide encoding the fusion protein of claim 48 and a second cloning vector or expression cassette comprising a second polynucleotide; wherein the second polynucleotide encodes an antibody heavy chain or fragment thereof if the fusion protein comprises an antibody light chain or fragment thereof, and the second polynucleotide encodes an antibody light chain or fragment thereof if the fusion protein comprises an antibody heavy chain or fragment thereof.

268. A host cell expressing the molecule of claim 1.

269. A method of producing a molecule comprising:
(a) a target-binding domain; and
(b) a complement-activating serine protease effector domain; the method comprising culturing the host cell of claim 268 under conditions allowing for expression of the molecule and isolating the molecule.

270. (canceled)

271. (canceled)

272. (canceled)

273. (canceled)

274. (canceled)

275. (canceled)

276. (canceled)

277. A composition comprising the molecule of claim 1 and one or more excipients.

278. A method of activating at least one complement pathway in a mammalian subject by administering the molecule of claim 1.

279. The method of claim 278, wherein the activation of the at least one complement pathway comprises:
a) activation of the complement classical pathway;
b) activation of the complement lectin pathway;
c) activation of the complement alternative pathway; or
d) two or more of (a)-(c).

280. A method of inducing complement dependent cell death (CDC) in a target cell, comprising contacting the target cell with the molecule of claim 1, wherein said contacting results in complement deposition on the target cell, thereby leading to complement-mediated cell death.

281. A method of inducing complement-dependent cell-mediated cytotoxicity (CDCC) or complement-dependent cellular phagocytosis (CDCP) toward a target cell, comprising contacting the target cell with the molecule of claim 1, wherein said contacting results in complement deposition on the target cell, thereby leading to complement-mediated cell death.

282. A method of treating cancer, comprising administering the molecule of claim 1 to a mammalian subject in need thereof.

283. The method of claim 282, wherein the cancer is a solid tumor cancer.

284. The method of claim 282 wherein the cancer is a hematological cancer.

285. A method of treating an autoimmune disease, comprising administering the molecule of claim 1 to a mammalian subject in need thereof.

286. A method of treating a microbial infection in a mammalian subject, comprising administering the molecule of claim 1 to the subject.

287. The method of claim 286, wherein the infection is a bacterial infection, a viral infection, a fungal infection, or a parasitic infection.

288. The method of claim 287, wherein the bacterial pathogen is Neisseria meningitidis, Staphylococcus aureus, Borrelia burgdorferi, Escherichia coli, Klebsiella pneumoniae, Streptococcus pneumoniae, Serratia marcenscens, Haemophilus influenzae, Mycobacterium tuberculosis, Treponema pallidum, Neisseria gonorrhea, Clostridium dificile, a Salmonella species, a Helicobacter species, a Shigella species, a Campylobacter species, or a Listeria species.

289. (canceled)

290. (canceled)

291. (canceled)

292. The method of claim 287, wherein the viral pathogen is an Epstein-Barr virus, a Human Immunodeficiency Virus 1 (HIV-1), a Herpesvirus, an Influenza virus, a West Nile virus, a Cytomegalovirus, or a Coronavirus.

293. (canceled)

294. (canceled)

295. The method of claim 287, wherein the fungal pathogen is Candida albicans or an Aspergillus species.

296. (canceled)

297. The method of claim 287, wherein the parasitic pathogen is Schistosoma mansoni, Plasmodium falciparum, or Trypanosoma cruzei.

298. (canceled)

299. (canceled)

300. (canceled)

301. A polynucleotide encoding the fusion protein of claim 49.


SEQUENCE LISTING (151 sequences)

SEQ ID NO: 1 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 450 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 854 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 5 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 6 - protein, 854 aa, synthetic construct
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDFGGGGSGGGGSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 616 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 8 - protein, 616 aa, synthetic construct
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDFGGGGSGGGGSGGGGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 10 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGQIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 11 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDAGGALVFLDSETERWFVGGIVSWGSMNCGEAGQYGVYTKVINYIPWIENIISDF

SEQ ID NO: 12 - protein, 894 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 13 - protein, 895 aa, synthetic construct
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVERGGGGSGGGGSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 657 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 15 - protein, 657 aa, synthetic construct
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVERGGGGSGGGGSGGGGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 865 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLQDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 17 - protein, 628 aa, synthetic construct
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLQDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRNGGGGSGGGGSGGGGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 19 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 20 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 22 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDAGGVFAVRDPNTDRWVATGIVSWGIGCSRGYGFYTKVLNYVDWIKKEMEEED

SEQ ID NO: 23 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 24 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDAGGAFAVQDPNDKTKFYAAGLVSWGPQCGTYGLYTRVKNYVDWIMKTMQENSTPRED

SEQ ID NO: 25 - protein, 973 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGKIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 26 - protein, 969 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGKIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 27 - protein, 692 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 28 - protein, 693 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 455 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 30 - protein, 455 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 693 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDAGGPLVCGGVLEGVVTSGSRVCGNRKKPGIYTRVASYAAWIDSVLAGGGGSGGGGSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 699 aa, synthetic construct
APPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTQGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 34 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAQYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 35 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILQDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 36 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLQSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 37 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQQDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 38 - protein, 853 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDQPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 39 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIQDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 40 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPQNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 41 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAQAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 42 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAQYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 43 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFQDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 44 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGQVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 45 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGQWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 46 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSQLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 47 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLQCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 48 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIWGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 49 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHWRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 50 - protein, 863 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGWGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 51 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFWNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 52 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDWPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 53 - protein, 861 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNWWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 54 - protein, 450 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTQGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 450 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTQGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 389 aa, Homo sapiens
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 58 - protein, 389 aa, synthetic construct
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 59 - protein, 389 aa, synthetic construct
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGQIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 60 - protein, 389 aa, synthetic construct
QPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGRIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDAGGALVFLDSETERWFVGGIVSWGSMNCGEAGQYGVYTKVINYIPWIENIISDF

SEQ ID NO: 61 - protein, 389 aa, synthetic construct
QPCPYPMAPPNGHVSPVQAQYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 62 - protein, 389 aa, synthetic construct
QPCPYPMAPPNGHVSPVQAKYILQDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 63 - protein, 389 aa, synthetic construct
QPCPYPMAPPNGHVSPVQAKYILKDSFSIFCETGYELLQGHLPLQSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 64 - protein, 389 aa, synthetic construct
QPCPYPMAPPNGHVSPVQAKYILKDSFSIFCETGYELLQGHLPLKSFTAVCQQDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 65 - protein, 389 aa, synthetic construct
QPCPYPMAPPNGHVSPVQAKYILKDSFSIFCETGYELLQGHLPLKSFTAVCQKDGSWDQPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 66 - protein, 430 aa, Homo sapiens
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 67 - protein, 401 aa, Homo sapiens
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLRDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 68 - protein, 401 aa, synthetic construct
NECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPVCGLPKFSRKLMARIFNGRPAQKGTTPWIAMLSHLNGQPFCGGSLLGSSWIVTAAHCLHQSLDPEDPTLQDSDLLSPSDFKIILGKHWRLRSDENEQHLGVKHTTLHPQYDPNTFENDVALVELLESPVLNAFVMPICLPEGPQQEGAMVIVSGWGKQFLQRFPETLMEIEIPIVDHSTCQKAYAPLKKKVTRDMICAGEKEGGKDACAGDSGGPMVTLNRERGQWYLVGTVSWGDDCGKKDRYGVYSYIHHNKDWIQRVTGVRN

SEQ ID NO: 69 - protein, 399 aa, Homo sapiens
IKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 70 - protein, 399 aa, synthetic construct
IKCPQPKTLDEFTIIQNLQPQYQFRDYFIATCKQGYQLIEGNQVLHSFTAVCQDDGTWHRAMPRCKIQDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 71 - protein, 399 aa, synthetic construct
IKCPQPKTLDEFTIIQNLQPQYQFRDYFIATCKQGYQLIEGNQVLHSFTAVCQDDGTWHRAMPRCKIKDCGQPQNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 72 - protein, 399 aa, synthetic construct
IKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIWGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 73 - protein, 399 aa, synthetic construct
IKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHWRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 74 - protein, 399 aa, synthetic construct
IKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGWGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 75 - protein, 399 aa, synthetic construct
IKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDAGGVFAVRDPNTDRWVATGIVSWGIGCSRGYGFYTKVLNYVDWIKKEMEEED

SEQ ID NO: 76 - protein, 397 aa, Homo sapiens [sequence omitted]

SEQ ID NO: 77 - protein, 397 aa, synthetic construct
MPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDAGGAFAVQDPNDKTKFYAAGLVSWGPQCGTYGLYTRVKNYVDWIMKTMQENSTPRED

SEQ ID NO: 78 - protein, 397 aa, synthetic construct
MPCPKEDTPNSVWEPAQAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 79 - protein, 397 aa, synthetic construct
MPCPKEDTPNSVWEPAKAQYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 80 - protein, 397 aa, synthetic construct
MPCPKEDTPNSVWEPAKAKYVFQDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 81 - protein, 397 aa, synthetic construct
MPCPKEDTPNSVWEPAKAKYVFRDVVQITCLDGFEVVEGQVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 82 - protein, 397 aa, synthetic construct
MPCPKEDTPNSVWEPAKAKYVFRDVVQITCLDGFEVVEGRVGATSFYSTCQSNGQWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 83 - protein, 397 aa, synthetic construct
MPCPKEDTPNSVWEPAKAKYVFRDVVQITCLDGFEVVEGRVGATSFYSTCQSNGKWSNSQLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 84 - protein, 397 aa, synthetic construct
MPCPKEDTPNSVWEPAKAKYVFRDVVQITCLDGFEVVEGRVGATSFYSTCQSNGKWSNSKLQCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 85 - protein, 397 aa, synthetic construct
MPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFWNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 86 - protein, 397 aa, synthetic construct
MPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDWPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 87 - protein, 397 aa, synthetic construct
MPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNWWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 88 - protein, 509 aa, Homo sapiens
KIQIQRSGHLNLYLLLDCSQSVSENDFLIFKESASLMVDRIFSFEINVSVAIITFASEPKVLMSVLNDNSRDMTEVISSLENANYKDHENGTGTNTYAALNSVYLMMNNQMRLLGMETMAWQEIRHAIILLTDGKSNMGGSPKTAVDHIREILNINQKRNDYLDIYAIGVGKLDVDWRELNELGSKKDGERHAFILQDTKALHQVFEHMLDVSKLTDTICGVGNMSANASDQERTPWHVTIKPKSQETCRGALISDQWVLTAAHCFRDGNDHSLWRVNVGDPKSQWGKEFLIEKAVISPGFDVFAKKNQGILEFYGDDIALLKLAQKVKMSTHARPICLPCTMEANLALRRPQGSTCRDHENELLNKQSVPAHFVALNGSKLNINLKMGVEWTSCAEVVSQEKTMFPNLTDVREVVTDQFLCSGTQEDESPCKGESGGAVFLERRFRFFQVGLVSWGLYNPCLGSADKNSRKRAPRSKVPPPRDFHINLFRMQPWLRQHLGDVLNFLPL

SEQ ID NO: 89 - protein, 505 aa, Homo sapiens
KIVLDPSGSMNIYLVLDGSDSIGASNFTGAKKCLVNLIEKVASYGVKPRYGLVTYATYPKIWVKVSEADSSNADWVTKQLNEINYEDHKLKSGTNTKKALQAVYSMMSWPDDVPPEGWNRTRHVIILMTDGLHNMGGDPITVIDEIRDLLYIGKDRKNPREDYLDVYVFGVGPLVNQVNINALASKKDNEQHVFKVKDMENLEDVFYQMIDESQSLSLCGMVWEHRKGTDYHKQPWQAKISVIRPSKGHESCMGAVVSEYFVLTAAHCFTVDDKEHSIKVSVGGEKRDLEIEVVLFHPNYNINGKKEAGIPEFYDYDVALIKLKNKLKYGQTIRPICLPCTEGTTRALRLPPTTTCQQQKEELLPAQDIKALFVSEEEKKLTRKEVYIKNGDKKGSCERDAQYAPGYDKVKDISEVVTPRFLCTGGVSPYADPNTCRGDSGGPLIVHKRSRFIQVGVISWGVVDVCKNQKRQKQVPAHARDFHINLFQVLPWLKEKLQDEDLGFL

SEQ ID NO: 90 - protein, 228 aa, Homo sapiens
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 91 - protein, 228 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDAGGPLVCGGVLEGVVTSGSRVCGNRKKPGIYTRVASYAAWIDSVLA

SEQ ID NO: 92 - protein, 234 aa, Homo sapiens
APPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLA

SEQ ID NO: 93 - protein, 451 aa, synthetic construct
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 214 aa, synthetic construct
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 693 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGQVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 694 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGEVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 5 aa, synthetic construct
GGGGS

SEQ ID NO: 100 - protein, 14 aa, synthetic construct
GGGGSGGGGSGGGG

SEQ ID NO: 101 - protein, 451 aa, Mus musculus
EVQLQQSGAELVRPGALVKLSCKASGFNIKDFYMHWVKQRPDQGLEWIGWIDPENDNTIYDPKFQGKASITADTSSNTAYLQLSSLTSEDTAVYYCAYRSPMITTGAMDFWGPGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 218 aa, Mus musculus
DIVLTQSPASLAVSLGQRATISCRASESVDNYGIGFMNWFQQKPGQPPKLLIYVASNQGSGVPARFSGSGSGTDFSLNIHPMEEDDAAMYFCQQSKEVPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 448 aa, Mus musculus
EVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 219 aa, Mus musculus
DIVMTQAAPSVSVSLGESVSIYCRSNKSLLYSNGNTYLYWFLQRPGQSPQLLIYRMSNLASGVPDRFSGSGSGTAFTLRISRVEAEDVGVYYCLQHLEYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 451 aa, Mus musculus
EVQLQQSGAELVRPGALVKLSCKASGFNIKDYYMHWVRQRPEQGLEWIGWIDPENGNTIYDPKFQGKASITADTSSNTAYLQLSSLTSEDTAVYYCAYRSTMITTGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 218 aa, Mus musculus
DIVLTQSPASLAVSLGQRATISCRASESVDNYGISFMNWFQQKPGQSPKLLIYAASNQGSGVPARFSGSGSGTDFSLNIHPMEEDDTAMYFCQQSKEVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 863 aa, synthetic construct
EVQLQQSGAELVRPGALVKLSCKASGFNIKDFYMHWVKQRPDQGLEWIGWIDPENDNTIYDPKFQGKASITADTSSNTAYLQLSSLTSEDTAVYYCAYRSPMITTGAMDFWGPGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 108 - protein, 860 aa, synthetic construct
EVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 109 - protein, 863 aa, synthetic construct
EVQLQQSGAELVRPGALVKLSCKASGFNIKDYYMHWVRQRPEQGLEWIGWIDPENGNTIYDPKFQGKASITADTSSNTAYLQLSSLTSEDTAVYYCAYRSTMITTGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 110 - protein, 861 aa, synthetic construct
EVQLQQSGAELVRPGALVKLSCKASGFNIKDFYMHWVKQRPDQGLEWIGWIDPENDNTIYDPKFQGKASITADTSSNTAYLQLSSLTSEDTAVYYCAYRSPMITTGAMDFWGPGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 111 - protein, 858 aa, synthetic construct
EVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 112 - protein, 861 aa, synthetic construct
EVQLQQSGAELVRPGALVKLSCKASGFNIKDYYMHWVRQRPEQGLEWIGWIDPENGNTIYDPKFQGKASITADTSSNTAYLQLSSLTSEDTAVYYCAYRSTMITTGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 113 - protein, 450 aa, synthetic construct
EVQLQQSGAELVRPGALVKLSCKASGFNIKDFYMHWVKQRPDQGLEWIGWIDPENDNTIYDPKFQGKASITADTSSNTAYLQLSSLTSEDTAVYYCAYRSPMITTGAMDFWGPGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 114 - protein, 447 aa, synthetic construct
EVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 450 aa, synthetic construct
EVQLQQSGAELVRPGALVKLSCKASGFNIKDYYMHWVRQRPEQGLEWIGWIDPENGNTIYDPKFQGKASITADTSSNTAYLQLSSLTSEDTAVYYCAYRSTMITTGAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 850 aa, synthetic construct
EVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGQPCPYPMAPPNGHVSPVQAKYILKDSFSIFCETGYELLQGHLPLKSFTAVCQKDGSWDRPMPACSIVDCGPPDDLPSGRVEYITGPGVTTYKAVIQYSCEETFYTMKVNDGKYVCEADGFWTSSKGEKSLPVCEPVCGLSARTTGGKIYGGQKAKPGDFPWQVLILGGTTAAGALLYDNWVLTAAHAVYEQKHDASALDIRMGTLKRLSPHYTQAWSEAVFIHEGYTHDAGFDNDIALIKLNNKVVINSNITPICLPRKEAESFMRTDDIGTASGWGLTQRGFLARNLMYVDIPIVDHQKCTAAYEKPPYPRGSVTANMLCAGLESGGKDSCRGDSGGALVFLDSETERWFVGGIVSWGSMNCGEAGQYGVYTKVINYIPWIENIISDF

SEQ ID NO: 117 - protein, 891 aa, synthetic construct
EVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGNECPELQPPVHGKIEPSQAKYFFKDQVLVSCDTGYKVLKDNVEMDTFQIECLKDGTWSNKIPTCKIVDCRAPGELEHGLITFSTRNNLTTYKSEIKYSCQEPYYKMLNNNTGIYTCSAQGVWMNKVLGRSLPTCLPECGQPSRSLPSLVKRIIGGRNAEPGLFPWQALIVVEDTSRVPNDKWFGSGALLSASWILTAAHVLRSQRRDTTVIPVSKEHVTVYLGLHDVRDKSGAVNSSAARVVLHPDFNIQNYNHDIALVQLQEPVPLGPHVMPVCLPRLEPEGPAPHMLGLVAGWGISNPNVTVDEIISSGTRTLSDVLQYVKLPVVPHAECKTSYESRSGNYSVTENMFCAGYYEGGKDTCLGDSGGAFVIFDDLSQRWVVQGLVSWGGPEECGSKQVYGVYTKVSNYVDWVWEQMGLPQSVVEPQVER

SEQ ID NO: 118 - protein, 690 aa, synthetic construct
ILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGEVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 696 aa, synthetic construct
APPRGRILGGREAEAHARPYMASVQLNGAHLCGGVLVAEQWVLSAAHCLEDAADGKVQVLLGAHSLSQPEPSKRLYDVLRAVPHPDSQPDTIDHDLLLLQLSEKATLGPAVRPLPWQRVDRDVAPGTLCDVAGWGIVNHAGRRPDSLQHVLLPVLDRATCNRRTHHDGAITERLMCAESNRRDSCKGDSGGPLVCGGVLEGVVTSGSRVCGNRKKPGIYTRVASYAAWIDSVLAGGGGSGGGGSGGGGEVQLVESGGGLVQPKGSLKLSCAASGFTFNMYVMNWVRQAPGKGLEWVARIRSKSYNFGTYYADSVKDRFTISRDDSQSVLYLTMNNLKTEDTAMYYCVRLDDDFAYWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 447 aa, Mus musculus
EVMLVESGGGLVKPGGSLKLSCAASGFTFSSYTMSWVRQTPEKRLEWVATISGGGGNTYYSDSVKGRFTISRDNAKNTLYLQMSSLRSEDTALYYCARHDYGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 214 aa, Mus musculus
DIKMTQSPSSMYASLGERVTITCKASQDINRYLSWFQQKPGKSPKTLIYRANRLVDGVPSRFSGSGSGQDYSLTISSLEYEDMGIYYCLQYDEFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 859 aa, synthetic construct
EVMLVESGGGLVKPGGSLKLSCAASGFTFSSYTMSWVRQTPEKRLEWVATISGGGGNTYYSDSVKGRFTISRDNAKNTLYLQMSSLRSEDTALYYCARHDYGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 123 - protein, 857 aa, synthetic construct
EVMLVESGGGLVKPGGSLKLSCAASGFTFSSYTMSWVRQTPEKRLEWVATISGGGGNTYYSDSVKGRFTISRDNAKNTLYLQMSSLRSEDTALYYCARHDYGSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 124 - protein, 442 aa, Mus musculus
EVQLQQSGPELVKPGASVKMSCKASGYTFTDYYMKWVKQSHGKSLEWIGDINPNNGDTFYNQKFKGKATLTVDKSSSTAYMQLNSLTSEDSAVYYCARGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 219 aa, Mus musculus
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 854 aa, synthetic construct
EVQLQQSGPELVKPGASVKMSCKASGYTFTDYYMKWVKQSHGKSLEWIGDINPNNGDTFYNQKFKGKATLTVDKSSSTAYMQLNSLTSEDSAVYYCARGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 127 - protein, 852 aa, synthetic construct
EVQLQQSGPELVKPGASVKMSCKASGYTFTDYYMKWVKQSHGKSLEWIGDINPNNGDTFYNQKFKGKATLTVDKSSSTAYMQLNSLTSEDSAVYYCARGDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 128 - protein, 442 aa, Ovis aries
QVRLQESGPSLVKPSQTLSLTCTVSGFGLTTYSIEWVRQAPGKALEWVGAVNNNGRTFYNPALKSRLSITRDTSKSQVSLSLSSVTTEDTAVYYCVRTWDVWGRGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 216 aa, Ovis aries
QAVLTQPSSVSRSLGQSVSITCSGSSSNIGSWNYVDWFQVIPGSAPRTLITAATSRTSGVPDRFSGSRSGNTATLTITSLQAEDEADYYCAAWDRSNSKIFGSGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 130 - protein, 854 aa, synthetic construct
QVRLQESGPSLVKPSQTLSLTCTVSGFGLTTYSIEWVRQAPGKALEWVGAVNNNGRTFYNPALKSRLSITRDTSKSQVSLSLSSVTTEDTAVYYCVRTWDVWGRGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 131 - protein, 852 aa, synthetic construct
QVRLQESGPSLVKPSQTLSLTCTVSGFGLTTYSIEWVRQAPGKALEWVGAVNNNGRTFYNPALKSRLSITRDTSKSQVSLSLSSVTTEDTAVYYCVRTWDVWGRGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 132 - protein, 448 aa, synthetic construct
EVQLVESGGGLVQPGGSLKLSCAASGFDFSRYWMSWVRQAPGKGLEWVAEINPDSSKINYMPSLKDRFTISRDNAKNSLYLQMNSLRAEDTAVYYCARPRGYYAMDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
DVVMTQSPSSLSASVGDRVTITCKASQDVSTAVAWYQQKPGKAPKPLIYSASYQYTGVPSRFSGSGSGTDFTFTITSLQPEDIAIYYCQQHYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 860 aa, synthetic construct
EVQLVESGGGLVQPGGSLKLSCAASGFDFSRYWMSWVRQAPGKGLEWVAEINPDSSKINYMPSLKDRFTISRDNAKNSLYLQMNSLRAEDTAVYYCARPRGYYAMDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 135 - protein, 858 aa, synthetic construct
EVQLVESGGGLVQPGGSLKLSCAASGFDFSRYWMSWVRQAPGKGLEWVAEINPDSSKINYMPSLKDRFTISRDNAKNSLYLQMNSLRAEDTAVYYCARPRGYYAMDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 136 - protein, 450 aa, Homo sapiens
EVQLVQSGAEVKKPGSSVKVSCKASGGTFSNYAINWVRQAPGQGLEWMGGIIPIFATTNYAQKFQGRVTITADESTSTAYMELSSLRSEDTAVYFCARDKHSWSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 216 aa, Homo sapiens
QSVLTQPPSASGTPGLRVTISCSGSSSNIGSNTVNWYQHLPGTAPKLLIHSNNQRPSGVPDRFSGSKSGTSASLAISGLQSEDEADYYCAAWDDSLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 138 - protein, 862 aa, synthetic construct
EVQLVQSGAEVKKPGSSVKVSCKASGGTFSNYAINWVRQAPGQGLEWMGGIIPIFATTNYAQKFQGRVTITADESTSTAYMELSSLRSEDTAVYFCARDKHSWSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 139 - protein, 860 aa, synthetic construct
EVQLVQSGAEVKKPGSSVKVSCKASGGTFSNYAINWVRQAPGQGLEWMGGIIPIFATTNYAQKFQGRVTITADESTSTAYMELSSLRSEDTAVYFCARDKHSWSYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 140 - protein, 459 aa, Homo sapiens
QVQLVQSGAEVKKPGASVRVSCKASGYTFTSYGISWVRQAPGQGLEWMGWISGYDGNTNYAQKLQGRVTMTTDTSTSTAYMELRSLRSDDTAVYYCARDGPQVGDFDWQVYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 214 aa, Homo sapiens
AIRMTQSPSTLSASVGDRVTITCRASQSINTWLAWYQQKPGKAPNLLISKASSLESGVPSRFSGSGSGTEFTLTISSLQPDDFATYFCQQYNSYLYTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 871 aa, synthetic construct
QVQLVQSGAEVKKPGASVRVSCKASGYTFTSYGISWVRQAPGQGLEWMGWISGYDGNTNYAQKLQGRVTMTTDTSTSTAYMELRSLRSDDTAVYYCARDGPQVGDFDWQVYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 143 - protein, 869 aa, synthetic construct
QVQLVQSGAEVKKPGASVRVSCKASGYTFTSYGISWVRQAPGQGLEWMGWISGYDGNTNYAQKLQGRVTMTTDTSTSTAYMELRSLRSDDTAVYYCARDGPQVGDFDWQVYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 144 - protein, 462 aa, Homo sapiens
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 211 aa, Homo sapiens
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 146 - protein, 874 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 147 - protein, 872 aa, synthetic construct
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED

SEQ ID NO: 148 - protein, 449 aa, synthetic construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 215 aa, synthetic construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 150 - protein, 861 aa, synthetic construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGIKCPQPKTLDEFTIIQNLQPQYQFRDYFIATCKQGYQLIEGNQVLHSFTAVCQDDGTWHRAMPRCKIKDCGQPRNLPNGDFRYTTTMGVNTYKARIQYYCHEPYYKMQTRAGSRESEQGVYTCTAQGIWKNEQKGEKIPRCLPVCGKPVNPVEQRQRIIGGQKAKMGNFPWQVFTNIHGRGGGALLGDRWILTAAHTLYPKEHEAQSNASLDVFLGHTNVEELMKLGNHPIRRVSVHPDYRQDESYNFEGDIALLELENSVTLGPNLLPICLPDNDTFYDLGLMGYVSGFGVMEEKIAHDLRFVRLPVANPQACENWLRGKNRMDVFSQNMFCAGHPSLKQDACQGDSGGVFAVRDPNTDRWVATGIVSWGIGCSRGYGFYTKVLNYVDWIKKEMEEED

SEQ ID NO: 151 - protein, 859 aa, synthetic construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED